(19) United States
(12) Patent Application Publication
(10) Pub. No.: US 2024/0000935 A1
(43) Pub. Date: Jan. 4, 2024

(54) ANTIGEN SPECIFIC T CELLS AND METHODS OF MAKING AND USING SAME

(71) Applicant: GENEIUS BIOTECHNOLOGY, INC., Natick, MA (US)

(72) Inventors: Alfred E. SLANETZ, Natick, MA (US); Walter BARRY, Natick, MA (US)

(73) Assignee: GENEIUS BIOTECHNOLOGY, INC., Natick, MA (US)

(21) Appl. No.: 18/038,834

(22) PCT Filed: Nov. 24, 2021
(86) PCT No.: PCT/US21/60873

§ 371 Date: May 25, 2023

Related U.S. Application Data
(60) Provisional application No. 63/147,718, filed on Feb. 9, 2021.
(60) Provisional application No. 63/118,554, filed on Nov. 25, 2020.

(51) Int. Cl. A61K 39/00; C12N 5/0783; A61P 35/00; A61P 31/12

(52) U.S. Cl. CPC A61K 39/4611; A61K 39/4632; C12N 5/0636; A61P 35/00; A61P 31/12; C12N 2510/00

(57) ABSTRACT

In various embodiments, the present disclosure provides T cell compositions comprising T cells that encode and/or express a T cell receptor (TCR) that binds to a neoantigen associated with a subject's cancer, and are useful for adoptive immunotherapy. Also disclosed are methods for making and/or using T cell compositions described herein.

### CROSS-REFERENCE TO RELATED APPLICATIONS

This International Application claims priority to U.S. Provisional Patent Applications No. 63/118,554, filed on Nov. 25, 2020, and No. 63/147,718, filed on Feb. 9, 2021, the entire contents of each of which are incorporated herein by reference.

### BACKGROUND

Cancer is a growing threat to the health of society. On average, the current population is growing older as medical technology increasingly extends lives, leading to increased cancer incidence. One out of every four deaths globally are attributed to cancer.

Cancer is a challenging disease to treat due to tremendous heterogeneity across patients and types. Even now, many cancer centers are having each patient's genome sequenced to identify cancerous mutations. As such, unique treatments are required for each cancer, and personalized medicine may eventually become the standard of care.

Sequencing has revealed that every person's cancer contains changes to their genome. Otherwise known as the multi-hit hypothesis, these changes occur in a stepwise manner. The body counteracts these changes via the immune system by eliminating mutant cells. This system contains both generally and specifically targeted effector cells, referred to as the innate and adaptive immune system, respectively. The innate cells, such as natural killer cells and monocytes, use pathogen associated molecules or the absence of self-signals on human cells to identify foreign material. The adaptive immune system must be activated by antigen presenting cells (APCs). T cells have a T cell receptor (TCR) to identify antigens presented in the context of the major histocompatibility complex I or II (MHC) by an APC. Targeting is stringent and can be used against the patient's cells expressing that antigen rather than killing all cells. Together, these cells apply selection pressure to cancer cells in a process referred to as immunoediting (Cross 2018; Oldfield 2017). However, changes in sequence and expression pattern that do not provoke a strong response from the immune system appear on a cancer cell as neoantigens. Tumors formed of cancer cells evolve to produce a tumor microenvironment with immunosuppressive attributes and continue to proliferate, resulting in severely dysfunctional immune systems (Spranger 2015; Blank 2016; Poch 2007; Ohm & Carbone 2002).

As such, there is a need for new cancer therapies which are designed to treat each patient's unique cancer by targeting their specific neoantigens.

### SUMMARY

In some aspects, the present technology provides methods of generating a population of T cells expressing one or more T cell receptors (TCRs) that specifically bind one or more antigens, comprising: (i). obtaining a blood sample from a subject with cancer or a viral infection; (ii). identifying one or more antigens associated with the cancer or the viral infection; (iii). preparing one or more mRNA molecules encoding the one or more antigens associated with the cancer or the viral infection; (iv). isolating monocytes from peripheral blood mononuclear cells (PBMCs) of the blood sample and preserving a remainder of cells from the sample, the remainder of cells comprising T cells; (v). differentiating the isolated monocytes into dendritic cells; (vi). transfecting the dendritic cells with the one or more mRNA molecules; and (vii). stimulating the T cells from the remainder of cells by contacting them with the transfected dendritic cells, thereby generating a population of T cells that express one or more TCRs that specifically bind the one or more antigens associated with the cancer or the viral infection. In some embodiments, provided are populations of T cells derived from methods according to various embodiments disclosed herein.

In some aspects, the present technology provides methods of generating a population of T cells expressing one or more T cell receptors (TCRs) that specifically bind an antigen, comprising: (i). transfecting a population of dendritic cells with one or more mRNA molecules encoding one or more antigens; and (ii). stimulating a population of naïve T cells by contacting them with the transfected dendritic cells of step (i), thereby generating a population of T cells that express one or more T cells receptors that specifically bind the one or more antigens encoded by the one or more mRNA molecules.

In some aspects, the present technology provides isolated engineered T cells comprising T cell receptors (TCRs) targeting a plurality of cancer neoantigens selected from the neoantigens set forth in Tables 1-9 and 11.

In some aspects, the present technology provides populations of engineered T cells comprising T cell receptors (TCRs) targeting one or more antigens, the population comprising less than 5% regulatory T cells, less than 5% exhausted T cells, and more memory T cells than effector T cells.

In some aspects, the present technology provides methods of treating cancer in a subject in need thereof, comprising: (i). obtaining a blood sample from the subject; (ii). identifying one or more neoantigens associated with the subject's cancer; (iii). preparing one or more mRNA molecules encoding the one or more neoantigens; (iv). isolating monocytes from peripheral blood mononuclear cells (PBMCs) of the blood sample and preserving a remainder of cells from the sample, the remainder of cells comprising T cells; (v). differentiating the isolated monocytes into dendritic cells; (vi). transfecting the dendritic cells with the one or more mRNA molecules; (vii). stimulating the T cells from the remainder of cells by contacting them with the transfected dendritic cells, thereby generating a population of T cells that express one or more T cells receptors (TCRs) that specifically bind the one or more neoantigens associated with the cancer; and (viii). administering all or a portion of the resultant population of T cells to the subject.

In some aspects, the present technology provides methods of treating cancer in a subject in need thereof, comprising: (i). identifying two or more neoantigens associated with the subject's cancer; and (ii). administering to the subject a population of T cells, the population of T cells comprising a plurality of T cells that each express two or more T cell receptors (TCRs) that specifically bind at least two of the two or more neoantigens and further comprise a deletion or disruption in an endogenous β2-microglobulin (B2M) gene.

In some aspects, the present technology provides methods of treating a viral infection in a subject in need thereof, comprising: (i). identifying two or more viral antigens associated with the subject's viral infection; and (ii). administering to the subject a plurality of T cells expressing two or more T cell receptors (TCRs) that specifically bind the two or more viral antigens.

In some aspects, the present technology provides methods of transiently expressing one or more pro-inflammatory proteins and/or one or more exogenous enzymes that alter an extracellular matrix in a T cell, comprising transfecting the T cell with one or more mRNA molecules encoding the one or more pro-inflammatory proteins and/or the one or more exogenous enzymes that alter an extracellular matrix.

In some aspects, the present technology provides methods of altering a tumor microenvironment in a subject, comprising administering to the subject a population of T cells transiently expressing one or more pro-inflammatory proteins and/or one or more exogenous enzymes that alter an extracellular matrix

In some aspects, the present technology provides methods of preparing a composition comprising dendritic cells encoding and/or expressing one or more neoantigens associated with a subject's cancer, comprising: (i). obtaining a blood sample from the subject; (ii). sequencing cell free deoxyribonucleic acid (cfDNA) derived from the blood sample to identify one or more neoantigens associated with the subject's cancer; (iii). preparing an mRNA encoding the one or more neoantigens associated with the subject's cancer or a peptide corresponding to the one or more neoantigens associated with the subject's cancer; (iv). isolating monocytes from peripheral blood mononuclear cells (PBMCs) of the blood sample; (v). differentiating the isolated monocytes into dendritic cells; and (vi). combining the dendritic cells with the mRNA or peptide from step (iii) to obtain dendritic cells encoding and/or expressing the one or more neoantigens associated with the subject's cancer.

In some aspects, the present technology provides compositions comprising one or more T cells encoding and/or expressing a T cell receptor (TCR) that binds to a neoantigen associated with a subject's cancer, wherein the one or more T cells comprise one or more CD4+ T cell, one or more CD8+ T cell, one or more CD3+ T cell, and wherein the CD4+ T cells and CD8+ T cells are present in the composition in a ratio of about 1:1, about 1:2, or about 1:4.

In some aspects, the present technology provides compositions comprising one or more T cells encoding and/or expressing a TCR that binds to a neoantigen associated with a subject's cancer.

In some aspects, the present technology provides compositions comprising one or more T cells encoding and/or expressing a TCR that binds to one or more neoantigens associated with a cancer, wherein (a) the one or more neoantigens are associated with a specific type of cancer, or (b) the one or more neoantigens are associated with a cancer specific to a subject, and wherein the cancer is selected from the group consisting of colon cancer, lung cancer, pancreatic cancer, acute myeloid leukemia (AML), melanoma, bladder cancer, hematologic cancer, pancreatic cancer, and glioblastoma.

In some aspects, the present technology provides compositions comprising one or more T cells encoding and/or expressing a TCR that binds to a neoantigen associated with a subject's cancer, wherein the one or more T cells comprise CD4⁺ T cells, a CD8⁺ T cells, a CD3⁺ T cells, and wherein the CD4⁺ T cells and CD8⁺ T cells are present in the composition in a ratio ranging from about 1:4 to about 1:1, e.g., in a ratio of about 1:1, about 1:2, or about 1:4.

In some embodiments, the composition comprises about 80%, by weight, of a total weight of the composition, the one or more T cells encoding and/or expressing the TCR.

In some embodiments, the composition comprises less than 20%, by weight, of any cell other than the one or more T cells encoding and/or expressing the TCR.

In some embodiments, the one or more T cells is a CD4⁺ T cell, a CD8⁺ T cell, a CD3⁺ T cell, or combination thereof.

In some embodiments, the one or more T cells is a naïve T cell, a central memory T cell, a stem cell memory T cell, an effector memory T cell, an NK cell, or any combination thereof.

In some embodiments, the composition comprises greater than about 70%, by weight, of a total weight of the composition, CD3⁺ and CD8⁺ T cells or CD3⁺ and CD4+ T cells.

In some embodiments, the composition comprises greater than about 70%, by weight, of the total weight of the composition, central memory T cells.

In some embodiments, the composition comprises greater than about 70%, by weight, of the total weight of the composition, effector memory T cells.

In some embodiments, the composition comprises greater than about 70%, by weight, of a total weight of the composition, CD4⁺ T cells.

In some embodiments, the composition comprises greater than about 70%, by weight, of a total weight of the composition, CD8⁺ T cells.

In some embodiments, the composition comprises greater than about 70%, by weight, of a total weight of the composition, CD3⁺ T cells.

In some embodiments, the composition comprises T cells, wherein the T cells display minimal exhaustion markers including PD-1, LAG3, TIM-3, CTLA4, BTLA, TIGIT.

In some embodiments, the compositions further comprise a pharmaceutically acceptable carrier, pharmaceutically acceptable excipient, and/or pharmaceutically acceptable diluent.

In some embodiments the cells are infused into the patient for treatment or prophylaxis.

In other embodiments, the RNA used to make the T cell product can be administered to the same patient before or after the T cell product as a prime boost.

In some embodiments, the RNA or T cells can be a neoantigen vaccine.

In some embodiments, the neoantigen is one or more of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53 R273C, TP53 273H, TP53 R282W, and TP53 V157F.

In some aspects, the present technology provides methods of treating cancer in a subject in need thereof, comprising administering to the subject the composition according to any of the embodiments of the present technology.

In some aspects, the present technology provides methods of preparing a composition comprising T cells encoding and/or expressing a TCR that binds to a neoantigen associated with a subject's cancer, the method comprising: (a) obtaining a blood sample from the subject; (b) sequencing cell free deoxyribonucleic acid (cfDNA) derived from the blood sample to identify one or more neoantigens associated with the subject's cancer; (c) preparing a messenger ribonucleic acid (mRNA) encoding the one or more neoantigens associated with the subject's cancer or a peptide corresponding to the one or more neoantigens associated with the subject's cancer; (d) isolating monocytes from the blood sample and preserving a remainder of cells in the blood sample, wherein the remainder of cells comprise T cells; (e) differentiating the isolated monocytes into dendritic cells (“DCs”); (f) combining the DCs with the mRNA or peptide from (c) to obtain DCs encoding and/or expressing one or more neoantigens associated with the subject's cancer; (g) stimulating the T cells from (d) by contacting the T cells from (d) with the DCs from (f); and (h) obtaining a composition comprising T cells encoding and/or expressing a TCR that binds to the neoantigen associated with the subject's cancer.

In some embodiments, the mRNA encodes all neoantigens associated with the subject's cancer.

In some embodiments, the mRNA encodes a plurality of neoantigens associated with the subject's cancer.

In some embodiments, the peptide further comprises a plurality of peptides that includes all neoantigens associated with the subject's cancer.

In some embodiments, the mRNA encodes all common neoantigens associated with the subject's cancer.

In some embodiments, the peptide further comprises a plurality of peptides that includes all common neoantigens associated with the subject's cancer.

In some embodiments, the neoantigen is a KRAS gene, a TP53 gene, or both.

In some embodiments, the neoantigen is one or more of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53 R273C, TP53 273H, TP53 R282W, and TP53 V157F.

In some embodiments, the neoantigen is one of listed in Tables 1-10 and 11.

In some embodiments, the antigens are tumor associated antigens.

In some embodiments, the mRNA and/or peptide are at least about 80% pure and/or mRNA has a cap1 5′ structure and substitution of chemically modified uracil nucleotides such as 5-methoxy-uracil.

In some embodiments, the mRNA and/or peptide comprises less than 20% of any other material.

In some embodiments, step (g) is repeated at least once.

In some embodiments, differentiating the monocytes into DCs includes contacting the monocytes with a plurality of cytokines.

In some embodiments, all or substantially all of the monocytes are differentiated into DCs.

In some embodiments, differentiating the monocytes into DCs further comprises maturing the DCs by contacting the DCs with a maturation composition.

In some embodiments, the DC's and T cells are cultured in a single or multiple closed system bioreactors.

In some embodiments, the RNA can be introduced by nucleofection, preferably 4D nucleofection.

In some embodiments, the RNA can be introduced by lipid nanoparticles.

In other embodiments the DC's can be seeded and released from the cartridge in a closed system.

In some embodiments, stimulating T cells further comprises introducing cytokines to the cells.

In some embodiments, stimulating T cells promotes expansion of CD4⁺, CD3⁺, and/or CD8⁺ T cells.

In some embodiments the full genetic diversity of MHC and TCR present within the patient are used to target multiple neoantigens in a single bioreactor

In other embodiments the capacity of T cells to kill tumor cells is measured through the killing of cells expressing tumor antigens in a Real time Cell Adhesion assay (RTCA)

In some embodiments, the capacity of T cells to kill tumor cells is assayed by killing cells transfected with RNA

In other embodiments, the capacity of T cells to be activated to antigens can be assayed by ELISpot where RNA expresses the antigen targets

In some aspects, the present technology provides methods of preparing a composition comprising DCs encoding and/or expressing one or more neoantigens associated with a subject's cancer, the method comprising: (a) obtaining a blood sample from the subject; (b) sequencing cfDNA derived from the blood sample to identify one or more neoantigens associated with the subject's cancer; (c) preparing an mRNA encoding the one or more neoantigens associated with the subject's cancer or a peptide corresponding to the one or more neoantigens associated with the subject's cancer; (d) isolating monocytes from the blood sample; (e) differentiating the isolated monocytes into DCs; (f) combining the DCs with the mRNA or peptide from (c) to obtain DCs encoding and/or expressing one or more neoantigens associated with the subject's cancer.

In some aspects, the present technology provides methods of treating and/or preventing cancer in a subject in need thereof, the method comprising administering to the subject the composition comprising T cells encoding and/or expressing a TCR that binds to a neoantigen associated with a subject's cancer, wherein the T cells are derived from the subject.

In some embodiments, the cancer treatment comprises inhibiting cancer cell growth in the subject, reducing a number of cancer cells in the subject, slowing the progression of cancer in the subject, decreasing the likelihood of recurrence of cancer in the subject, or reducing one or more symptoms associated with the cancer in the subject.

In some embodiments, the cancer is selected from the group consisting of colon cancer, lung cancer, pancreatic cancer, AML, melanoma, bladder cancer, hematologic cancer, and glioblastoma.

In some embodiments, the cancer comprises a solid tumor.

In some embodiments, the subject is administered a dose of T cells between about 1×10⁵ to about 5×10⁵ cells/kg of the subject's body weight.

In some embodiments, the subject's cancer is treated after a first administration of the composition.

In some embodiments, the methods further comprise transferring one or more genes into the T cells.

In some embodiments, transferring one or more genes into the T cells includes transferring one or more vectors comprising nucleic acids that correspond to the one or more genes into the T cells, where in the one or more vectors is a lentivirus vector, a plasmid vector, and/or an adenovirus vector.

In some embodiments, the methods further comprise treating the T cells with an apoptosis inhibitor such as a Rho kinase (ROCK) inhibitor in step (g) or vaccinia virus B18R recombinant protein.

In some embodiments, the ROCK inhibitor is ROCK1 inhibitor, ROCK2 inhibitor, or both.

In some embodiments, the methods further comprise stimulating the T cells by seeding the T cells with additional T cells.

In some embodiments, the methods further comprise transfecting the T cells with RNA.

In some embodiments, the transfection increases the longevity and activity of the T cells through transient expression of molecules.

In some embodiments, the methods further comprise using CRISPR, Talen, Zinc Finger, Meganucleases, sleeping beauty or other gene editing technologies to knockout the β2-microglobulin (B2M) gene in T cells.

In some embodiments allogeneic cell products with the β2-microglobulin knockout administered to patients without the need for (or only needing only low levels) conditioning by chemotherapy, radiation or immunosuppressive agents.

In other embodiments, they can be administered to manage a patient before administration of an autologous T cell product.

In some embodiments allogeneic cell products with the β2-microglobulin knockout are administered to patients without the need for (or only needing only low levels) conditioning by chemotherapy, radiation or immunosuppressive agents having a longer half-life in the blood than those cells with wild type β2-microglobulin.

In some embodiments allogeneic cell products with the β2-microglobulin knockout demonstrate longer survival in the presence of partial MHC matched or fully mismatched T cells than those cells with wild type β2-microglobulin.

In still other embodiments, such allogeneic cell products with the β2-microglobulin knockout demonstrate a longer half-life in a patient's blood.

In other embodiments, the T cells can be modified by nucleofection, transfection with lipid nanoparticles or by other means of RNA to enhance the T cell's ability to suppress, overcome or modify a tumor microenvironment. In some embodiments, the introduced nucleic acids results in proinflammatory changes in the tumor microenvironment.

In some embodiments the introduced nucleic acids consist of circularized RNA, self-replicating RNA or chemically synthesized mRNAs, all with or without substituted or modified nucleosides in order to extend the half-life of the introduced RNA for prolonged expression.

In some embodiments this half-life can be 3 to 5 days. In other embodiments, this half-life can be 1 to 3 weeks. In other embodiments, the half-life can be a month, 2 months, 3 months, 6 months, 12 months or anything in between.

In other embodiments, T cells nucleofected with such RNA have survival advantages in the tumor microenvironment.

In other embodiments, T cells nucleofected with such RNA act as delivery vehicles for such microenvironment modifying molecules across the tumor.

In other preferred embodiments, T cells reactive to multiple cancer antigens nucleofected with such RNA act as delivery vehicles for such microenvironment modifying molecules across the heterogenous tumor.

In other embodiments, the T cells are stimulated against multiple neoantigens, their T cells single cell sequenced for TCR, and the repertoire of TCR's transfected into fresh T cells.

In some embodiments, transferring one or more genes into the T cells includes inserting one or more nucleic acids that correspond to the one or more genes into the genome of the T cells via clustered regularly interspaced short palindromic repeat (CRISPR)-mediated insertion.

In some embodiments, the insertion is by a knock out of the endogenous TCR with sequential or simultaneous knock in of the transfected TCR.

In other embodiments, the RNA encoding the viral, neoantigens or antigens is transfected directly into the PBMC's using lipid nanoparticle formulations or nucleofection and T cells are then exposed to cytokines and anti-CD3, anti-CD28 antibody to expand the T cell product.

In some aspects, the present technology provides methods of treating a viral infection in a subject in need thereof, the method comprising administering to the subject the composition comprising T cells encoding and/or expressing a TCR that binds to a viral antigen associated with a virus, wherein the T cells are derived from the subject.

In some embodiments, the viral antigen is a protein expressed by one or more of cytomegalovirus, Epstein-Barr virus, hepatitis B virus, human papillomavirus, adenovirus, herpes virus, human immunodeficiency virus, influenza virus, human respiratory syncytial virus, vaccinia virus, varicella-zoster virus, yellow fever virus, Ebola virus, coronavirus (e.g., SARS-CoV, MERS-CoV, SARS-CoV-2), Eastern equine encephalitis virus, Polyomavirus hominis1 (BKV), SV40 and Zika virus.

In some embodiments, the subject's viral infection is treated after a first administration of the composition.

In other embodiments, a vaccine containing antigens from multiple viral proteins

In preferred embodiments this vaccine is RNA or DNA.

In other preferred embodiments this vaccine targets antigens from multiple viral proteins of SARS-COV-2

In preferred embodiments the antigens selected reflect the effective clearance response in natural immunity to a virus

In other preferred embodiments this vaccine targets antigens from multiple viral proteins of SARS-COV-2 including two or more of the following: Cov-2 S, M, N, 3a, 7a, 8.

In some embodiments T cells reactive to viral and neoantigens are both present in the T cell product to treat or prevent cancer.

In other embodiments the T cell products and/or DCs can be manufactured in a closed system.

### BRIEF DESCRIPTION OF THE DRAWINGS

FIG. 1 shows an exemplary method of neoantigen-based autologous cell transfer for cancer treatment (“mRNA T cell production process”) in accordance with embodiments of the present technology. For “peptide T cell production process” step 109 is synthesis of neoantigen peptides and at step 110 these peptides are introduced.

FIG. 2 shows an exemplary method of stimulating and expanding heterogenous T cells ex vivo against RNA encoding a personalized combination of neoantigens presented by dendritic cells (“DCs”) in accordance with embodiments of the present technology. The entire manufacturing process lasts 35 days with twenty-one days of DC and T cell co-culture.

FIG. 3 shows an exemplary method of identifying clinically relevant oncogenic frameshift and missense mutations associated with cancer in accordance with embodiments of the present technology.

FIGS. 4A-4B show exemplary images and flow cytometry plots that verify differentiation of monocytes into dendritic cells (“DCs”). The differentiated cells have the typical appearance of DCs at the end of the six-day differentiation process in FIG. 1 (FIG. 4A) and are larger as determined by flow cytometry (FSC v. SSC) (FIG. 4B).

FIGS. 5A-5F show exemplary flow cytometry plots that verify differentiation of monocytes into DCs by expression of the surface markers CD209, CD80, HLA-DR, CD1a, CCR7, and CD83 for the DC phenotype, respectively.

FIGS. 6A-6D show exemplary plots comparing the efficacy of the stimulation process in FIG. 1 over a six-hour period for donor-matched DCs and T cells. The plots show T cells sourced from peripheral blood mononuclear cell (PBMC) previously cultured with LMP2A peptide and include T cells alone (FIG. 6A), T cells with LMP2A peptide (FIG. 6B), T cells with DCs (FIG. 6C), and T cells with dendritic cells (“DCs”) and LMP2A peptide (FIG. 6D).

FIGS. 7A-7D show exemplary plots demonstrating that DC mediated priming using peptides can produce an enriched T cell population of TNFα and IFNγ releasing cells in response to a peptide antigen. The plots show PBMCs combined with DMSO vehicle control (FIG. 7A), LMP2A peptide (FIG. 7B), DC mediated priming DMSO vehicle (FIG. 7C), and DC mediated priming LMP2A peptide (FIG. 7D).

FIGS. 8A-8E show exemplary plots depicting the fraction of T cells responding to KRAS G12D neoantigen pepmix as measured by an intracellular cytokine staining (ICCS) FACS assay. The images show DC mediated priming against G12D from a day 14 culture process shown in FIG. 1 when combined with pepmix and include normal KRAS G12 (FIG. 8A) and KRAS G12D (FIG. 8B). A separate donor matched culture against LMP2a was conducted side by side. At day 14 this culture was tested for cytokine release against LMP2A (FIG. 8C). The fraction of CD8⁺IFNγ+ cells for KRAS G12D (FIG. 8D) and LMP2A (FIG. 8E) is provided.

FIG. 9 shows an exemplary plot depicting KRAS G12D tetramer analysis of cells such as those in FIG. 8B.

FIG. 10 shows results from an exemplary carboxyfluorescein succinimidyl ester (CSFE) based cytotoxicity assay of effector T cells against the KRAS G12D peptide expressed by target cells or normal KRAS G12. The data shows that only the mutant peptide is killed and not normal sequences.

FIGS. 11A-11C show exemplary plots depicting release of TNFα and IFNγ during DC priming simultaneously carried out with LMP2A (FIG. 10B) and KRAS G12D (FIG. 10C) as compared to a vehicle control (FIG. 10A).

FIGS. 12A-12B show exemplary plots of PBMCs from glioblastoma patient donors used to generate DCs and prime T cells simultaneously against the tumor associated antigen NY-ESO-1 and the CMV protein pp65.

FIG. 13 Electropherogram of 20neomut21 aapoly+polyA+meoUTP+CleanCap post silica column RNA cleanup and RP-HPLC. The electropherogram displays low incomplete transcript counts. Purity is greater than 80%.

FIGS. 14A-14D show exemplary plots depicting parameters associated with transfecting DCs with eGFP mRNA demonstrating that following transfection, DCs have strong GFP expression and are viable for use in priming.

FIGS. 15A-15C show an exemplary schematic of a mRNA poly-neoantigen construct for simultaneous translation and presentation of a plurality of neoantigens discovered using the process illustrated in FIG. 3 in accordance with embodiments of the present technology.

FIG. 16 shows an exemplary plot of an amino acid sequence of the poly-neoantigen construct of FIGS. 15A-15C (sequence in FIG. 19A) that has been entered into the net major histocompatibility complex (NetMHC) calculator and associated binding affinities along the length of an 8 amino acid window with a single amino acid translocation.

FIG. 17 is an exemplary plot of DCs transfected with LMP2A mRNA that show a robust response by T cells against encoded LMP2A antigen.

FIGS. 18A-18B show exemplary images of IFNγ ELISpot and a plot of IFNγ producing T cells primed by DCs that were transfected with mRNA encoding a 27 amino acid sequence with the TP53 mutation R248W (FIG. 18A) and automated spot count analysis confirming a specific response target in comparison to vehicle control (FIG. 18B). FIGS. 18C-18F show exemplary plots depicting the efficacy of stimulation conditions over a six-hour period by DCs transfected with mRNA for LMP2a for the same donor as in FIGS. 6A-6D. Transfection of DCs with mRNA demonstrates similar or better stimulation as compared to transfected DCs combined with peptides (FIG. 6D).

FIG. 19 shows a table enumerating 21 neoantigens used for the poly-neoantigen construct of FIGS. 15A-15C and their wild-type (normal) and mutant amino acid sequences.

FIGS. 20A-20B show a nucleic acid and amino acid sequence of the poly-neoantigen construct with elements from FIGS. 14A-14C, respectively.

FIGS. 21A-21E show exemplary graphs depicting the identification of a multiple neoantigen priming from the poly-neoantigen construct of FIGS. 20A-20B. In FIGS. 21A-21B two donors under the mRNA T cell production process using the mRNA in FIG. 20A were assessed by IFNγ ELISpot at day 21 of T cell culture for each of the included neoantigens. In FIGS. 21C-21E another three donors underwent the same process and analysis as shown in FIGS. 21A-21B with the modification of the addition of the Rho kinase inhibitor present at the start of priming (methods) and serially diluted out with feedings. There was a total of nine antigens that had spot counts above vehicle control (FIG. 21A), three neoantigens that had spot counts above vehicle control (FIG. 21B) and for ROCK inhibitor, there were 16 neoantigens (FIG. 21C), 20 neoantigens (FIG. 21D), and 11 neoantigens (FIG. 21E) that were above vehicle control.

FIGS. 22A-22I show exemplary plots depicting priming efficiency of T cells by post transfection treatment with the apoptosis inhibitors Y-27632 and protein B18R. Three healthy donors underwent the mRNA T cell production process with mRNA gene transfer of the model polylinker neoantigens and assessed at day 14 by IFNγ ELISpot KP108020, KP59714, KP59626. The graphs depict control (FIGS. 22A-22C), Y-27632 (FIGS. 22D-22F), and B18R (FIGS. 22E-22I).

FIG. 23 show exemplary images of selection wells that the data on the graphs of FIG. 21B were generated from and show the loci of T cells that release IFNγ.

FIG. 24A shows results of cytotoxicity assay of a production run against a mix of 21 neoantigens from mRNA in FIG. 20A that had been positive on IFNγ+ ELISpot for KRAS G12D and EGFR T790M. Effector cells from the run combined with fluorescently labeled target donor matched PHA blasts that had been loaded with one of four peptides: KRAS G12D, EGFR T790M, wild-type KRAS G12, wild-type EGFR T790 or DMSO (vehicle) in a 10:1 ratio of effector cells to targets cells and incubated for 20 hours under cell culture conditions (37 C, 5% CO2). The fraction of dead target cells at the end of 20 hours is provided. Background is considered below 10% as there is natural cell death in a culture. FIG. 24B shows results of cytotoxicity assays of production runs against a mix of 21 neoantigens from mRNA in FIG. 20A that had been positive on IFNγ+ ELISpot for the indicated neoantigen for each of four healthy donors. Effector cells from the run combined with fluorescently labeled target donor matched PHA blasts that had been loaded with a single pepmix of the indicated neoantigen or DMSO (vehicle) in a 10:1 ratio of effector cells to targets cells and incubated for six hours under cell culture conditions (37 C, 5% CO2). For NPM1_W288Cfs*12 using donor 4 the wild type pepmix was also tested to demonstrate specificity. The fraction of dead target cells at the end of six hours is provided.

FIG. 25A is an exemplary graph depicting the day 21 IFNγ ELISpot results of an mRNA T cell production process performed using blood from a colorectal cancer patient targeting the mutations detected using the Guardant OMNI Panel (wells in triplicate, background subtracted, wild-type indicates germline sequences, mutant indicates somatic mutations). FIG. 25B is an exemplary plot depicting the functional impact as assessed by a cytotoxicity analysis based on the production run in FIG. 25A.

FIG. 26 is an exemplary graph of cytotoxicity of T cell product as measured by the xCelligence RTCA platform. T cell product has TCRs specific to one of 21 neoantigens presented by HLA matched plated monocytes in which the 21 neoantigens are introduced either by mRNA transfection of the polylinker construct or pepmixes in equal mass ratios. Monocyte death causes a loss of adhesion. The killing of cells presenting endogenously produced antigen is greater than exogenously added peptides.

FIG. 27 is an exemplary graph of FACS based assessment of markers for T cell exhaustion on a day 21 final T cell product. The positive control, for comparison, is repeatedly overstimulated T cells from PBMCs.

FIGS. 28A-28B Viability and cell counts from lipofectamine based transfection of PBMCs with EBV mRNA compared to the present peptide T cell production process.

FIGS. 29A-29C FACS based assessment of cell phenotypes from the lipofection based antigen transfer experiment.

FIGS. 30A-30B Cytotoxic activity assay based on CSFE labelled targets and 7AAD for viability. Annexin V indicates programmed cell death in targets as a result of T cell activity. Targets are lymphoblastic cell line (LCL) immortalized with EBV.

FIG. 31A Shows the average number of CD3+ cells in the T cell product that express the chemokine CXCR3 and L-selectin (CD62L). FIG. 31B shows the average CD4 and CD8 in the T cell product in addition or in combination to the memory markers CD45RO. FIGS. 31C-31D shows the frequency of regulatory T cells (T_{reg}) present in the mRNA T cell product. FIG. 31C shows representative flow cytometry plots for CD3, CD4, and intracellular staining for Foxp3. FIG. 31D shows a box and whiskers plot for the percentage of CD3+ T cells that are T_{reg} (defined as CD3+CD4+ Foxp3+) in the final T cell product from 4 independent donors. FIG. 31E shows exemplary graphs showing that the major memory T cell subsets as defined by flow cytometry are central memory (CM) and effector memory (EM) as well as cells not significantly exhausted (PD1). The memory phenotype is substantially different than a patient's circulating T cells.

FIG. 32 depicts a process of producing purified T cells using a closed system method in accordance with embodiments of the present technology.

FIG. 33A is an image of an exemplary closed system DC device where the device includes a pump system to move the media from a media storage container, through the cassette, and ending at a waste container. FIG. 33B is an exemplary schematic showing DC preparation with antigens in separate sections of the chamber in the closed system shown that ensures parity in the representation of antigens and a broader antigen response profile. FIG. 33C is an exemplary graph showing a potential design mock-up with a prototype cassette for the differentiation and maturation of dendritic cells (“DCs”). FIG. 33D is an exemplary design of a multifunctional cassette culture system. When the cassette is in position #1, this will allow for the differentiation and maturation of dendritic cells (“DCs”) followed by antigen presentation to T-cells. After 5 days, the cassette is rotated/flipped into position #2. This allows for the rapid expansion of T-cells that can be harvested or moved into larger culture system or moved to additional cassettes if the cell density is too high.

FIG. 34 is an exemplary graph depicting the fold increase over starting T cell number from day 0 to day 14 at seeding densities of 4×10⁶ (SD1), 2×10⁶ (SD2), 1×10⁶ (SD3), and 0.5×10⁶ (SD4) for three donors (see Tables 16 and 17).

FIGS. 35A-35B are exemplary graphs depicting fold increase over starting T cell number at different time points varied by seeding density, antigen concentration, starting T cell amount, and volume for one donor (see Tables 16, 18A, 18B).

FIGS. 36A-36G show exemplary plots depicting flow cytometry surface stain gating strategy FSC-H (FIGS. 36A-36B), using Donor 259 at day 14 as an example (the percentages are of the fraction of the parent population indicated and not total percent of cells). This analysis identifies the cell phenotypes typical of lymphocytes including CD3+ T cells 29D, CD3+CD8+ Cytotoxic T-cells 29E, CD3+CD4+ helper T-cells 29E, B-cells 29F, Natural killer cells 29G and monocytes 29G.

FIG. 37 shows exemplary plots depicting flow cytometry analysis of memory T cell phenotypes using Donor 259 at day 14 as an example (the percentages are of the fraction of the parent population indicated and not total percent of cells) (see Table 16).

FIGS. 38A-38B show exemplary graphs of T cell phenotypes as measured by flow cytometry. The graphs show variation in seeding density for three donors (FIG. 32A) and illustrate T-cell types. A single donor is separated out for detailed analysis. Donor 201 with variations in seeding density, antigen concentration, and volume (FIG. 32B) (see Table 16).

FIGS. 39A-39B show exemplary graphs depicting the identification of other minor fraction cell types as measured by flow cytometry for FIGS. 38A-38B. The graphs show variation in seeding density resulting in changes in cell types for three donors (FIG. 39A) and illustrate minor cell phenotypes of a single Donor 201 with variations in seeding density, antigen concentration, and volume (FIG. 39B).

FIGS. 40A-40B show exemplary graphs of memory T cell phenotypes at different seeding densities for three donors for FIGS. 38A-38B.

FIG. 41 shows exemplary plots depicting flow cytometry analysis for identifying cytokine producing T cells and CD107 with an illustrative example of T cells reactive to a viral antigen LMP2A (the percentages are of the fraction of the parent population indicated and not total percent of T cells).

FIGS. 42A-42C show exemplary graphs depicting the fraction of cytokine producing cells to EBV LMP1, LMP2, and EBNA-1 in response to designated antigen at day 14 at different seeding densities as measured by flow cytometry (see Table 16).

FIGS. 43A-43D show exemplary graphs depicting the fraction of cytokine producing T cells and identity of the cytokine in response to designated antigen at day 14 as measured by flow cytometry in T cells from a single donor for different seeding densities with variation in antigen concentration and volume of starting media (see Table 16).

FIGS. 44A-44B show exemplary graphs depicting a minimal fraction of CD3+T regulatory cells and low levels of exhaustion present in the culture after seeding PBMCs at different seeding densities (see Tables 19 and 20).

FIG. 45 shows an exemplary graph depicting IFNγ release in response to antigen as measured by ELISpot at day 21 (see Tables 19 and 20).

FIG. 46 shows exemplary plots depicting flow cytometry surface staining analysis for cells derived from a single donor at day 21 as an example. T-regs are being measured here by markers of T-cell activation CD25 (IL2R), CD137 (4-1-BB) and CD154 (CD40L). Activated T cells are measured by CD25 and then divided into T-regs and non-T-regs CD3+ T-cells by CD154−CD137+. Percentages are of the fraction of the parent population indicated and not total percentage of cells.

FIG. 47 shows results from an exemplary carboxyfluorescein succinimidyl ester (CSFE) based cytotoxicity assay of effector T cells against the viral antigen LMP2a. Data is after five hours and a 10:1 effector to target ratio.

FIGS. 48A-48F T cells were derived from human PBMCs by cell culture in hlL-2 and stimulation with anti-CD2/CD3/CD28 for 3 days. T cells were transfected with mRNA encoding eGFP using Lonza's Amaxa 4D-Nucleofection protocol. FIG. 48A shows the viability of transfected T cells from two donors 24 hours after nucleofection measured as the percent of Propidium Iodide (PI) negative cells. FIG. 48B shows flow cytometry for eGFP expression 24 hours after nucleofection (green histograms) compared to untransfected cells (grey histogram). Transfected T cells were frozen at either 3 hours or 24 hours after nucleofection. Viability was measure immediately after thawing FIG. 48C. Cells were then cultured for 72 hours in media containing hlL-2 and assessed for GFP fluorescence by flow cytometry every 24 hours (colored histograms) compared to untransfected cells (grey histograms) FIG. 48D. FIG. 48E shows the mean fluorescence intensity (MFI) for eGFP for cells frozen at 3 hours (red) or 24 hours (blue) at the indicated times after the cells were thawed. FIG. 48F shows the expected results for transfection of mRNA T cell process product with modified mRNA for eGFP. Shown are representative graphs for unmodified linear mRNA (linear), linear mRNA modified with CleanCapAG (Trilink) and 5-methoxy-UTP (modified), circular RNA, and self-replicating RNA.

FIG. 49A is an exemplary graph showing the in vitro viability, by Real Time Cell Analyzer, of T-cells with and without P2-microglobulin knocked out in the presence of mismatched, partial match, and full match PBMCs. FIG. 49B shows an exemplary graph of fraction of transplanted cells indicating rate of clearance of human T-cell lines modified with CRISPR to no longer express MHC class I on the cell surface in BALB/c mice.

FIGS. 50A-50D show the efficacy of adoptively transferred T cell product in Cell line Derived Xenograft (CDX) and Patient Derived Xenograft (PDX) human cancer models. FIG. 50A shows a time course for CDX and PDX mice. FIGS. 50B-50D show exemplary graphs for the percentage of surviving mice transplanted with human tumor cells as a function of time. FIG. 50B shows the results for mice with tumors derived from patient Z treated with different doses of T cells derived from patient Z using the mRNA T cell process. FIG. 50C shows the results for the same T cells from patient Z transiently transfected with mRNA immediately prior to adoptive transfer. In this example T cells are transfected with either human IL-7 mRNA, IL-7R mRNA, mRNA for a secreted single chain antibody (scFvs) against αvβ8 integrin, or Fas-4-1 BB fusion protein mRNA. FIG. 50D shows the results for mice transplanted with EBV+ lymphoma cells from patient Y treated with T cells from patient Y using the mRNA T cell process using either mRNA for EBV antigens, mRNA for neoantigens, or both. FIGS. 50E-50H in vitro killing was assessed using the Real Time Cell Analyzer of Raji EBV+ lymphoma cells by T cell product reactive to LMP1, LMP2, EBNA1 using the mRNA T cell process that are transiently transfected with human IL-7 mRNA (FIG. 50E), IL-7R mRNA (FIG. 50F), IL-15 together with IL-15R-Fc fusion protein mRNA (FIG. 50G), or Fas-4-1 BB fusion protein mRNA (FIG. 50H) compared to no T cells and mock transfected T cells.

FIG. 51 shows an exemplary method of the single cell sequencing of T cells found in a given germinal center to be used for repertoire TCR transgenic treatments.

FIGS. 52A-52C show exemplary activation induced marker (AIM) results and percentage of central memory cells in the T cell product demonstrating that the manufacturing process with DCs creates a T cell product with a recognition pattern of a patient who has successfully cleared SARS-CoV-2 virus. To probe the reactivity of various peptides (S, M, N, 3a, 7a, 8, and S+: all antigens together), TCR dependent AIM assays were used to identify and quantify SARS-CoV-2-specific CD4⁺ and CD8⁺ T cells in unexposed donors, and DC T cell process and PBMC no DCs derived T cells were compared. SARS-CoV-2-specific CD4⁺ T cells were measured as percentage of AIM⁺ (OX40⁺CD137⁺) CD4⁺ T cells (FIG. 52A) and SARS-CoV-2-specific CD8⁺ T cells were measured as percentage of AIM⁺(CD69⁺CD137⁺) CD8⁺ T cells (FIG. 52B), after background subtraction. SARS-CoV-2 immunological memory was measured as a percentage of CD3⁺CD62L+CD197⁺ T Cell populations (FIG. 52C).

FIGS. 53A-53B show exemplary peptides with AIM response for COVID-19 positive patients in CD4⁺(FIG. 53A) and CD8⁺(FIG. 53B) cells.

FIG. 54 shows an exemplary timeline of when mRNA vaccine inoculations would occur in relation to the autologous adoptive T cell therapy process and infusion.

FIG. 55 shows an exemplary SARS Cov-2 mRNA vaccine. Immunogenic epitopes for S, M, N proteins were selected and placed into the cassette detailed in FIG. 15A-15C.

### DETAILED DESCRIPTION

As disclosed herein, applicants have developed novel methods for generating T cells targeting a plurality of antigens from a single manufacturing process. These methods utilize antigen mRNA rather than antigen peptides or polypeptides for priming. Specifically, naïve T cells are stimulated with dendritic cells (DCs) that have been transfected with mRNA encoding one or more target antigens. The resultant T cells have a higher killing capacity than T cells generated using peptides or polypeptides, and unexpectedly exhibit levels of activity against cancer neoantigens similar to those observed with T cells targeting viral antigens. The disclosed process can be used to generate T cells for use in autologous therapy or, by additionally deleting or disrupting an endogenous β2-microglobulin (B2M) gene, allogeneic therapy.

Applicants have further developed novel methods for transiently altering T cell protein expression patterns to confer or enhance the ability of the T cells to suppress, overcome, or modify a tumor microenvironment. For example, T cells can be engineered to transiently express one or more pro-inflammatory signals, e.g., chemokine or chemokine receptors, cytokines or cytokine receptors, or costimulatory molecules, or one or more proteins that alter the extracellular matrix.

Based on the experimental results set forth herein, the present disclosure provides methods of generating T cells targeting specific antigens, e.g., cancer neoantigens or viral antigens, that utilize mRNA rather than peptides or polypeptides for T cell stimulation. In certain embodiments, the resultant T cells comprise a plurality of T cell receptors (TCRs) that recognize different antigens. The present disclosure also provides systems and apparatuses for use in these methods, T cell populations comprising the resultant T cells, and methods of using these T cells and T cell populations in both autologous and allogeneic cancer therapies or treatment of viral infections. The present disclosure further provides methods of transiently altering expression of one or more proteins in a T cell or a population of T cells, wherein these transient alterations result in improved targeting and/or alterations to the tumor microenvironment. While the present disclosure is capable of being embodied in various forms, the description below of several embodiments is made with the understanding that the present disclosure is to be considered as an exemplification of the invention and is not intended to limit the invention to the specific embodiments illustrated.

Headings are provided for convenience only and are not to be construed to limit the invention in any manner. Embodiments illustrated under any heading may be combined with embodiments illustrated under any other heading.

### Definitions

The term “about” as used herein with regard to a numerical designation, e.g., temperature, time, amount, concentration, and such other, including a range, indicates approximations which may vary by (+) or (−) 10%, 5% or 1%.

The term “neoantigen” as used herein refers to a tumor-specific antigen, i.e., an antigen found on tumor cells but not on non-tumor cells. Neoantigens may arise from one or more tumor-specific alterations in a native protein or from non-native proteins such as viral proteins. Alterations in a native protein that give rise to a neoantigen may be the result of one or more mutations, including for example point mutations, rearrangements, insertions, deletions, or frameshift mutations in the gene encoding the protein or a proximal non-coding region, and/or one or more post-translational modifications such as glycosylation, lipidation, phosphorylation, acetylation, ubiquitination, or sumoylation. In some cases, post-translational modifications may be the result of an underlying mutation. Mutations giving rise to a neoantigen sometimes result in altered protein expression, for example overexpression, underexpression, or differently timed expression.

The term “viral antigen” as used herein refers to a virus-specific antigen, i.e., an antigen associated with a virus and specified by the viral genome. In some cases, a viral antigen is a protein encoded by the viral genome that can elicit a specific immunological response.

In some embodiments, the present technology provides T cells and populations of T cells capable of targeting one or more cancer-specific neoantigens associated with a subject's cancer, and methods of making the same. In certain of these embodiments, T cells isolated from a subject are activated by DCs isolated from the subject's PBMCs and transfected with mRNA encoding a neoantigen or contacted with a neoantigen peptide. After activation of the T cells, the cells are injected back into the subject to treat and/or prevent cancer in the subject.

The methods of the present technology apply an advancement in sequencing technology using cell free DNA (cfDNA), also referred to as circulating tumor DNA, to identify all available neoantigens present in a patient rather than just neoantigens and/or antigens from a single tumor (Zill 2018). This can be accomplished by next generation sequencing (NGS) panels, such as GuardantOMNI™ panel, Guardant360© panel, Foundation Medicine liquid biopsy panel, and other liquid biopsy panels used for detecting antigens. However, the present technology is not limited to the use of cfDNA but can also be applied to any genetic material including, but not limited to, a tissue-based broad companion diagnostic (CDx) referred to as a FoundationOne© tumor biopsy sequencing panel that is clinically and analytically validated for all solid tumors. As one of ordinary skill in the art would recognize, the present technology requires identification of targets having mutations, which may be achieved using immune histochemistry, mass spectrometry, or other sequencing technologies including but not limited to genomic sequencing and RNA seq. In some embodiments, the neoantigens are share neoantigens (e.g., antigens commonly found in cancer patients). In other embodiments, the neoantigens are personal neoantigens (e.g., found only in that patient's cancer).

In some embodiments, the methods provided herein produce an enriched population of antigen specific T cells with a significant T cell memory component. The methods allow for the differentiation of a single amino acid change neoantigen from the healthy sequence and of targeting multiple different neoantigens in one culture. In some embodiments, the present technology further comprises reintroduction of activated effector and memory T cells that reverses immune dysfunction typical of cancer patients.

In other embodiments, the methods provided herein produce an enriched population of antigen specific T cells without T regulatory cells. In other embodiments, the methods provided herein produce an enriched population of antigen specific T cells with de minimis T regulatory cells. In other embodiments, the methods provided herein produce an enriched population of antigen specific T cells with de minimis T cell exhaustion. In other embodiments, the methods provided herein produce an enriched population of antigen specific T cells with high percentages of homing and trafficking receptors such as CXCR3, CCR7 or CD62L. In other embodiments, the methods provided herein produce an enriched population of antigen specific T cells with high percentages of Central, Stem Cell and Effector Memory. In other embodiments, the methods provided herein produce an enriched population of antigen specific T cells wherein the population is predominantly Central and Effector Memory.

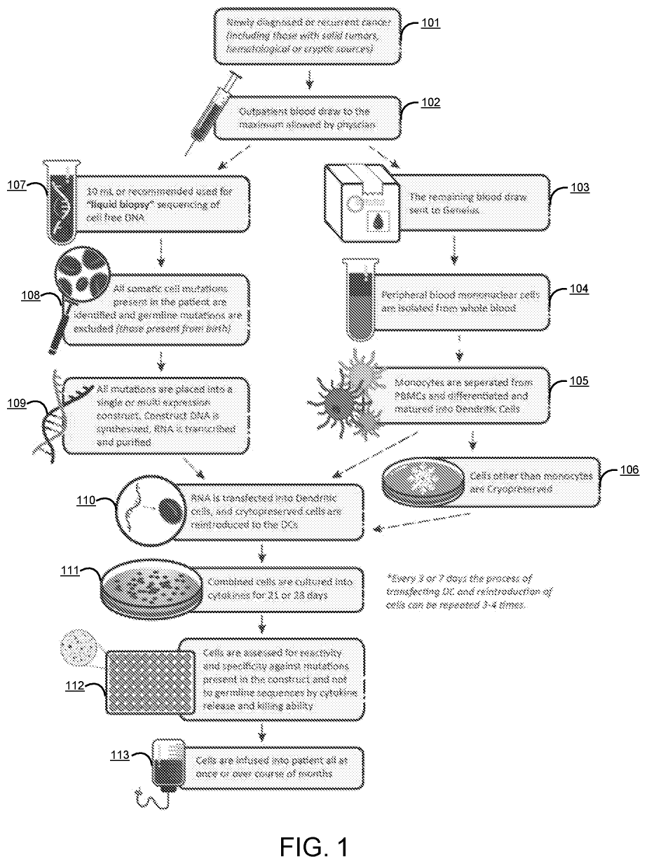

The present technology provides methods for neoantigen autologous cell transfer for cancer treatment (mRNA T cell production process). FIG. 1 is a flow chart of a method 100 of producing autologous T cells specific for a neoantigen useful for treatment of cancer in accordance with embodiments of the present technology. At step 101, the method 100 begins by diagnosing a patient with cancer and/or recurrent cancer. The method 100 can continue in step 102 where blood is drawn from the patient diagnosed with cancer in step 101. The blood drawn from the patient can include a combination of peripheral blood mononuclear cells (PBMCs), memory and naïve T cells, monocytes, and genomic DNA shed from tumor cells, all of which can be obtained from a single blood draw in step 102. In alternative embodiments, 2 or more blood draws can be combined, where one can be used for sequencing while the other can be used for isolation of PBMC's, monocytes, dendritic cells (“DCs”), T cells, or B cells. In some embodiments, blood can be collected by apheresis. After step 102, the method 100 can continue in step 103 where a portion of the blood used for producing DCs from monocytes is obtained and can continue in step 107 where another portion of the blood is obtained and used for sequencing cfDNA.

Following step 103, the method 100 can continue in step 104 where the PBMCs from a portion of the blood are isolated from the whole blood sample. The method 100 can continue in step 105 where the monocytes are then separated from the PBMCs for differentiation and maturation into dendritic cells (“DCs”). The method 100 can further include a step 106 where the remainder of the cells (i.e., cells other than monocytes) from the PBMCs are cryopreserved for later use.

Following step 107, the method 100 can continue in step 108 in which the somatic mutations present in the patient are identified and germline mutations are excluded (i.e., those mutations present at birth). The method 100 can continue in step 109 in which all of the mutations are placed into a single- or multi-expression RNA construct. The RNA construct is then purified. In other embodiments, the methods do not include purification of the RNA.

The method 100 continues in step 110 where the dendritic cells (“DCs”) derived in step 105 are combined with the purified RNA from step 109 to transfect the DCs with the RNA. Step 110 further comprises introducing the cryopreserved cells from step 106 to the RNA transfected DCs. The method 100 includes a step 111 where the combined cryopreserved cells and transfected DCs are cultured into T cells for about 21 to about 28 days. In other embodiments PBMC's can be substituted for DC's. In some embodiments B-cells are substituted for DCs. The method 100 includes a step 112 where the T cells are assessed for reactivity and specificity against mutations present in the RNA construct and not to germline sequences by cytokine release and/or killing ability. The method 100 includes a final step 113, where the cultured T cells are reinfused into the patient to treat cancer.

In some embodiments, the patient requires no further treatment. In some embodiments, the patient requires neither chemotherapy, radiation conditioning, or both. In some embodiments, the patient does not require IL-2 treatment or treatment with other T cell supportive cytokines.

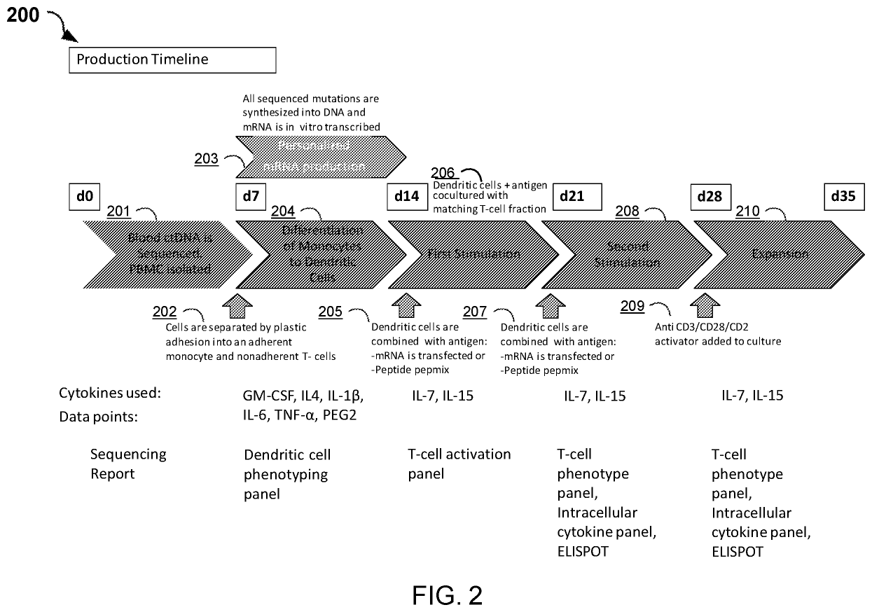

In some embodiments, the process described in FIG. 1 provides methods for producing T cells having mutations specific to an individual patient for use in cancer treatment as shown in FIG. 2. In other embodiments, the process described in FIG. 1 provides methods for generating DCs expressing neoantigens useful for priming the T cells. In further embodiments, the process described in FIG. 1 provides methods for identifying neoantigens in a patient's cancers.

FIG. 2 is a flow chart of a method 200 of producing T cells having mutations specific to an individual patient for use in cancer treatment in accordance with embodiments of the present technology. The method 200 can begin in step 201 of isolating circulating tumor DNA (cfDNA) and PBMCs from a tumor biopsy obtained from a patient. The cfDNA can be used to identify common cancer mutations of a cancer genome and/or neoantigens for use in generating and expanding targeted T cells directed to the cancer mutation and/or the neoantigen. The PBMCs are then used to differentiate monocytes into DCs.

The method 200 can continue in step 202 by isolating monocytes from the PBMCs. In some embodiments, isolating the monocytes from the PBMCs includes using plastic adhesion (alternatively CD14 beads or cell sorting) to separate adherent monocytes from nonadherent T cells in the PBMCs. The method 200 can continue in step 203 to produce personalized mRNA with mutations specific to the individual patient for use in generating T cells for cancer treatment. In some embodiments, all sequenced mutations in step 201 are synthesized into DNA and transcribed into mRNA. In some embodiments, mRNA is transcribed from DNA in vitro.

The method 200 can continue in step 204 by differentiating monocytes isolated from PBMCs to DCs. The method 200 can continue in step 205 by combining the DCs with an antigen either by transfecting the DCs with mRNA produced in step 203 or combining the DCs with a peptide pepmix. The method 200 can continue in step 206 by stimulating a T cell fraction with the DCs in a first stimulation step. The stimulation step comprises co-culturing the DCs with a matching T cell fraction. In some embodiments, the stimulation step 206 includes stimulating the T cell faction with the dendritic cells (“DCs”) in the presence of IL-7 and IL-15. In some embodiments, the DCs are transfected with DNA, rather than mRNA.

The method 200 can continue in step 207 by combining DCs with an antigen by either transfecting the DCs with mRNA produced in step 203 or combining the DCs with a peptide pepmix. The method 200 can continue in step 208 by stimulating the T cell fraction with the DCs in a second stimulation step. The second stimulation steps comprise co-culturing the DCs with the matching T cell fraction from step 206. In some embodiments, the stimulation step 206 includes stimulating the T cell faction with the DCs in the presence of IL-7 and IL-15. In some embodiments, the methods include multiple stimulation steps (e.g., 1, 2, 3, 4, or more). In some embodiments, the methods include a single stimulation step. In some embodiments the stimulation procedure with transfected DCs can be repeated every day, every other day, every third day, every fourth day, every fifth day, every sixth day, or every seventh day.

The method 200 can continue in step 209 where anti-CD3, CD28, and CD2 activators (e.g., antibodies and/or fragments thereof) are combined with the T cell culture after the second stimulation step 208. The method 200 concludes at step 210 by expanding the T cell population after the addition of the anti-CD3, CD28, and CD2 activators in step 209. In some embodiments, the T cell population is expanded by contacting the T cell population with antiCD3/feeder cells or CD3 beads. The expanded T cell population can then be transfused back into the patient to begin cancer treatment. Cell Types

A variety of cells are used in accordance with the embodiments of the present technology, including PBMCs, monocytes, T cells, and dendritic cells (DCs). Each of these cell types are characterized by expression of particular markers on the surface of the cell (or lack of expression of other markers) that enable identification of the cell type.

PBMCs are isolated from peripheral blood and identified as any blood cell having a round nucleus. PBMCs include lymphocytes (e.g., T cells, B cells, natural killer (NK) cells), monocytes, and DCs. In mammals, the frequencies of these populations within PBMCs vary but commonly include lymphocytes in a range of 70-90%, monocytes from 10 to 20%, and DCs accounting for only 1-2%.

Monocytes are a type of leukocyte (e.g., white blood cells). Monocytes can differentiate into different cell types such as macrophages, DCs, liver Kupffer cells, or even microglia in the central nervous system.

In some embodiments, the monocytes are one or more subsets selected from classical (CD14⁺CD16⁻), non-classical (CD14dimCD16⁺), and intermediate (CD14⁺CD16⁺) monocytes.

In some embodiments, the monocytes are classical monocytes expressing a surface marker selected from one or more of CD14⁺, CD16⁻, CCR2⁺, CCR5⁺, and CD62L⁺.

In some embodiments, the monocytes are non-classical monocytes expressing a surface marker selected from one or more of CD14⁺, CD16⁺+, CX3CR1⁺, and HLA-DR⁺.

In some embodiments, the monocytes are intermediate monocytes expressing a surface marker selected from one or more of CD14⁺, CD16⁺, CCR2⁺, HLA-DR⁺, CD11c⁺, and CD68⁺.

DCs are antigen-presenting cells, which process and present antigenic peptides to naïve T cells or memory T cells to initiate an adaptive immune response. DCs undergo a series of functional changes through a maturation process. Once mature, DCs present antigenic peptides in the context of MHC to a T cell expressing a T cell receptor (TCR). Mature DCs are characterized by the production of cytokines (e.g., IL-2) and by the expression of homing receptors (e.g., CCR7) which direct the migration of DCs.

In some embodiments, the DCs are one or more subsets selected from plasmacytoid DCs (pDCs), CD1c⁺ myeloid DCs (cDC2 or MDC2), and CD141⁺ myeloid DCs (cDC1 or MDC1).

In some embodiments, DCs express a surface marker selected from MHC class I and MHC class II molecules.

In some embodiments, the DCs are pDCs expressing a surface marker selected from one or more of CD123, CD303, CLEC4C, BDCA-2, CD304, NRP1, BDCA-4, CD141, FCER1, ILT3, ILT7, DR6, and BDCA-1.

In some embodiments, the DCs are cDC1s expressing a surface marker selected from one or more of CD141, BDCA-1, CLEC9A, CADM1, XCR1, BTLA, CD26, DNAM-1, and CD226.

In some embodiments, the DCs are cDC2s expressing a surface marker selected from one or more of CD1c, BDCA-1, CD11c, CD11b, CD2, FCER1, SIRPA, ILT1, DCIR, CLEC4A, CLEC10A.

T cells refer to a population of monoclonal or polyclonal cells that express TCRs recognizing a tumor antigen peptide. Following activation by various cytokines, T cells can bind to and kill cancer cells. However, the frequency of naïve T cells specific for a given antigen is low, ranging between 0.01 and 0.001% of the total T cell count, depending on the respective specificity. When a naïve T cell encounters its cognate antigen and is consequently activated, clonal expansion begins, boosting the frequency of those antigen-specific T cells by several orders of magnitude. This allows T cells to efficiently fulfill their role as effectors in the immune response.

In some embodiments, the T cells are one or more subtypes selected from the group consisting of killer T cells, effector T cells, helper T cells (helper Th1 or helper Th2), regulatory T cells, and memory T cells.

In some embodiments, the T cells are killer T cells expressing a surface marker selected from one or more of CD8, IFNγ, and EOMES.

In some embodiments, the T cells are effector T cells expressing a surface marker selected from one or more of CD197⁻, CD45RO⁺, CD62L⁻ and CD95⁺.

In some embodiments, the T cells are helper T cells expressing a surface marker CD4. In some embodiments, the T cells are helper Th1 T cells expressing a marker selected from one or more of CXCR3, IFNγ, IL-2, IL-12, IL-18, STAT4, and STAT1. In some embodiments, the T cells are helper Th2 T cells expressing a marker selected from one or more CCR4, IL-2, and IL-4.

In some embodiments, the T cells are a regulatory T cells expressing a marker selected from one or more of CD4, CD25, CD127, CD152, TGFβ, IL-10, IL-12, FoxP3, and STAT5.

In some embodiments, the T cells are memory T cells selected from CD4⁺, CD8⁺, or both. In some embodiments, the memory T cells express surface markers selected from CCR7, CD44, CD69, CD103, CD45RO⁺, and CD62L⁺.

In some embodiments, the T cells produced in accordance with the embodiments of the present disclosure specifically recognize antigens on cancer cells, so that said T cells can treat a cancerous or neoplastic condition or prevent recurrence, progression, or metastasis of cancer while avoiding the defense mechanism of cancer cells.

### Cell Collection

In some embodiments, the methods in accordance with embodiments of the present technology include collecting cells from a patient having cancer as shown in FIG. 1 (step 102).

In some embodiments, the subject is diagnosed with cancer, has recurrent cancer, and/or a high risk of developing cancer. In some embodiments, the subject has a cancer selected from the group consisting of colon cancer, lung cancer, pancreatic cancer, acute myeloid leukemia (AML), melanoma, bladder cancer, hematologic cancer, and glioblastoma.

In some embodiments, the cells are isolated from whole blood obtained from the subject. In some embodiments, the cells are extracted from cancerous tissue (e.g., biopsy) from the subject. In some embodiments, the source of the cancerous cells is a solid tumor or tumor cryptic peptides.

In some embodiments, the solid tumor is one or more of solid tumors fibrosarcoma, myxosarcoma, liposarcoma, chondrosarcoma, osteogenic sarcoma, and other sarcomas, synovial sarcoma, mesothelioma, Ewing's tumor, leiomyosarcoma, rhabdomyosarcoma, colon carcinoma, lymphoid malignancy, pancreatic cancer, breast cancer, lung cancers, ovarian cancer, prostate cancer, hepatocellular carcinoma, squamous cell carcinoma, basal cell carcinoma, adenocarcinoma, sweat gland carcinoma, sebaceous gland carcinoma, papillary carcinoma, papillary adenocarcinomas, medullary carcinoma, bronchogenic carcinoma, renal cell carcinoma, hepatoma, bile duct carcinoma, choriocarcinoma, Wilms' tumor, cervical cancer, testicular tumor, bladder carcinoma, and CNS tumors (such as a glioma, astrocytoma, medulloblastoma, craniopharyngioma, ependymoma, pinealoma, hemangioblastoma, acoustic neuroma, oligodendroglioma, meningioma, melanoma, neuroblastoma and retinoblastoma).

In some embodiments, the blood and/or tissue obtained from the subject can include a plurality of T cells (e.g., memory T cells and/or naïve T cells), DCs, and monocytes. In some embodiments, the blood and/or tissue obtained from the subject comprises genomic DNA shed from tumor cells.

In some embodiments, the methods include obtaining at least about 10 mL, about 20 mL, about 30 mL, about 40 mL, about 50 mL, about 60 mL, about 70 mL, about 80 mL, about 90 mL, about 100 mL, about 110 mL, about 120 mL, about 130 mL, about 140 mL, about 150 mL, about 160 mL, about 170 mL, about 180 mL, about 190 mL, about 200 mL, about 210 mL, about 220 mL, about 230 mL, about 240 mL, about 250 mL, about 260 mL, about 270 mL, about 280 mL, about 290 mL, about 300 mL, about 310 mL, about 320 mL, about 330 mL, about 340 mL, about 350 mL, about 360 mL, about 370 mL, about 380 mL, about 390 mL, or about 400 mL of blood from the subject. In some embodiments, this blood is drawn in a single blood draw. In other embodiments, is the blood is combined from multiple blood draws. In some embodiments, the cells are collected through apheresis.

In some embodiments, the method includes using the blood to perform a liquid biopsy. A liquid biopsy includes obtaining a blood sample to identify cancer cells from a tumor that are circulating in the blood or from DNA from tumor cells in the blood. In some embodiments, about 10 mL to about 30 mL of blood is used for the liquid biopsy.

### Cell Differentiation and Cell Compositions

In some embodiments, the methods in accordance with embodiments of the present technology include differentiating monocytes into DCs as shown in FIG. 1 (step 105).

In some embodiments, the methods comprise isolating PBMCs from a whole blood sample as shown in FIG. 1 (step 104). In some embodiments, the PBMCs are separated from whole blood by one or more of density centrifugation with Ficoll-Paque, isolation by cell preparation tubes (CPTs), SepMate tubes with Lymphoprep, and Sepax C-Pro system (Cytiva) or separation by centrifugation and optical detection, thermogenesis, Miltenyi Biotec, and MicroMedicine Sortera.

In some embodiments, the methods comprise isolating monocytes from the PBMCs. In some embodiments, the monocytes are isolated from the PBMCs by incubating fresh or frozen PBMC on tissue culture grade plastic in media in the absence of cytokines. Non-adherent cells can be frozen and used later. In some embodiments, monocytes are isolated from PBMCs separation technologies to include by not limited to CD14 positive selection beads. In another embodiment, monocytes are isolated from PBMCs by plastic adherence.

In some embodiments, the method of differentiating isolated monocytes into DCs includes contacting the monocytes with a plurality of cytokines. Non-limiting examples of cytokines that induce differentiation of the monocytes into DCs include one or more of granulocyte-macrophage colony stimulating factor (GM-CSF), interleukins (e.g., IL-1, IL-2, IL-4), and interferons (IFNs).

In some embodiments, the methods for differentiating monocytes into DCs includes culturing the isolated monocytes in a medium comprising GM-CSF and IL-4. In some embodiments, the medium is an RPMI medium.

In some embodiments, the DCs are enriched using a DC cassette. In these embodiments, monocytes are transferred into the DC cassette and adhere to a substrate. In some embodiments, lateral flow is applied to the monocytes within the DC cassette thereby converting the monocytes into DCs.

In some embodiments, the monocytes are cultured in medium until all or substantially all are differentiated into DCs. In some embodiments, the monocytes are cultured in a medium for at least about one day, at least about two days, at least about three days, at least about four days, or at least about five days until monocyte differentiation is complete.

In some embodiments, all or substantially all monocytes are differentiated into DCs. In some embodiments, at least about 80%, at least about 85%, at least about 90%, at least about 92%, at least about 94%, at least about 96%, at least about 97%, at least about 98%, or at least about 99% of all the monocytes are differentiated into DCs. In some embodiments, less than about 15%, less than about 10%, less than about 8%, less than about 6%, less than about 4%, less than about 2%, or less of the monocytes have not differentiated into DCs.

In some embodiments, the differentiated cells comprise DCs and no or substantially no other cell types (e.g., monocytes, non-DC PBMCs, and/or T cells). In some embodiments, the DCs comprise at least about 80%, at least about 85%, at least about 90%, at least about 92%, at least about 94%, at least about 96%, at least about 97%, at least about 98%, or at least about 99% DCs, by weight, of the differentiated cells. In some embodiments, the differentiated cells comprise less than about 15%, less than about 10%, less than about 8%, less than about 6%, less than about 4%, less than about 2%, or less of any other cell type excluding DCs.

In some embodiments, the methods comprise confirming differentiation of monocytes into DCs. In some embodiments, monocyte differentiation is determined by assessing a visual difference in the monocytes as compared to DCs, such as by noting the presence of a compact nucleus, protrusions, and/or other phenotypic features recognizable by one of ordinary skill in the relevant art.

In some embodiments, monocyte differentiation is determined to be complete by identifying the surface markers expressed by the cells. In some embodiments, monocyte differentiation is complete when the surface markers expressed by the cells are surface markers associated with one or more subtypes of DCs rather than surface markers associated with one or more subtypes of monocytes. In some embodiments, the surface markers associated with one or more DCs is selected from MHC class I and class II molecules, CD123, CD303, CLEC4C, BDCA-2, CD304, NRP1, BDCA-4, FCER1, ILT3, ILT7, DR6, BDCA-1, CD141, CLEC9A, CADM1, XCR1, BTLA, CD26, DNAM-1, CD226, CD1c, BDCA-1, CD11c, CD11b, CD2, FCER1, SIRPA, ILT1, DCIR, CLEC4A, and CLEC10A. In some embodiments, the differentiated cells do not express a surface marker associated with a monocyte selected from the group consisting of CD14⁺⁺, CD16⁻, CCR2⁺, CCR5⁺, CD62L⁺, CD14⁺, CD16⁺+, CX3CR1⁺, HLA-DR⁺, CD16⁺, CCR2⁺, CD11c⁺, and CD68⁺.

In some embodiments, differentiation of monocytes to DCs is confirmed by flow cytometry (FACS) analysis, production of interleukin 12 (IL-12), enzyme-linked immunosorbent assay (ELISAs), or combination thereof.

In some embodiments, the methods further comprising maturing the differentiated DCs. The maturation step matures the DCs into antigen presenting DCs and allows for cell surface expression of costimulatory molecules for T cell primary, absent maturation, the DCs will not generate an effective response to T cells. In some embodiments, maturing the differentiated DCs includes stimulating the DC with “maturation cocktail.” In some embodiments, the “maturation cocktail” includes one more of TNFα, IFNα, IL-1β, IL-6, PGE₂, IFNγ, pIC, MPLA, and CL097. In some embodiments, the “maturation cocktail” includes TNFα, IL-1β, IL-6, and PGE₂. In some embodiments, the “maturation cocktail” includes TNFα, IL-1β, IFNγ, IFNα, and pIC. In some embodiments, the “maturation cocktail” includes IFNγ and MPLA. In some embodiments, the “maturation cocktail” includes TNFα, IL-1β, IFNγ, IFNα, and CL097.

In some embodiments, the DCs are matured in a “maturation cocktail” until the DCs mature into antigen presenting mature DCs. In some embodiments, the “maturation cocktail” is applied to the DCs for at least about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, or about 48 hours.

In some embodiments, the DCs are matured by exposing the cells lipopolysaccharide (LPS) and IFNγ to activate alternate signaling pathways.

In some embodiments, all or substantially all DCs are matured into antigen presenting mature DCs. In some embodiments, at least about 80%, at least about 85%, at least about 90%, at least about 92%, at least about 94%, at least about 96%, at least about 97%, at least about 98%, or at least about 99% of all the DCs are matured into antigen presenting mature DCs. In some embodiments, less than about 15%, less than about 10%, less than about 8%, less than about 6%, less than about 4%, less than about 2%, or less of the DCs are not converted into antigen presenting mature DCs.

In some embodiments, the DCs comprise antigen presenting mature DCs and no or substantially no other cell types (e.g., non-matured DCs, monocytes, PBMCs, and/or T cells). In some embodiments, the DCs comprise at least about 80%, at least about 85%, at least about 90%, at least about 92%, at least about 94%, at least about 96%, at least about 97%, at least about 98%, or at least about 99% DCs, by weight, of the DCs. In some embodiments, the differentiated cells comprise less than about 15%, less than about 10%, less than about 8%, less than about 6%, less than about 4%, less than about 2%, or less of any other cell type excluding antigen presenting mature DCs.

In some embodiments, cells from the PBMCs other than monocytes are preserved for later use as shown in FIG. 1 (step 106). In some embodiments, cells from the PBMCs other than monocytes are not cryopreserved and used immediately for stimulation and priming of T cells. In some embodiments, cells other than monocytes include depleted cells or non-adherent cells. In some embodiments, cells other than monocytes include lymphocytes (e.g., T cells, B cells, natural killer (NK) cells) and DCs.

In some embodiments, the cells other than the monocytes are cryopreserved. In some embodiments, the cells other than the monocytes are cryopreserved at a temperate of about −80° C. In some embodiments, the cells other than the monocytes are cryopreserved for at least about 12 hours, about 14 hours, about 16 hours, about 18 hours, about 20 hours, about 22 hours, about 24 hours, about 26 hours, about 28 hours, about 30 hours, about 32 hours, about 34 hours, about 36 hours, about 38 hours, about 40 hours, about 42 hours, about 44 hours, about 46 hours, or about 48 hours prior to use in stimulation and priming of T cells.

In alternative embodiments, the RNA encoding antigen targets is introduced into the PBMC's by cationic lipids including but not limited to lipofectamine or other lipid nanoparticles in this disclosure. Alternatively, the RNA may be introduced into the PBMC's by nucleofection. In a further alternative, it can be introduced into the PBMC's in the presence of an apoptosis inhibitor according to the current disclosure.

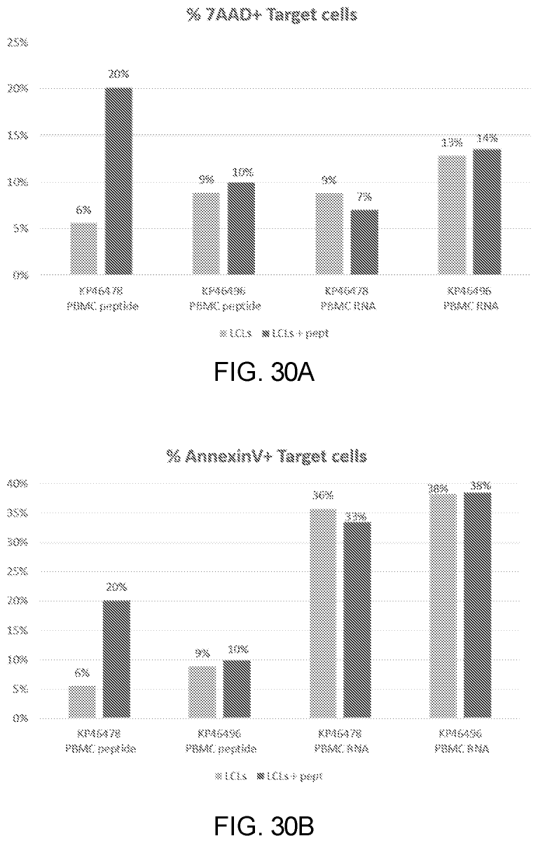

Here RNA made encoding antigen is mixed with cationic lipids and added directly to PBMCs in culture. This is alternative to the use of nucleofection of RNA. It is assumed that APCs in the PBMCs will take up the RNA and present antigen to the rest of the T cells. This method can be performed in the closed system PBMC process described by simply substituting the cationic lipid formulated RNA for each of the peptide antigens without the need for a DC cassette. The cationic lipid RNA PBMC method produced T cells with greater cytotoxicity against antigen expressing targets than did peptide priming. FIG. 30A-30B.

### Sequencing Process

In some embodiments, the methods in accordance with embodiments of the present technology include sequencing DNA of cancerous cells obtained from a subject as shown in FIG. 1 (step 107).

In some embodiments, sequencing DNA of cancerous cells includes identifying neoantigens present in the blood of the subject. A neoantigen may be either a mutation or overexpression of a protein, metabolite, nucleic acid, glycosylation specific to an individual's cancer. The neoantigens correspond to specific changes that are not germline mutations but rather, ones found only in the somatic cancer cell or support the function and growth of cancer cells not limited to the principal tumor (e.g., proximal cells such as mesenchymal cells responsible for the tumor microenvironment). In some embodiments, the neoantigens differ from wild-type and/or native counterparts by one or more of the following: point mutations, rearrangements, insertions, deletions, frameshift mutations in the amino acid sequence, differential glycosylation, lipidation, phosphorylation, or acetylation, and dimerization. In some embodiments, the mutations are encoded in a reading frame. In some embodiments, the neoantigens are encoded in proximal sequence elements directing post translational changes.

In some embodiments, neoantigens are determined by sequencing of cfDNA, tumor DNA or from tumor material. Circulating tumor DNA is representative of all the metastatic lesions rather than just the primary tumor as is the case in traditional sequencing. cfDNA from tumors also better represents truncal tumor neoantigens whereas traditional methods are representative of only branched (subclonal) tumor neoantigens. In some embodiments, the neoantigens are identified through a technique selected from the group consisting of mass spectrometry, LC-MS, GC-MS/MS and immunoassay-based identification of post translational modifications.

In some embodiments, the methods include sequencing DNA using any conventional DNA sequencing technique. In some embodiments, DNA is sequenced using a sequencing technique selected from the group consisting of a Maxam and Gilbert method, chain termination method, semiautomated method, pyrosequencing, whole-genome shotgun sequencing, clone by clone sequencing, and next-generation sequencing. In some embodiments, the sequencing DNA includes use of a sequencing platform selected from the group consisting of single-molecule real-time (RNAP) sequencing, single-molecule SMRT™ sequencing, Helioscope™ single-molecule sequencing, DNA nano ball sequencing, SOLiD sequencing, Illumina sequencing, colony sequencing, massively parallel signature sequencing (MPSS), and high throughput sequencing.

In some embodiments, the methods include sequencing cfDNA isolated from a whole blood sample from a subject. In some embodiments, neoantigens are determined for an individual subject by sequencing the subject's cfDNA. In some embodiments, the methods of sequencing cfDNA includes drawing a 10 mL blood sample from a subject's plasma and isolating the blood from the plasma to eliminate naturally occurring leukocyte mutations. In some embodiments, one or more mutations identified from sequencing the subject's cfDNA is selected from the group consisting of PREX1 Q802E, PREX1 I1003I, XPA D5Y, SETD2 P1141L, POLE R52Q, LIG4 A11A, APC E1286, BRCA1 R1726G, FZD5 L511L, SOX2 A133T, ERBB2 P1147P, POLD1E803E, KDM5B R863Q, TP53 R248Q, EGFR P848S, MEN1 A467A, PPARG V478A, NF1 K428T, FZD6 G350G, KDM6A A48V, IKZF1 N149T, LRP1B R363W, DEPTOR L88P, ALK D49D, FAT1 T207T, and FAT1 S3753T.

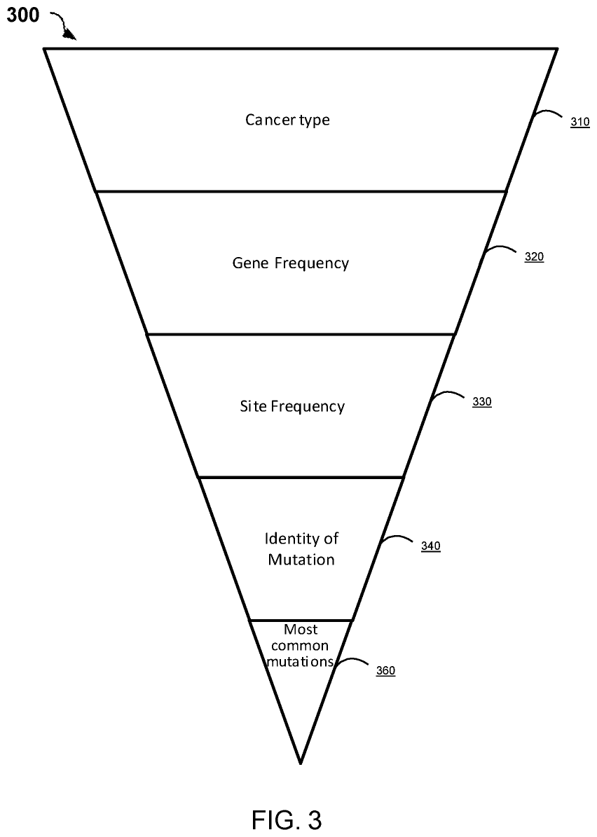

In some embodiments, neoantigens are determined from pre-defined panel (e.g., from a database). In some embodiments, the neoantigens are determined using one or more of the pre-defined panels, such as GuardantOMNI™ Panel, MSK-IMPACT™ panel, Foundation Medicine FoundationOne© Panel, and Personal Genome Diagnostics Panel. In some embodiments, the present technology provides methods for identifying common cancer mutations found in specific cancer types to develop a model of the types of targets (i.e., mutations) that are typical in cancer. FIG. 3 is a diagram of a method 300 of identifying common mutations associated with a particular cancer type. Following the diagram provided in FIG. 3 and through the process of elimination, the most common mutations associated with a given cancer were identified. The method 300 can begin in step 301 of identifying a specific type of cancer. In some embodiments, the cancer type is selected from the group consisting of colon cancer, lung cancer, pancreatic cancer, AML, melanoma, bladder cancer, hematologic cancer, and glioblastoma.

The method 300 can continue in step 320 and 321 where the gene frequency and site frequency of cancer mutations is determined by analysis of the sequencing data associated with each cancer type provided in the database. The database can include data from over 10,000 patients and therefore, is representative of neoantigens in cancer patient populations. Databases that could be used include but are not limited to TGCA, NIH, MSKCC, Dana Farber, Foundation, Guardant, Caris, or any cancer center, clinic, company or organization that has sequencing data from a statistically significant number of patients with cancer or a particular form of cancer. The method 300 then continues in step 340 where the identity of a specific mutation associated with the cancer is determined and mutations not associated with the cancer are eliminated. The method 300 can then continue in a final step 360 where the most common mutations associated with each cancer type is determined.

In some embodiments, the methods include an additional step of selecting mutations with a potential functionally significant oncogenic mechanism. In some embodiments, the additional step comprises selecting clonal or truncal mutations. In some embodiments, the methods include identifying the most common mutations associated with all forms of cancer.

In some embodiments, the methods include previously synthesizing a peptide library comprising the most common mutations in cancer or a form of cancer. In some embodiments, the methods include selecting a patient for therapy based upon that patient's sequencing results containing one or more of the common mutations for which there were presynthesized peptides to improved manufacturing time and cost. In some embodiments, the patient having the common mutations could be identified in a database of sequencing results (e.g., in a national database, a genomics companies data base, a hospital systems database, a hospitals database, an oncology clinic's data base, an insurance companies database, and individual clinician's database or patient records). In other embodiments, the patients having the common mutations is identified from individual sequencing results collected for any purpose or sequencing tests performed with the purpose of determining eligibility for the T cell therapy. In some embodiments, the methods include synthesizing peptides with mutations for a given patient based upon that patient's sequencing results.

In some embodiments, the most common mutations associated with colon cancer are selected from the group consisting of KRAS G12, KRAS G13 and BRAF V600E. In some embodiments, the mutations associated with lung cancer are selected from the group consisting of KRAS G12 and EGFR E760_A750del L858R. In some embodiments, the mutations associated with pancreatic cancer is KRAS G12. In some embodiments, the mutations associated with DLBCL cancer are selected from the group consisting of MYD88 L256P and EZH2 Y641. In some embodiments, the mutation associated with AML is FLT3 D835. In some embodiments, the mutation associated with AML is NPM1 W288Cfs*12. In some embodiments, the mutation associated with melanoma is BRAF V600E and NRAS Q61. In some embodiments, the mutations associated with bladder cancer are selected from the group consisting of FGFR3 S249C, FGFR3 Y373C, and PIK3CA E545K. In some embodiments, the mutations associated with glioblastoma are selected from the group consisting of IDH1 R132H, EGFR A289V, and EGFR G598V. In some embodiments, genes associated with all cancers (e.g., colon cancer, lung cancer, pancreatic cancer, AML, melanoma, bladder cancer, hematologic cancer, and glioblastoma) are TP53 and KRAS.

In some embodiments, a mutation associated with all cancers is selected from the group consisting of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53 R273C, TP53 273H, TP53 R282W, and TP53 V157F. In some embodiments, the common mutations are TP53 R248W and KRAS G12D.

In some embodiments, overexpressed proteins that are tumor associated antigens (TAA) are selected from the group of overexpressed TAAs including but not limited to CEA, BING-4, Cyclin B1, 9D7, Ep-CAM, EphA3, Her2/neu, Telomerase, Mesothelin, SAP-1, Survivin, BAGE, CAGE, GAGE, MAGE, SAGE, XAGE, NYESO-1, PRAME, SSX-2, Melan-A/MART-1, Gp100, Tyrosinase, TRP1, TRP2, PSA, PSMA and MUC1.

mRNA Compositions and Production

In some embodiments, the present technology provides mRNA compositions and methods of making the same as shown in FIG. 1 (step 109).

In some embodiments, the mRNA comprise at least one mutation associated with a specific type of cancer relative to a wild-type and/or native nucleic acid and/or peptide. In some embodiments, the at least one mutation is selected based on the frequency by which the mutation occurs in a given cancer type.

In some embodiments, the mRNA has a combination of mutations that enable the mRNA to be used on a large patient population. In some embodiments, the peptide can have one or more mutations identified by sequencing the subject's genome (i.e., a “fully personalized” approach).

In some embodiments, the mRNA has one or more mutations associated with a subject's cancer. In some embodiments, the mRNA has one, two, three, four, five, six, seven, eight, nine, ten, or more mutations. In some embodiments, the mRNA has one mutation.

In some embodiments, the mRNA has a mutation associated with a KRAS gene, TP53 gene, or both. In some embodiments, the mRNA has one or more mutations selected from the group consisting of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53 R273C, TP53 273H, TP53 R282W, and TP53 V157F. In some embodiments, the mRNA has a TP53 R248W mutation, KRAS G12D mutation, or both.

In some embodiments, the mRNA includes a 5′ untranslated region (UTR). In some embodiments, the mRNA includes a 3′ UTR. In some embodiments, the mRNA includes a 5′ UTR at one end of the mRNA sequence and a 3′ UTR at the other end of the mRNA sequence. In some embodiments, the 3′ UTR includes one or more human beta globin. In some embodiments, the 3′ UTR includes a poly A binding protein. In some embodiments, the mRNA includes a polylinker.

In some embodiments, the mRNA includes a signal peptide. The signal peptide is necessary as all proteins begin with a methionine residue. In some embodiments, the signal peptide directs amino acids to the MHC class I compartment. In some embodiments, the signal peptide directs the amino acids to the MHC class II compartment. In some embodiments, the signal peptide is followed by amino acid sequence with neoantigen (i.e., mutation) located at the center and germline sequence flanking it. In some embodiments, the amino acid sequence is a 21 or 27 amino acid sequence. In other embodiments, the amino acid sequence is a 15 amino acid sequence. In some embodiments, the construct has 120 residue polyadenine tail (poly (A)) that is added by PCR just before in vitro transcription.

In some embodiments, the mRNA comprises of a 5′ untranslated region (UTR), a signal peptide, a repeating unit of antigen and polylinker, a 3′ UTR containing two repeats of the human beta globin 3′ UTR and a poly A tract to hard code a polyadenylation sequence. In other embodiments, the 3′ UTR is selected from the group consisting of alpha globin and beta globin from Rattus norvegicus or Pan troglodytes 3′ UTRs. In some embodiments, the mRNA further includes a consensus Kozak sequence at the start and the translated region begins with a 24 aa signal domain taken from HLA-A. In some embodiments, the signal domain is selected from the group consisting of HLA-B, HLA-C, HLA-DRB1, LAMP1, LAMP2, TAP1, and TAP2.

In some embodiments, the sequence has furin cleavage sites. In some embodiments, the sequence has poly(G) cleavage sites. In yet another embodiment, the sequence has a 2A or GGSGGGSS sequence. In some embodiments, the sequence can be made with polycistronic having multiple start sites on single RNA.

In some embodiments, the mRNA includes one or more mutations associated with a subject's cancer. In some embodiments, when the mRNA includes two or more mutations, a polylinker aa sequence is added between the mutations. In some embodiments, the polylinker aa sequence is GGSGGGSS. The linker GGSGGGSS has low immunogenicity and is used as a NetMHC MHC I binding affinity tool. In some embodiments, the mutation sequences of interest are wholly contained in the areas in which binding affinity is below the 50^{th} percentile, 40^{th} percentile, 30^{th} percentile, or lower where lower percentile indicates better binding. In some embodiments, the linker is a Furan cleavage site. In some embodiments, the linker is the 2A self-cleavage site. In some embodiments, the linker is a non-coding RNA sequence which forces ribosome skipping between neoantigens.

In some embodiments, the methods include synthesizing mRNA having one or more mutations associated with a subject's cancer. In some embodiments, the mRNA is transcribed from DNA having one or more mutations associated with the subject's cancer.

In some embodiments, the methods include purifying the mRNA prior to use. The purity of mRNA is significant as it impacts the number of cells translating the mRNA and how much protein the cells can produce. In some embodiments the mRNA is purified by reverse phase HPLC. In some embodiments, the mRNA is purified using poly thymidine coated beads after in vitro transcription. In some embodiments, the beads are coated in single stranded poly thymidine DNA sequences and the mRNA after transcription is completed, binds to the beads. Full length RNA will have a poly (A) tail that will bind the beads whereas RNA that has not reached the end of the template where the poly A tail is added will be excluded. In some embodiments, once bound, the mRNA is washed and then eluted with a chaotropic agent to eliminate non polyadenylated sequences leading to more pure RNA. In some embodiments, the coated beads will select for single stranded RNA. This process is significantly cheaper than using other purification processes (e.g., HPLC) as each patient will have to have their purification column for GMP purposes whereas poly (T) beads can be easily produced on a large-scale. In some embodiments, the mRNA will undergo phosphatase treatment to avoid innate immune signaling triggered by free phosphate groups.

In some embodiments, the methods include the use of peptides or “pepmixes” consisting of a mixture of 4 15 amino acid long peptides that tile across a 28 amino acid long amino acid sequence encompassing the mutation. The peptide sequence of the 15 amino acid long sequence moves along the 28 amino acid sequence in an interval of 4 amino acids. In FIG. 1 these peptides are substituted for mRNA at step 109.

In some embodiments, the purified mRNA may be used for vaccines or therapeutics, including but not limited to RNA vaccines. Typically, an RNA vaccine introduces an mRNA or fragment thereof into a cell (e.g., a human cell), which then produces antigens sourced from a pathogen (e.g., viral antigens) or neoantigens encoded by the mRNA to stimulate an adaptive immune response against the pathogen (e.g., cancer cells or viruses). The mRNA can be introduced into a cell in a variety of ways, for example, via injection, lipid nanoparticle delivery, or viral delivery (e.g., retrovirus, lentivirus, alphavirus, or rhabdovirus). In these embodiments, the purified mRNA is used to improve uptake of the mRNA and decrease the incidence of fever, swelling and flu like side effects.

In some embodiments, the RNA vaccine encoding one or more antigens sourced from a pathogen (e.g., viral antigens) or neoantigens can be introduced to a person to stimulate an immune response against those antigens before production of an autologous adoptive T cell therapy against that person's disease. The RNA vaccine stimulates an immune response thereby increasing the number and activity of T cells targeting those antigens to improve the success rate and efficacy of an autologous adoptive T cell therapy. In some embodiments, a patient's PBMCs can be screened against peptides or RNAs encoding one or more antigens sourced from a pathogen or neoantigens to determine which antigens are reactive and which antigens should be encoded in an RNA vaccine. In some embodiments, only the unreactive antigens will be included in the RNA vaccine.

In some embodiments, the RNA vaccine encoding one or more antigens sourced from a pathogen (e.g., viral antigens) or neoantigens can be introduced to a person with disease after the person has received an autologous adoptive T cell therapy, to act as a booster to the T cells introduced as a part of the autologous adoptive cell therapy against that person's disease. It is common for vaccines to require multiple “booster shots” for an effective response. The RNA vaccine would stimulate the T cells of the therapy to prolong or improve the response of the adopted cells. In some embodiments, a patient's PBMCs can be screened against peptides or RNAs encoding one or more antigens sourced from a pathogen or neoantigens to determine which antigens are reactive and which antigens should be encoded in the RNA vaccine. In some embodiments, only the unreactive antigens will be included in the RNA vaccine.

In some embodiments, the mRNA has a purity of at least about 80%, at least about 85%, at least about 90%, at least about 92%, at least about 94%, at least about 96%, at least about 97%, at least about 98%, or at least about 99% pure. In some embodiments, the mRNA comprises less than about 20%, less than about 15%, less than about 10%, less than about 8%, less than about 6%, less than about 4%, or less than about 2% any other material (e.g., cellular material, culture medium, chemical precursors for synthesizing DNA).

In some embodiments, the mRNA contains a eukaryotic compatible 5′ cap such as Trilink™ clean cap AG or ARCA. In some embodiments the mRNA has uracil fully substituted with 5-methoxyUracil or pseudouridine.

In other embodiments, a vaccine containing antigens or epitopes from multiple viral proteins is produced. One advantage of a vaccine targeting multiple viral antigens is that it is hard for the virus to mutate away. Such is particularly being observed with the current Cov-2 vaccines that only target Spike (S).

In preferred embodiments this vaccine is RNA or DNA.

In other preferred embodiments this vaccine targets antigens or epitopes from multiple viral proteins of SARS-COV-2

In In other preferred embodiments this vaccine targets antigens from multiple viral proteins of SARS-COV-2 including two or more of the following: Cov-2 S, M, N, 3a, 7a, 8.

In one preferred embodiment an mRNA vaccine simultaneously targeting Cov-2 Spike (S), VME1 (M), NCAP (N), 3a, 7a, 8 is produced. In an alternative preferred embodiment, an mRNA vaccine simultaneously targeting two or more of Cov-2 Spike (S), VME1 (M), NCAP (N), 3a, 7a, 8 is produced. In still another embodiment, an mRNA vaccine simultaneously targeting Cov-2 Spike (S), and one or more of the following: VME1 (M), NCAP (N), 3a, 7a, 8 is produced. In other preferred embodiments this vaccine targets antigens from multiple viral proteins of SARS-COV-2 including two or more of the following: Cov-2 S, M, N, 3a, 7a, 8. In still another embodiment, an mRNA vaccine simultaneously targeting one or more of the following: Cov-2 VME1 (M), NCAP (N), 3a, 7a, 8 is produced.

In another preferred embodiment a DNA vaccine simultaneously targeting Cov-2 Spike (S), VME1 (M), NCAP (N), 3a, 7a, 8 is produced. In an alternative preferred embodiment, an DNA vaccine simultaneously targeting two or more of Cov-2 Spike (S), VME1 (M), NCAP (N), 3a, 7a, 8 is produced. In still another embodiment, a DNA vaccine simultaneously targeting Cov-2 Spike (S), and one or more of the following: VME1 (M), NCAP (N), 3a, 7a, 8 is produced. In still another embodiment, an DNA vaccine simultaneously targeting one or more of the following: Cov-2 VME1 (M), NCAP (N), 3a, 7a, 8 is produced.

In another preferred embodiment, such vaccine targets Nsp6.

In another preferred embodiment, RNA or DNA vaccines against antigens or epitopes on any one of these viral proteins are administered in addition to a vaccine targeting Cov-2 Spike (S).

In other preferred embodiments the viral proteins are from Eastern Equine Encephalitis or other viruses in this disclosure.

In preferred but not limiting embodiments the antigens for the vaccine are selected reflect the effective clearance response in natural immunity to a virus. By determining the relative CD8 and CD4 T cell response to multiple viral proteins from any given virus in a population of healthy donor T cells versus a population of patients who have successfully cleared the virus, the relevant antigens and epitopes were selected for the multiantigen vaccine.

### Peptide Compositions and Production

In some embodiments, the present disclosure provides peptide compositions (“pepmixes”) and methods of making the same.

In some embodiments, the peptide can have all common mutations associated with a specific type of cancer. In some embodiments, the common mutations are selected based on the frequency by which the mutation occurs in a given cancer type. In some embodiments, the peptide is a pepmix having one or more common mutations associated with a given cancer. In some embodiments, each neoantigen in the pepmix corresponds to a germline sequence with a mutation at the center of a 27 amino acid sequence tiled by 15 amino acids with 11 amino acid overlap. In some embodiments, an entire protein is targeted, and 15 amino acids are tiled across the entire sequence or a selected portion of the protein.

In some embodiments, the peptide has a combination of mutations that enable the peptide to be used on a large patient population. In some embodiments, the peptide can have one or more mutations identified by sequencing the subject's genome (i.e., a “fully personalized” approach). In some embodiments, the peptide has all of the most common mutations and rearrangements across all forms of cancer. In some embodiments, the peptide has the most common mutations in a specific cancer type. In some embodiments, the peptide has the most common mutations associated predisposing to a specific cancer. In another embodiment, the peptide has the most common mutations, rearrangements, and frameshift mutations associated with cancer. In some embodiments, peptides are selected from a pre-synthesized library of the most common mutations and rearrangements based upon a sequence database where patients have more than one mutation and rearrangement in that patient's cancer.

In some embodiments, the peptide has one or more mutations associated with a subject's cancer. In some embodiments, the peptide has one, two, three, four, five, six, seven, eight, nine, ten, or more mutations. In some embodiments, the peptide has one mutation.

In some embodiments, the peptide has a mutation associated with a KRAS gene, TP53 gene, or both. In some embodiments, the peptide has one or more mutations selected from the group consisting of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53 R273C, TP53 273H, TP53 R282W, and TP53 V157F. In some embodiments, the peptides have a TP53 R248W mutation, KRAS G12D mutation, or both.

In some embodiments, the combination of mutations used in the peptide are effective at preventing recurrence of chemotherapy and/or radiation treatment induced cancers.

In some embodiments, the peptide is synthesized to include all relevant mutations and is purified. In some embodiments, the peptide is purified by column chromatography (e.g., HPLC). In some embodiments the peptide is at least about 90%, about 95%, about 96%, about 97%, about 98%, about 99%, or about 99.5% pure. In some embodiments, mass spectrometry is used to determine if the peptide is stable. In some embodiments, the pepmix can be synthesized on a milligram (mg) to gram (g) scale.

In some embodiments, the one or more target antigens used in the above methods comprises a plurality of overlapping peptides derived from a target antigen. In some embodiments of the invention, the overlapping peptides are 15-50 amino acids in length. In a preferred embodiment, the polypeptides are 15 amino acids in length. In some embodiments of the invention, the one or more target antigens used in the above methods comprises a plurality of overlapping peptides derived from a target antigen. In some embodiments of the invention, the overlapping peptides are 15-50 amino acids in length. In a preferred embodiment, the polypeptides are 15 amino acids in length. In some embodiments, the peptides are 8 amino acids to 100 amino acids in length.

In some embodiments of the invention, the one or more target antigens comprises polypeptides derived from one or more target viral antigens. In further embodiments, the target antigen is a protein expressed by one or more of cytomegalovirus, Epstein-Barr virus, hepatitis B virus, human papillomavirus, adenovirus, herpes virus, human immunodeficiency virus, influenza virus, human respiratory syncytial virus, vaccinia virus, varicella-zoster virus, yellow fever virus, Ebola virus, coronavirus (e.g., SARS-CoV, MERS-CoV, SARS-CoV-2), Eastern equine encephalitis virus, Polyomavirus hominis1 (BKV), (VP1, VP2, VP3, large T antigen, and small t antigen) and Zika virus. In some embodiments, the one or more target antigens comprise polypeptides derived from one or more of the Epstein-Barr virus antigens LMP1, LMP2, and EBNA1. In other embodiments, one or more of peptide mixes for the LMP1, LMP2, EBNA1 can be from multiple strains of the Epstein-Barr virus. In some embodiments, the one or more target antigens comprise polypeptides derived from one or more of the Epstein-Barr virus antigens selected from one or more of LMP1, LMP2, EBNA1 and BARF proteins. In some embodiments, the peptides from EBV LMP1, LMP2, EBNA1 and BARF proteins can be from one of the six strains of Epstein-Barr virus or some combination thereof. In other embodiments, the one or more target antigens comprise polypeptides derived from one or more of the cytomegalovirus antigens, pp65, Cancer/testis antigen 1 (NY-ESO-1), and Survivin. In other embodiments, any cancer associated antigen can be used. Non-limiting examples of cancer associated antigens include human papillomavirus proteins E6, E7, and others, hepatitis B or C antigens associated with hepatocellular carcinoma hepatitis B or C surface antigen. In some embodiments, these and other antigens can be targeted with peptide or RNA/DNA libraries including multiple strains selected from the group consisting of HPV 16, HPV 18 and HPV 16, HPV 18, HPV 31, HPV 33, HPV 35, HPV 39, HPV 45, HPV 51, HPV 52, HPV 56, HPV 58, HPV 59, HPV 66 and HPV 68.

In some embodiments, the peptide and/or pepmixes have a purity of at least about 50%, about 55%, about 60%, about 65%, about 70%, about 75%, about 80%, at least about 85%, at least about 90%, at least about 92%, at least about 94%, at least about 96%, at least about 97%, at least about 98%, or at least about 99% pure. In some embodiments, the peptide and/or pepmixes comprises less than about 15%, less than about 10%, less than about 8%, less than about 6%, less than about 4%, less than about 2%, or less of any other material.

Transfection with mRNA or Combination with Peptides

In some embodiments, the present disclosure provides mRNA compositions for use in transfecting DCs as shown in FIG. 1 (Step 110) and peptide compositions for use in combining with DCs as shown in FIG. 2 (Steps 205 and 207).

In some embodiments, the present disclosure provides methods of transfecting DCs with mRNA encoding one or more mutations associated with a subject's cancer. In some embodiments, the methods include transfecting DCs with mRNA by any conventional transfection technique. Non-limiting examples of conventional transfection techniques include lipofection, electroporation, calcium phosphate transfection, liposome transfection, viral transduction, and nucleofection as well as physical methods such as microinjection and biolistic particle delivery.

In some embodiments, the transfection technique is nucleofection. Nucleofection may be performed by any useful means in the art, including, for example, with an Amaxa® nucleofection system or InVitrogen™ nucleofection system. In some the nucleofection is performed with the 4D nucleofection core unit, X unit, Y units. In some the nucleofection is performed closed system in sequential pulses of cells. In some the program used is CB-105 for human DCs., EO 115 for human stimulated T-cells, F1115 for human unstimulated T-cells. In some embodiments, the nucleofection includes combining about 100,000 to 40,000,000 DCs with mRNA. In some embodiments, about 2 μg to about 10 μg of mRNA is added to the 100,000 to 5,000,000 DCs. For example, about 2 μg, about 3 μg, about 4 μg, about 5 μg, about 6 μg, about 7 μg, about 8 μg, about 9 μg, or about 10 μg of mRNA is added to the 100,000 to 5,000,000 DCs.

In some embodiments, the transfection is lipofectamine, lipid nanoparticles or electroporation. In some embodiments, the transfection can take place in the same chamber in which the monocytes are differentiated into DCs. In yet another embodiment, the transfection can take place in the same bioreactor in which the T cells are simulated and primed by the DCs.

In some embodiments, DCs are transfected with mRNA at a ratio of about 1 μg of mRNA per 1 million DCs, about 2 μg of mRNA per 1 million DCs, about 4 μg of mRNA per 1 million DCs, about 4 μg of mRNA per 1 million DCs, about 5 μg of mRNA per 1 million DCs, about 6 μg of mRNA per 1 million DCs, about 7 μg of mRNA per 1 million DCs, about 8 μg of mRNA per 1 million DCs, about 9 μg of mRNA per 1 million DCs, or about 10 μg of mRNA per 1 million DCs. In some embodiments, DCs are transfected with mRNA at a ratio of about 1 μg of mRNA per 2 million DCs, about 2 μg of mRNA per 2 million DCs, about 3 μg of mRNA per 2 million DCs, about 4 μg of mRNA per 2 million DCs, about 5 μg of mRNA per 2 million DCs, about 6 μg of mRNA per 2 million DCs, about 7 μg of mRNA per 2 million DCs, about 8 μg of mRNA per 2 million DCs, about 9 μg of mRNA per 2 million DCs, or about 10 μg of mRNA per 2 million DCs. In some embodiments, DCs are transfected with mRNA at a ratio of about 1 μg of mRNA per 3 million DCs, about 2 μg of mRNA per 3 million DCs, about 3 μg of mRNA per 3 million DCs, about 4 μg of mRNA per 3 million DCs, about 5 μg of mRNA per 3 million DCs, about 6 μg of mRNA per 3 million DCs, about 7 μg of mRNA per 3 million DCs, about 8 μg of mRNA per 3 million DCs, about 9 μg of mRNA per 3 million DCs, or about 10 μg of mRNA per 3 million DCs.

In some embodiments, the present disclosure provides methods of combining peptides having one or more mutations associated with a subject's cancer with DCs. In some embodiments, combining the peptides with DCs includes incubating the peptides with the DCs in order to incorporate the peptides with the DCs.

In some embodiments, DCs are incubated with peptides at a ratio of about 1 μg of peptide per 1 million DCs, about 2 μg of peptide per 1 million DCs, about 3 μg of peptide per 1 million DCs, about 4 μg of peptide per 1 million DCs, about 5 μg of peptide per 1 million DCs, about 6 μg of peptide per 1 million DCs, about 7 μg of peptide per 1 million DCs, about 8 μg of peptide per 1 million DCs, about 9 μg of peptide per 1 million DCs, or about 10 μg of peptide per 1 million DCs. In some embodiments, DCs are transfected with peptides at a ratio of about 1 μg of peptide per 2 million DCs, about 2 μg of peptide per 2 million DCs, about 3 μg of peptide per 2 million DCs, about 4 μg of peptide per 2 million DCs, about 5 μg of peptide per 2 million DCs, about 6 μg of peptide per 2 million DCs, about 7 μg of peptide per 2 million DCs, about 8 μg of peptide per 2 million DCs, about 9 μg of peptide per 2 million DCs, or about 10 μg of peptide per 2 million DCs. In some embodiments, DCs are transfected with peptides at a ratio of about 1 μg of peptide per 3 million DCs, about 2 μg of peptide per 3 million DCs, about 3 μg of peptide per 3 million DCs, about 4 μg of peptide per 3 million DCs, about 5 μg of peptide per 3 million DCs, about 6 μg of peptide per 3 million DCs, about 7 μg of peptide per 3 million DCs, about 8 μg of peptide per 3 million DCs, about 9 μg of peptide per 3 million DCs, or about 10 μg of peptide per 3 million DCs.

In some embodiments, DCs are pulse primed in a closed system (“Pizza Pie” Closed System). In Pizza Pie Closed System, the DCs are grown on a circular or multisided cassette with multiple sections of a single compartment (e.g., like slices of a Pizza). In an alternative embodiment, the DCs are grown on a circular or multisided cassette with multiple compartments. In some embodiments, the flow of the DC peptides or RNA is pumped in with flow from the outside into the center and then out of the compartment of the closed system. In other embodiments, the flow of the DC peptides or RNA is pumped in with flow from the center to the outer edge of the section or compartment and then out of the compartment of the closed system. In some embodiments, while priming the DCs, peptides or RNA (e.g., in the presence of lipofectamine/other agent) are individually (or in small groups) introduced by fluidics over one of the segments. Following this method, the DCs in that area are only loaded with peptides from one antigen. In some embodiments, the DCs are harvested, pooled and then, combined with PBMC's or non-adherent compartment and used to prime the T cells. This method allows for efficient priming without competition amongst epitopes T cell priming to one antigen at a time at the T cell to DC cell level. In some embodiments, the T cells are infused equally across the segments for priming+/− early expansion in the segment. In some embodiments, after priming for a number of hours, media is added, and the rest of the steps outlined in the disclosed processes can be followed.

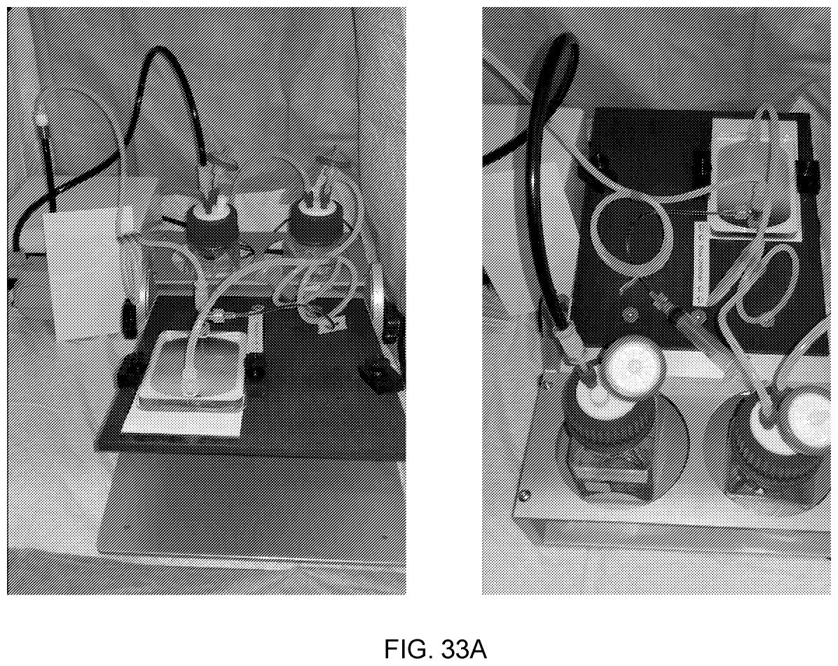

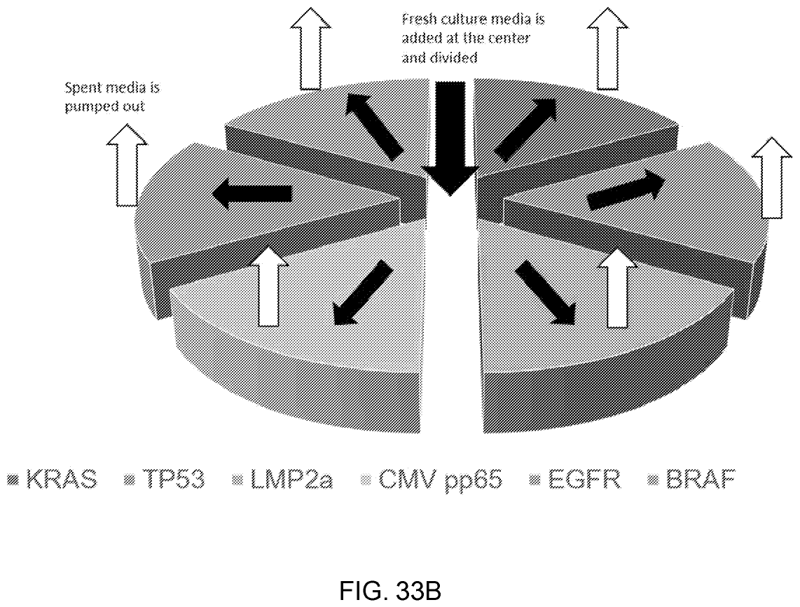

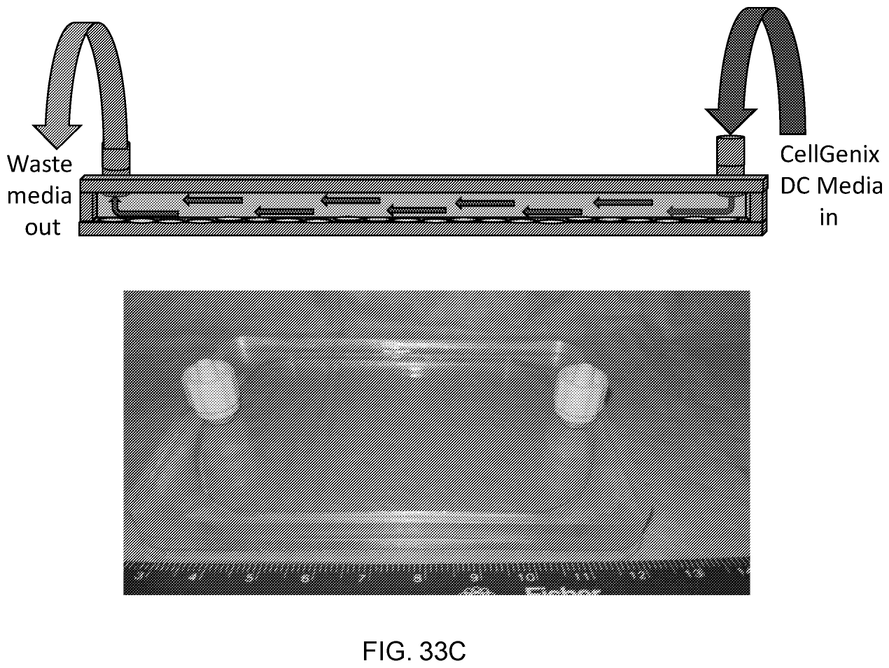

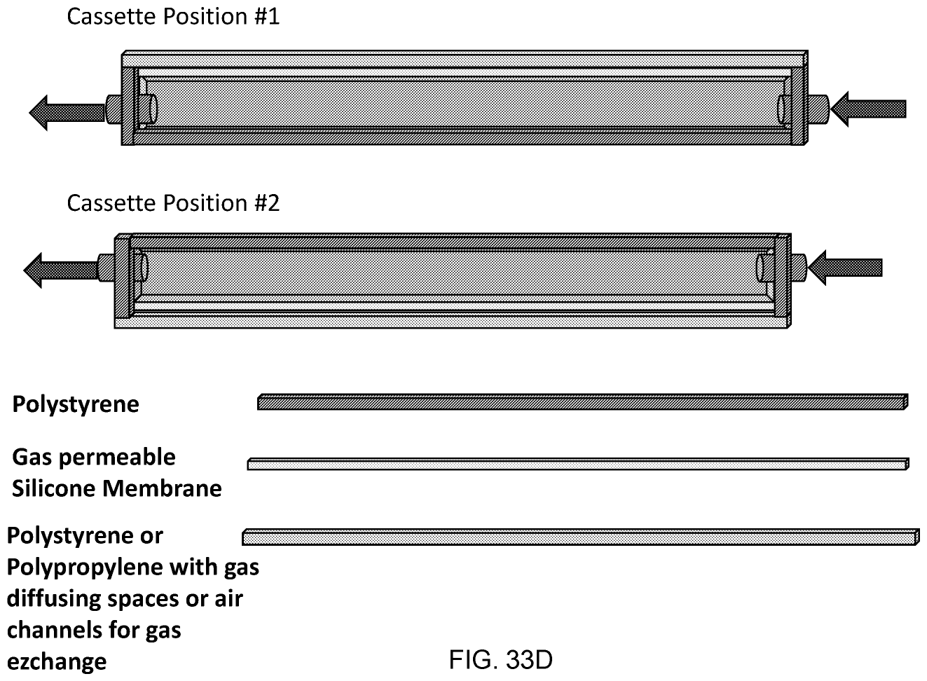

In other embodiments, the closed system can be a cartridge in which the DC's are seeded, produced and released within a polystyrene cassette without the need for open steps (FIG. 33C). A system in which all steps are performed with tubing connected to a pump, preferable a peristaltic pump which easily releases tubing. In some embodiments, a sterile air bubble can be introduced and moved by forward/reverse cycling of the peristaltic pump or rocking or orbital shaking. Alternate release agents include but are not limited to trypsin, collagenase I-IV, EDTA, EGTA, Accutase, PBS minus calcium minus magnesium.

In other embodiments, the closed system can be a cartridge with a polystyrene surface on one side and a silicon membrane on the other side. FIG. 33D In this process the DC's can be first grown and matured on the polystyrene using the methods described herein. The PBMCs can then be added and primed on that surface. After the priming for 1 hour to 72 hours, the cartridge is flipped, and the T cells are expanded on the gas permeable membrane side according to the methods described herein.

### Stimulation and Priming

In some embodiments, the present disclosure provides methods of stimulating and priming T cells with the DCs transfected with mRNA as shown in FIG. 1 (Step 111) and DCs combined with peptide as shown in FIG. 2 (Steps 206 and 207).

In some embodiments, the methods include obtaining a population of cells comprising T cells from a subject diagnosed with cancer, having recurrent cancer, and/or a high risk of developing cancer. In some embodiments, the blood is collected before surgical resection of a tumor. In some embodiments, the blood is collected before surgical resection of the primary tumor. In some embodiments, the blood is collected before a patient has cancer. In some embodiments, the blood is collected by apheresis. In some embodiments, the population of cells comprising T cells were previously frozen. In some embodiments, the population of cells comprising T cells are freshly isolated. In some embodiments, the methods comprise obtaining a population of cells derived from the same subject in which the DCs are obtained to proliferate T cells specific to one or more mutations associated with the subject's cancer.

In some embodiments, the methods include exposing the population of cells comprising T cells to the DCs having one or mutations associated with the subject's cancer and one or more cytokines to stimulate T cell production. In embodiments, the cells are sequentially stimulated with individual DCs. In other embodiments, the cells are stimulated with multiple DCs simultaneously.

In some embodiments, the concentration of DCs exposed to the population of cells comprising T cells is between about 1 nanogram to 10 micrograms per mL of culture medium. For example, about 1 nanogram, about 2 nanograms, about 3 nanograms, about 4 nanograms, about 5 nanograms, about 6 nanograms, about 7 nanograms, about 8 nanograms, about 9 nanograms, about 10 nanograms of DCs per mL of culture medium.

In some embodiments, the methods include one or more stimulations of T cells in which the population of cells comprising T cells are re-exposed to DCs having one or more mutations associated with the subject's cancer and one or more cytokines. In some embodiments, following monocyte differentiation into DCs and transfection with mRNA and/or combination with a peptide, a portion of the DCs is preserved for an additional stimulation step. In some embodiments, a portion of the DCs is frozen (e.g., in a CryoStor® CS10) at a cell density 1×10⁶ cells/mL. In some embodiments, a portion of the DCs are frozen in another cryoprotectant (e.g., CryoStor® CS5). In some embodiments, the DCs are frozen in the cryoprotectant at a cell density of about 1×10⁶ cells/mL. In some embodiments, the second stimulation step includes introducing 1 to 2 million of the preserved DCs to the population of cells comprising the stimulated T cells. The additional stimulations increase the T cell number and fraction of reactive T cells.

In some embodiments, the methods include expanding T cells in a cell culture comprising exposing the population of cells comprising T cells to a cytokine selected from the group consisting of IL-2, IL-7, IL-12, IL-15, and IL-21. In some embodiments, the T cells in a cell culture are exposed to IL-7 and IL-15. In some embodiments, the T cells in a cell culture are exposed to one or more of IL-2, IL-15, and IL-21. In other embodiments the cytokine cocktails are IL-7, IL-12, IL-15, and IL-6. In other embodiments, the cytokine is IL-15 alone. In still other embodiments, the cytokine cocktails are IL-4 and IL-7. In some embodiments, the cytokines are added at the same time. In other embodiments, the cytokines are added in stepwise (e.g., not at the same time).

In some embodiments, the stimulation promotes expansion of the CD4⁺ T cell population. In some embodiments, the stimulation and expansion promote expansion of the CD3⁺ T cell population. In some embodiments, the stimulation and expansion promote expansion of the CD8⁺ T cell population. In other embodiments, the stimulation and expansion promote expansion of both the CD8⁺ and CD4⁺ T cell populations.

In some embodiments, the methods include generating multiple sub-populations of cells from the population of cells, which are each stimulated by exposure to one or more DCs having one or more mutations associated with a subject's cancer.

In one embodiment, testing the cell population for antigen-specific reactivity comprises detection of T cell activation markers. In one embodiment, detection of T cell activation markers is accomplished by one or more of flow cytometry and measurement of antigen induced cytokine production by intracellular cytokine staining, ELISA, or enzyme-linked immunospot (ELISpot). Markers for T cell activation measure by flow cytometry include one or more of CD45RO, CD137, CD25, CD279, CD179, CD62L, HLA-DR, CD69, CD223 (LAG3), CD134 (OX40), CD183 (CXCR3), CD127 (IL-7Rα), CD366 (TIM3), CD80, CD152 (CTLA-4), CD28, CD278 (ICOS), CD154 (CD40L). Antigen induced cytokines (e.g., TNFα, IFNγ, IL-2) as well as CD107a are mobilized, alone or in combination in CTLs, in response to stimulation and can also be measured along with the cytokines by flow cytometry.

In some embodiments, the population of cells comprising T cells are exposed cytokines TNFα and IFNγ and DCs having one or more mutations associated with the subject's cancer to produce CD8⁺ T cells and for directing an immune response within the subject following infusion of the T cells.

In some embodiments, the methods comprise screening of the population of cells comprising T cells for PD-1 expression, selecting the PD-1 positive cells, and propagating the T cells in cell culture conditions that will allow robust expansion of the cells. In another embodiment, sorting with other activation markers or multiple activation markers can be performed to select the T cells expressing those activation markers and propagating the T cells in cell culture conditions that will allow robust expansion of the cells.

In some embodiments, the methods comprise screening of the population of cells comprising T cells for the expression of CD137 on isolated cells in culture for antigen exposure marker and subjecting the cells bearing CD137 marker to cell culture conditions that will allow robust expansion of the cells. In another embodiment, a multitude of expression markers including CD-137 and PD-1 are used to select the cells for expansion ex vivo. The expression markers for screening the cells that have been antigen-primed in vivo include one or more members selected from the group comprising CD8, CD274, CD62L, CD45RA, CD45RO, CD27, CD28, CD69, CD107, CCR7, CD4, CD44, CD137 (4-1BB), CD137L (4-1BBL), CD279 (PD-1), CD223 (LAG3), CD134 (OX40), CD278 (ICOS), CD183 (CXCR3), CD127 (IL-7R), CD366 (TIM3), CD25 (IL-2RA), CD80 (B7-1), CD86 (B7-2), VISTA (B7-H5), CD152 (CTLA-4), CD154 (CD40L), CD122 (IL-15R), CD360 (IL-21R), CD71 (Transferrin receptor), CD95 (Fas), CD95L (FasL), CD272 (BTLA), CD226 (DNAM-1), CD126 (IL-6R), and adenosine A2A receptor (A2AR).

In some embodiments, the methods include polyclonal stimulation of the T cells. In one embodiment, the polyclonal stimulation comprises exposing the cell to tetrameric antibodies that bind CD3, CD28, and/or CD2. Other non-specific T cell activators can be used for polyclonal expansion of T cells including but not limited to PHA (phytohemagglutinin), PMA/lonomycin, anti CD3, anti CD3 beads, and anti CD3/anti CD28.

In some embodiments, the tetrameric antibodies are added to the cells at a ratio of about 10 μl to 2 million cells/ml, about 15 μl to 2 million cells/ml, about 20 μl to 2 million cells/ml. In some embodiments, following three weeks, the culture volume of media is increased to 3 mL per 3 million of initial starting cells. In some embodiments, following three weeks, the culture volume is increased to 4 mL per 3 million of initial starting cell number. In some embodiments, using these concentrations and relative volumes a culture can range in density from 2 million cells in 1 mL to an excess of 1 billion cells in 5 mL. In some embodiments, polyclonal stimulation occurs after the T cells have been stimulated to expand by exposure to one or more target antigens and to certain cytokines. In some embodiments, the polyclonal stimulation is performed at least about two weeks prior to harvesting the cells.

In some embodiments, the methods include priming the T cells to increase the fraction of memory T cells while decreasing the number of effector T cells in the population of cells where a higher fraction of memory T cells can improve the longevity and efficacy of the treatment. In some embodiments, priming the population of cells comprising T cells increases the fraction of memory T cells having a phenotype selected from the group consisting of CD197⁺, CD45RO⁺, CD62L⁺, and CD95⁺.

In some embodiments, small molecules can be used in the priming process to increase the fraction of responding cells. The addition of small molecules can improve the T cell response to the DCs having one or more mutations associated with the subject's cancer.

In some embodiments, the small molecules are apoptosis or cell death inhibitors. In some embodiments, the small molecules are Rho-associated protein kinase (ROCK) inhibitors. In some embodiments, the ROCK inhibitor is a ROCK1 inhibitor, ROCK2 inhibitor, or both. Non-limiting examples of the apoptosis or ROCK inhibitors include Y-27632 2HCI, Thiazovivin, Fasudil (HA-1077) HCI, GSK429286A, RKI-1447, Azaindole 1 (TC-S 7001), GSK269962A HCI, Netarsudil (AR-13324), Y-39983 HCI, ZINC00881524, KD025 (SLx-2119), Ripasudil (K-115), Hydroxyfasudil (HA-1100) AT13148, AMA-0076, AR-1286, ATS907, DE-104, INS-115644, INS-117548, PG324, Y-39983; RKI-983, SNJ-1656, Wf-563, Azabenzimidazole-aminofurazans, H-1152P, XD-4000, HMN-1152, Rhostatin, 4-(1-aminoakyl)-N-(4-pyridl)cyclohexane-carboamides, BA-207, BA-215, BA-285, BA-1037, Ki-23095, VAS-012, quinazoline, Netarsudil, and ITRI-E-212. Without intending to be limiting on the present technology, it is thought that Y-27632 competes with ATP at the active site of ROCK1 and ROCK2. Blocking activity of ROCK1/2 may improve the recovery of cells (e.g., embryonic stem cells) after thawing or replating from one dish to another by blocking apoptosis via reduced caspase 3 cleavage. Without intending to be limiting on the present technology, it is thought that vaccinia protein B18R inhibits apoptosis. For example, introduction of foreign RNA can result in signaling through RIG-1 of interferon type I responses leading to apoptosis. B18R may inhibit the release of type I interferon thereby preventing primary cells from apoptosis after transfection procedures. In some embodiments, the methods include using 5-methoxyuridine to eliminate RIG-1 triggered signaling resulting in apoptosis. In another embodiment, the methods include use of a 5′ cap that mimics a natural cap such as Trilink's CleanCap® AG.

In some embodiments, the apoptosis inhibitors are selected from the group consisting of 10058-F4, 4′-methoxyflavone, AZD5438, BAG1 (72-end) protein, BAX Inhibiting peptide, BEPP monohydroxychloride, BI-6C9, BTZO, Bongkrekic acid, CTP inhibitor, CTX1, Calpeptin, Clofarabine, Clusterin nuclear form protein, Combretastatin A4, Cyclic Pifithrin-a hydroxybromide, EM20-25, Fasentin, Ferrostatin-1, GNF-2, IM-54, Ischemin-CalbiochemA cell permeable azobenezene, Liproxstatin-1, MDL28170, Mdivi-1, Mitochondrial Fusion Promoter, N-Ethylmaleimide, N-Ethylmaleimide, NS3694, NSCI, Necrostatin-1, Oridonin, PD151746, PDI inhibitor 16F16, Pentostatin, Pifithrin-a, Pifithrin-a p-Nitro Cyclic, Pifithrin-u, S-15176 difumarate, UCF-101, p53-Snail binding inhibitor GH25, TW-37, and Z-VAD-FMK.

In some embodiments, the apoptosis inhibitors, Rock inhibitors, or B18R can be used to achieve higher viability transfections of RNA or DNA into cells. In some embodiments, transfection of RNA or DNA is carried out using nucleofection, electroporation, or viral vectors. In some embodiments, the small molecules (e.g., ROCK inhibitors) could be co-administered with RNA vaccines to improve uptake the RNA vaccines and decrease the incidence of fever, swelling, and flu-like side effects.

In some embodiments the small molecules would be added to culture pre or post transfection and can vary from no further treatment or extended treatment from day 1 for part or all of the culture time.

In some embodiments, after stimulation and priming, the population of cells comprising T cells comprises no or substantially no other cell type. In some embodiments, the population of cells comprising T cells comprises less than about 20%, less than about 15%, less than about 10%, less than about 5%, less than about 3%, less than about 1% of no other cell type. In some embodiments, the other cell type includes monocytes, DCs, or PBMCs.

In some embodiments, the methods stimulating and priming T cells with the DCs having one or more mutations associated with the subject's cancer comprises “seeding” the population of cells comprising T cells with T cells. In some embodiments, the methods comprise seeding the population of cells comprising PBMCs at a seeding density of about 0.2×10⁶, about 0.4×10⁶, about 0.6×10⁶, about 0.8×10⁶, about 1.0×10⁶, about 1.2×10⁶, about 1.4×10⁶, about 1.6×10⁶, about 1.8×10⁶, or about 2.0×10⁶, about 1×10⁷, about 5×10⁷, about 1×10⁸, about 5×10⁸, about 1×10⁹, about 5×10⁹, about 1×10¹⁰, about 5×10¹⁰, about 1.0×10¹¹, about 5×10¹¹, about 1×10¹², or about 5×10¹² T cells per mL of median volume.

### Compositions of Primed Cells

In some embodiments, the present disclosure provides a composition comprising T cells that can be used in adoptive cell therapy.

In some embodiments, the composition comprises of one or more of killer T cells, effector memory T cells, helper T cells (helper Th1 or helper Th2), regulatory T cells, and memory T cells. In some embodiments, the composition comprises one or more of CD3⁺, CD4⁺, and CD8⁺ T cells. In some embodiments, the composition comprises effector memory T cells and central memory T cells.

In some embodiments, the composition comprises T cells having a TCRs specific to a patient's neoantigens. In some embodiments, the presence of the TCR is determined by TCR sequencing. In some embodiments, the composition comprises T cells that respond to mutant peptides but not the wild-type peptides. In some embodiments, the response of the T cells to mutant peptides is determined by ELISpot or cytotoxicity assay.

In some embodiments, the composition comprises T cells, wherein the T cells kill patient derived cells transfected with neoantigen mutant RNA but not cells transfected with corresponding wild-type RNA (e.g., not containing the mutation, or rearrangement or other abnormality). In some embodiments, the transfection takes place in the presence of anti-apoptosis agent to prevent cell death and enhance the percent of transfected cells.

In some embodiments the final product is tested for tumor killing capacity by combining the product with donor matched monocytes transfected with mRNA in a real time cell adhesion assay. 1×10⁶ Donor matched monocytes are nucleofected using the 4D with 1 ug, 2 ug, 3 ug, 4 ug of mRNA either encoding all the tumor antigens of a patient or mRNA encoding individual tumor antigens. Monocytes to T cell product cells are plated in a 1:1, 1:2, 1:5, 1:10, 1:20, 1:30, 1:40, 1:50 ratio per well of an RTCA plate. Loss of monocyte adhesion as an indicator of killing capacity is measured for 24, 48, 72, 96, 120, 144, 168 hours.

In some embodiments the final product is tested for tumor killing capacity by combining the product with donor matched B cells, Macrophages, T cells, PHA Blasts, or other patient cells transfected with mRNA in a real time cell adhesion assay. 1×10⁶ Donor matched monocytes are nucleofected using the 4D with 1 ug, 2 ug, 3 ug, 4 ug of mRNA either encoding all the tumor antigens of a patient or mRNA encoding individual tumor antigens. Monocytes to T cell product cells are plated in a 1:1, 1:2, 1:5, 1:10, 1:20, 1:30, 1:40, 1:50 ratio per well of an RTCA plate. Loss of monocyte adhesion as an indicator of killing capacity is measured for 24, 48, 72, 96, 120, 144, 168 hours.

In some embodiments nucleofected donor matched white blood cells such as monocytes, B cells, macrophages, DCs or T cells can be used as vehicle for antigen presentation in an ELISpot assay for cytokines such as TNF-α, IFN-γ, IL-4, IL-10, IL-15. The cells for nucleofection are isolated by plastic (polystyrene) adhesion or antibody linked magnetic beads.

In other embodiments, T cells can be assayed using the RNA RTCA or RNA ELISpot assays in the current disclosure.

In some embodiments, the composition comprises CD3⁺ T cells. In some of these embodiments, the composition comprises about 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than 50,000,000 CD3⁺ T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater CD3⁺ T cells.

In some embodiments, the composition comprises CD8⁺ T cells. In some embodiments, the composition comprises about 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 CD8⁺ T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater CD8⁺ T cells.

In some embodiments, the composition comprises CD4⁺ T cells. In some embodiments, the composition comprises about, 5,000 about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 CD4⁺ T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater CD4⁺ T cells.

In some embodiments, the composition comprises CD8⁺ and CD4⁺ T cells where the number of CD8⁺ T cells is greater than the number of CD4⁺ T cells. In some embodiments, the ratio of CD8⁺ to CD4⁺ T cells is about 1:1, about 2:1, about 4:1, about 6:1, about 8:1, or about 10:1.

In some embodiments, the composition comprises memory T cells. In some embodiments, the composition comprises about, 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 memory T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater memory T cells. Memory T cells include effector memory, central memory and stem cell memory.

In some embodiments, the composition comprises effector memory T cells. In some embodiments, the composition comprises about, 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 effector memory T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater effector memory T cells. In some embodiments, the effector memory T cells present in the composition have a surface marker selected from one or more of CD197⁻, CD45RO⁺, CD62L⁻, and CD95⁺.

In some embodiments, the composition comprises central memory T cells. In some embodiments, the composition comprises about, 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 central memory T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater central memory T cells. In some embodiments, the central memory T cells present in the composition have a surface marker selected from one or more of CD197⁻, CD45RO⁺, CD62L⁻, and CD95⁺.

In some embodiments the stem cell memory present in the composition have a surface marker selected from one or more of CD197⁻, CD45RO⁻, CD45RA⁺CD62L⁺, and CD95⁺. In some embodiments, the composition comprises about 1% or greater, 2% or greater, about 3% or greater, about 4% or greater, about 5% or greater, about 6% or greater, about 7% or greater, about 7.5% or greater, about 8% or greater, about 8.5% or greater, about 9% or greater, or about 9.5% or greater stem cell memory T cells.

In some embodiments, the composition comprises central memory T cells and effector memory T cells and stem cell memory T cells. In some embodiments, the composition comprises about, 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 central memory T cells and effector memory T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater central memory T cells and effector memory T cells.

In some embodiments, the composition comprises CD3⁺ and CD8⁺ T cells. In some embodiments, the composition comprises about, 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 CD3⁺ and CD8⁺ T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater CD3⁺ and CD8⁺ T cells.

In some embodiments, the composition comprises CD3⁺ and CD4⁺ T cells. In some embodiments, the composition comprises about, 5,000, about 10,000, about 20,000, about 25,000, about 30,000, about 35,000, about 40,000, about 45,000, about 50,000, about 55,000, about 60,000, about 65,000, about 70,000, about 75,000, about 80,000, about 85,000, about 90,000, about 100,000, about 150,000, about 250,000, about 300,000, about 350,000, about 400,000, about 450,000, about 500,000, about 550,000, about 600,000, about 650,000, about 700,000, about 750,000, about 800,000, about 850,000, about 900,000, about 950,000, about 1,000,000, about 2,000,000, about 3,000,000, about 4,000,000, about 5,000,000, about 6,000,000, about 7,000,000, about 8,000,000, about 9,000,000, about 10,000,000, about 15,000,000, about 20,000,000, about 25,000,000, about 30,000,000, about 35,000,000, about 40,000,000, about 45,000,000, about 50,000,000, or more than about 50,000,000 CD3⁺ and CD8⁺ T cells. In some embodiments, the composition comprises about 20% or greater, about 30% or greater, about 40% or greater, about 50% or greater, about 60% or greater, about 70% or greater, about 75% or greater, about 80% or greater, about 85% or greater, about 90% or greater, or about 95% or greater CD3⁺ and CD4⁺ T cells.

In some embodiments, the composition comprises T cells, wherein the T cells display minimal exhaustion markers. In further embodiments of the invention, the T cell composition comprises effector memory T cells with minimal exhaustion as measured by flow cytometry for cell surface markers for memory and exhaustion. In some embodiments, the exhaustion markers are selected from the group consisting of CD3, CD4, CD8, CD45RO, CD45RA, CD197, CD28, CD122, CD127, CD183, CD95, and CD62L.

In some embodiments, the composition comprises no or substantially no PD-1, CTLA4, LAG3 positive cells. In some embodiments, the composition comprises less than about 5%, less than about 4%, less than about 3%, less than about 2%, or less than about 1% PD-1, CTLA4, LAG3 positive cells.

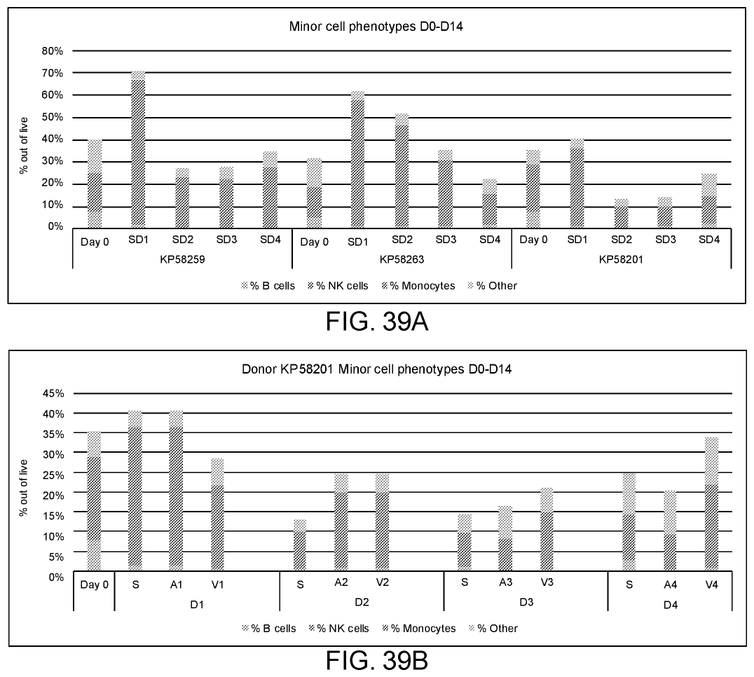

In some embodiments, the composition comprises T cells, wherein the T cells display high expression levels of lymphocyte homing and trafficking markers selected from one or more of CXCR3 (CD183), CCR7 (CD197) and the L-selectin CD62L. In some embodiments, the cells having high percentage of T effector memory cells which have higher levels of trafficking and homing capability. As the T cell product routinely achieves 60% effector memory T cells, this is the ideal phenotype for homing and extravasation. In other embodiments, the cells have 60% effector memory and 40% central/stem cell memory facilitating a durable response than other T cell products that are mostly T effector cells. As shown in FIG. 39 this percentage of short- and long-term memory can be modified by culture conditions.

In some embodiments, the composition comprises T cells, wherein the T cells display high antigen reactivity. In further embodiments of the invention, the T cell composition has high antigen reactivity as measured by ELISpot assay. In other embodiments the T cells kill targets which have been transfected with RNA or DNA. In other embodiments, the T cells release IFNγ or other cytokines in an Elisa assay after stimulation with RNA or DNA transfected targets.

In some embodiments, the T cells exhibit high antigen reactivity based on a production of one or more cytokines selected from IFNγ, TNFα, IL-2, and the cytolytic capacity indicator CD107a. In some embodiments such IFNγ and TNFα dual secretors modify the tumor microenvironment to be proinflammatory, encouraging immune cells to kill the cancer cells, epitope spreading and recruitment of other cells.

In some embodiments, the compositions are pharmaceutical compositions. In some embodiments, the compositions may further comprise one or more pharmaceutically acceptable carriers, excipients, preservatives, or a combination thereof. A “pharmaceutically acceptable carrier or excipient” refers to a pharmaceutically acceptable material, composition, or vehicle that is involved in carrying or transporting a compound of interest from one tissue, organ, or portion of the body to another tissue, organ, or portion of the body. For example, the carrier or excipient may be a liquid, diluent, solvent, or some combination thereof. Each component of the carrier or excipient must be “pharmaceutically acceptable” in that it must be compatible with the other ingredients of the formulation. It also must be suitable for contact with any tissue, organ, or portion of the body that it may encounter, meaning that it must not carry a risk of toxicity, irritation, allergic response, immunogenicity, or any other complication that excessively outweighs its therapeutic benefits. Suitable excipients include water, saline, dextrose, glycerol, or the like and combinations thereof. In some embodiments, compositions comprising host cells as disclosed herein further comprise a suitable infusion media.

### Methods of Treatment

In some embodiments, the present technology provides methods for preventing or treating cancer, comprising administering to a subject a composition comprising T cells with TCR or TCRs specific to changes in sequence and expression pattern associated with a person's cancer.

In some embodiments, the composition is administered by intravenous, intraarterial, intraperitoneal, intrapulmonary, intravascular, intramuscular, intratracheal, subcutaneous, intraocular, intrathecal, or transdermal administration.

In some embodiments, the dose of cells administered to the subject depends on the route of administration and/or the particular type and stage of cancer being treated. The number of cells administered to the subject should produce a therapeutic response against the cancer without resulting in severe toxicity or adverse events. In some embodiments, the subject is administered a therapeutically effective amount of the T cells. In some embodiments, the amount of T cells administered to the subject reduces the size of a tumor, decreases the number of cancer cells, or decreases the growth rate of a tumor by at least about 5%, about 10%, about 20%, about 30%, about 40%, about 50%, about 60%, about 70%, about 80%, about 90%, about 95%, or about 100%, as compared with the corresponding tumor size, number of cancer cells, or tumor growth rate in the same prior to treatment or as compared with the corresponding activity of a subject who has not received the treatment but has a tumor size, number of cancer cells, or tumor growth rate.

In some embodiments, the methods comprise administering a dose of T cells to a subject between about 1×10⁵ to about 5×10⁵, about 5×10⁵ to about 1×10⁶, about 1×10⁶ to about 2×10⁶, about 2×10⁶ to about 3×10⁶, about 3×10⁶ to about 4×10⁶, about 4×10⁶ to about 5×10⁶, about 5×10⁶ to about 6×10⁶, about 6×10⁶ to about 7×10⁶, about 7×10⁶ to about 8×10⁶, about 8×10⁶ to about 1×10⁸, about 1×10⁶ to about 3×10⁶, about 3×10⁶ to about 5×10⁶, about 5×10⁶ to about 7×10⁶, about 2×10⁶ to about 4×10⁶, about 1×10⁶ to about 5×10⁶, or about 5×10⁶ to about 1×10⁷, about 1×10⁵ to about 5×10¹², about 1×10⁵ to about 5×10¹¹, about 1×10⁵ to about 5×10¹⁰, about 1×10⁵ to about 5×10⁹, or about 1×10⁵ to about 5×10⁸ cells/kg of the subject's body weight.

In some embodiments, the methods comprise treating and/or preventing a cancer in a subject selected from the group consisting of non-small-cell lung cancer and cancers of the colon, bladder, pancreas, prostate, the hematological cancers DLBCL and AML, melanoma, and glioblastoma.

In some embodiments, the methods comprise treating lung cancer and/or glioblastoma by administering to a subject in need thereof a composition comprising T cells, wherein the T cells are reactive to one or more of cytomegalovirus antigen, pp65, Cancer/testis antigen 1 (NY-ESO-1), and Survivin, CEA, BING-4, Cyclin B1, 9D7, Ep-CAM, EphA3, Her2/neu, Telomerase, Mesothelin, SAP-1, Survivin, BAGE, CAGE, GAGE, MAGE, SAGE, XAGE, NYESO-1, PRAME, SSX-2, Melan-A/MART-1, Gp100, Tyrosinase, TRP1, TRP2, PSA, PSMA and MUC1.

In some embodiments, the methods comprise administering to a subject in need thereof a composition comprising T cells as a preventative measure against cancer activity. In some embodiments, the subject is administered the composition comprising T cells before or after a surgery in which a cancerous tumor is excised. Tumors can release cancer cells into the subject's blood stream and by administering the composition comprising T cells, the T cells can target and kill the released cancer cells, serving as a means of preventing the cancer activity in the blood stream. In some embodiments, T cells targeting the most common cancer mutations can be administered to a patient who does not have cancer so as to enhance immunosurveillance and prevent cancer.

In some embodiments, the methods further comprise treating prophylaxis in a subject with an elevated risk of cancer but who does not have cancer. Subjects have certain mutations such as BRCA1 & 2 mutations are predisposed to breast cancer, certain mutations in Li-Fraumeni syndrome predispose to leukemia, Lynch syndrome predisposes to colorectal and endometrial cancer, or other genes mutated in families who have predisposition to cancer. A cell vaccine that can fight against those mutations can be administered to increase immune surveillance and prevent the development of clinical cancer. Subjects having an elevated risk for cancer include immunosuppressed subjects. Administration of the composition to an immunosuppressed subject allows the T cells to remain in the subject's system and kill any cancer cells appearing thereby, preventing cancer. In some embodiments, administration of the composition to an immunosuppressed subject can further serve to activate an immune system of the subject thereby causing epitope proliferation. Activation of the immune system can serve to target antigens that were not contemplated by the T cell therapy. In some embodiments, administration of the composition increases proinflammatory cytokines such as IL-15, IL-7, IL-21, and/or IFNγ to help treat the prophylaxis.

In some embodiments, the subject has a high risk of developing a proliferative disease, and administration of the composition inhibits and/or delays development of the proliferative disease. A proliferative disease can include any group of disease characterized by non-cancerous conditions that may increase and/or give rise to cancer. Non-limiting examples of proliferative disease include but are not limited to atherosclerosis, rheumatoid arthritis, psoriasis, idiopathic pulmonary fibrosis, and scleroderma and cirrhosis of the liver.

In some embodiments, the patient is infused with the cells once. In some embodiments, the patient is infused with the cells once a month for at least about 1 year. This contrasts with other conventional therapies that require lymphodepletion with chemotherapeutics or whole-body irradiation and weeks of IL-2 treatment leading to flu-like symptoms. In some embodiments, the T cells are infused in two biweekly infusions in a 30-day treatment cycle. In some embodiments, the patient can receive additional infusions. In some embodiments, the patient blood is drawn before surgery or alternative primary therapy and the T cell product is administered during, immediately following or days, weeks or months following surgery or alternative primary therapy. In some embodiments, the T cell product can be administered as primary therapy, as single therapy or as therapy in combination with other therapies in patients with early, mid-stage or advanced cancer or infections.

In some embodiments, the methods of treating or preventing cancer can include combination therapy such as surgery, radiation therapy, gene therapy, immunotherapy, bone marrow transplantation, stem cell transplantation, hormone therapy, targeted therapy, cryotherapy, ultrasound therapy, photodynamic therapy, chemotherapy, or the like.

In some embodiments, the methods include preventing cancer in a patient having advanced cancer. In some embodiments, the methods include administering to a subject in need thereof a composition comprising T cells as a first line therapy. In yet another embodiment, the methods include administering a composition comprising T cells to the subject immediately prior to surgery, as the T cells will not deter healing like chemotherapy and radiation adjuvant therapy. In some embodiments, in the case of treating or preventing cancer in a subject having solid tumors, the T cells can be manufactured before the patient undergoes surgery.

In some embodiments, the methods include preventing cancer by using the compositions comprising T cells as a preventative vaccine. In some embodiments, the compositions comprising T cells used as a preventative vaccine against the most frequent neoantigens. In some embodiments, the patient is predisposed by certain mutations (e.g., BRCA in breast cancer).

In some embodiments, the present technology provides methods for treating a viral infection, comprising administering to a subject a composition comprising T cells with TCRs specific to a viral antigen. In some embodiments, the methods include treating a viral infection in a subject in need thereof, the method comprising administering to the subject the composition comprising T cells encoding and/or expressing a T cell receptor (TCR) that binds to a viral antigen associated with a virus, wherein the T cells are derived from the subject. In some embodiments, the viral antigen is a protein expressed by one or more of cytomegalovirus, Epstein-Barr virus, hepatitis B virus, human papillomavirus, adenovirus, herpes virus, human immunodeficiency virus, influenza virus, human respiratory syncytial virus, vaccinia virus, varicella-zoster virus, yellow fever virus, Ebola virus, coronavirus (e.g., SARS-CoV, MERS-CoV, SARS-CoV-2), Eastern equine encephalitis virus, Polyomavirus hominis1 (BKV), VP1, VP2, VP3, large T antigen, and small t antigen and Zika virus.

### Expression of Molecules

In some embodiments, the methods further include transfer of a gene into one or more T cells of the T cell composition. In these embodiments, the gene includes, but is not limited to, IL-2, IL-7, IL-12, IL-15, and IL-21.

In some embodiments, gene transfer was carried out using one or more expression vectors, including but not limited to plasmids or viral vectors such as lentiviral, adenoviral, or AAV vectors. In certain embodiments, gene transfer was coupled with clustered regularly interspaced short palindromic repeat (CRISPR)-mediated DNA editing. For example, one or more CRISPR components, including guide RNAs (gRNAs) and nuclease proteins, may be delivered in conjunction with a gene, for example to facilitate insertion of the gene into the cell's genome. In certain embodiments, gene transfer is carried out before the composition has been administered to a subject. In other embodiments, gene transfer is carried out after administration of the T cell composition.

In some embodiments, these methods can be combined with allogeneic T cell compositions. Without intending to be limited by any particular theory, allogeneic compositions have the advantage of being fully characterized before use and can be selected for generating a T cell composition effective against cancers present in various subjects. In some embodiments, blood isolated from a subject who does not have cancer can be subject to the methods of the present disclosure, such as those illustrated in FIGS. 1-3 depicting the mRNA T-cell production Process. In these embodiments, the neoantigen can be G12D, a common neoantigen across a plurality of cancers. After confirming that the cells detect and respond to G12D, single cells may be expanded using a polyclonal stimulatory antibody such as CD2/CD28/CD3. After a sufficient cell number has been reached, such as but not limited to 5×10⁴ T cells, each clonal population may be screened for G12D reactivity. G12D reactive cell populations can be further expanded by stimulation and banked for use with a cancer patient having the G12D mutation. Unlike autologous cells, allogeneic cells are not HLA matched, and subjects reject the cells. Thus, with today's allogeneic approaches, the patient must be immunosuppressed either by the cancer therapy or by active administration of immunosuppressive agents such as Cytoxan fludarabine or radiation before receiving an infusion of cells. This relegated allogeneic cell therapy to later stage patients who have failed multiple therapies is not practical for the therapy of viral infections other than those which are post transplanted due to the immunosuppressed state. Rather, patients transplanted with allogeneic CAR T must be preconditioned with Cytoxan, fludarabine and lymphodepletion in order for the allogeneic CAR T to demonstrate any efficacy. In contrast, the allogeneic T cells of the present disclosure can be used in an immunocompetent patient (e.g., a patient with Cancer or viral infections (such as Cov-2)).

In some embodiments, a patient diagnosed with cancer can be treated immediately with allogeneic cells having neoantigens or viral antigens associated with the patient's cancer prior to administration of a composition comprising T cells having one or more mutations associated with the patient's cancer. Such an allogeneic response could provide “conditioning” to prepare for the administration of the composting comprising T cells having one or more mutations associated with the patient's cancer. In some embodiments, treating a patient with allogenic cells can confer T cell memory more effectively after administration of the composition comprising autologous T cells responding to one or more mutations associated with the patient's cancer. In some embodiments, the patient does not require lymphodepletion or bone marrow conditioning prior to administration of the allogeneic cells.

In some embodiments, the allogeneic T cells are made to respond to one or more viral antigens. The allogenic T cell response can limit symptoms and infection by attacking infected cells. In some embodiments, the viral antigens are one or more COVID-19 antigens. Non-limiting examples of COVID-19 antigens include Nsp 6, Spike (S1, S2), N, M, N, 8, 3a, 7a. In some embodiments, the allogeneic T cells target only one of Nsp 6, Spike (S1, S2), N, M, 8, 3a, and 7a. In some embodiments, the allogenic cells can be used to target Eastern Equine Encephalitis or any acute viral infection.

In some embodiments, allogeneic cell lines can be produced from numerous donors using the mRNA or peptide T-cell production process so as to cover the highest frequency of HLA in the human population. In some embodiments, the allogenic cell lines are produced from at least about 10, about 11, about 12, about 13, about 14, about 15, about 20, about 30, about 40, about 50, about 60, about 70, about 80, about 90, about 100, or more. In some embodiments, the allogenic cell lines are derived from 15 donors and match 15% of the human population. In some embodiments, the allogenic cell lines are derived from about 60 to about 80 donors and match 90% of the human population. In some embodiments, PBMCs isolated by apheresis are used as a starting material for synthesis in large scale bioreactors (e.g., Grex 500). In some embodiments, a manifold in the final filling of bags with T cell product are used to produce multiple doses per batch to create a cell bank. In some embodiments, each batch of donor T cells is characterized to determine the MHC to which the T cell recognition of the antigen is restricted. In some embodiments, HLA locus specific (i.e.—HLA A, B, C, DR, DQ, DP) antibodies are used to identify HLA type and the antigen specificity of that HLA type for a given cell line In some embodiments, cell lines are rested for a partial match in a killing assay without peptide to reduce the chance of graft versus host disease.

In some embodiments, rejection of the allogeneic T cells in an immunocompetent host may be delayed by generating allogeneic cells specific to certain HLA combinations and after T cells responding to one or more mutations associated with the patient's cancer is produced, using gene editing technologies, e.g., CRISPR, to delete MHC class I molecules. In some embodiments, expression of beta 2 microglobulin can be inhibited by at least partially deleting the gene encoding beta 2 microglobulin. In some embodiments, CRISPR-based editing was used to remove Class I MHC in a step before the wash, fill and finish in bags for freezing of the final product. In some embodiments, CRISPR was used after second antigen specific stimulation but before the addition of anti-CD3, CD28, CD2 antibodies are added for polyclonal expansion. This approach can increase the survival and half-life of allogeneic T cells in the immunocompetent animal model and patient.

In another embodiment, allogeneic T cells can be prepared on-the-shelf and are ready for administration immediately. The major value of allogeneic, as opposed to autologous T cells, is that they can be prepared in advance and thus taken “off-the-shelf” to be used in therapy without waiting a production period of a few weeks and the ability to reduce the cost of goods by using a cell line for more than one patient.

In some embodiments, the half-life of allogeneic cells can be increased by the surface expression of PD-L1. PD-L1 can act to inhibit immune activation that results in the death of the expressing cell. In some embodiments, the methods include transfecting an mRNA encoding PD-L1 into the allogenic cells. In some embodiments, include transfecting an mRNA encoding PD-L1 into the allogenic cells prolongs the life of the allogenic cells leading to more effective treatment.

In some embodiments allogeneic cell products with the β2-microglobulin knockout administered to patients without the need for (or only needing only low levels) conditioning by chemotherapy, radiation or immunosuppressive agents.

In other embodiments, they can be administered to manage a patient before administration of an autologous T cell product.

In some embodiments allogeneic cell products with the β2-microglobulin knockout are administered to patients without the need for (or only needing only low levels) conditioning by chemotherapy, radiation or immunosuppressive agents having a longer half-life in the blood than those cells with wild type β2-microglobulin.

In some embodiments allogeneic cell products with the β2-microglobulin knockout demonstrate longer survival in the presence of partial MHC matched or fully mismatched T cells than those cells with wild type β2-microglobulin.

In some embodiments, graft-versus-host disease may be prevented by generating allogeneic cells specific to certain HLA combinations and/or using gene editing technologies, e.g., CRISPR, to delete MHC class I molecules. In some embodiments, expression of beta 2 microglobulin was inhibited by at least partially deleting the gene encoding beta 2 microglobulin, e.g., using CRISPR. In other embodiments, gene editing technologies was used to induce expression of a certain HLA molecule thereby resulting in a match to prevent graft versus host disease. These gene editing technologies include but are not limited to CRISPR, TALENs, Zinc fingers, Meganucleases and Sleeping Beauty.

In some embodiments, the methods further include allogeneic stem cell transplantation. Allogeneic transplantation includes transferring stem cells from a healthy person to a subject after chemotherapy and/or radiation. In some embodiments, the methods further comprise a multiplexed TCR T approach representing the TCR repertoire to tumor specific neoantigens. This method includes use of a single T cell dissection of germinal centers which have been created in tissue culture where the cells have been stimulated with a mixture of neoantigens by the methods described in this disclosure.

In some embodiments, stimulation is performed with neoantigens from the patients identified and stimulated with peptides, RNA or DNA encoding the neoantigens. As germinal center involves synapsing of a DC or other APC with a TCR specific for the presented antigen. Once the TCR recognizes the presented antigen, the T-cell will proliferate and generate a clump of cells around the APC. These are similar to a germinal center. In some embodiments, the single cell dissection can be combined with single cell sequencing to identify the TCRs present in the clump of activated cells. The sequences may be engineered into expression constructs, such as but not limited to, single constructs or multiple individual constructs. In some embodiments, the combined single construct or the multiple individual constructs may be delivered into a T cell. In some embodiments, the T cells can be grown as allogeneic cell lines or into an autologous T cell composition. In some embodiments, multiple TCRs that react to an antigen are combined and clonal selection is eliminated.

In some embodiments, the methods include T Cell Receptor Engineered-T Cell (TCR T)-based approaches, e.g., generating engineered T cells by identifying a TCR repertoire responding to neoantigens by TCR sequencing and transfecting said TCRs into a patient's T cells. In some embodiments, the disclosed process is performed using viral vectors such as lentiviral or retroviral vectors. In some embodiments, after simulation of the T cells, the methods include isolating a clump of cells and/or multiple clumps from a “germinal center” culture. In some embodiments, the methods then include diluting and transferring the cell plate (e.g., 96 well plate) such that each well has a single cell (e.g., Fluidigm C1 single cell sequencing 96 cells/run). In some embodiments, alternative single cell sequencing technologies are used.

In some embodiments, the methods include sequencing a single cell from the well plate. In some embodiments, a TCR α and β (or in alternative embodiment γ and δ) of the cell are sequenced. In some embodiments, CD8 and CD4 of RNA from the cell are sequenced. The method allows for identification of TCR chains for each cell as well as determination of whether the TCR is in a CD8 or a CD4 cell (e.g., seeing the neoantigen in the context of class I or Class II MHC). Unlike other methods that identify TCR one at a time, this method simultaneously identifies the entire TCR repertoire including relative frequency of each in the response to the patient specific neoantigens and does so with the proper TCR chains paired and associated with CD8 or CD4. In addition, TCRs have been through the patient's own positive and negative selection and are therefore not alloreactive.

In some embodiments, the methods further include expanding the TCRs by PCR followed by cloning into a suitable expression vector and then inserting the TCRs into CD8 or CD4 cells from the patient depending upon whether TCRs were in CD8 or CD4 cells. In some embodiments, the insertion is into an appropriate α, β, γ, or δ gene to replace the endogenous TCR gene. In some embodiments, the methods include inserting the TCRs into CD8 or CD4 cells using CRISPR-mediated DNA editing or viral mediated insertion, e.g., using a retroviral vector. In other embodiments, CRISPR was used to knockout the endogenous TCR from patient CD8 and CD4 cells before introducing the TCR identified. In some embodiments, the insertion replaces the T cells endogenous TCR gene.

In some embodiments, T cell is characterized by flow cytometry or other methods that allow for the identification of chain pairing with the original chain vs other endogenous TCR chains. In some embodiments, the genes for both chains are in the same construct. In yet another embodiment, the genes for each chain are in separate constructs. In some embodiments, the genes encoding TCR as or yb chain constant domains are modified to increase interchain disulfide bonding between the transfected chains to enhance association over random association with other endogenous TCR chains.

### Transient Modification of T Cells

The T cell product is designed to produce an effective anti-tumor T cell response that consists of cells capable of killing tumor cells and, upon binding to its target antigen, secretes cytokines capable of converting the tumor microenvironment (TME) to an inflammatory state. Specifically, IFN-γ and TNF-α, which are both produced by the T cell product may be sufficient to remodel the TME. However, in many cases it will be necessary and advantageous to improve the ability of T cells to remodel the TME.

The transient nature of the RNA prevents its integration into the genome. This is in strong contrast to previous approaches in T cell therapy which involved stable integration of DNA into the cell's genome using either viral vectors or CRISPR/Cas9 which has considerable safety concerns for transformation, leukemia, and lymphoma.

In some embodiments, the T cells can be modified by nucleofection, transfection with lipid nanoparticles, or by other means to introduce RNA into these cells to enhance survival, tumor homing, tumor cytotoxicity, or the T cell's ability to suppress, overcome or modify a tumor microenvironment.

In some embodiments, the introduced nucleic acids results in proinflammatory changes in the tumor microenvironment.

In some embodiments, the T cells are modified with one or more RNA construct encoding one or more pro-inflammatory protein.

In some embodiments, the T cells are modified with one or more RNA construct encoding one or more protein that blocks one or more immune checkpoint or anti-inflammatory receptor.

In some embodiments, RNA stability can be increased by modifying the 5′ or 3′ untranslated region (UTR) of the RNA such as modifying AU-rich elements (AREs) or CU-rich elements (CREs) which target RNA for rapid degradation. Some ARE and CRE elements normally found on the 3′ UTRs of mRNAs encoding cytokines confer specific mRNA stability in activated T cells. In a preferred embodiment, these 3′ UTRs including ARE or CRE are incorporated into the 3′UTR of the RNA synthesized according to our methods. Alternatively, mRNA can be stabilized using the 3′UTR from IL-2.

In some embodiments, mRNA can be stabilized by using an alternative cap or modified nucleotides such as such as 5methoxyUTP.

In some embodiments the introduced nucleic acids consist of linear RNA, circularized RNA, self-replicating RNA or chemically synthesized mRNAs, all with or without substituted or modified nucleosides in order to extend the half-life of the introduced RNA for prolonged expression.

In some embodiments the half-life of the RNA can be 3 to 5 days. In other embodiments, this half-life can be 1 to 3 weeks. In other embodiments, the half-life can be a month, 2 months, 3 months, 6 months, 12 months or anything in between.

In other embodiments, T cells nucleofected with such RNA have survival advantages in the tumor microenvironment.

In other embodiments, T cells nucleofected with such RNA act as delivery vehicles for such microenvironment modifying molecules across the tumor.

In other preferred embodiments, T cells reactive to multiple cancer antigens nucleofected with such RNA act as delivery vehicles for such microenvironment modifying molecules across the heterogenous tumor.

In other embodiments, T cells are reactive to 1 to 5, 5 to 10, 10 to 15, 15 to 20, 20 to 30, 30 to 40, 40 to 50, 50 to 100, 100 to 1000, 1000 to 5000, 5000 to 10000 or more antigens across the tumor.

In some embodiments, the T cells are modified with RNA during the last step of T cell stimulation and priming.

In other embodiments, the T cells are modified after CD3/CD28 or CD3/CD28/CD2 stimulation.

In other embodiments, the T cells are modified after antigen specific stimulation.

In other embodiments, the T cells are modified after incubation with cytokines.

In other embodiments the T cells are modified before freezing. In some embodiments, transfection includes using one or more of the following techniques: lipofectamine, lipid nanoparticles, electroporation, and nucleofection all described is the current disclosure.

In some embodiments, the T cells are modified with one or more RNA constructs encoding proteins that alter the tumor microenvironment, including but not limited to pro-inflammatory proteins, proteins that block anti-inflammatory proteins or pathways, and proteins that alter the extracellular matrix. In certain embodiments these proteins are antibodies or fusion proteins.

In some embodiments, the T cells are modified with one or more RNA constructs encoding one or more pro-inflammatory cytokine including but not limited to IL-2, IL-7, IL-12, IL-15, IL-18, IL-21, IFNα, IFNβ, IFNγ, and TNFα.

In other embodiments, the T cells are modified with one or more RNA constructs encoding one or more pro-inflammatory cytokine receptors including but not limited to IL-2R, IL-7R, IL-12R, IL-15R, IL-18R, IL-21R, IFNα receptor, IFNβ receptor, IFNγ receptor and TNFα receptors.

In other embodiments, the T cells are modified with one or more RNA constructs encoding both a pro-inflammatory cytokine and the corresponding cytokine receptor including but not limited to IL-2, IL-7, IL-12, IL-15, IL-18, IL-21, IFNα, IFNβ, IFNγ, and TNFα.

In some embodiments, the T cells are modified with one or more RNA constructs encoding one or more pro-inflammatory chemokines including but not limited to CCL2, CCL5, CCL9, CCL10, CCL11, CCL12, CCL13, CCL19, and CCL21.

In some embodiments, the T cells are modified with one or more RNA constructs encoding one or more pro-inflammatory chemokine receptors including but not limited to CCR2b, CCR2, CCR7, CXCR3, CXCR4.

In some embodiments, the T cells are modified with one or more RNA constructs encoding one or more pro-inflammatory costimulatory proteins including but not limited to CD28, CD40L, 4-1 BB, OX40, CD46, CD27, ICOS, HVEM, LIGHT, DR3, GITR, CD30, TIM1, SLAM, CD2, and CD226.

In some embodiments, the T cells are modified with one or more RNA constructs encoding for one or more fusion proteins. Negative immune checkpoint regulators including but not limited to PD-1, PD-L1, CTLA-4, Fas, FasL, LAG3, B7-1, B7-H1, CD160, BTLA, LAIR1, TIM3, 2B4, TIGIT, TGFβ receptor, IL-4 receptor, IL-10 receptor, and VEGF receptor can be converted to proinflammatory molecules through fusion of their extracellular domain with the intracellular signaling domain of a costimulatory protein including but not limited to CD28, CD40L, 4-1BB, OX40, CD46, CD27, ICOS, HVEM, LIGHT, DR3, GITR, CD30, TIM1, SLAM, CD2, and CD226 (for example the extracellular region of Fas fused with intracellular region of CD28, CD40L, 4-1 BB, OX40, or ICOS; VEGFR Fusion with 4-1 BB signaling domain; TGFβ Receptor fused with NGFR signaling domain; TGFβ Receptor fused with 4-1 BB stimulatory signal domain; TGFβ receptor fused with IL-12 receptor signaling domain; II-4 receptor fused to the β□domain of IL-2, IL-7 or 15, IL-7 receptor modified to constitutively activate STAT; IL-10 receptor fused with 41BB stimulatory domain; II-10 receptor fused with the IL-12 receptor signaling domain).

In another embodiment, the T cells are modified with one or more RNA constructs encoding one or more secreted antibody, single chain antibody (scFv), FAB fragment, or bispecific T cell engager to block targets including but not limited to αvβ8 integrin, PD-1, PD-L1, CTLA-4, Fas, FasL, LAG3, B7-1, B7-H1, CD160, BTLA, LAIR1, TIM3, 2B4, TIGIT, TGFβ, TGFβ receptor, IL-4 receptor, IL-10 receptor, and VEGF receptor.

In another embodiment, the T cells are modified with one or more RNA constructs encoding one or more enzyme that directly modifies the tumor microenvironment including but not limited to heparinase, catalase, matrix metalloproteinases, hyaluronidase, and RHEB.

In some embodiments, the T cell product can be used in conjunction with drugs that target PD-1 or PD-L1 to modify the T cells to be more resilient to the tumor microenvironment and/or to make the tumor microenvironment proinflammatory. In a preferred embodiment, T cells are modified with an RNA construct encoding a secreted inhibitor of PD-1 or PD-L1. This has the benefit of localized secretion into the tumor environment reducing off-target effects as compared to systemically administered anti-PD1 or anti-PD1 L antibodies.

In another embodiment, T cells are transiently transfected with RNA to increase viability after thawing. For example, T cells expressing IFNγ that are transiently transfected using RNA above have better viability post thaw than if they were not transfected with RNA. In some embodiments, post thaw viability can be measured immediately, 30 minutes, 1 hour, 2 hours, 3 hours, 4 hours, 5 hours, 6 hours, 7 hours, 8 hours, 9 hours, 10 hours, 11 hours, 12 hours, 24 hours, 36 hours, 48 hours, or 62 hours post thaw. In another embodiment, mRNA for IL-12 is loaded into the CD4+ cells to enhance the production of TH1 help post administration.

In some embodiments, the T cells can be modified to deliver a therapeutic payload specifically to the tumor. Delivery of molecules can either be persistent through continued release of molecules in route to the tumor or inducible by linking exocytosis of molecules to tumor specific antigen TCR activation. Molecules can be proteins such as diphtheria toxin, throbospondin-1, Fas, aquaporins, constituents of complement, collagenase, a disintegrin and metalloproteinase with thrombospondin motifs (ADAMTS), lumcorin, syndecan-1-2-3, cytokines including IL-2, IL-7, IL-12, IL-15, IL-18, IL-21, IFNα, IFNβ, IFNγ, TNFα. Small molecules such as the c-Met inhibitors include foretinib (XL880/GSK1363089), glesatinib (MGCD265), BMS-777607, and S49076, which target c-Met/RON/VEGFR-2/KIT/TIE2/PDGFR, c-Met/TIE2/RON/VEGFR-1/2/3, c-Met/RON/AXL, and c-Met/AXL/MER/FGFR, or EGFR inhibitors Erlotinib (Tarceva), Lapatinib (Tykerb), Osimertinib (Tagrisso), or VEGFR/FGFR/PDGFR inhibitors Brigatinib (Alunbrig), Axitinib (Inlyta), Pemigatinib (Pemazyre).

In some embodiments, RNA encoding one of these molecules is nucleofected into the T cells and then are cultured in a Rho kinase (ROCK) inhibitor immediately after transfection to improve viability.

In some embodiments, multiple of these molecules are nucleoporated into the T cells.

### Production and Co-Administration of T Cells Reactive to Viral Antigens or Neoantigens

In some embodiments, T cells targeting EBV+ lymphoma can be produced in two ways using the mRNA T cell process. First, using mRNA for EBV genes including the combination of LMP1, LMP2, and EBNA1.

In other embodiments, T cells targeting EBV+ lymphoma can be produced using the mRNA T cell process using mRNA for Second, using neoantigens to mutated endogenous genes specific to each patient's lymphoma.

In other embodiments, a combination therapy may be advantageous as it allows for further diversity of the lymphoma-specific T cell repertoire and makes it makes it more difficult for the lymphoma cells to acquire resistance be silencing any individual genes. T cells can be primed separately using dendritic cells (“DCs”) transfected with mRNA to EBV antigens and DCs transfected with neoantigens. These T cells would then be combined and administered together.

In another embodiment, dendritic cells are transfected with mRNA to both EBV antigens and neoantigens together using separate mRNAs or one mRNA containing both sets of antigens resulting in a single T cell product with specificity to both EBV antigens and neoantigens. This results in a broader antigen response by the T cells than each response individually.

### Protection Against Viral Infections

In some embodiments, the present technology provides methods for preventing or treating viral infections and/or diseases associated with viral infections, comprising administering to a subject a composition comprising T cells with TCR or TCRs specific to one or more viral antigens. In some embodiments, the methods include creating a protective immune response to a virus by administration of T cells or compositions comprising T cells disclosed herein to a patient who has not been exposed to the virus, or a patient who has been infected with the virus but has a low viral load (e.g., the virus has infected only a relatively small number of cells). In some embodiments, the virus is SARS-CoV-2, which causes COVID-19.

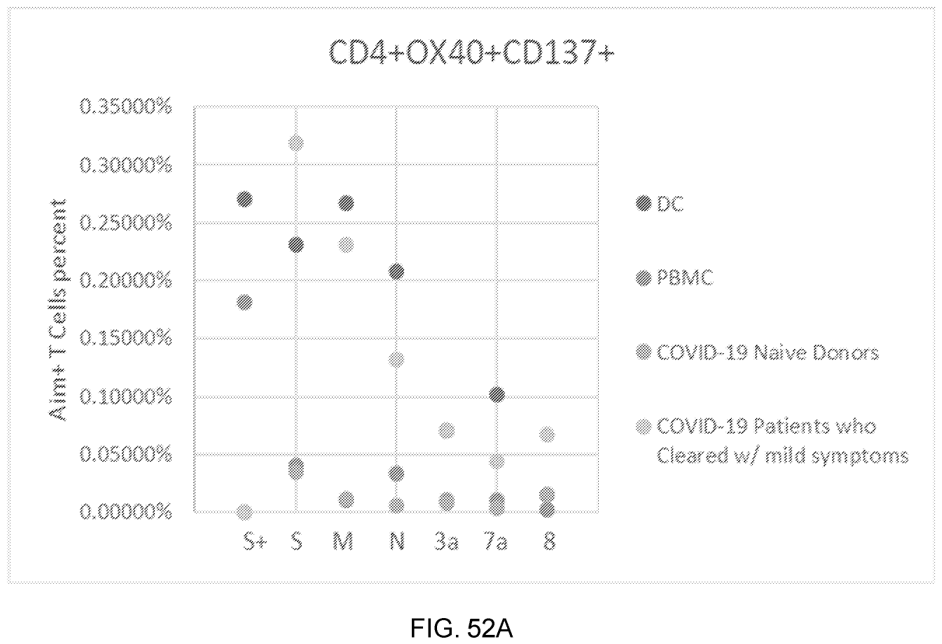

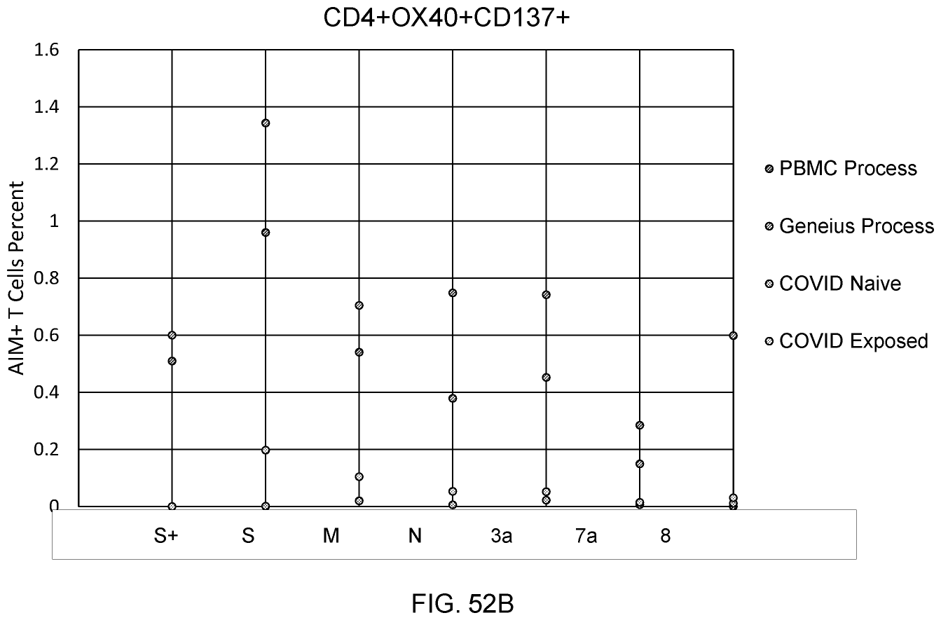

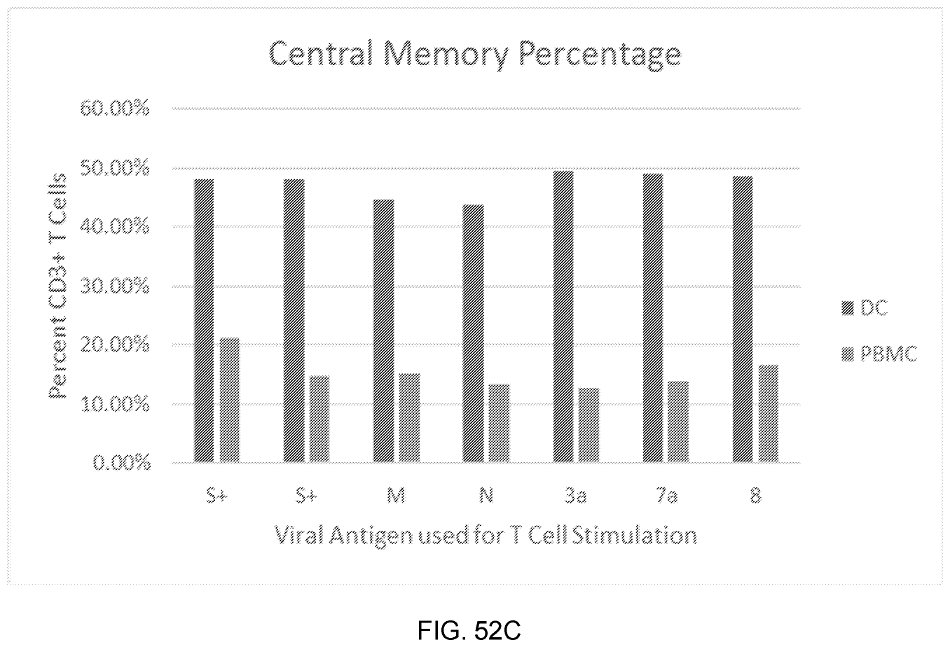

In some embodiments, the present technology provides methods for generating therapeutic T cells according to various embodiments disclosed herein that recognize one or more proteins associated with a virus (e.g., SARS-CoV-2) (see FIG. 52). In some embodiments, the method includes identification of viral antigens by comparing the immune responses towards viral proteins in subjects who have cleared the virus versus I subjects (e.g., those who have not been exposed to the virus) to identify immunodominant antigens and potential T cell targets. Studying the antigen stimulation and T cell (e.g., CD4⁺ and/or CD8⁺ T cells) populations from patients who have successfully recovered from a viral infection (e.g., COVID-19) or cleared the virus (e.g., SARS-CoV-2) with minimal side effects versus naïve donors (e.g., those who have not contracted COVID-19) allows for the identification of viral antigens associated with T cells that provided a successful immune response. In some embodiments, the identified viral antigens associated with COVID-19 are one or more of S, M, N, 3a, 7a, and 8 of the SARS-CoV-2 virus.

In some embodiments, the present technology provides methods for producing therapeutic T cells through the mRNA or peptide T cell production process as described herein that are specific to the identified viral antigens (see FIG. 52). In the embodiment of COVID-19, the method includes engineering a diverse T cell response from SARS-CoV-2 naïve, healthy donor's PBMCs to these antigens with a comparable response pattern to that of the T cells from patients who have cleared of COVID-19. Any of the DC-based T cell production methods disclosed herein can be used, for example, the autologous and allogeneic open system methods, closed system methods, pulse pool, or “pizza pie” methods. In some embodiments, peptides corresponding to one or more of the identified immunodominant viral antigens are synthesized, and the DCs are loaded with peptides or RNAs associated with all or portions of the viral antigens (e.g., SARS-CoV-2 S, M, N, 3a, 7a, and 8) and then used to prime the T cells. In some embodiments, DCs are loaded with peptides or RNAs associated with one antigen (e.g., SARS-CoV-2 S, M, N, 3a, 7a, or 8) at a time and then used to prime the T cells. In some embodiments, the DCs are made and loaded using a closed system as described. In other embodiments, the T cells are made in the “Pizza Pie” closed system where separate compartments or chambers in a compartment are loaded with different antigens. In some embodiments, the priming of the T cells can occur in these compartments or the DCs so made can be harvested and combined before contacting the T cells. In other embodiments, the DC's, priming and T cell production are performed in the same bioreactor which has a polystyrene surface on one side and a gas permeable membrane on the other. In any of these embodiments, the T cells targeting the one or more viral antigens are expanded and optionally subject to further processes as described herein.

In some embodiments, the T cells can be further modified to be compatible with prevalent MHC alleles in a certain population, e.g., in the U.S. population or a subgroup therein. In these embodiments, the modified T cells can be pre-made and administered to a subject in need thereof with MHC compatibility, thereby eliminating the need for immunosuppression and enabling those who are otherwise immunocompromised (e.g., cancer patients) to receive the T cells.

In some embodiments, the T cells can be further modified to have a longer half-life in the absence of conditioning/immunosuppression in the case of allogeneic cells or enhanced by the methods described in the case of autologous cells. In some embodiments modification of allogeneic T, this includes using CRISPR-based gene editing techniques to disrupt P2 microglobulin, resulting in the removal or reduction of HLA Class I levels. In some embodiments, the modifications increase the half-life of T cells to avoid rejection and/or graft versus host disease.

As SARS-CoV-2 Nsp6 is the most common HLA I restricted viral antigen in the case of patients who have successfully cleared the virus, in some cases the T cells have been expanded to respond to this antigen alone. In some embodiments, a bank of allogeneic T cells recognizing this antigen in the context of common MHCs can be made. In some embodiments, T cells are produced and/or collected from patients who test positive for SARS-CoV-2 and the cells are then shipped to the clinic, thawed, and infused into the patient using methods described in this disclosure. In some embodiments, this infusion can occur early in the disease, for example, within days of a positive test. In some embodiments, this treatment can rapidly eliminate the virally infected cells, thereby reducing the production of viral particles and limiting the severity of the disease while the patient's own immune system creates memory. In some embodiments, the treatment would limit the development of severe diseases and would reduce hospitalizations, the need for ventilators and ICU care, and/or death rate. In some embodiments, autologous cells to NSP 6 or the S, M, N, 3a, 7a, and 8 antigens could be manufactured from that patient's blood and administered following the prior administration of the allogeneic T cell product. In some embodiments, the T cells are made from a healthy donor's blood, manufactured to NSP 6 or the S, M, N, 3a, 7a, and 8 antigens and administered into the donor as a T cell vaccine to prevent the development of COVID-19 and/or the carrier state. This would be particularly helpful in first responders, healthcare workers, essential workers, members of the military or others living in close quarters, and individuals who are at high risk including but not limited to those over 60 years old, with diabetes, cancer, heart disease, or immunosuppression.

In some embodiments, the methods include stimulating T cells with COVID-19 specific viral antigens in the described DC-dependent processes and injecting the T cells into a patient to provide a durable T cell activity before the patient is exposed to or infected with SARS-CoV-2. In some embodiments, the T cell product is autologous and can be a T cell vaccine preventing infection of high-risk individuals or those who failed to produce a protective response following vaccination. In some embodiments, the T cell product is allogeneic and used to treat a patient infected with SARS-CoV-2. In some embodiments, the patient is HLA typed, and the T cell line reactive to SARS-CoV-2 is selected with at least one matched MHC. In some embodiments, the cells are then shipped to the patient and infused into the patient. In some embodiments, the T cell products can be modified as described in the methods in this application to be administered with little or no chemotherapy conditioning.

In some embodiments, the present technology provides methods for identifying viral antigen targets and generating a vaccine (e.g., an RNA vaccine) based on the identified viral antigens to be administered to a subject to afford immune-protection against the virus (e.g., SARS-CoV-2). In some embodiments, the viral antigens can be identified by comparing the clearance response and the naïve response associated with the virus as described herein. RNA vaccines can be generated by synthesizing an RNA construct encoding one or more of the identified viral antigens. In some embodiments, the synthesized RNA is further purified, for example, with poly-thymidine coated beads, prior to vaccine production. In some embodiments, the virus is SARS-CoV-2, and the identified immunodominant viral antigens include S, M, N, 3a, 7a, and 8 of the SARS-CoV-2 virus. In some embodiments, an RNA vaccine against the SARS-CoV-2 virus is generated by synthesizing an RNA construct corresponding to one or more of the viral antigens selected from S, M, N, 3a, 7a, and 8. In some embodiments, an RNA vaccine against the SARS-CoV-2 virus is generated by synthesizing an RNA construct corresponding to all of the viral antigens S, M, N, 3a, 7a, and 8. Without wishing to be bound by a particular theory, generating an RNA vaccine targeting all identified immunodominant viral antigens (e.g., S, M, N, 3a, 7a, and 8) may be advantageous because it would mitigate the possibility of reduced vaccine efficacy against viral mutations.

In some embodiments, the RNA vaccine targeting a virus (e.g., SARS-CoV-2) according to various embodiments disclosed herein can be administered as a stand-alone therapy or in conjunction with the autologous adoptive T cell therapy as described herein for increased efficacy of the therapy. In some embodiments, the RNA vaccine can be used when multiple viral antigens are used with a “linker”. In some embodiments, the RNA vaccine can be used when multiple viral antigens are used with a “linker” and/or scrambled sequences to limit the chance of creating a pseudovirus.

In some embodiments, when used in combination, the RNA vaccine can induce in vivo T-cell responses either by priming the collected PBMCs against the antigens encoded by the RNA vaccine or by boosting the responses of adopted T-cells in vivo. The boost can occur by two mechanisms, either by re-stimulation of adopted T-cells that are known to have a previous response to encoded antigens or by generation of endogenous immune responses that not previously been known to be responsive in the adopted T-cells.

In some embodiments, the methods according to various embodiments of the present technology can be applied to afford protection to other viruses and infections. In some embodiments, the one or more target antigens comprises polypeptides derived from one or more target viral antigens. In some embodiments, the one or more target antigens comprises RNAs or DNAs that encode peptides or proteins from one or more target viral antigens. In some embodiments, the target antigen(s) is/are a protein expressed by one or more of cytomegalovirus, Epstein-Barr virus, hepatitis B virus, hepatitis C virus, hepatitis D virus, human papillomavirus, adenovirus, herpes virus, human immunodeficiency virus, influenza virus, human respiratory syncytial virus, vaccinia virus, varicella-zoster virus, yellow fever virus, Ebola virus, Dengue virus, coronavirus (e.g., SARS-CoV, MERS-CoV, SARS-CoV-2), Eastern equine encephalitis virus, BKV, and Zika virus. In some embodiments, the target antigen(s) include but are not limited to a protein expressed by one or more of the virus or bacteria associated smallpox, Ebola, Marburg, with anthrax, plague, brucellosis, glanders, melioidosis, botulism, and tuberculosis.

In some embodiments, a closed culture system could be used to differentiate and mature dendritic cells (“DCs”), deliver mRNA and/or peptides to the DCs for the processing and presentation to T-cells then the expansion of T-cells to form the composition. In some embodiments, the closed culture system is composed of polystyrene, a silicone membrane for gas exchange, and a polystyrene and/or polypropylene base for the support, protection, and openings for gas exchange. A nonlimiting exemplary cassette is described in FIG. 33D. The cassette will 1) Adhere monocytes to the polystyrene, followed by differentiation and maturation to DCs. 2) The delivery of mRNA by liposome vesicles to matured DCs for mRNA processing and presentation. 3) Dendric cell processing and presentation of 15-27mer peptides. 4) T-cell stimulation by presentation and costimulatory activity of the DC. 5) The expansion of the T-cells to increase the cell numbers. In some embodiments, lipid composition of lipid-based nanoparticles used for the mRNA delivery may contain single and/or multiple lipid groups within the formulation. The lipid groups include:
1) Cationic lipids: DOSPA 2,3-dioleyloxy-N-[2-(sperminecarboxamido)ethyl]-N,N-dimethyl-1-propanaminium trifluoroacetate, DOTMA 1,2-di-O-octadecenyl-3-trimethyl ammonium propane, DOTAP 1,2-Dioleoyl-3-trimethy alammoniumpropane, DC-Cholesterol 3β-[N—(N′,N′-dimethylaminoethane)-carbamoyl] cholesterol,
2). Ionizable lipids: SM-102 9-Heptadecanyl 8-((2-hydroxyethyl)(6-oxo-6-(undecyloxy)hexyl)amino)octanoate, ALC-0315 4-hydroxybutyl)azanediyl)bis(hexane-6,1-diyl)bis(2-hexyldecanoate), DLin-MC3-DMA (6Z,9Z,28Z,31Z)-heptatriaconta-6,9,28,31-tetraen-19-yl 4-(dimethylamino) butanoate, DODMA 1,2-Dioleyloxy-3-dimethylamino propane.
3) Helper lipids: Cholesterol (1R,3aS,3bS,7S,9aR,9bS,11aR)-9a,11a-Dimethyl-1-[(2R)-6-methylheptan-2-yl]-2,3,3a,3b,4,6,7,8,9,9a,9b,10,11,11a-tetradecahydro-1H-cyclopenta[a]phenanthren-7-ol. DSPC 1,2-distearoyl-sn-glycero-3-phosphocholine, DOPE 1,2-Dimyristoyl-sn-glycerophosphoethanolamine.
4) Stealth lipids: PEG2000-DMG, (R)-2,3-bis(myristoyloxy)propyl-1-(methoxy poly(ethylene glycol) 2000) carbamate and ALC-0159 2-[(polyethylene glycol)-2000]-N,N-ditetradecylacetamide.
5) In some embodiments, mannose carbohydrates will be included to the formulation. The addition of mannose carbohydrates assists in the binding to the DCs by using the mannose receptor on the DCs.

### EXAMPLES

The capacity to generate large numbers of functional T cells without genetic engineering has tremendous clinical applications and can fundamentally change the way in which cancer is currently treated. The following examples demonstrate methods of generating functional T cells without using genetic engineering by transient transfection of dendritic cells (DCs) with mRNA encoding antigen or added peptides. Without intending to be limited by any particular theory, the use of an mRNA construct shortens the timeframe for T cell production and provides an approach for expanding autologous or allogeneic T cells targeted specifically to a patient's cancer with minimal risk to the patient and potential for lasting remission. Further disclosures demonstrate improvements to T-cell product manufacturing and other applications of mRNA technology in T-cell therapies. Further disclosures also demonstrate how to produce TCR T transfection at the level of the T cell repertoire.

Examples 1-7 describe steps of the methods used to produce the autologous T cells with a summary of the methods provided in the exemplary flow diagram shown in FIGS. 1-2. Examples 8-9 describes further uses of mRNA technology. Example 10 and 11 is an adaptation of methods in Examples 1-7.

### Example 1: Cell Collection

A patient is diagnosed with stage 4 colon cancer and before beginning chemotherapy or tumor excision, the patient goes has 100 to 500 mL of blood drawn with heparin as the anticoagulant at an outpatient clinic. 10 mL of whole blood is sent to Guardant Health for their liquid biopsy OMNI sequencing panel sequencing panel which typically takes a week to complete. The remaining 490 mL of whole blood is sent for processing where centrifugation using Ficoll gradient is carried out to isolate PBMCs within 2-3 hours. The cells are moved into CryoStor® and a container suitable for a controlled rate freezer such as an infusion bag or vials and frozen using the manufacturer's protocol for freezing T cells down to −80° C. or liquid nitrogen −150° C. and stored until a sequencing report is generated from a liquid biopsy test.

### Example 2: Cancer Genomics

The following example demonstrates the ability to detect all cancer mutations/rearrangements, referred to as neoantigens, present in a patient from a single blood draw and to receive results within a week, allowing for fully personalized cancer treatment. In developing this treatment model, typical mutations of a cancer genome were first identified and then adapted for use with the disclosed process of growing targeted T cells by the introduction of autologous dendritic cells (DCs).

Initial efforts included conducting an analysis on the most common mutations found in cancer in order to model the types of targets for a typical cancer treatment. The analysis included using The Cancer Genome Atlas (TCGA), which is a curated collection of genome sequencing, next generation sequencing, and RNA sequencing of various types of tumors. TCGA draws from well over 10,000 patients and is, therefore, representative of neoantigens in cancer patient populations. Using a process of elimination (FIG. 3), the most common oncogenic mutations were identified. The oncogenic mutations are significant as they are most likely founder antigens (i.e., the first mutation that occurs in the cell as the cells become cancerous) and therefore should be common to all cancer cells. Additionally, these mutations tend to be critical for the growth of cancer cells, and, if the treatment eliminates these mutations from the body, then the cancer may lose its propagation potential. While each type of cancer has a set of mutations that are commonly associated with that cancer, there are some mutations common to all cancer types. To ensure that every cancer is represented, and not just a common mutation in a common form of cancer, each cancer type was analyzed independently.

This analysis provided gene frequency, site frequency, identification of individual mutation frequency, and eliminated mutations not believed to be oncogenic (FIG. 3). All data was drawn from TCGA (www.genome.gov/Funded-Programs-Projects/Cancer-Genome-Atlas). The database also provided information on whether a mutation is a hotspot or believed to be oncogenic. Data for each cancer type, gene, and mutation was accessed by using the filter sets to specify cancer type, frequency of mutated genes, and frequency of mutation sites. The data was aggregated manually in Excel and further analysis was done to select mutations that could be functionally significant with regards to oncogenic potential. Site frequencies, not sample number, was used to rank all the mutations found using the algorithm outlined in FIG. 3.

From this analysis, the most common genes and mutations for colon cancer, lung cancer, pancreatic cancer, diffuse large B cell lymphoma (DLBCL), acute myeloid leukemia (AML), melanoma, bladder cancer, and glioblastoma were determined (Tables 1-8).

Table 1 shows the genes and corresponding mutations associated with colon cancer, with the most represented determined to be KRAS G12, KRAS G13, and BRAF V600E.

TABLE 1
Cancer Mutations Associated with Colon Cancer

| | | | # | | | | | |
|---|---|---|---|---|---|---|---|---|
| | | # | Samples | | | | | |
| | | Patient | with this | | | Mutation | Functionally | |
| Gene | Cancer | samples | mutation | Frequency | Mutation | type | significant? | Hotspot? |
| KRAS | Colorectal | 2453 | 279 | 11.4% | G12D | Missense | Oncogenic | Y |
| BRAF | Colorectal | | 250 | 10.2% | V600E | Missense | Oncogenic | Y |
| KRAS | Colorectal | | 198 | 8.1% | G12V | Missense | Oncogenic | Y |
| KRAS | Colorectal | | 194 | 7.9% | G13D | Missense | Oncogenic | Y |
| TP53 | Colorectal | 2453 | 168 | 6.8% | R175H | Missense | Oncogenic | Y |
| PIK3CA | Colorectal | | 122 | 5.0% | E545K | Missense | Oncogenic | Y |
| TP53 | Colorectal | | 85 | 3.5% | R282W | Missense | Likely | Y |
| | | | | | | | Oncogenic | |
| TP53 | Colorectal | | 78 | 3.2% | R248Q | Missense | Likely | Y |
| | | | | | | | Oncogenic | |
| PIK3CA | Colorectal | | 77 | 3.1% | H1047R | Missense | Oncogenic | Y |
| TP53 | Colorectal | | 76 | 3.1% | R273H | Missense | Oncogenic | Y |
| KRAS | Colorectal | | 76 | 3.1% | G12C | Missense | Oncogenic | Y |
| TP53 | Colorectal | | 75 | 3.1% | R273C | Missense | Likely | Y |
| | | | | | | | Oncogenic | |
| PIK3CA | Colorectal | | 73 | 3.0% | E542K | Missense | Oncogenic | Y |
| TP53 | Colorectal | | 62 | 2.5% | R248W | Missense | Likely | Y |
| | | | | | | | Oncogenic | |
| KRAS | Colorectal | | 48 | 2.0% | G12A | Missense | Oncogenic | Y |
| FBXW7 | Colorectal | | 45 | 1.8% | R465C | Missense | Oncogenic | Y |
| TP53 | Colorectal | | 43 | 1.8% | G245S | Missense | Oncogenic | Y |
| SMAD4 | Colorectal | | 39 | 1.6% | R361H | Missense | Likely | Y |
| | | | | | | | Oncogenic | |
| FBXW7 | Colorectal | | 37 | 1.5% | R465H | Missense | Oncogenic | Y |
| KRAS | Colorectal | | 36 | 1.5% | G12S | Missense | Oncogenic | Y |
| PIK3CA | Colorectal | | 30 | 1.2% | R88Q | Missense | Oncogenic | Y |
| FBXW7 | Colorectal | | 26 | 1.1% | R505C | Missense | Oncogenic | Y |
| SMAD4 | Colorectal | | 24 | 1.0% | R361C | Missense | Likely | Y |
| | | | | | | | Oncogenic | |

TABLE 1
Cancer Mutations Associated with Colon Cancer (cont'd.)

| WT 27 mer | Mut 27 mer |
|---|---|
| MTEYKLVVVGAGGVGKSALTIQLIQNH | MTEYKLVVVGADGVGKSALTIQLIQNH |
| DLTVKIGDFGLATVKSRWSGSHQFEQL | DLTVKIGDFGLATEKSRWSGSHQFEQL |
| MTEYKLVVVGAGGVGKSALTIQLIQNH | MTEYKLVVVGAVGVGKSALTIQLIQNH |
| MTEYKLVVVGAGGVGKSALTIQLIQNH | MTEYKLVVVGAGDVGKSALTIQLIQNH |
| IYKQSQHMTEVVRRCPHHERCSDSDGL | IYKQSQHMTEVVRHCPHHERCSDSDGL |
| KAISTRDPLSEITEQEKDFLWSHRHYC | KAISTRDPLSEITKQEKDFLWSHRHYC |
| SFEVRVCACPGRDRRTEEENLRKKGEP | SFEVRVCACPGRDWRTEEENLRKKGEP |
| NYMCNSSCMGGMNRRPILTIITLEDSS | NYMCNSSCMGGMNQRPILTIITLEDSS |
| EALEYFMKQMNDAHHGGWTTKMDWIFH | EALEYFMKQMNDARHGGWTTKMDWIFH |
| SSGNLLGRNSFEVRVCACPGRDRRTEE | SSGNLLGRNSFEVHVCACPGRDRRTEE |
| MTEYKLVVVGAGGVGKSALTIQLIQNH | MTEYKLVVVGACGVGKSALTIQLIQNH |
| SSGNLLGRNSFEVRVCACPGRDRRTEE | SSGNLLGRNSFEVCVCACPGRDRRTEE |
| EQLKAISTRDPLSEITEQEKDFLWSHR | EQLKAISTRDPLSKITEQEKDFLWSHR |
| NYMCNSSCMGGMNRRPILTIITLEDSS | NYMCNSSCMGGMNWRPILTIITLEDSS |
| MTEYKLVVVGAGGVGKSALTIQLIQNH | MTEYKLVVVGAAGVGKSALTIQLIQNH |
| ECIHTLYGHTSTVRCMHLHEKRVVSGS | ECIHTLYGHTSTVCCMHLHEKRVVSGS |
| IHYNYMCNSSCMGGMNRRPILTIITLE | IHYNYMCNSSCMGSMNRRPILTIITLE |
| VTVDGYVDPSGGDRFCLGQLSNVHRTE | VTVDGYVDPSGGDHFCLGQLSNVHRTE |
| ECIHTLYGHTSTVRCMHLHEKRVVSGS | ECIHTLYGHTSTVHCMHLHEKRVVSGS |
| MTEYKLVVVGAGGVGKSALTIQLIQNH | MTEYKLVVVGASGVGKSALTIQLIQNH |
| QEAEREEFFDETRRLCDLRLFQPFLKV | QEAEREEFFDETRQLCDLRLFQPFLKV |
| QCLHVLMGHVAAVRCVQYDGRRVVSGA | QCLHVLMGHVAAVCCVQYDGRRVVSGA |
| VTVDGYVDPSGGDRFCLGQLSNVHRTE | VTVDGYVDPSGGDCFCLGQLSNVHRTE |

Table 2 shows the genes and corresponding mutations associated with lung cancer, with the most represented determined to be KRAS G12 and EGFR E760_A750del L858R.

TABLE 2
Cancer Mutations Associated with Lung Cancer

| | | | # | | | | | |
|---|---|---|---|---|---|---|---|---|
| | | # | Samples | | | | | |
| | | Patient | with this | | | Mutation | Functionally | Hotspot? |
| Gene | Cancer | samples | mutation | Frequency | Mutation | type | significant? | |
| KRAS | Lung | 4006 | 376 | 9.4% | G12C | Missense | Oncogenic | Y |
| KRAS | Lung | | 155 | 3.9% | G12V | Missense | Oncogenic | Y |
| EGFR | Lung | | 141 | 3.5% | E746_A750del | In-frame | Oncogenic | Y |
| | | | | | | deletion | | |
| EGFR | Lung | | 120 | 3.0% | L858R | Missense | Oncogenic | Y |
| KRAS | Lung | | 109 | 2.7% | G12D | Missense | Oncogenic | Y |
| TP53 | Lung | | 60 | 1.5% | R158L | Missense | Likely | Y |
| | | | | | | | Oncogenic | |
| TP53 | Lung | | 60 | 1.5% | V157F | Missense | Likely | Y |
| | | | | | | | Oncogenic | |
| TP53 | Lung | 4006 | 53 | 1.3% | R273L | Missense | Likely | Y |
| | | | | | | | Oncogenic | |
| EGFR | Lung | | 52 | 1.3% | T790M | Missense | Oncogenic | Y |
| PIK3CA | Lung | | 50 | 1.2% | E545K | Missense | Oncogenic | Y |
| KRAS | Lung | | 39 | 1.0% | G13C | Missense | Oncogenic | Y |

TABLE 2
Cancer Mutations Associated with Lung Cancer (cont'd.)

| WT 27 mer | Mut 27 mer |
|---|---|
| MTEYKLVVVGAGGVGKSALTIQLIQNH | MTEYKLVVVGACGVGKSALTIQLIQNH |
| MTEYKLVVVGAGGVGKSALTIQLIQNH | MTEYKLVVVGAVGVGKSALTIQLIQNH |
| EGEKVKIPVAIKELREATSPKANKEILDE | EGEKVKIPVAIKE-ATSPKANKEILDE |
| VKTPQHVKITDFGLAKLLGAEEKEYHA | VKTPQHVKITDFGQAKLLGAEEKEYHA |
| MTEYKLVVVGAGGVGKSALTIQLIQNH | MTEYKLVVVGADGVGKSALTIQLIQNH |
| LWVDSTPPPGTRVRAMAIYKQSQHMTE | LWVDSTPPPGTRVLAMAIYKQSQHMTE |
| QLWVDSTPPPGTRVRAMAIYKQSQHMT | QLWVDSTPPPGTRFRAMAIYKQSQHMT |
| SSGNLLGRNSFEVRVCACPGRDRRTEE | SSGNLLGRNSFEVLVCACPGRDRRTEE |
| LLGICLTSTVQLITQLMPFGCLLDYVR | LLGICLTSTVQLIMQLMPFGCLLDYVR |
| KAISTRDPLSEITEQEKDFLWSHRHYC | KAISTRDPLSEITKQEKDFLWSHRHYC |
| MTEYKLVVVGAGGVGKSALTIQLIQNH | MTEYKLVVVGAGCVGKSALTIQLIQNH |

Table 3 shows the genes and corresponding mutations associated with pancreatic cancer, with the most represented determined to be KRAS G12.

TABLE 3
Cancer Mutations Associated with Pancreatic Cancer

| | | | # | | | | | |
|---|---|---|---|---|---|---|---|---|
| | | # | Samples | | | | | |
| | | Patient | with this | | | Mutation | Functionally | |
| Gene | Cancer | samples | mutation | Frequency | Mutation | type | significant? | Hotspot? |
| KRAS | Pancreatic | 1021 | 263 | 25.8% | G12D | Missense | Oncogenic | Y |
| KRAS | Pancreatic | | 220 | 21.5% | G12V | Missense | Oncogenic | Y |
| KRAS | Pancreatic | | 118 | 11.6% | G12R | Missense | Oncogenic | Y |
| TP53 | Pancreatic | | 26 | 2.5% | R175H | Missense | Oncogenic | Y |
| GNAS | Pancreatic | | 19 | 1.9% | R201C | Missense | Oncogenic | Y |
| TP53 | Pancreatic | | 15 | 1.5% | R282W | Missense | Oncogenic | Y |
| TP53 | Pancreatic | | 14 | 1.4% | R273H | Missense | Oncogenic | Y |
| ZNF814 | Pancreatic | | 14 | 1.4% | A337V | Missense | Oncogenic | Y |
| TP53 | Pancreatic | | 13 | 1.3% | R248Q | Missense | Oncogenic | Y |
| TP53 | Pancreatic | | 11 | 1.1% | R273C | Missense | Oncogenic | Y |

TABLE 3
Cancer Mutations Associated with Pancreatic Cancer (cont'd.)

| WT 27 mer | Mut 27 mer |
|---|---|
| MTEYKLVVVGAGGVGKSALTIQLIQNH | MTEYKLVVVGADGVGKSALTIQLIQNH |
| MTEYKLVVVGAGGVGKSALTIQLIQNH | MTEYKLVVVGAVGVGKSALTIQLIQNH |
| MTEYKLVVVGAGGVGKSALTIQLIQNH | MTEYKLVVVGARGVGKSALTIQLIQNH |
| IYKQSQHMTEVVRRCPHHERCSDSDGL | IYKQSQHMTEVVRHCPHHERCSDSDGL |
| ADYVPSDQDLLRCRVLTSGIFETKFQV | ADYVPSDQDLLRCCVLTSGIFETKFQV |
| SFEVRVCACPGRDRRTEEENLRKKGEP | SFEVRVCACPGRDWRTEEENLRKKGEP |
| SSGNLLGRNSFEVRVCACPGRDRRTEE | SSGNLLGRNSFEVHVCACPGRDRRTEE |
| YECGECGKSFSKYASFSNHQRVHTEKK | YECGECGKSFSKYVSFSNHQRVHTEKK |
| NYMCNSSCMGGMNRRPILTIITLEDSS | NYMCNSSCMGGMNQRPILTIITLEDSS |
| SSGNLLGRNSFEVRVCACPGRDRRTEE | SSGNLLGRNSFEVCVCACPGRDRRTEE |

Table 4 shows the genes and corresponding mutations associated with DLBCL, with the most represented determined to be MYD88, L256P, and EZH2 Y641.

TABLE 4
Cancer Mutations Associated with DLBCL Cancer

| | | | # | | | | | |
|---|---|---|---|---|---|---|---|---|
| | | | Samples | | | | | |
| | | # Patient | with this | | | Mutation | Functionally | |
| Gene | Cancer | samples | mutation | Frequency | Mutation | type | significant? | Hotspot? |
| MYD88 | DLBCL | 1266 | 138 | 10.9% | L265P | Missense | Likely | Y |
| | | | | | | | Oncogenic | |
| EZH2 | DLBCL | 1266 | 44 | 3.5% | Y641N | Missense | Yes, in domain | Y |
| EZH2 | DLBCL | 1266 | 33 | 2.6% | Y641F | Missense | Yes, in domain | Y |
| MYD88 | DLBCL | 1266 | 26 | 2.1% | S243N | Missense | Oncogenic | Y |
| PIM1 | DLBCL | 1266 | 23 | 1.8% | E135K | Missense | No | N |
| PIM1 | DLBCL | 1266 | 23 | 1.8% | G28D | Missense | Likely | Y |
| | | | | | | | Oncogenic | |
| PIM1 | DLBCL | 1266 | 22 | 1.7% | L184F | Missense | No | N |
| CD79B | DLBCL | 1266 | 22 | 1.7% | Y196H | Missense | Yes, in domain | Y |
| PIK3CD | DLBCL | 1266 | 22 | 1.7% | R38C | Missense | No | N |
| PIM1 | DLBCL | 1266 | 20 | 1.6% | S97N | Missense | Likely | Y |
| | | | | | | | Oncogenic | |
| MTOR | DLBCL | 1266 | 17 | 1.3% | E25336A | Missense | No | N |
| BCL2 | DLBCL | 1266 | 16 | 1.3% | A131V | Missense | Likely | Y |
| | | | | | | | Oncogenic | |

TABLE 4
Cancer Mutations Associated with DLBCL Cancer (cont'd.)

| Gene | WT 27 mer | Mut 27 mer |
|---|---|---|
| MYD88 | FALSLSPGAHQKRLIPIKYKAMKKEFP | FALSLSPGAHQKRPIPIKYKAMKKEFP |
| EZH2 | IKDPVQKNEFISEYCGEIISQDEADRR | IKDPVQKNEFISENCGEIISQDEADRR |
| EZH2 | IKDPVQKNEFISEYCGEIISQDEADRR | IKDPVQKNEFISEFCGEIISQDEADRR |
| MYD88 | RRMVVVVSDDYLQSKECDFQTKFALSL | RRMVVVVSDDYLQNKECDFQTKFALSL |
| PIM1 | — | — |
| PIM1 | — | — |
| PIM1 | — | — |
| CD79B | — | — |
| PIK3CD | — | — |
| PIM1 | — | — |
| MTOR | — | — |
| BCL2 | — | — |

Table 5 shows the genes and corresponding mutations associated with AML, with the most represented determined to be FLT3 D835.

TABLE 5
Cancer Mutations Associated with AML

| | | | # | | | | | |
|---|---|---|---|---|---|---|---|---|
| | | | Samples | | | | | |
| | | # Patient | with this | | | Mutation | Functionally | |
| Gene | Cancer | samples | mutation | Frequency | Mutation | type | significant? | Hotspot? |
| FLT3 | AML | 200 | 11 | 6% | D835Y | Missense | Oncogenic | Y |
| FLT3 | AML | 200 | 3 | 2% | D835E | Missense | Oncogenic | Y |
| FLT3 | AML | 200 | 2 | 1% | D835H | Missense | Oncogenic | Y |
| NPM1 | AML | 200 | 52 | 26% | W288Cfs*12 | FS ins | Oncogenic | Y |
| DNMT3A | AML | 200 | 21 | 11% | R882H | Missense | Oncogenic | Y |
| DNMT3A | AML | 200 | 7 | 4% | R882C | Missense | Oncogenic | Y |
| IDH2 | AML | 200 | 17 | 9% | R140Q | Missense | Oncogenic | Y |
| IDH1 | AML | 200 | 12 | 6% | R132C | Missense | Oncogenic | Y |
| NRAS | AML | 200 | 2 | 1% | Q61K | Missense | Oncogenic | Y |
| NRAS | AML | 200 | 5 | 3% | G13D | Missense | Oncogenic | Y |
| NRAS | AML | 200 | 4 | 2% | G12D | Missense | Oncogenic | Y |
| KIT | AML | 200 | 5 | 3% | D816Y | Missense | Oncogenic | Y |

TABLE 5
Cancer Mutations Associated with AML (cont'd.)

| WT 27 Mer | Mutant 27 mer |
|---|---|
| GKVVKICDFGLARDIMSDSNYVVRGNA | GKVVKICDFGLARYIMSDSNYVVRGNA |
| GKVVKICDFGLARDIMSDSNYVVRGNA | GKVVKICDFGLAREIMSDSNYVVRGNA |
| GKVVKICDFGLARDIMSDSNYVVRGNA | GKVVKICDFGLARHIMSDSNYVVRGNA |
| FINYVKNCFRMTDQEAIQDLWQWRKSL | FINYVKNCFRMTDQEAIQDLCLAVEEVSLRK |
| GFPVHYTDVSNMSRLARQRLLGRSWSV | GFPVHYTDVSNMSHLARQRLLGRSWSV |
| GFPVHYTDVSNMSRLARQRLLGRSWSV | GFPVHYTDVSNMSCLARQRLLGRSWSV |
| KLKKMWKSPNGTIRNILGGTVFREPII | KLKKMWKSPNGTIQNILGGTVFREPII |
| RLVSGWVKPIIIGRHAYGDQYRATDFV | RLVSGWVKPIIIGCHAYGDQYRATDFV |
| GETCLLDILDTAGQEEYSAMRDQYMRT | GETCLLDILDTAGKEEYSAMRDQYMRT |
| MTEYKLVVVGAGGVGKSALTIQLIQNH | MTEYKLVVVGAGDVGKSALTIQLIQNH |
| MTEYKLVVVGAGGVGKSALTIQLIQNH | MTEYKLVVVGADGVGKSALTIQLIQNH |
| GRITKICDFGLARDIKNDSNYVVKGNA | GRITKICDFGLARYIKNDSNYVVKGNA |

Table 6 shows the genes and corresponding mutations associated with melanoma, with the most represented determined to be BRAF V600E and NRAS 061.

TABLE 6
Cancer Mutations Associated with Melanoma

| | | | # Samples | | | | | |
|---|---|---|---|---|---|---|---|---|
| | | # Patient | with this | | | Mutation | Functionally | |
| Gene | Cancer | samples | mutation | Frequency | Mutation | type | significant? | Hotspot? |
| BRAF | Melanoma | 955 | 475 | 49.7% | V600E | Missense | Oncogenic | Y |
| NRAS | Melanoma | 955 | 73 | 7.6% | Q61R | Missense | Oncogenic | Y |
| NRAS | Melanoma | 955 | 71 | 7.4% | Q61K | Missense | Oncogenic | Y |
| NRAS | Melanoma | 955 | 24 | 2.5% | Q61L | Missense | Oncogenic | Y |
| CDKN2A | Melanoma | 955 | 20 | 2.1% | P114L | Missense | Oncogenic | Y |
| CDH6 | Melanoma | 955 | 18 | 1.9% | S524L | Missense | N | N |
| CNTNAP2 | Melanoma | 955 | 15 | 1.6% | G362E | Missense | N | N |
| ASTN1 | Melanoma | 955 | 15 | 1.6% | E502K | Missense | N | N |
| MUC16 | Melanoma | 955 | 14 | 1.5% | P5119S | Missense | N | N |
| ASXL3 | Melanoma | 955 | 14 | 1.5% | P1370S | Missense | N | N |
| GRIN2A | Melanoma | 955 | 13 | 1.4% | G1322E | Missense | Likely | N |
| TP63 | Melanoma | 955 | 13 | 1.4% | R379C | Missense | Oncogenic | Y |
| TP63 | Melanoma | 955 | 12 | 1.3% | D3236N | Missense | N | N |
| APOB | Melanoma | 955 | 12 | 1.3% | R1388C | Missense | N | N |
| XIRP2 | Melanoma | 955 | 12 | 1.3% | E831K | Missense | N | N |
| TPTE | Melanoma | 955 | 12 | 1.3% | S447L | Missense | N | N |
| C6 | Melanoma | 955 | 12 | 1.3% | R145C | Missense | N | N |
| CNTN5 | Melanoma | 955 | 12 | 1.3% | S379F | Missense | N | N |
| TRRAP | Melanoma | 955 | 12 | 1.3% | S722F | Missense | N | N |
| GRID2 | Melanoma | 955 | 12 | 1.3% | E487K | Missense | N | N |
| TMC5 | Melanoma | 955 | 12 | 1.3% | R276C | Missense | N | N |
| DNAH5 | Melanoma | 955 | 11 | 1.2% | G920E | Missense | N | N |
| PAK5 | Melanoma | 955 | 11 | 1.2% | M173I | — | Likely oncogenic | Y |
| SNCAIP | Melanoma | 955 | 11 | 1.2% | E627K | Missense | N | N |
| EPHA6 | Melanoma | 955 | 11 | 1.2% | R268C | Missense | N | N |
| EPHA6 | Melanoma | 955 | 11 | 1.2% | E609K | Missense | Oncogenic | Y |
| BCLAF1 | Melanoma | 955 | 11 | 1.2% | E163K | Missense | — | — |
| RP1 | Melanoma | 955 | 10 | 1.0% | G962E | Missense | N | N |
| DNAH9 | Melanoma | 955 | 10 | 1.0% | E2368K | Missense | N | N |
| PTPRT | Melanoma | 955 | 10 | 1.0% | E324K | Missense | N | N |
| TP53 | Melanoma | 955 | 10 | 1.0% | R248W | Missense | N | N |
| TNR | Melanoma | 955 | 10 | 1.0% | E930K | Missense | N | N |
| CPAMD8 | Melanoma | 955 | 10 | 1.0% | R568C | Missense | N | N |
| KDR | Melanoma | 955 | 10 | 1.0% | S1100F | Missense | Likely Oncogenic | Y |

Table 7 shows the genes and corresponding mutations associated with bladder cancer, with the most represented determined to be FGFR3 S249C, FGFR3 Y373C, and PIK3KA E545K.

TABLE 7
Cancer Mutations Associated with Bladder Cancer

| | | | # Samples | | | | | |
|---|---|---|---|---|---|---|---|---|
| | | # Patient | with this | | | Mutation | Functionally | |
| Gene | Cancer | samples | mutation | Frequency | Mutation | type | significant? | Hotspot? |
| FGFR3 | Bladder | 1429 | 121 | 8.5% | S249C | Missense | Oncogenic | Y |
| PIK3CA | Bladder | 1429 | 107 | 7.5% | E545K | Missense | Oncogenic | Y |
| FGFR3 | Bladder | 1429 | 45 | 3.1% | Y373C | Missense | Oncogenic | Y |
| PIK3CA | Bladder | 1429 | 44 | 3.1% | E542K | Missense | Oncogenic | Y |
| TP53 | Bladder | 1429 | 43 | 3.0% | R248Q | Missense | Oncogenic | Y |
| ERBB2 | Bladder | 1429 | 40 | 2.8% | S310F | Missense | Oncogenic | Y |
| TP53 | Bladder | 1429 | 35 | 2.4% | E285K | Missense | Oncogenic | Y |
| TP53 | Bladder | 1429 | 35 | 2.4% | R280T | Missense | Oncogenic | Y |
| FGFR3 | Bladder | 1429 | 18 | 1.3% | R248C | Missense | Oncogenic | Y |
| TP53 | Bladder | 1429 | 15 | 1.0% | R280K | Missense | Oncogenic | Y |
| ERBB3 | Bladder | 1429 | 12 | 0.8% | V140L | Missense | Oncogenic | Y |
| ERCC2 | Bladder | 1429 | 12 | 0.8% | N238S | Missense | Oncogenic | Y |
| TP53 | Bladder | 1429 | 11 | 0.8% | R248W | Missense | Oncogenic | Y |
| ERBB3 | Bladder | 1429 | 11 | 0.8% | M91I | Missense | Oncogenic | Y |
| CDKN2A | Bladder | 1429 | 11 | 0.8% | D108Y/N/H | Missense | Oncogenic | Y |
| FGFR3 | Bladder | 1429 | 10 | 0.7% | G370C | Missense | Oncogenic | Y |
| ERBB3 | Bladder | 1429 | 10 | 0.7% | E332K | Missense | Oncogenic | Y |
| PIK3CA | Bladder | 1429 | 9 | 0.6% | E545Q | Missense | Oncogenic | Y |
| FBXW7 | Bladder | 1429 | 9 | 0.6% | R505G | Missense | Oncogenic | Y |
| ERBB2 | Bladder | 1429 | 8 | 0.6% | S310Y | Missense | Oncogenic | Y |
| ERBB3 | Bladder | 1429 | 3 | 0.2% | V104M | Missense | Oncogenic | Y |

Table 8 shows the genes and corresponding mutations associated with glioblastoma, with the most represented determined to be IDHW R1 32H, EGFR A289V, and EGFR G598V.

TABLE 8
Cancer Mutations Associated with Glioblastoma

| | | | # Samples | | | | | |
|---|---|---|---|---|---|---|---|---|
| | | # Patient | with this | | | Mutation | Functionally | Hot- |
| Gene | Cancer | samples | mutation | Frequency | Mutation | type | significant? | spot? |
| PIK3CA | Glioblastoma | 585 | 1 | 0.2% | E545A | Missense | Oncogenic | Y |
| PTEN | Glioblastoma | 585 | 2 | 0.3% | R173C | Missense | Oncogenic | Y |
| SYNE1 | Glioblastoma | 585 | 2 | 0.3% | R8468H | Missense | Oncogenic | N |
| PTEN | Glioblastoma | 585 | 3 | 0.5% | R130Q | Missense | Oncogenic | Y |
| PTEN | Glioblastoma | 585 | 3 | 0.5% | R173H | Missense | Oncogenic | Y |
| PIK3CA | Glioblastoma | 585 | 3 | 0.5% | E545K | Missense | Oncogenic | Y |
| TP53 | Glioblastoma | 585 | 4 | 0.7% | R248Q | Missense | Oncogenic | Y |
| TP53 | Glioblastoma | 585 | 4 | 0.7% | R273H | Missense | Oncogenic | Y |
| TP53 | Glioblastoma | 585 | 5 | 0.9% | R248W | Missense | Oncogenic | Y |
| EGFR | Glioblastoma | 585 | 5 | 0.9% | A289D | Missense | Oncogenic | Y |
| EGFR | Glioblastoma | 585 | 6 | 1.0% | A289T | Missense | Oncogenic | Y |
| EGFR | Glioblastoma | 585 | 6 | 1.0% | R222C | Missense | Oncogenic | Y |
| PIK3R1 | Glioblastoma | 585 | 6 | 1.0% | G376R | Missense | Oncogenic | Y |
| TP53 | Glioblastoma | 585 | 8 | 1.4% | R248Q | Missense | Oncogenic | Y |
| TP53 | Glioblastoma | 585 | 8 | 1.4% | R175H | Missense | Oncogenic | Y |
| EGFR | Glioblastoma | 585 | 14 | 2.4% | G598V | Missense | Oncogenic | Y |
| EGFR | Glioblastoma | 585 | 16 | 2.7% | A289V | Missense | Oncogenic | Y |
| IDH1 | Glioblastoma | 585 | 23 | 3.9% | R132H | Missense | Oncogenic | N |

Table 9 shows the genes and corresponding mutations associated with pancreatic cancer. Pancreatic cancers are known to have fewer mutations than other cancer types, but as shown there are still numerous detected mutations.

TABLE 9
Cancer Mutations Associated with Pancreatic Cancer

| Gene | Chromosome | Position | Exon | Mut_aa | Mut_nt | Mut_cdna | Transcript | Percentage |
|---|---|---|---|---|---|---|---|---|
| FZD4 | 11 | 86665923 | 1 | H69Y | G>A | c.205C>T | NM_012193.3 | 50.27 |
| FZD9 | 7 | 72848967 | 1 | S210S | G>A | c.630G>A | NM_003508.2 | 50.24 |
| TERT | 5 | 1295349 | 1 | | A>G | c.-245T>C | NM_198253.2 | 50.18 |
| CEP295 | 11 | 93430669 | 15 | T864R | CT>GA | c.2591_2592 | NM_033395.1 | 50.18 |
| | | | | | | delCTinsGA | | |
| WRN | 8 | 31015036 | 33 | P1324P | C>T | c.3972C>T | NM_000553.4 | 50.1 |
| NOTCH3 | 19 | 15297997 | 11 | R587C | G>A | c.1759C>T | NM_000435.2 | 49.69 |
| MET | 7 | 116423427 | 19 | Y1234Y | C>T | c.3702C>T | NM_000245.2 | 49.52 |
| NTRK3 | 15 | 88576185 | 14 | A496A | G>T | c.1488C>A | NM_001012338.2 | 49 |
| FLT1 | 13 | 29012441 | 4 | E144K | C>T | c.430G>A | NM_002019.4 | 48.89 |
| ARID1B | 6 | 157099209 | 1 | S49F | C>T | c.146C>T | NM_020732.3 | 48.37 |
| PALB2 | 16 | 23632761 | 10 | T1012I | G>A | c.3035C>T | NM_024675.3 | 48.33 |
| DDR2 | 1 | 162741869 | 14 | H520H | C>T | c.1560C>T | NM_001014796.1 | 48.08 |
| PALB2 | 16 | 23646594 | 4 | V425M | C>T | c.1273G>A | NM_024675.3 | 47.59 |
| BRCA1 | 17 | 41244488 | 10 | P1020P | T>C | c.3060A>G | NM_007294.3 | 47.56 |
| PTCH1 | 9 | 98231061 | 14 | A741V | G>A | c.2222C>T | NM_000264.3 | 47.47 |
| GAS6 | 13 | 114538581 | 7 | A206V | G>A | c.617C>T | NM_000820.3 | 47.08 |
| JAK3 | 19 | 17947984 | 13 | S580S | C>T | c.1740G>A | NM_000215.3 | 46.92 |
| KAT6A | 8 | 41798413 | 15 | S996G | T>C | c.2986A>G | NM_006766.4 | 44.51 |
| ATM | 11 | 108117799 | 8 | R337H | G>A | c.1010G>A | NM_000051.3 | 1.59 |
| TET2 | 4 | 106190797 | 9 | R1359C | C>T | c.4075C>T | NM_001127208.2 | 1.11 |
| PCDH15 | 10 | 55955492 | 12 | N424S | T>C | c.1271A>G | NM_001142763.1 | 0.65 |
| CDH1 | 16 | 68853292 | 11 | S559G | A>G | c.1675A>G | NM_004360.3 | 0.57 |
| MED12 | X | 70349221 | 26 | S1211S | C>T | c.3633C>T | NM_005120.2 | 0.49 |
| CCND3 | 6 | 41903759 | 5 | A266A | C>T | c.798G>A | NM_001760.4 | 0.46 |
| TBX3 | 12 | 115112283 | 7 | T486M | G>A | c.1457C>T | NM_016569.3 | 0.44 |
| TERT | 5 | 1295250 | 1 | | G>A | c.-146C>T | NM_198253.2 | 0.43 |
| IDO1 | 8 | 39775722 | 3 | R100H | G>A | c.299G>A | NM_002164.5 | 0.42 |
| NOTCH2 | 1 | 120510201 | 8 | T436T | C>T | c.1308G>A | NM_024408.3 | 0.38 |
| EPHA3 | 3 | 89445077 | 6 | I466T | T>C | c.1397T>C | NM_005233.5 | 0.29 |
| TERT | 5 | 1294277 | 2 | A242T | C>T | c.724G>A | NM_198253.2 | 0.27 |
| POLE | 12 | 133249282 | 15 | Y539Y | G>A | c.1617C>T | NM_006231.3 | 0.25 |
| DDR2 | 1 | 162724572 | 6 | A115V | C>T | c.344C>T | NM_001014796.1 | 0.24 |
| DNMT3A | 2 | 25470908 | 7 | E285* | C>A | c.853G>T | NM_022552.4 | 0.23 |
| PPM1D | 17 | 58740819 | 6 | V576fs | C>CT | c.1725dupT | NM_003620.3 | 1.34 |

| COSMIC | dbSNP | Mol_count | TMB_score | TMB_category | MSI_High | cfDNA_ng | Cancer type |
|---|---|---|---|---|---|---|---|
| | rs80358282 | 4937 | 17.86 | Low | Not Detected | 115.7 | Pancreas |
| | | 4215 | 17.86 | Low | Not Detected | 115.7 | Pancreas |
| | rs2853669 | 4476 | 17.86 | Low | Not Detected | 115.7 | Pancreas |
| | rs386756272 | 5742 | 17.86 | Low | Not Detected | 115.7 | Pancreas |
| COSM3648358 | rs370253199 | 4765 | 17.86 | Low | Not Detected | 115.7 | Pancreas |
| | rs754554486 | 6753 | 17.86 | Low | Not Detected | 115.7 | Pancreas |
| | rs201747580 | 3710 | 17.86 | Low | Not Detected | 115.7 | Pancreas |
| | rs2229910 | 6883 | 17.86 | Low | Not Detected | 115.7 | Pancreas |
| COSM1366253 | rs55974987 | 4600 | 17.86 | Low | Not Detected | 115.7 | Pancreas |
| | rs375486386 | 909 | 17.86 | Low | Not Detected | 115.7 | Pancreas |
| | rs761032954 | 3883 | 17.86 | Low | Not Detected | 115.7 | Pancreas |
| | rs55875050 | 4223 | 17.86 | Low | Not Detected | 115.7 | Pancreas |
| COSM1286951 | rs576081828 | 4181 | 17.86 | Low | Not Detected | 115.7 | Pancreas |
| | rs781435355 | 3873 | 17.86 | Low | Not Detected | 115.7 | Pancreas |
| | rs2227971 | 4577 | 17.86 | Low | Not Detected | 115.7 | Pancreas |
| | rs555084854 | 5253 | 17.86 | Low | Not Detected | 115.7 | Pancreas |
| | rs143605793 | 2082 | 17.86 | Low | Not Detected | 115.7 | Pancreas |
| | rs183255462 | 4863 | 17.86 | Low | Not Detected | 115.7 | Pancreas |
| COSM21301 | rs202160435 | 3083 | 17.86 | Low | Not Detected | 115.7 | Pancreas |
| COSM41649 | | 4698 | 17.86 | Low | Not Detected | 115.7 | Pancreas |
| | rs143827620 | 3151 | 17.86 | Low | Not Detected | 115.7 | Pancreas |
| | | 5476 | 17.86 | Low | Not Detected | 115.7 | Pancreas |
| | | 4175 | 17.86 | Low | Not Detected | 115.7 | Pancreas |
| | rs375843578 | 3884 | 17.86 | Low | Not Detected | 115.7 | Pancreas |
| | rs1018135320 | 2388 | 17.86 | Low | Not Detected | 115.7 | Pancreas |
| | | 5130 | 17.86 | Low | Not Detected | 115.7 | Pancreas |
| COSM3374925 | rs200244502 | 4823 | 17.86 | Low | Not Detected | 115.7 | Pancreas |
| | rs587728761 | 6718 | 17.86 | Low | Not Detected | 115.7 | Pancreas |
| | | 5069 | 17.86 | Low | Not Detected | 115.7 | Pancreas |
| | | 5049 | 17.86 | Low | Not Detected | 115.7 | Pancreas |
| COSM3458359 | rs775930793 | 6065 | 17.86 | Low | Not Detected | 115.7 | Pancreas |
| | | 6511 | 17.86 | Low | Not Detected | 115.7 | Pancreas |
| COSM4383607 | rs201882909 | 5128 | 17.86 | Low | Not Detected | 115.7 | Pancreas |
| | | 5777 | 17.86 | Low | Not Detected | 115.7 | Pancreas |

The results shown in Tables 1-9 indicate that two genes, TP53 and KRAS, were present in all cancers. KRAS G12D was the most common mutation across all cancers (Table 10).

TABLE 10
The Gene and Mutation
Frequency Found in All Cancers

| | | Mutational |
|---|---|---|
| Gene | Mutation | Frequency |
| KRAS | G12A | 0.4% |
| KRAS | G12C | 3.8% |
| KRAS | G12D | 5.5% |
| KRAS | G12R | 1.0% |
| KRAS | G12S | 0.3% |
| KRAS | G12V | 4.8% |
| KRAS | G13D | 1.7% |
| KRAS | G13C | 0.3% |
| KRAS | Q61K | 0.6% |
| TP53 | E285K | 0.3% |
| TP53 | G245S | 0.4% |
| TP53 | R158L | 0.5% |
| TP53 | R175H | 1.8% |
| TP53 | R248Q | 1.3% |
| TP53 | R248W | 0.7% |
| TP53 | R273C | 0.7% |
| TP53 | R273H | 0.8% |
| TP53 | R282W | 0.8% |
| TP53 | V157F | 0.5% |
| Total | | 26.3% |

Previously, cancer genome sequencing was based upon DNA sourced directly from tumors (e.g., tumor biopsy) because only tumors had enough cells present to perform traditional sequencing or gene chip technology. The TCGA data described above is sourced in this way.

However, the present example further describes the use of a new technology recently of which it has applications approved by the FDA for clinical use in cancer genome sequencing that uses cell free DNA (cfDNA) sequencing rather than tumors. cfDNA is comprised of smaller amounts of DNA and provides a new type of sequencing as compared to traditional approaches, which rely upon longer primers and annealing to the target DNA strand. In addition, cfDNA often not used for the purposes of sequencing cancerous mutations and has traditionally been used for testing potential genetic abnormalities in embryos. The present example utilizes cfDNA obtained from patient's blood to sequence and identify cancerous mutations within that patient. A version of the liquid biopsy NGS panel by Guardant (Onco 360) has 75 genes and was recently approved by FDA. In these studies, we used Guardant's OMNI panel which sequences 500 genes. The genes assayed on this panel are provided in Table 11.

TABLE 11
List of Genes Assayed on OMNI Panel for Coding or Copy number Changes

| ABL1 | ABL2 | ACVR1B | ACVR2A | ADARB2 | ADGRA2 |
|---|---|---|---|---|---|
| ALB | ALK | ALOX12B | ALOX15B | ALOX5 | AMER1 |
| ARID1A | ARID1B | ARID2 | ASXL1 | ATM | ATR |
| B2M | BAP1 | BARD1 | BCL2 | BCL2L1 | BCL2L2 |
| BRAF | BRCA1 | BRCA2 | BRD2 | BRD3 | BRD4 |
| CASP8 | CBFB | CBL | CBLB | CCND1 | CCND2 |
| CDC73 | CDH1 | CDK12 | CDK4 | CDK6 | CDK8 |
| CEP295 | CHEK1 | CHEK2 | CIC | CNOT3 | CREBBP |
| CTNNB1 | CUL3 | CUX1 | CYLD | DAXX | DDIT3 |
| DNMT3A | DOT1L | DYRK2 | E2F3 | ECT2L | EGFR |
| EIF4E2 | ELF3 | EML4 | EMSY | EP300 | EPCAM |
| ERBB4 | ERCC1 | ERCC2 | ERCC3 | ERCC4 | ERCC5 |
| ETV4 | ETV5 | ETV6 | EWSR1 | EXO1 | EZH2 |
| FANCB | FANCC | FANCD2 | FANCE | FANCF | FANCG |
| FEN1 | FGF10 | FGF14 | FGF19 | FGF23 | FGF3 |
| FH | FLCN | FLT1 | FLT3 | FLT4 | FOXA1 |
| FZD10 | FZD2 | FZD3 | FZD4 | FZD5 | FZD6 |
| GATA3 | GATA6 | GEN1 | GID4 | GNA11 | GNA13 |
| HELQ | HES1 | HEY1 | HEYL | HGF | HIST3H3 |
| IFNG | IFNGR1 | IFNGR2 | IGF1 | IGF1R | IGF2 |
| INPP4B | IRF1 | IRF4 | IRS2 | JAK1 | JAK2 |
| KDM5C | KDM6A | KDR | KEAP1 | KIT | KLHL6 |
| LGR6 | LIG1 | LIG4 | LMO1 | LRP1B | LRP2 |
| MAP3K1 | MAP4K3 | MAPK1 | MAPK3 | MAPKAP1 | MAX |
| MEN1 | MERTK | MET | MITF | MLH1 | MLH3 |
| MSH6 | MTOR | MUTYH | MYB | MYC | MYCL |
| NFKBIA | NHEJ1 | NKX2-1 | NOTCH1 | NOTCH2 | NOTCH3 |
| NTRK1 | NTRK2 | NTRK3 | NUMB | NUP93 | NUTM1 |
| PBRM1 | PCDH15 | PDCD1 | PDCD1LG2 | PDGFRA | PDGFRB |
| PIK3CD | PIK3CG | PIK3R1 | PIK3R2 | PIK3R3 | PIM1 |
| POLE | POLH | POLQ | POU2F2 | PPARG | PPM1D |
| PREX1 | PREX2 | PRKAR1A | PRKCI | PRKDC | PTCH1 |
| RAD50 | RAD51 | RAD51B | RAD51C | RAD51D | RAD52 |
| RET | REV3L | RGS1 | RHEB | RHOA | RHOB |
| RPA1 | RPS27A | RPS6KA3 | RPS6KB1 | RPTOR | RRAGC |
| SDHD | SESN2 | SETD2 | SF3B1 | SHFM1 | SLC34A2 |
| SMARCA4 | SMARCB1 | SMO | SOCS1 | SOCS3 | SOS1 |
| SRSF2 | SRY | STAG2 | STAT3 | STAT4 | STK11 |
| TERT | TET2 | TGFBR2 | TMPRSS2 | TNFAIP3 | TNFRSF14 |
| TP63 | TP73 | TRAF3 | TSC1 | TSC2 | TSHR |
| VHL | WEE1 | WISP3 | WRN | WT1 | XBP1 |
| XRCC4 | XRCC5 | XRCC6 | YAP1 | ZNF217 | ZNF703 |
| ADGRG4 | AFDN | AKT1 | AKT1S1 | AKT2 | AKT3 |
| APC | APEX1 | AR | ARAF | ARFRP1 | ARHGAP35 |
| ATRX | AURKA | AURKB | AXIN1 | AXIN2 | AXL |
| BCL6 | BCOR | BCORL1 | BCR | BIRC5 | BLM |
| BRIP1 | BTG1 | BTG2 | BTK | BUB1B | CARD11 |
| CCND3 | CCNE1 | CD274 | CD79A | CD79B | CDC7 |
| CDKN1A | CDKN1B | CDKN2A | CDKN2B | CDKN2C | CEBPA |
| CRKL | CRTC1 | CSF1R | CTCF | CTLA4 | CTNNA1 |
| DDR1 | DDR2 | DEPDC5 | DEPTOR | DICER1 | DLL4 |
| EIF1AX | EIF4A1 | EIF4A2 | EIF4A3 | EIF4B | EIF4E |
| EPHA3 | EPHA5 | EPHA7 | EPHB1 | ERBB2 | ERBB3 |
| ERCC6 | ERCC8 | ERG | ERRFI1 | ESR1 | ETV1 |
| FAAP100 | FAAP20 | FAAP24 | FAM175A | FAM46C | FANCA |
| FANCI | FANCL | FANCM | FAS | FAT1 | FBXW7 |
| FGF4 | FGF6 | FGFR1 | FGFR2 | FGFR3 | FGFR4 |
| FOXL2 | FOXO1 | FOXP1 | FRS2 | FUBP1 | FZD1 |
| FZD7 | FZD8 | FZD9 | GAS6 | GATA1 | GATA2 |
| GNAQ | GNAS | GRIN2A | GSK3B | H3F3A | HDAC2 |
| HNF1A | HRAS | HSP90AA1 | IDH1 | IDH2 | IDO1 |
| IGF2R | IKBKE | IKZF1 | IL2RG | IL7R | INHBA |
| JAK3 | JUN | KAT6A | KDM4A | KDM5A | KDM5B |
| KMT2A | KMT2D | KNSTRN | KRAS | LGR4 | LGR5 |
| LRP5 | LRP6 | MAD2L2 | MAP2K1 | MAP2K2 | MAP2K4 |
| MCL1 | MDC1 | MDM2 | MDM4 | MED12 | MEF2B |
| MLST8 | MPL | MRAS | MRE11 | MSH2 | MSH3 |
| MYCN | MYD88 | NBN | NF1 | NF2 | NFE2L2 |
| NOTCH4 | NPM1 | NPRL2 | NPRL3 | NRAS | NSD1 |
| PAK3 | PALB2 | PARG | PARP1 | PARP2 | PAX5 |
| PDK1 | PHF6 | PIAS4 | PIK3C2B | PIK3CA | PIK3CB |
| PIN1 | PKM | PLEKHS1 | PMS1 | PMS2 | POLD1 |
| PPP2CA | PPP2R1A | PPP2R2A | PPP3CA | PPP6C | PRDM1 |
| PTEN | PTPN11 | PTPRD | RAC1 | RAD18 | RAD21 |
| RAD54L | RAF1 | RARA | RASA1 | RB1 | RBM10 |
| RICTOR | RIT1 | RNF43 | ROBO1 | ROBO2 | ROS1 |
| RSPO1 | RSPO4 | RUNX1 | RUNX1T1 | SDHB | SDHC |
| SLFN11 | SLIT2 | SMAD2 | SMAD3 | SMAD4 | SMARCA2 |
| SOX10 | SOX2 | SOX9 | SPEN | SPOP | SRC |
| STK19 | SUFU | SYK | TBC1D7 | TBX3 | TEK |
| TNFRSF1A | TNK2 | TOP1 | TOPAZ1 | TP53 | TP53BP1 |
| TSHZ2 | TYRO3 | U2AF1 | UBE2T | USP9X | VEGFA |
| XPA | XPC | XPO1 | XRCC1 | XRCC2 | XRCC3 |
| ZNRF3 | ZRSR2 | | | | |

Complete
Partial
Fusion
CNV

The process of sequencing from cfDNA includes drawing 10 mL of blood from a cancer patient and isolating plasma from the leukocyte and RBC fractions, thereby eliminating naturally occurring leukocyte mutations. This sample is also known as a liquid biopsy. Next, generation sequencing is performed that is sensitive enough to detect the equivalent of a single cancer genome in 10 mL of plasma. An example of the sequencing result from the Guardant Omni NGS Panel used in this process is shown below in Table 12.

TABLE 12
Test results from a Patient with adenocarcinoma cancer using Guardant NGS OMNI Panel

| Customer_ | Sample_ | Variant_ | Indel_ | | | | | | | |
|---|---|---|---|---|---|---|---|---|---|---|
| SampleID | status | type | type | Gene | Chromosome | Position | Exon | Mut_aa | Mut_nt | Mut_cdna |
| GENE01 | SUCCESS | SNV | | PREX1 | 20 | 47269187 | 21 | Q802E | G>C | c.2404C>G |
| GENE01 | SUCCESS | SNV | | PREX1 | 20 | 47266553 | 24 | 110031 | G>A | c.3009C>T |
| GENE01 | SUCCESS | SNV | | XPA | 9 | 100459562 | 1 | D5Y | C>A | c.13G>T |
| GENE01 | SUCCESS | SNV | | SETD2 | 3 | 47162704 | 3 | P1141L | G>A | c.3422C>T |
| GENE01 | SUCCESS | SNV | | POLE | 12 | 133257773 | 2 | R52Q | C>T | c. 155G>A |
| GENE01 | SUCCESS | SNV | | LIG4 | 13 | 108863584 | 2 | A11A | T>C | c.33A>G |
| GENE01 | SUCCESS | SNV | | APC | 5 | 112175147 | 16 | E1286* | G>T | c.3856G>T |
| GENE01 | SUCCESS | SNV | | BRCA1 | 17 | 41215367 | 18 | R1726G | T>C | c.5176A>G |
| GENE01 | SUCCESS | SNV | | FZD5 | 2 | 208631933 | 2 | L511L | G>A | c. 1531C>T |
| GENE01 | SUCCESS | SNV | | SOX2 | 3 | 181430545 | 1 | A133T | G>A | c.397G>A |
| GENE01 | SUCCESS | SNV | | ERBB2 | 17 | 37883970 | 27 | P1147P | C>T | c.3441C>T |
| GENE01 | SUCCESS | SNV | | POLD1 | 19 | 50917079 | 18 | E803E | G>A | c.2409G>A |
| GENE01 | SUCCESS | SNV | | KDM5B | 1 | 202711628 | 19 | R863Q | C>T | c.2588G>A |
| GENE01 | SUCCESS | SNV | | TP53 | 17 | 7577538 | 7 | R248Q | C>T | c.743G>A |
| GENE01 | SUCCESS | SNV | | EGFR | 7 | 55259484 | 21 | P848S | C>T | c.2542C>T |
| GENE01 | SUCCESS | SNV | | MEN1 | 11 | 64572253 | 10 | A467A | G>T | c. 1401C>A |
| GENE01 | SUCCESS | SNV | | PPARG | 3 | 12475559 | 7 | V478A | T>C | c. 1433T>C |
| GENE01 | SUCCESS | SNV | | NF1 | 17 | 29533280 | 12 | K428T | A>C | c.1283A>C |
| GENE01 | SUCCESS | SNV | | FZD6 | 8 | 104337384 | 4 | G350G | A>C | c. 1050A>C |
| GENE01 | SUCCESS | SNV | | KDM6A | X | 44732940 | 1 | A48V | C>T | c. 143C>T |
| GENE01 | SUCCESS | SNV | | IKZF1 | 7 | 50450352 | 5 | N179T | A>C | c.536A>C |
| GENE01 | SUCCESS | SNV | | LRP1B | 2 | 141777555 | 12 | R636W | G>A | c. 1906C>T |
| GENE01 | SUCCESS | SNV | | DEPTOR | 8 | 120940780 | 2 | L88P | T>C | c.263T>C |
| GENE01 | SUCCESS | SNV | | ALK | 2 | 30143244 | 1 | D94D | G>A | c.282C>T |
| GENE01 | SUCCESS | SNV | | FAT1 | 4 | 187630361 | 2 | T207T | A>C | c.621T>G |
| GENE01 | SUCCESS | SNV | | FAT1 | 4 | 187524423 | 19 | S3753T | A>T | c.11257T>A |
| GENE01 | SUCCESS | CNV | | GNAS | 20 | | | | | |
| GENE01 | SUCCESS | CNV | | ZNF217 | 20 | | | | | |
| GENE01 | SUCCESS | CNV | | PREX2 | 8 | | | | | |
| GENE01 | SUCCESS | CNV | | MYC | 8 | | | | | |
| GENE01 | SUCCESS | CNV | | BCL2L1 | 20 | | | | | |
| GENE01 | SUCCESS | CNV | | FGFR3 | 4 | | | | | |
| GENE01 | SUCCESS | CNV | | FLT3 | 13 | | | | | |
| GENE01 | SUCCESS | CNV | | KDR | 4 | | | | | |
| GENE01 | SUCCESS | CNV | | PDGFRA | 4 | | | | | |
| GENE01 | SUCCESS | CNV | | KIT | 4 | | | | | |

| | | Somatic | Copy | Amplification | | | Mol_ | TMB_ | TMB_ |
|---|---|---|---|---|---|---|---|---|---|
| Transcript | Percentage | status | number | type | COSMIC | dbSNP | count | score | category |
| NM_020820.3 | 74.77 | germline | | | | rs148057033 | 15630 | 12.45 | Low |
| NM_020820.3 | 74.03 | germline | | | | rs142843026 | 10549 | 12.45 | Low |
| NM_000380.3 | 73.18 | germline | | | | rs574504791 | 2838 | 12.45 | Low |
| NM_014159.6 | 67.47 | germline | | | | rs142723093 | 4608 | 12.45 | Low |
| NM_006231.3 | 65.98 | germline | | | | rs372459649 | 3851 | 12.45 | Low |
| NM_001098268.1 | 57.26 | germline | | | | rs748718655 | 3714 | 12.45 | Low |
| NM_000038.5 | 50.24 | somatic | | | COSM18772 | | 3792 | 12.45 | Low |
| NM_007294.3 | 49.85 | germline | | | | rs80357501 | 3016 | 12.45 | Low |
| NM_003468.3 | 49.38 | germline | | | | rs150237984 | 5987 | 12.45 | Low |
| NM_003106.3 | 48.78 | germline | | | | rs377110178 | 5430 | 12.45 | Low |
| NM_004448.2 | 48.54 | germline | | | | rs112561362 | 4898 | 12.45 | Low |
| NM_001308632.1 | 48.53 | germline | | | | | 6806 | 12.45 | Low |
| NM_001314042.1 | 47.71 | germline | | | | rs753883605 | 6860 | 12.45 | Low |
| NM_000546.5 | 37.47 | somatic | | | COSM10662 | rs11540652 | 7104 | 12.45 | Low |
| NM_005228.3 | 28.32 | somatic | | | | | 10320 | 12.45 | Low |
| NM_000244.3 | 27.33 | somatic | | | | rs771827808 | 5580 | 12.45 | Low |
| NM_015869.4 | 23.61 | somatic | | | | | 6577 | 12.45 | Low |
| NM_001042492.2 | 12.96 | somatic | | | | | 5085 | 12.45 | Low |
| NM_001164615.1 | 8.69 | somatic | | | | | 13121 | 12.45 | Low |
| NM_001291415.1 | 6.11 | somatic | | | | | 1824 | 12.45 | Low |
| NM_006060.5 | 2.85 | somatic | | | | | 8226 | 12.45 | Low |
| NM_018557.2 | 2.02 | somatic | | | COSM1007075 | rs766132339 | 4832 | 12.45 | Low |
| NM_022783.3 | 1.56 | somatic | | | | | 8170 | 12.45 | Low |
| NM_004304.4 | 0.51 | somatic | | | | | 6267 | 12.45 | Low |
| NM_005245.3 | 0.33 | somatic | | | | | 4582 | 12.45 | Low |
| NM_005245.3 | 0.2 | somatic | | | | | 4468 | 12.45 | Low |
| | | somatic | 4.54 | aneuploidy | | | | 12.45 | Low |
| | | somatic | 4.44 | aneuploidy | | | | 12.45 | Low |
| | | somatic | 3.01 | aneuploidy | | | | 12.45 | Low |
| | | somatic | 2.87 | aneuploidy | | | | 12.45 | Low |
| | | somatic | 2.74 | aneuploidy | | | | 12.45 | Low |
| | | somatic | 2.68 | focal | | | | 12.45 | Low |
| | | somatic | 2.56 | aneuploidy | | | | 12.45 | Low |
| | | somatic | 2.55 | aneuploidy | | | | 12.45 | Low |
| | | somatic | 2.53 | aneuploidy | | | | 12.45 | Low |
| | | somatic | 2.48 | aneuploidy | | | | 12.45 | Low |

TABLE 12
Test results from a Patient with adenocarcinoma
cancer using Guardant NGS OMNI Panel (cont'd)

| MSI_High | cfDNA_ng | Cancer type |
|---|---|---|
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |
| Not Detected | 23.79 | Adenocarcinoma Colon |

This assay provides germline mutations, ones present at the start of life and thus present in all cells, and somatic mutations which are likely sourced from cancer cells. It can also provide an idea of how much of the mutation was present in cfDNA. The amount of DNA being shed is directly proportional to the number of cancer cells containing that mutation. For example, Table 11 above shows that the percent of cancer genomes containing a TP53 mutation R248Q is 37.47%. It is therefore likely that this mutation occurred the earliest in oncogenesis of all mutations recorded. TP53 is a critical tumor suppressor with R248Q occurring in 1.3% of cancers. Because R248Q is present in a high fraction of cells and plays an important role in oncogenesis, R248Q can potentially serve as a neoantigen target.

The cfDNA panel results from the blood are representative of all of the cancer lesions in the patient-metastatic as well as primary- and thus the findings of these tests are more relevant than that provided by sequencing a lesion that has been surgically removed. Importantly, the results from Example 1 indicate that coding changes in cancer are ubiquitous and do not only consist of mutations leading to loss of gene expression or truncating mutations resulting in an incomplete protein. The T cell targeting approach can be used against any targets that can be presented by a major histocompatibility complex (MHC). This approach is independent of whether the MHC is a class I or II MHC. Accordingly, it is contemplated that this therapy can apply to any cancer patient.

### Example 3: Prefabricated Peptide Panels

The following example provides details regarding how to prefabricate peptide panels for use in generating T cells. Peptide manufacturing can take up to 3-6 months from the time of ordering the peptides from the manufacturer to receipt of the peptides. The cost is also significant with one peptide costing between $400-$500. With each mutation having 4 peptides that are 15 amino acids long and scanning a 27 aa region containing the mutation, costs can be significant. Additionally, a non-mutant version needs to be produced for testing final products in ELISpot and/or cytotoxicity assay to ensure the cells will not react to the germline sequence. Therefore, there is a need to develop a more affordable treatment that would enable the production of select peptides encoding mutations that are able to treat the greatest number of people.

There are two approaches to antigen selection for potential treatments based on the genetic analyses. The first approach includes selecting mutations on how commonly they occur whereas the second approach is a fully personalized approach where all detected mutations are used. The source of antigen is important as it influences how patients are selected and how long manufacturing may take. Adequate care for cancer patients includes not only the type of drug they receive, but also the timing. For example, patients at an advanced stage will need their treatment on the order of weeks, not months. However, some cell therapies can take six months or more to manufacture before they are ready to administer to a patient. Preselecting antigens allows for rapid treatment options as reagents can be prepared in advance and reduce the cost of large-scale manufacturing. In contrast, current fully personalized approaches do not have these advantages because the peptides still need to be produced after sequencing.

Using information derived from Example 1 (e.g., Tables 1-10), an off the shelf peptide approach has been developed wherein neoantigen “pepmixes” are produced as the source of antigen for T cell manufacturing. Each neoantigen and corresponding germline sequence is made up in the form of a pepmix with the mutation at the center of a 27 amino acid sequence tiled by 15 amino acids with 11 amino acid overlap. If an entire protein is targeted, then 15 amino acids are tiled across the entire sequence or a selected portion of the protein. A GMP peptide has to be synthesized in a GMP-compliant facility. Each peptide is made and purified on separate HPLC columns. Mass spectrometry is used to determine if the protein has been stably produced and purified to 99.5%, and the process can produce the neoantigen pepmixes on a mg or gram scale. If pepmixes are not purified, they can produce false positives and false negatives due to off target sequences.

The data shown in Table 10 indicate that 26.3% of patients will carry at least one of the listed mutations. As discussed above in Example 1, the mutations in TP53 and KRAS were further identified as critical tumor suppressors and oncogenes, respectively.

There are also several methods by which patients having these mutations can be identified, for example, in databases of sequenced patients, by companies that provide sequence information, via commercial tests that identify cancer associated mutations in KRAS of a given patient, and by patients requesting sequencing of their own tumors or blood. Oncologists have the sequence data from patients and can determine which patients have multiple mutations covered by the presynthesized peptide library. However, regardless of the method used to identify the patient, once identified as having one of the mutations enumerated in Tables 1-10, the patient will then be eligible for treatment.

Either a peptide mix of all the common mutations in Tables 1-10 or a single peptide matching the patient's mutation from all of the KRAS and TP53 mutations in Table 10 could be used. Alternatively, another approach could include the use of specific combinations of peptides such as TP53 R248W and KRAS G12D that, when combined, can cover a greater number of patients. Some combinations may be particularly effective at preventing recurrence or chemotherapy- or radiation-treatment-induced cancers.

### Example 4: Monocyte Derived Dendritic Cell Differentiation

The present example provides details regarding the use of monocyte derived dendritic cell (DC) differentiation. PBMCs rely on naturally occurring DCs that comprise <0.1% of the total cells and other less effective antigen presenting cells such as monocytes to begin T cell stimulation. The following example provides details regarding the use of monocyte derived DCs that enable a ratio of one DC to two or four T cells.

Monocytes can be differentiated into DCs by multiday culturing in concentrated GM-CSF and IL-4. The monocytes are isolated from PBMCs either by CD14 positive selection beads or by plastic adherence. PBMCs adhere to tissue culture plastic and are thoroughly washed. The depleted cells or non-adherent cells are saved for later introduction to DCs. While either of these methods or others are suitable, the present example utilizes the adherence method.

To differentiate monocytes into DCs, isolated PBMCs were suspended in RPMI 1640 (Millipore Sigma-Aldrich) at 10 million cells per mL and transferred to 6-well or 24-well plates at 2 mL or 0.5 mL, respectively. Cells were incubated for 1 hour at 37° C., 5% CO₂ humidified chamber to allow for adherence, and the nonadherent fraction was removed and cryopreserved while the adherent cells had media exchange to CellGenix GMP DC Medium (Freiburg, Germany) with 10% AB male human sera (Access Biologics, Vista, CA), 2 mM L-Glutamine (Gibco, ThermoFisher) and GM-CSF and IL-4 (Miltenyi Biotec, Somerville, MA) at 800 U/mL and 500 U/mL respectively. Media was exchanged every other day until day five when the media was exchanged for DC media with 10% human AB sera with glutamine and a maturation cocktail of PGE₂ 1 μg/mL, human IL-6, IL-1β, TNFα at 1000 U/mL. Cells are incubated overnight and harvested from plates with still adherent cells being collected by incubating on ice for 30 minutes. Cells are cryopreserved or used directly.

A full panel of cell markers can be used to measure all the leukocytes present in a sample to ensure that the right cells and enough cells are present, e.g., CD14⁺ cells for monocytes and CD3⁺ for T cells. All normal cell donors had between 0.5%-14% CD14⁺ of leukocytes and successfully generated DCs. A full panel of the different targets is provided below in Table 13.

TABLE 13
Surface Markers for Co-Staining to Identify Matured DCs

| PBMC, memory | | | |
|---|---|---|---|
| (DO, D14, D21, D28) | | Target | Purpose |
| 1 | Alexa fluor 488 | CD197 (CCR7) | T naïve and central memory or Effector |
| | | | and effector memory |
| 2 | PE | CD56 | NK cells |
| 3 | PE-Dazzle 594 | CD19 | B cells |
| 4 | PE-Cy5 | CD62L | T naïve and central memory or Effector |
| | | | and effector memory |
| 5 | PE-Cy7 | CD8 | CD8⁺ T cells |
| 6 | APC | CD16 | NK cells |
| 7 | APC-Cy7 | CD3 | T cells |
| 8 | BV421 | CD183 (CXCR3) | T cell Differentiation stage |
| 9 | BV510 | Zombie Aqua | Live/Dead |
| 10 | BV570 | CD4 | CD4⁺ T cells |
| 11 | BV605 | CD45RO | Naïve and effector or Memory cells |
| 12 | BV650 | CD14 | Monocytes |
| 13 | BV785 | CD45RA | Naïve and effector or Memory cells |

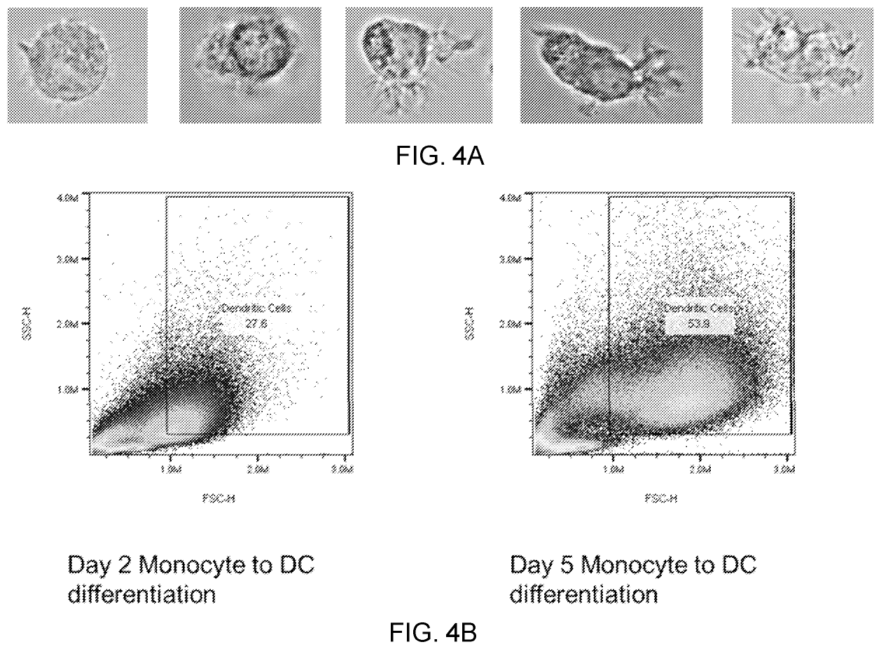

Timing of the differentiation and subsequent maturation before antigen introduction was found to be particularly sensitive and an important parameter. The optimized conditions for generating DCs is five days of culture. Before this point, cells do not appear to be typical DCs (FIG. 4A) and are not large enough as determined by FSC-H and SSC-H (FIG. 4B). After day 5, viability without maturation begins to drop significantly. On day 5, DCs are matured by prostaglandin E2 (PGE₂), IL-6, IL-1β, and TNFα overnight. This is significant as maturation of DCs is required for cell surface expression of costimulatory molecules necessary for T cell priming. If maturation is not performed, then DOs will not generate an effective response in T cells.

A DC identification panel for a set of surface markers was used to identify matured DOs. A full panel of the surface markers is provided in Table 14 below.

TABLE 14
Surface Markers for Co-Staining to Identify Matured DCs

| DC Panel | | Target | Purpose |
|---|---|---|---|
| 1 | Alexa fluor 488 | CD11c | Dendritic cell marker |
| 2 | PE | CD209 | Dendritic Cell-Specific Intercellular |
| | | | adhesion molecule-3-Grabbing |
| | | | Non-integrin |
| 3 | PE-Dazzle 594 | CD197(CCR7) | Migration Marker |
| 4 | PE-Cy5 | CD1a | Lipid and glycolipid |
| 5 | PE-Cy7 | CD3 | T-cells |
| 6 | APC | CD83 | Maturation marker |
| 7 | APC-Cy7 | CD8 | DC1 marker |
| 8 | BV421 | CD14 | Monocyte |
| 9 | BV510 | Zombie Aqua | Dead cell exclusion |
| 10 | BV570 | CD103 | DC1 marker |
| 11 | BV605 | CD11b | DC2 marker |
| 12 | BV650 | HLA-DR | Dendritic cell marker/ |
| | | | maturation marker |
| 13 | BV785 | CD80 | Maturation marker |

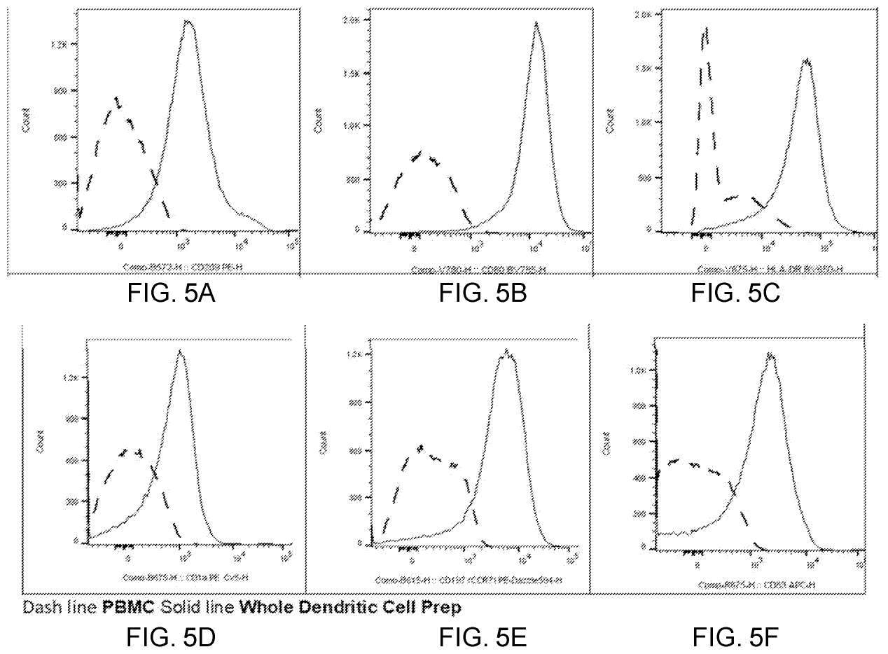

Starting PBMCs are very low for most of these markers as the number of circulating DCs is less than 1% of the total mononuclear cells (Alter 2004; Betts & Koup 2004). FIGS. 5A-5F show flow cytometry analyses of six surface markers that confirm the harvested cells as having a high fraction of cells with markers typical of DCs and how they contrast with the starting PBMC. FIG. 5A shows the marker CD209 (DC-SIGN). DC-SIGN is unique in that it regulates adhesion processes, such as DC trafficking and T cell synapse formation, as well as antigen capture (Alter 2004). FIGS. 5B and 5D show the markers CD80 and CD1a, respectively to identify differentiated DCs (Alter 2004). FIG. 5E shows the marker C—C chemokine receptor 7 (CCR7), a marker known to be critical for the direction and motility of immune cells to secondary lymph nodes (Alter 2004). This is significant for the adaptive immune response, which includes the development of cytotoxic-cells and the Th1 response, which are important for cancer therapy. FIG. 5C shows the marker HLA-DR, a class II MHC and the high expression of which typifies DCs. Lastly, FIG. 5F shows the marker CD83, a member of the Ig superfamily, which is expressed on activated immune cells but is highly expressed on DCs (Alter 2004). Taken together, the results shown in FIGS. 5A-5F indicate that cells harvested on day 6 were significantly enriched with DCs over starting PBMCs.

### Example 5: Dendritic Cell Mediated Stimulation and T Cell Priming

The following example demonstrates that the DCs produced in Example 3 are capable of more efficiently stimulating T cells than the endogenous APCs in PBMCs alone (a model for the peptide based PBMC no DC or mRNA process), indicating that the DCs are functional for T cell priming. The example also shows that DCs allow for expansion in the number of potential antigenic targets.

### Pepmix

For these experiments, LMP2A pepmix and the NY-ESO-1 pepmix was sourced from the JPT Peptide Technologies (Berlin, Germany) catalogue. The pepmixes were resuspended in DMSO at 250 ng/μl. The KRAS G12D pepmix was custom synthesized. A 27 amino acid sequence corresponding to KRAS G12 and G12D with flanking upstream 11 amino acids and downstream 14 was used as the basis of the pepmix. Four peptides each 15 amino acids long tiling across this sequence with an 11 aa overlap were produced and purified to 99% by HPLC. These were resuspended with DMSO at 4 μg/μl and then pooled in a 1:1:1:1 ratio. For experiments involving the polyneoantigen RNA construct, pepmixes followed the same format as KRAS G12D with four 15 amino acid peptides mixed.

### Methods of Generating Dendritic Cells

The next steps included differentiating DCs from monocytes in the patient's whole blood sample.

For PBMCs, the media (T cell Media) is CellGenix GMP DC Medium, 10% human AB sera, 2 mM L-Glutamine with human IL-7 and IL-15 at 3753 U/mL and 525 U/mL, respectively. For PBMCs, 5 million cells per 1 mL of growth media is used with all experiments described occurring at 1 mL initial scale. The plates used were the G-REX 24 well from Wilson Wolf, New Brighton, MN. Peptides are added on day 1 at 1 μg/mL. Every two days, half of the media is exchanged for fresh media without disturbing the cells. For the first week, 3×10⁶ cells/mL volume of media is used per well. For the second week, the density is doubled to 1.5×10⁶ cells/mL and accompanied by a gentle mixing. During the third week, 1×10⁶ starting cells/mL is used. In the fourth week, 0.7×10⁶ starting cells/mL is used. Cultures were assayed at day 14 but the final process goes to day 21 to maximize cell numbers. A polyclonal stimulation using ImmunoCult Human CD3/CD28/CD2 T cell Activator (STEMCELL Technologies) can be performed on day 14 of culture by the addition of 15 μl/2 million cells/ml.

The PBMCs are thawed and plated onto tissue culture grade plastic 6 well plates in RPMI 1640 media at a density of 700,000 cells/cm² and moved into a 5% CO₂ 37° C. humidified incubator for an hour. The anti-aggregate thawing reagent from the Immunospot Corporation (Shaker Heights, OH) was used according to manufacturer's instructions. Benzonase® or DNAse can also be used as an anti-aggregate in the thawing media. Non-adhered cells are then washed off using PBS twice at 2 mL per 10 cm². The saved washes, which contain non-adherent cells including T cells are collected and centrifuged at 330×g, resuspended in CryoStor® CS10 (STEMCELL Technologies), frozen in a control rate freezer to −150° C., and are stored at this temperature. Post washing DC differentiation media consisting of DC media as the base, 10% human sera, 2 mM Glutamax, human IL-4, human GMCSF at 1000 U/mL and 500 U/mL respectively is added to the wells containing the adherent cells at 2 mL per well of a 6-well plate. Cells are moved into a 5% CO₂ 37° C. humidified incubator. Starting the next day and then every other day after that, half of the media is removed, centrifuged at 330×g, and resuspended in fresh media of equal volume and added to the culture. On day 5, all the media is removed, centrifuged at 330×g, and resuspended with maturation media and added to the culture. Maturation media is CellGenix GMP DC media with 10% human AB sera with glutamine and a maturation cocktail of PGE₂ 1 μg/mL, human IL-6, IL-1β, TNFα at 1000 U/mL. Cells are incubated overnight in a 5% CO₂ 37° C. humidified incubator. The next day, media is removed, centrifuged at 330×g, and still-adherent cells having ice cold PBS 2 mL per well in a 6 well are added, incubated on ice for 30 minutes, vigorously washed using the PBS present in the well, and combined with the fraction removed from the well initially. The cells are then counted using the Nexcelom automated counting chamber using AOPI following the instructions for the AOPI cell number and viability stain given by the manufacturer.

Flow cytometer analysis, of surface markers, show that the generated DCs are functional. The FACS experiments included using 1×10⁶ collected DCs or 1×10⁶ cells from a culture that were then washed twice with 1 mL of −/− Dulbecco's Phosphate Buffered Saline (ThermoFisher), with 0.1% AB human sera (Access Biologics) by centrifugation at 330×g for five minutes. Cell pellets are resuspended with the pooled indicated amounts of fluorescently labeled antibodies. Cells are incubated for 15 minutes at room temperature and then washed twice with 1 mL PBS with 0.1% sera. Cells are resuspended in 200 μl flow buffer PBS 2% FBS and then run on the NovoCyte 3000 (Agilent). A minimum of 10,000 cells were collected for each sample and were analyzed using FlowJo software (Tree Star, Inc., San Carlos, CA). Cell debris was eliminated from the analysis by gating on forward scatter and side scatter. Single cells were selected by comparing forward scatter height and forward scatter area. To examine only DCs, a gate was drawn.

### Methods of Determining Functionality of Dendritic Cells

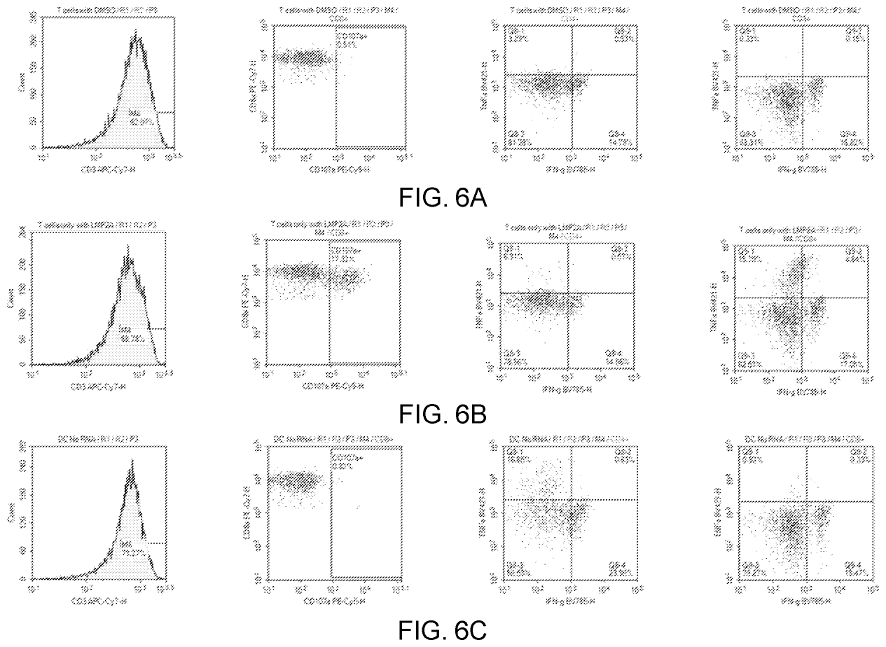

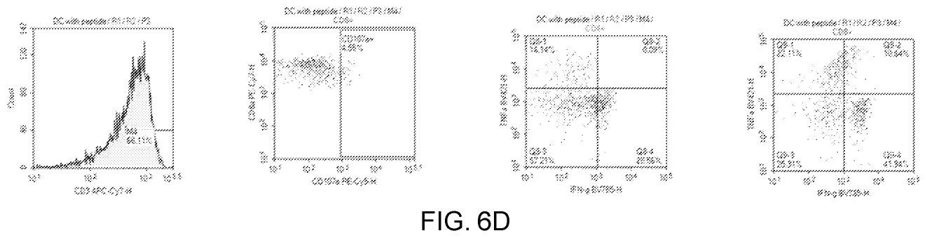

One function of DCs is to stimulate T-cells. T-cell stimulation is measured by their ability to release cytokines. To confirm that the DCs are functional, a cytokine release assay is performed on a flow cytometer using several conditions. A sample of T-cells that had previously been confirmed to produce TNFα and IFNγ in the presence of the EBV antigen LMP2A was used for the T-cells. A sample of DCs from this same donor (MHC matched) were produced. A mixture of 15-mer amino acid overlapping peptides corresponding to LMP2a was purchased from a commercial provider and resuspended in DMSO The following conditions were tested in serum-free cytokine-free media for six hours at 37° C. in a humidified chamber: T-cells with vehicle (DMSO) (FIG. 6A), T-cells with LMP2a peptide added at 1 μg/mL (FIG. 6B), T-cells with vehicle with no antigen added dendritic cells (“DCs”) (FIG. 6C), and T-cells with LMP2a peptide at 1 μg/mL with DCs (FIG. 6D).

The release of TNFα and IFNγ was 0.15% of CD3⁺ T cells alone (FIG. 5A), T cells plus peptide 4.64% (FIG. 6B), T cells plus DC 0.33% (FIG. 6C), and T cells plus DC LMP2A peptide 10.64% of CD3⁺ T cells (FIG. 6D). The results suggest that DCs are capable of more efficiently stimulating T cells than the T cells alone and are, therefore, functional DCs.

Next, the DCs were both tested for their ability to prime or stimulate T cells and their capacity to be integrated into the mRNA or peptide T-cell production process

### T Cell Production Process

The disclosed peptide T cell production process is one in which collected PBMCs are combined with tiling 15 aa peptides of the antigen of interest and cultured for 14-28 days in IL-7 and IL-15. Peptide is then added at 1 μg/mL on the first day of culture. The antigen presenting cells (APCs) present in PBMCs should prime T cell response. The DC method depletes the monocyte APCs, differentiates them into DCs, and then reintroduces them to the rest of the starting cell population. This is similar to the PBMCs with one of with the distinct differences in that the antigen presentation capacity has been greatly improved by enriching for the APCs. Therefore, the process post day one of culture could be the same for pepmix antigen targets such as EBV proteins LMP1, EBNA1, LMP2A that were previously successfully PBMC primed.

Priming T Cells with LMP2A Pepmix Using the Peptide T-Cell Production Process

An experiment using these principles was conducted with LMP2A pepmixes. A peptide PBMC based, and peptides added to previously differentiated and matured DC added based priming was conducted for three blood donors. Day one is considered the point at which either PBMCs were thawed into culture with LMP2A peptides or matured DCs were harvested and combined with thawed non-adherent cells and LMP2A peptides. The DCs were added at a ratio of 1 DC: 4 T cells; however other ratios are possible.

On day 6 of the monocyte to DC differentiation, the harvested DCs are combined with personalized neoantigen peptides. DCs are combined with the non-adherent cells that had been frozen down on the first day of DC production. They are combined in the ratio of 2:1 nonadherent cells (T cells) to dendritic cells. Cells are thawed using anti-aggregate from Immunospot. The total volume is 1 mL at a cell density of 3×10⁶ cells/mL using CellGenix GMP DC Medium, 10% human AB sera, 2 mM L-Glutamine with human IL-7 and IL-15 at 3753 U/mL and 525 U/mL respectively. Typically, half the volume is used to resuspend pelleted nonadherent cells and combine with DCs suspended in the other half. Peptides resuspended in DMSO are added so each peptide is at the final concentration of 1 μg/mL. DMSO concentration must not exceed 0.5%. Cells are cultured using the G-Rex® cell culturing device (Wilson Wolf, New Brighton, MN). The devices used depends on the cell scale needed G-Rex® 24 well (2 cm²), G-Rex® 6 well (10 cm²), G-Rex®10M-CS (10 cm²) or G-Rex® 100M-CS (100 cm²). The scaling is linear and translates into the closed system versions for manufacturing. The culture is moved to a 5% CO₂ 37° C. humidified incubator. Every two days, half of the media is exchanged for fresh media without disturbing the cells. For the first week, 3×10⁶ cells/mL volume of media is used per well. For the second week, the density is doubled to 1.5×10⁶ cells/mL and accompanied by a gentle mixing. On the third week 1×10⁶ starting cells/mL is used. On the fourth week 0.7×10⁶ starting cells/mL is used.

Counting the day in which T cells are combined with DCs as zero, the process may be complete on day 14, 21, or 28 depending on the number of cells present in the culture being enough but not limited to 100 million to above one billion cells. If there are equal to or above 70% CD3⁺ T cells measured by flow cytometry, the process product is considered adequate for infusion. Confirmation of reactivity is assessed after day 14 by cytokine release (IFNγ ELISpot) and cytotoxicity. With the use of the polyclonal antibody, final cell numbers are increased from a 3-fold increase over the starting number on day 21 to a tenfold increase over the starting cell number on day 21.

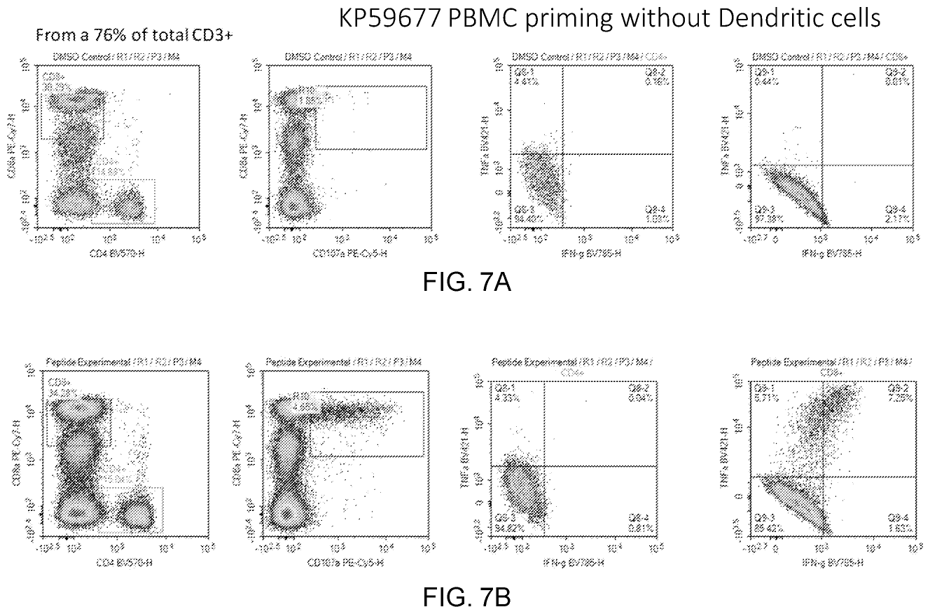

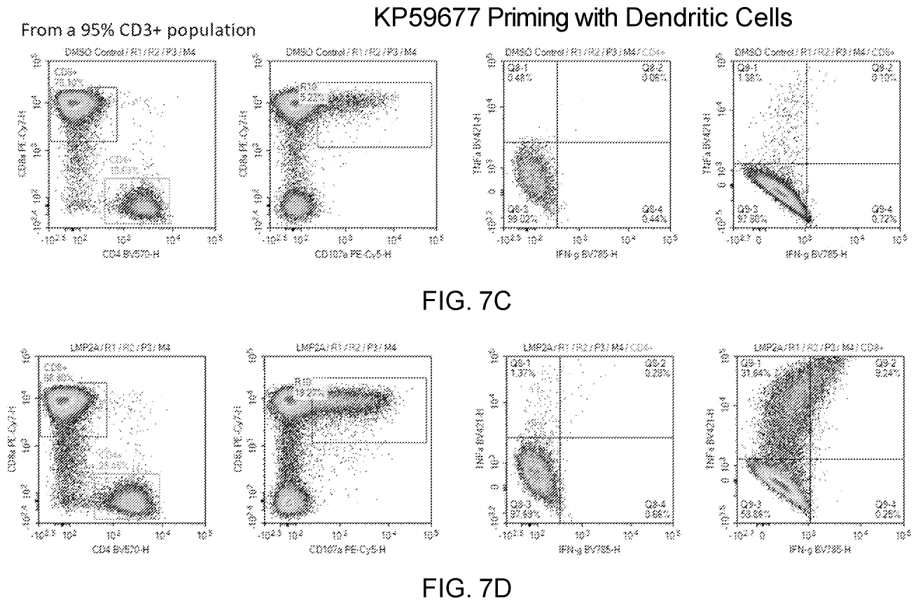

At day 14, a surface protein and intracellular cytokine FACS staining was performed (FIGS. 7A-7D). FIGS. 7A-7B show that priming was successful for a population having 76% total CD3⁺, and FIGS. 7C-7D similarly show that the priming was successful from a population having 95% of total CD3⁺. The priming was considered successful in both cases (FIGS. 7B and 7D) as there was an increase in percent of CD3⁺ T cells that were TNFα and IFNγ producing cells over the background vehicle control (FIGS. 7A and 7C). For cells producing in the PBMC priming fraction, there was 12.96% TNFα⁺ and 8.88% IFNγ⁺. In DC priming, there was 40.88% TNFα⁺ and 9.5% IFNγ⁺. These results are typical with the DC priming trending higher in both the fraction of CD3⁺ T cells produced and in cells that respond with cytokine release after antigen introduction.

It is contemplated that the observed improvements in priming are a result of the CD4⁺ populations of samples containing DCs, a significant TNFα release even without peptide added as was demonstrated previously in FIGS. 6A-6E. It is further possible that DCs have a means to activate CD4⁺ cells beyond the classic MHC synapse and the cytokine production of these CD4+ assists in priming other T-cell types.

This example further demonstrates that in addition to improved efficiency of stimulation, this method also affords an expansion in the number of potential targets. To date, there are no processes that are able to generate a population of double positive TNFα IFNγ producing T cells from PBMCs (e.g., nonadherent cell fraction) that are able to distinguish a single amino acid change within an antigen while not cross reacting with its wild-type version. This stimulation process therefore generates T cells having stringent antigenic specificity that avoids off-target effects common with immunologic therapeutic strategies resulting in safer and more effective therapies.

Priming T Cells with KRAS G12D Pepmix Using the Disclosed T Cell Process

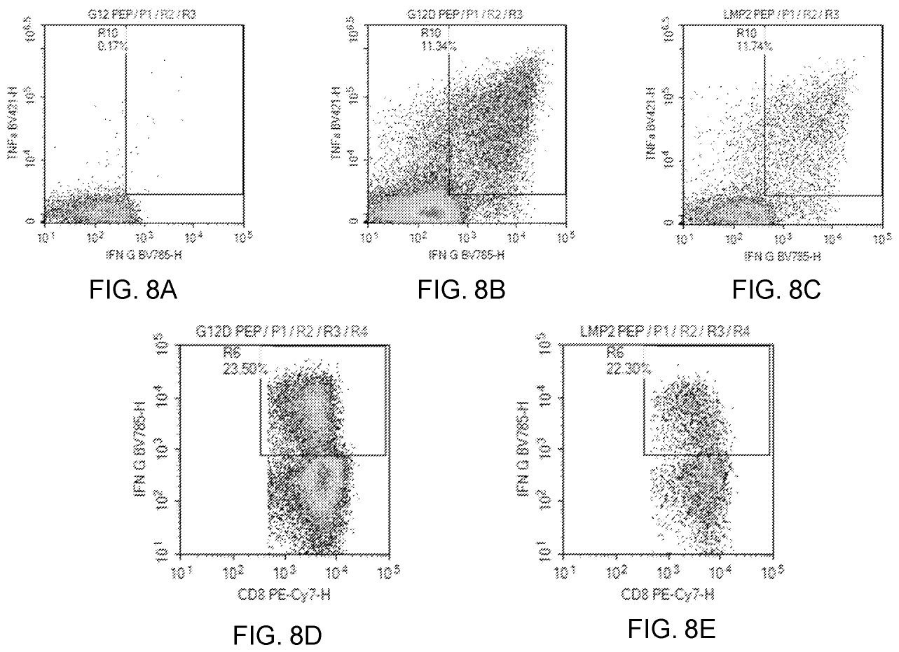

An experiment was conducted with a KRAS G12D neoantigen pepmix. The KRAS G12D pepmix was custom synthesized and purified to 99% by HPLC. As previously discussed, and identified in Table 10, this mutation is present in 1 in 20 cancer patients and is considered oncogenic. At the end of the peptide T-cell production process, CD3⁺ T cells do not react to germline sequence (FIG. 8A) but do produce TNFα⁺IFNγ⁺ response in 11.34% of cells upon addition of G12D (FIG. 8B). Even more impressive is that DCs also produce TNFα⁺IFNγ⁺ in a treatment scale within 14 days. In a separate control priming culture, a similar experiment was conducted for LMP2A and indicated a comparable response (FIG. 8C). This is significant as LMP2A is a viral antigen that 80% of people have encountered before and therefore, a strong response is expected.

The capacity for a T cell to replicate and be an effective immune cell is directly proportional to how long the T cell has been in culture. As such, cells that are produced may have better performance than ones that have been extensively cultured to produce enough cells for a treatment.

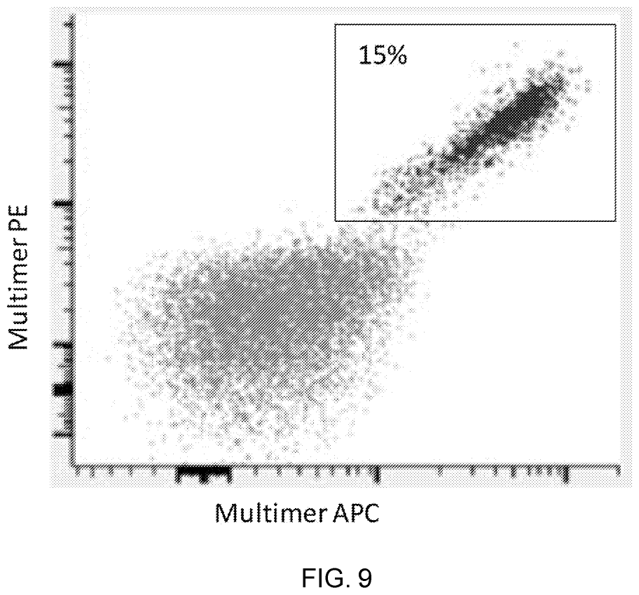

The peptides may also be used in combination with a viral peptide as in FIG. 9. Viral antigens have been shown to be closely associated with certain types of cancer and can serve as a helper antigen. FIG. 9 confirms the presence of an anti G12D TCR by MHC multimer FACS analysis.

The cytotoxicity analysis was performed on cells from the KRAS G12D culture. The 7-AAD/CFSE cell-mediated cytotoxicity assay kit was used for this analysis. The principle behind the assay is labeling all cells used as targets with CFSE followed by incubation with cytotoxic-cells (effectors) and assessment of fraction of target cells killed by a viability dye 7AAD. The assay is specific for dying target cells through use of flow cytometry analysis of CFSE⁺ live/dead cells. Target cells are donor matched PHA blasts loaded with peptide antigen. These are PBMCs stimulated with Phytohemagglutinin-L (Sigma-Aldrich) to induce proliferation for expansion. To generate PHA blasts, PBMCs are plat-d at 2-5×10⁶ cells/ml in RPMI 1640, 10% fetal bovine sera (PHA media) and 100 U/mL IL-2 (Miltenyi). PHA (Sigma) is added to 2.5 μg/mL. Every three days cells are washed and replat-d at 2-5×10⁶ cells/ml.

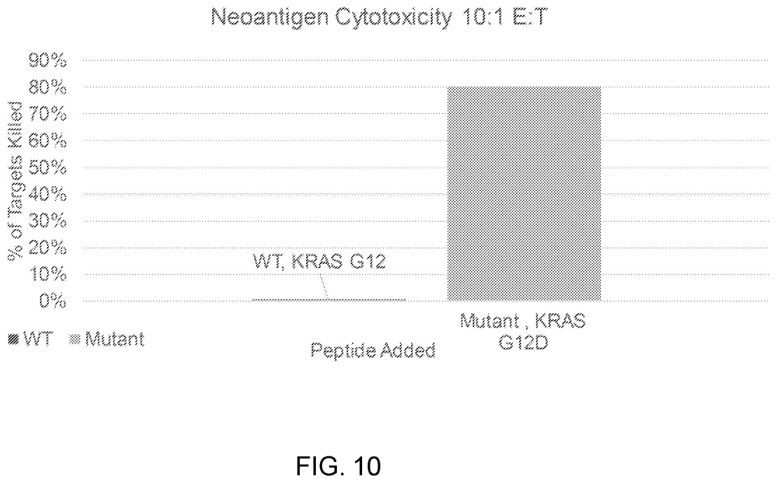

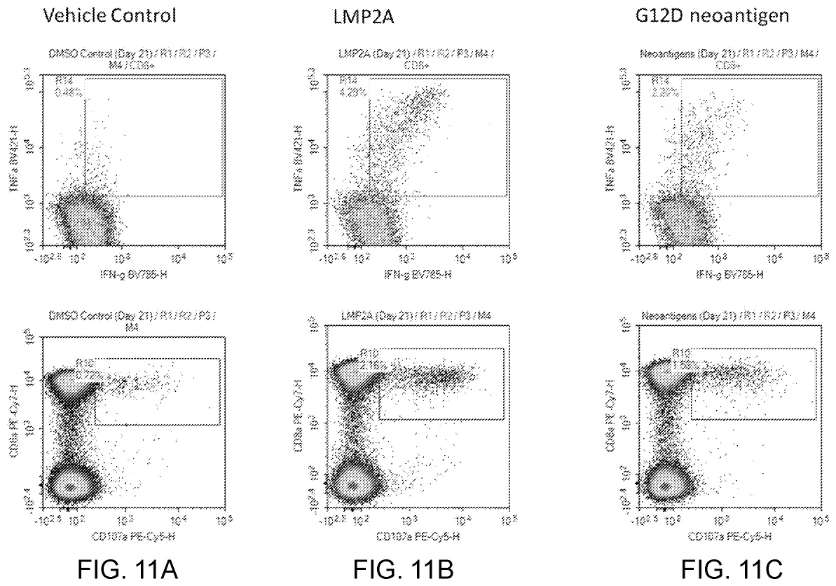

For this experiment, target cells are washed and stained with CSFE in assay buffer for 15 minutes. After washing, cells are incubated with 1 μg/mL of antigen such as LMP2A pepmix in PHA media for 90 minutes at 37° C. Effector cells are harvested at indicated timepoint from a T cell priming culture and washed. Effectors and targets are then combined in 1 mL assay buffer at the designated ratio, e.g., 5:1, 1:1 with a minimum number of 5×10⁵ targets. Combined cells are incubated for six hours, washed, and stained with 7-AAD. Samples were run on NovoCyte 3000 (Agilent). Data is analyzed by comparing effectors and targets with antigen and without antigen or to a sham antigen. Additional controls are included: targets without CFSE or 7AAD, targets with CFSE, targets with 7AAD, and targets with 7AAD and CFSE. FIG. 10 is a CSFE based cytotoxicity assay in which target PHA blasts from a matching donor are killed by effector cells from a KRAS G12D culture where the ratio of effectors to targets is 10:1. The cytotoxicity, of this culture, was also tested where the peptide is normal KRAS G12 and does not show significant killing of G12 targets. It is important to note that a priming reaction for KRAS G12D was tested on 12 different donors using the peptide PBMC no DC no mRNA process and the results were negative.

T Cells Targeting Multiple Antigens Using the mRNA T Cell Production Process

In a follow-up experiment, the capacity of the culture to target multiple types of antigens in a single well was assessed. The peptides can compete for loading with peptides at higher concentrations and low binding affinities overrepresented on MHC. FIGS. 11A-11C show results after priming was carried out on a combined KRAS G12D and LMP2A pepmixes at 1 μg/mL for each 80% CD3⁺ and 43% CD8⁺ population. Culture time was extended to 21 days to expand the number of cells available for testing. It was also observed that the fraction of responding cells increased over time.

A surface protein and intracellular cytokine FACS staining was performed with either LMP2A or KRAS G12D or vehicle control. Examining the CD3⁺CD8⁺ cells reveals a TNFα⁺IFNγ⁺ fraction for LMP2A of 4.29% and KRAS G12D of 2.2%. CD107a is lysosomal-associated membrane protein 1 (LAMP-1) and is used to measure cytotoxic potential of CD8⁺ cells (Alter 2004; Betts & Koup 2004). The fraction of CD107a CD3⁺CD8⁺ cells are 2.16% LMP2A and 1.58% G12D. The fraction of reactive cells for KRAS G12D and LPM2a is lower than that shown in FIGS. 8A-8B. This suggests that simultaneous priming and the characteristics of the peptides used may influence priming results.

Lastly, experiments were conducted to determine if the DC process could be applied to tumor associated antigens (TAA) NY-ESO-1. TAA are proteins that are over expressed in cancer and do not necessarily contain amino acid sequence changes. The NY-ESO-1 antigen used for the following experiment is the full-length consensus sequence provided by NCBI. The source of the cells is whole blood from two stage IV glioblastoma patients, and prior experiments were conducted with normal healthy donors. There are significant differences between the cells derived from healthy and glioblastoma patients. Cancer patients are typically older, and the patients sourced had started chemotherapy, both of which led to immune dysfunction. Accordingly, the use of cells from these patients tests the performance of the peptides added to DCs for presentation to T cells process for its intended purpose successfully.

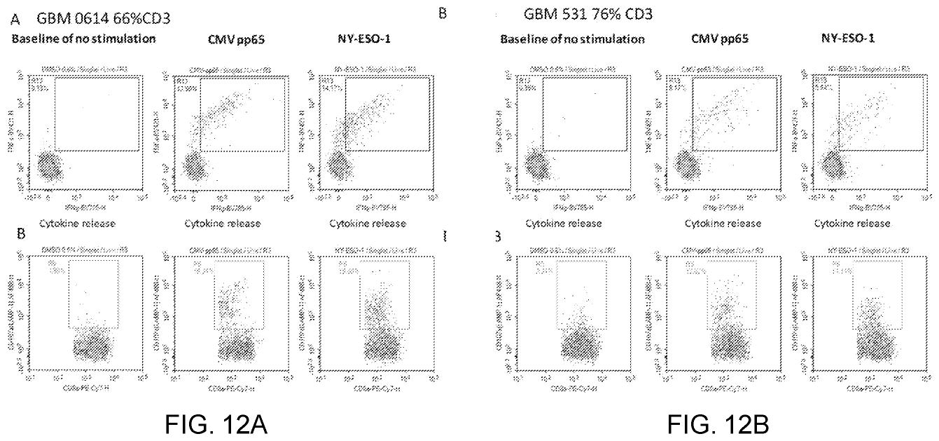

A control viral peptide CMV pp65, a commonly used and very immunogenic peptide, was included with the NY-ESO-1 pepmix. A surface protein and intracellular cytokine FACS staining was performed on day 14 and the response of the CD3⁺CD8⁺ cells was determined for each antigen. FIGS. 12A-12B show the response for the GBM 66% CD3 and GBM 75% CD3 population, respectively. There was a measurable response in both patients for both TNFα⁺IFNγ⁺ cytokine release and CD107a surface staining. The response to NY-ESO-1 indicates that the peptide T-cell production process is not limited to viral antigens and neoantigens and includes the potential to target “self” antigens if they are the selected antigen. This is unexpected as, for part of their production, T cells undergo negative selection for germline amino acid sequences in the thymus by stromal cells.

### Example 6: Antigen Gene Transfer to Dendritic Cells

Having established that the DC process can effectively prime T cells against an array of targets using peptides, the following example provides details regarding the incorporation of mRNA encoded antigens into the cells rather than peptides. mRNA

There are several advantages to the use of mRNA relative to peptides. A gene can be synthesized from DNA in 2-3 days, transcribed in one day, and transfected into DCs the next day. In contrast, the quickest a peptide can be produced is 6-8 weeks at only microgram amounts with no purification. Transferred mRNA produces proteins from the endogenous machinery of the cell whereas peptides must be added at comparatively high concentrations to the media to saturate all available binding sites. This allows mRNA to mimic a natural cancer cell more accurately. Additionally, mRNA enables post translational modifications because the machinery is in place at the time of production, and the DCs receive any potential instructions encoded upon the introduction of the mRNA. Further, modified peptides are rare as compared to other unmodified introduced peptides and are not well represented in the MHC. In contrast, mRNA can encode full length functional proteins, and any immunological processing based on the structure or function of the protein can be utilized. Lastly, molecules such as the DC costimulatory membrane surface proteins CD80/CD86 and antigens improve the efficiency of priming. Taken together, each of these advantages can lead to superior T cell treatment.

mRNA Production

To produce mRNA, DNA sequences are synthesized with a commercial synthesizer, which can take up to two days. This sequence is cloned into a plasmid that can be grown in bacteria in two days. Restriction enzyme sites are incorporated into the ends of the synthesized sequence. Complementary sites on the destination plasmid are cut with a restriction enzyme. The gene is then ligated into the plasmid using ligase and transformed into competent E. coli DH5a and plated on agar. Selection of colonies on the agar plate occurs because an antibiotic that only cells with the plasmid can grow on is included in the agar. Colonies are picked and placed into LB broth containing the same antibiotic as the plates and grown overnight. Silica membrane plasmid purification such as the Qiagen Maxi Prep can be carried out according to manufacturer's instructions, and samples of plasmid clones can be sent to a commercial sequencer for verification.

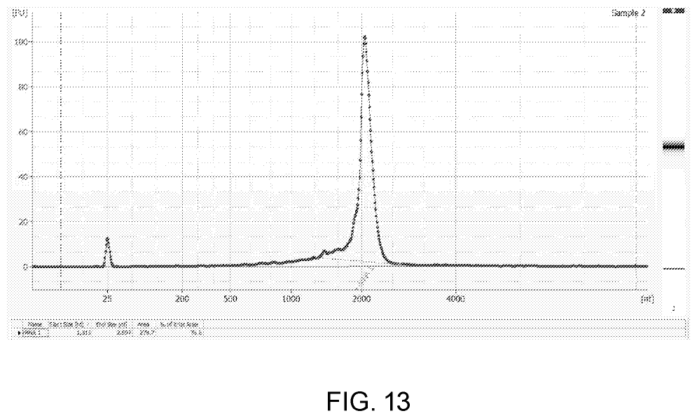

Purified plasmid is then used as a template for PCR using primers for the gene of interest that include a polyT tract at least 80, preferably 120 nucleotides long. Products are verified using agarose gel electrophoresis or the Agilent Bioanalyzer 2000 electrophoretic capillary system. The PCR product is then used as template for in vitro RNA transcription. The in vitro transcription reaction is water based and has final concentrations: ATP, CTP, GTP, 5 methoxyUTP at 5 mM; Cleancap™ AG 4 mM, 1× T7 transcription buffer (New England Biolabs) murine RNase inhibitor (NEB) 1U/ul, Yeast inorganic pyrophosphatase 0.002U/ul, T7 polymerase 8 U/ul and Template 1.25 ug/50 ul reaction. Phosphatase treatment of RNA followed by HPLC is performed. HPLC is performed with an AKTA 10 machine with a RNASep™ Prep Column with Buffer A 0.1M Hexylammonium Acetate in 10% acetonitrile and buffer B 0.1 M Hexylammonium Acetate in 25% acetonitrile. Alternatively, or subsequently, polyT coated beads could be used to find mRNAs that were fully transcribed. The RNA concentration is measured on a Nanodrop spectrophotometer. The purity after HPLC is determined via Bioanalyzer 2000 with RNA nano chip as in FIG. 13. From receiving sequence results to mRNA only takes around five days.

### Testing Cell Survival Post Transfection

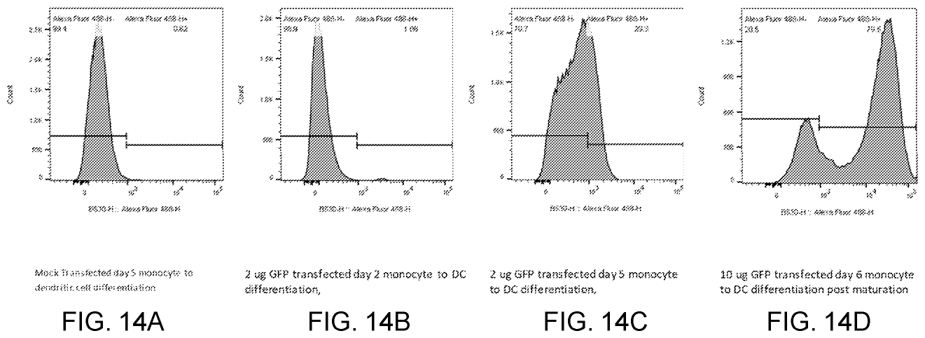

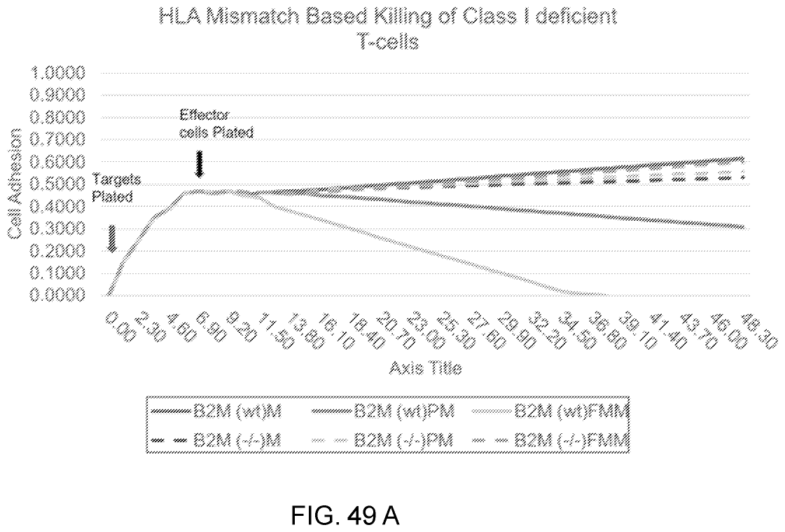

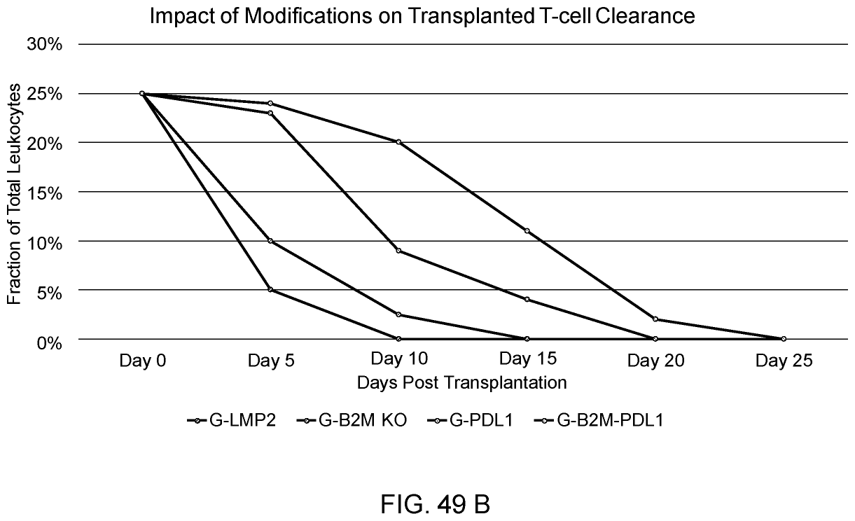

Despite the numerous advantages associated with using mRNA, mRNA is principally based on gene transfer, and transfection is frequently toxic to primary cells (e.g., not immortalized cells). To address the issue of potential toxicity, an experiment was conducted to examine if and when DCs can be transfected without causing toxicity. Using the Lonza nucleofector and a commercially available mRNA for eGFP (Trilink), DCs were tested under a mock transfection experiment. The Lonza nucleofector allows for mRNA expression that can read on a flow cytometer at each timepoint. To evaluate toxicity, cells were transfected with 2 μg or 10 μg of eGFP RNA (FIGS. 14A-14D). Only on day 6 of differentiation, post-maturation by PGE₂, IL-1β, TNFα, and IL-6, did the DCs have strong GFP expression and were viable enough for use in priming with 79% GFP⁺ versus 1% on day 2 and 29.3% pre-maturation on day 5. Within an allogenic environment, removing MHC Class I results in an increased half-live within the patient. This protects the patient early for a week or a few weeks while their own immune system mounts a response. Knocking out β2-microglobulin, on chromosome 6p21, using CRISPR/Cas9 was applied to cause a defect in the MHC class I structure. Previously selected COVID-19 T-cells are used for knocking out the β2-microglobulin by CRISPR/Cas9. A commercial kit was used to knockout β2-microglobulin (OriGene, KN207587RB). Used the β2 Microglobulin gRNA vector 1 with a target sequence of GAGTAGCGCGAGCACAGCTA in pCas-Guide CRISPR vector (OriGene, KN207587G1) and β2 Microglobulin gRNA vector 2 with a target sequence of ACTCACGCTGGATAGCCTCC in pCas-Guide CRISPR vector (OriGene, KN207587G1). The donor DNA containing left and right homologous arms and selection markers Red Fluorescent Protein and Blasticidin functional cassette (OriGene, KN207587RB-D). Turbofectin-8 was used to transfect the three vectors into T-cells in suspension. Following the manufacture's knock-out protocol, screened the final B2M knocked-out T-cells by comparing the half-life in a digital MLR assay. The cells were plated into xCelligence Real-Time Cell Analysis (RTCA) cartridge and then exposed to matched, partial matched, and fully mismatched allogenic PBMCs. Determined if there was a difference in the half-life between the β2-microglobulin knock-out vs the pre-knock-out/wild-type T-cells. FIG. 49A shows an exemplary result from the RTCA killing assay. The readout shows the cells voltage impedance (Cell Index) versus time.

mRNA Construct

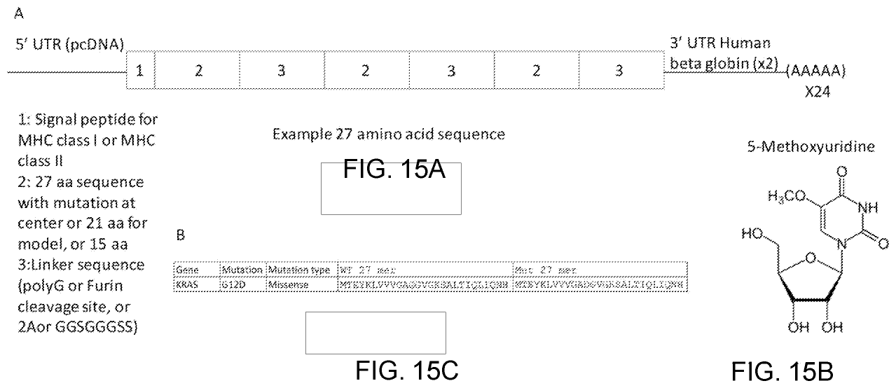

Having established that the mRNA transfection process is not toxic to primary cells, the next steps were to develop a suitable mRNA construct. FIG. 15A shows an exemplary mRNA construct consisting’ of a 5′ untranslated region (UTR), a signal peptide, a repeating unit of antigen and polylinker, a 3′ UTR containing two repeats of the human beta globin 3′ UTR and a poly A tract to hard code the polyadenylation sequence. A consensus Kozak sequence is present at the start, and the translated region begins with a 24 aa signal domain taken from HLA-A.24. The signal domain from HLA-B, HLA-C, HLA-DRB1, LAMP1, LAMP2, TAP1, TAP2 also can serve as embodiments of the signal/leader sequence. The 3′ UTR from the alpha globin, beta globin from Rattus norvegicus or Pan troglodytes are other embodiments. A signal peptide is required as all proteins have to start with methionine (FIG. 15B), and the number of epitopes would be severely limited without a signal peptide. The signal peptide is cleaved off and remains in the membrane so it should not compete with the antigen in class I HLA-ABC.25 (Lemberg 2001). The signal peptide also has a function of directing the amino acids to the MHC class I compartment. The signal peptide is followed by a 21 amino acid sequence with the neoantigen changes located at the center and germline sequence flanking it. A 21 amino acid sequence was selected because it is the greatest number of amino acids that can bind to an MHC I, 11, that can include the mutant on either flank. In an alternate arrangement, a 27 amino acid sequence could be used, consistent with the pepmixes, or 15 amino acids (FIG. 15C).

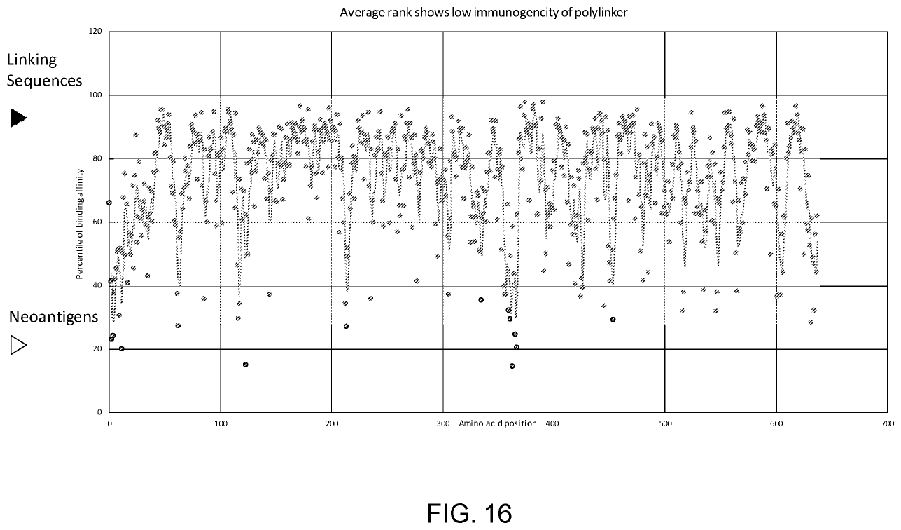

If multiple antigens are to be included, then the polylinker amino acid sequence (GGSGGGSS) is added between them. Importantly, this linker has low immunogenicity as indicated by use of the NetMHC MHC I binding affinity tool. The neoantigen sequences of interest are wholly contained in the areas in which binding affinity is below the 50 percentiles (FIG. 16) where lower rank indicates better binding. Other linking sequences are possible such as polyG, Furan cleavage sites, 2A sequences, other peptide sequences that are not immunogenic.

### Linking Sequences

To verify that the T cells produced do not target the linking sequences, the reactivities of the T cell's individual neoantigens vs the full polylinker construct containing the germline sequences was examined in vitro. If negative, then it is very unlikely to have off targets consisting of mixed polylinker and target sequence. MHC binding analysis such as by NetMHC (www.cbs.dtu.dk/services/NetMHC/) indicates that the polylinker sequence has poor binding capacity (FIG. 16).

Priming T Cells with mRNA

The DC priming process was modified to incorporate mRNA by transfecting matured DCs and immediately combining them with the non-adherent cell fraction in T cell media containing IL-7 and IL-15. The purity of the mRNA quality was assessed by agarose gel electrophoresis, measured by spectrometry, and stored at −80° C. The gene transfection method chosen for this series of experiments is nucleofection, which is comprised of a mix of electroporation and cationic lipids. For transfection, one to two million DCs are pelleted and then resuspended in 100 μl of the human dendritic cell nucleofection kit reagent from Lonza. After suspension, 2 μg of RNA is added, and the mix is transferred to an electroporation cassette. After nucleofection, 0.5 mL of T cell media was added, and cells transferred into a G-Rex 24 with 0.5 mL T cell media containing twice as many nonadherent cells as transfected DCs. Both must be derived from the same donor.

On day 6 of the monocyte to DC differentiation, the harvested DCs are transfected with mRNA encoding antigen. The Lonza nucleofector II/b was used according to the manufacturer's instructions using program U-003 or the Lonza 4D nucleofector program CB150. 2 μg of RNA per million DCs is used per transfection. This is total RNA transfected and includes a mix of mRNA constructs such as the neoantigen construct and the LMP2A full length sequence. To transfect on a larger scale such as the potentially 40 million DCs gathered from 500 mL of blood, the Lonza nucleofector 4D can be used with scaling of reagents. Immediately following transfection, no wash, DCs are combined with the non-adherent cells that had been frozen down on the first day of DC production. They are combined in the ratio of 4:1 nonadherent cells (T cells) to dendritic cells. Cells are thawed using anti-aggregate from Immunospot. The total volume is 1 mL at a cell density of 3×10⁶ cells/mL using CellGenix GMP DC Medium, 10% human AB sera, 2 mM L-Glutamine with human IL-7 and IL-15 at 3753 U/mL and 525 U/mL respectively. Typically, half the volume is used to resuspend pelleted nonadherent cells and combine with DCs suspended in the other half. The plate is the brand G-Rex from Wilson Wolf such as the G-24 or G-100 depending on size. The culture is moved to a 5% CO₂ 37° C. humidified incubator. Every two days, half of the media is exchanged for fresh media without disturbing the cells. For the first week, 3×10⁶ cells/mL volume of media is used per well. For the second week, the density is doubled to 1.5×10⁶ cells/mL and accompanied by a gentle mixing. During the third week, 1×10⁶ starting cells/mL is used. In the fourth week, 0.7×10⁶ starting cells/mL is used.

Assessment of T-cell reactivity is carried out by peptide challenge. Alternatively, reactivity testing is made peptide free by transfection of HLA matched monocytes, or DCs with mRNA encoding the peptides that are subsequently used for challenge. The RNA method has significant advantages as no peptides need to be produced for testing reactivity. The DMSO control sets the background levels and wells with more spots than control is positive. The germline (wild type) amino acid sequence is also tested. If a reactivity is found against these self-sequences, then the degree of response is taken into consideration. Marginal reactivity as compared to neoantigen sequence can still be used.

Activated T cells from the patient such as PHA blasts are loaded with peptide or transfected with mRNA encoding each of the target neoantigens. PHA blasts are used because they proliferate rapidly and have matching HLA to culture. Effector cells from the priming culture are then added to antigen loaded targets and apoptosis in the target cells is measured by a viability stain gated on CSFE labeled targets. The germline (wild type) amino acid sequence is also tested. If a reactivity is found against these self-sequences, then the degree of response is taken into consideration. Marginal reactivity as compared to neoantigen sequence can still be used. For example, a culture that is positive for several germline sequences on IFNγ but has no cytolytic activity against those sequences but does so against the neoantigen will be used.

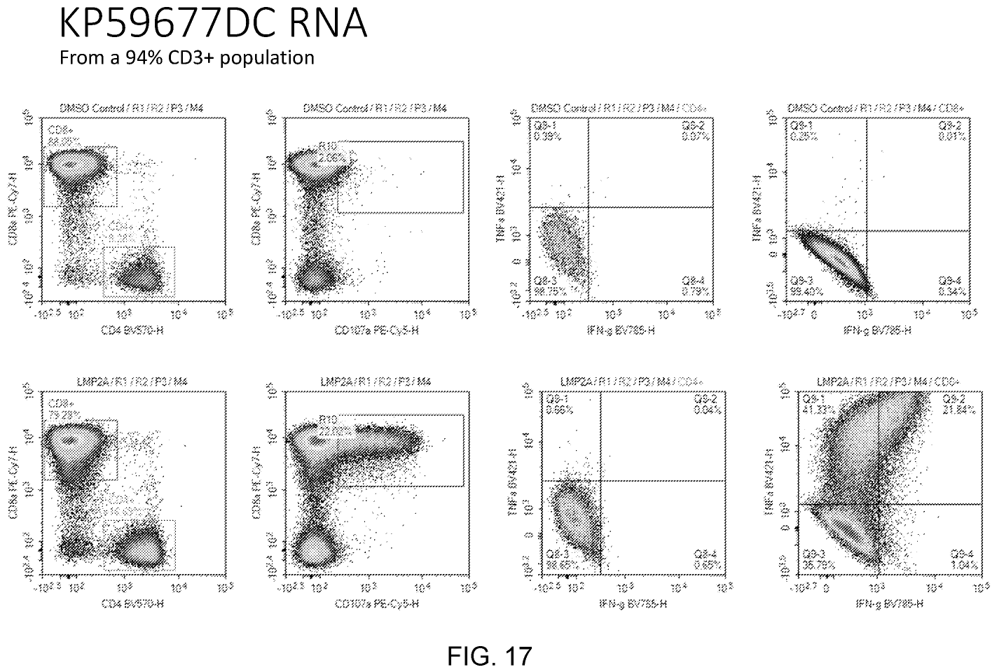

Using the methods described above, three donors were transfected. At day 14, a surface protein and intracellular cytokine FACS staining was performed. All three donors were found to respond to LMP2A on day 14 (FIG. 17). When considering the resulting fraction of CD8⁺, TNFα⁺, and IFNγ⁺ cells for three types of stimulation from a single donor the PBMC alone no DC Stim (FIGS. 7A-7B) to DC peptide stim (FIGS. 7C-7D), to DCs expressing exogenous LMP2A mRNA stim (FIG. 17), the highest fraction of responding cells, is in the mRNA stim at 21.84%. These results suggest that a robust T cell response can be generated from mRNA encoding antigen.

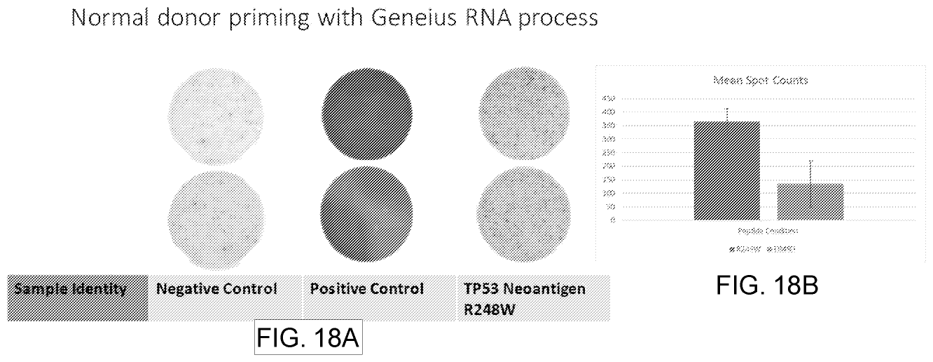

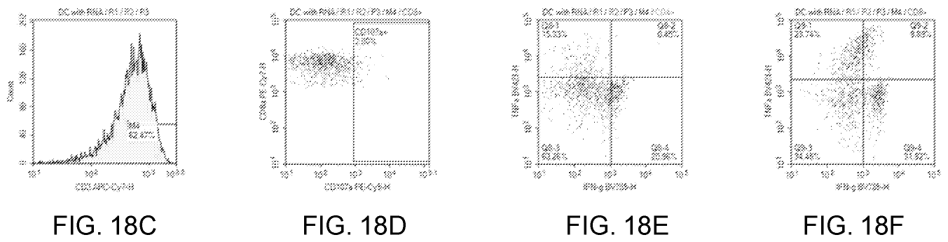

Next, an mRNA encoding a single neoantigen TP53 R248W was tested for its ability to transfect matured DCs (FIG. 18A-18B). The experiment was conducted using a colorimetric IFNγ ELISpot kit from Immunospot. Human IFNγ-1 M/2 was used according to the manufacturer's instructions. In brief, either 2×10⁵ or 1.5×10⁵ cells were plated per well in the Immunospot Test medium. Cells were incubated for 48 hours at 37° C., 5% CO₂ humidified chamber and were developed. Automated spot counting was conducted through Immunospot CRO service. The ELISpot for IFNγ producing cells encoded with a single neoantigen TP53 R248W indicated a greater number of spots than in the vehicle control (FIG. 18B). The results were 134±86 for vehicle and 364±49.

### Methods of Determining DC Functionality of Dendritic Cells

Similar experiments as those discussed above in relation to FIGS. 6A-6D for testing functionality of DCs after the introduction of mRNA is shown in FIGS. 18C-18F. An mRNA for LMP2a was produced. The amino acid sequence for the EBV latent membrane protein 2 (LMP2A) is taken from Swiss-Prot ID: P13285. The amino acid sequence was back translated to a DNA sequence with the EMBOSS Backtranslation tool (www.ebi.ac.uk/Tools/st/emboss_backtranseq/). The signal domain from the first 24 amino acids of HLA-A was also back translated with this tool (Kreiter 2008). The human beta globin 3′ UTR sequence is taken from NCBI Reference Sequence: NM_000518.5. A construct beginning with a Kozak sequence followed by the signal sequence followed by the full LMP2A sequence followed by the beta globin 3′ UTR was ordered from GeneArt, ThermoFisher and cloned by GeneArt into pcDNA3.1⁺. RNA was produced from in vitro transcription and nucleofected into DCs. As indicated in FIGS. 18C-18F, the release of TNFα and IFNγ shows that DCs are functional after introduction of the mRNA and are capable of more efficiently stimulating T cells.

T Cells Targeting Multiple Antigens Using the mRNA T-Cell Production Process

This experiment was followed by evaluating the ability to transfect the multi neoantigen polylinker construct described above with matured DCs. There are several advantages to placing all of a patient's identified mutation in one construct. The practical advantage is quality control is easier with less reagents, it saves cost by limiting the number of syntheses and cloning required, and only a single priming reaction is required. This means that all of a patient's collected cells can interrogate all available antigens thereby leading to a more reliable process. In terms of molecular biology, it is easier to optimize one transfection, and each cell is receiving the same genes instead of a mixture that can vary cell to cell. It is also believed that there is a cooperative effect in priming. Each activated T cell not only produces cytokines that trigger their growth but also the growth of other cells present. To model this, 21 neoantigens were selected based on frequency (Tables 1-10).

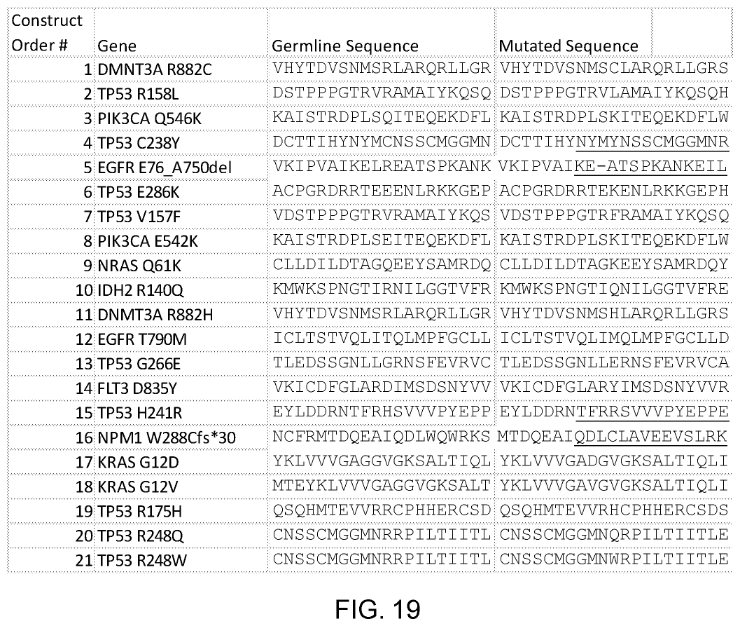

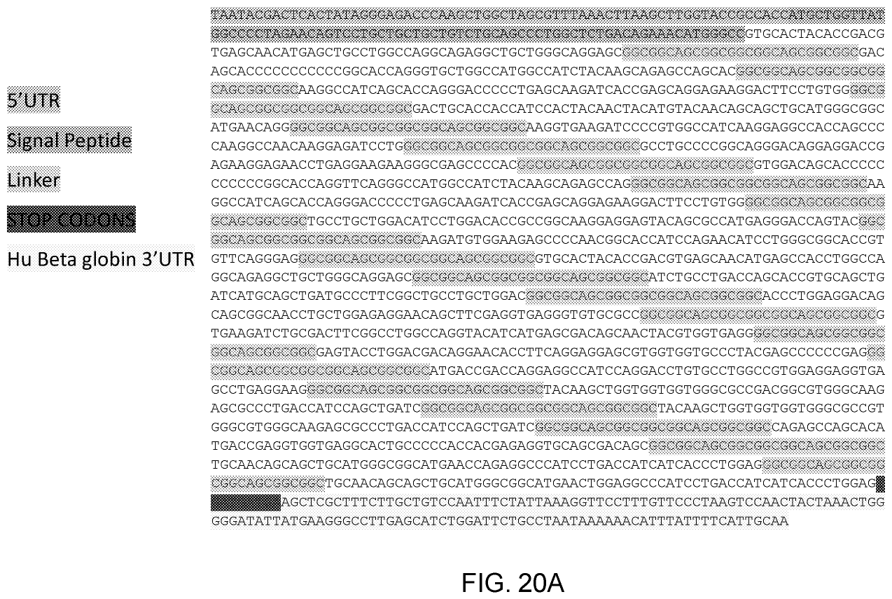

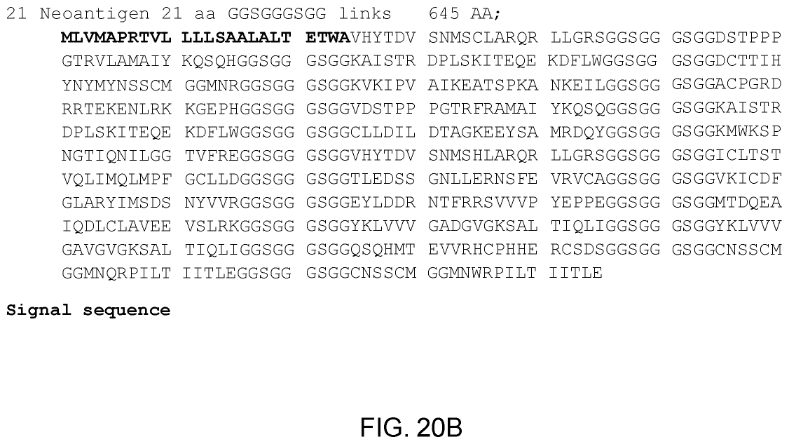

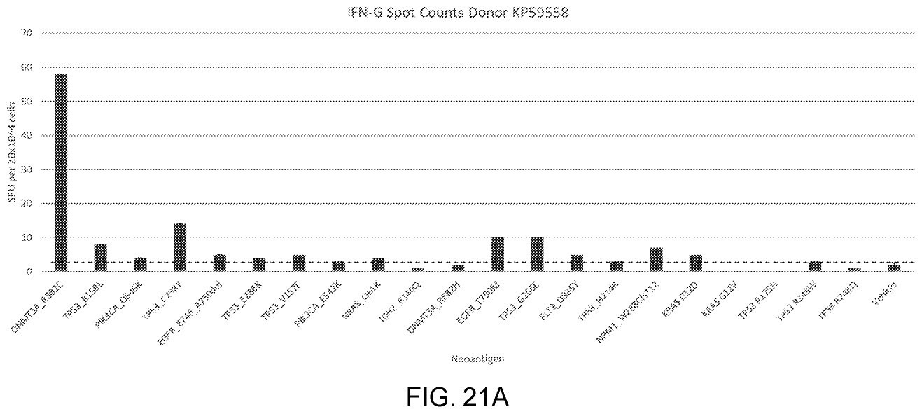

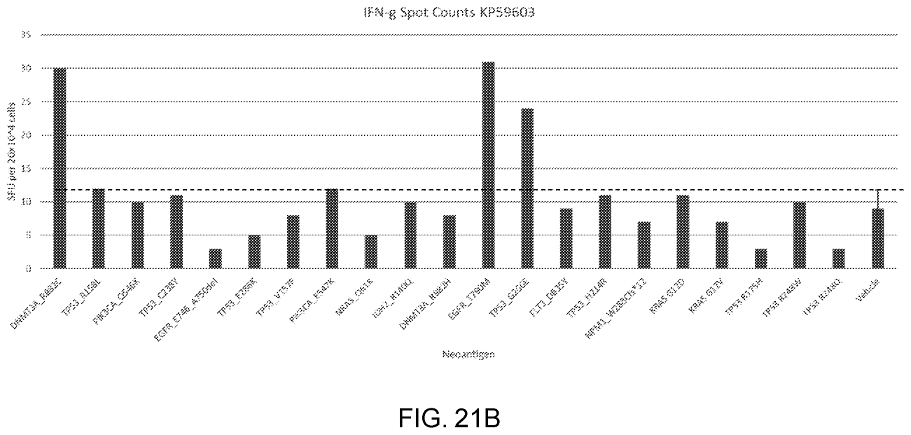

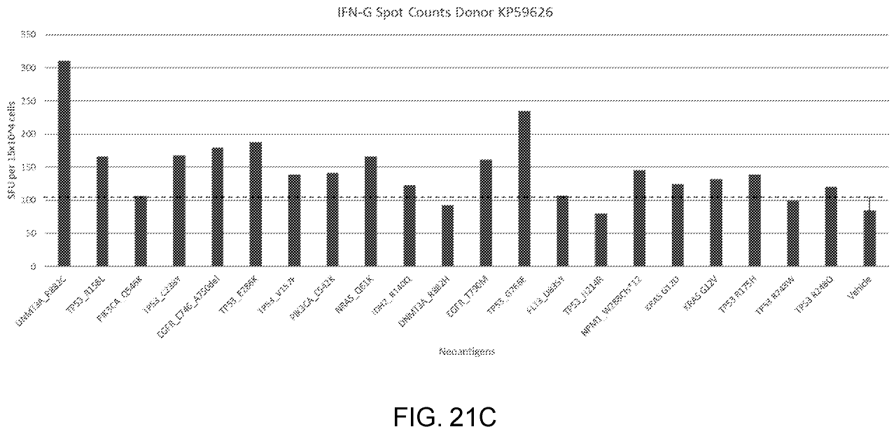

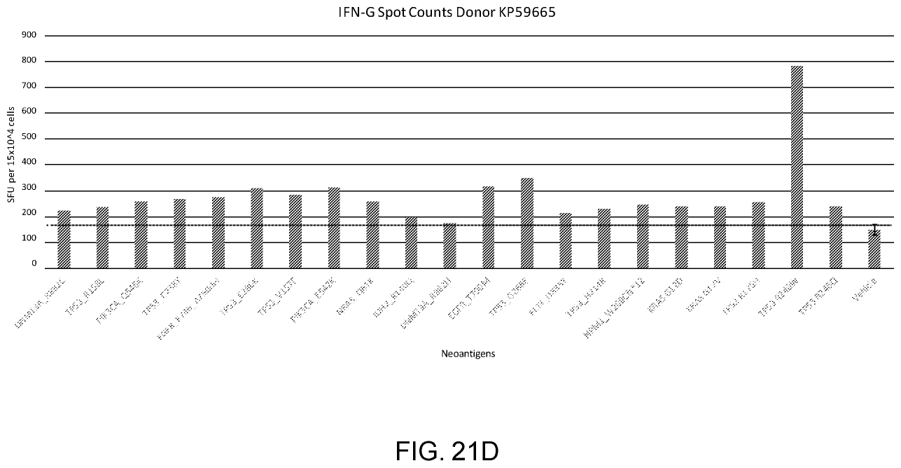

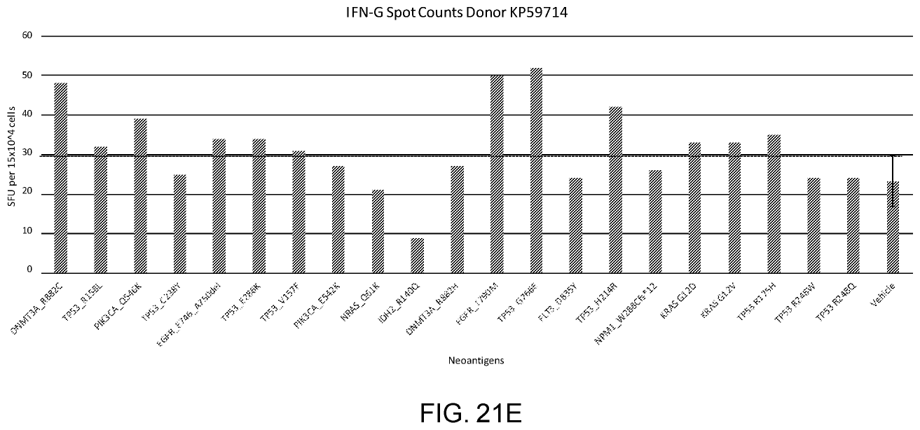

A multi-neoantigen construct according to the process described above with respect to FIGS. 15A-15B was generated and is shown in FIGS. 19 and 20A-20B. The mRNA or peptide T-cell production process was used for two donors (FIGS. 21A-21B), and, with another three donors, conditions were modified slightly by the use of a Rho kinase (ROCK) inhibitor present at the start of priming and serially diluted out with feedings (FIGS. 21C-21E). Chemical treatment of the DCs with ROCK inhibitors improves the viability or priming capacity of the DCs by preventing Rho kinase from triggering caspase activation (Moshirfar 2018; Rao & Epstein 2007).

In all samples, there were multiple neoantigen specificities. Common to most samples is reactivity to DMNT3A R882C, EGFR T790M, and TP53 G266E. As in FIGS. 21A-21E, each neoantigen included in the mRNA is assessed individually using crude peptides at total mass 1 μg/mL. These are not GMP quality, not purified, and are made at microgram scale; therefore, they are not suitable for the process itself but can be used for ELISpot. Alternatively, monocytes or DCs from the patient can be transfected with mRNA and used to present antigen to the cells. The DMSO control sets the background levels and wells with more spots than control is positive. The germline (wild type) amino acid sequence is also tested. This implies that each sample contains epitopes that are particularly immunogenic. There was a total of nine antigens that had spot counts above vehicle control (FIG. 21A), three neoantigens that had spot counts above vehicle control (FIG. 21B) and for ROCK inhibitor, there were 16 neoantigens (FIG. 21C), 20 neoantigens (FIG. 21D), and 11 neoantigens (FIG. 21E) that were above vehicle control. As shown in FIGS. 21C-21E, the ROCK inhibitor was particularly effective.

The examples provided for producing T cells that are directed against neoantigens and the examples where T cells are directed against viral antigens are not mutually exclusive. Just as how multiple neoantigens can be targeted at once, the same goes for the combination of any neoantigen with a viral antigen. All cancer associated antigens can simultaneously be targeted.

### ROCK Inhibitors

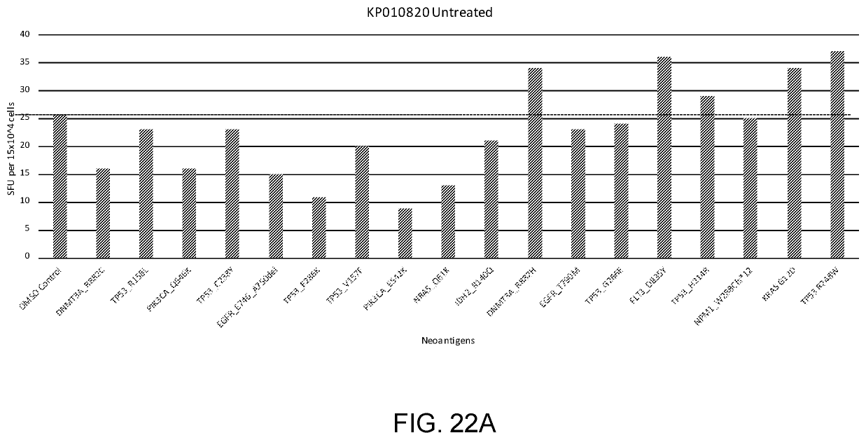

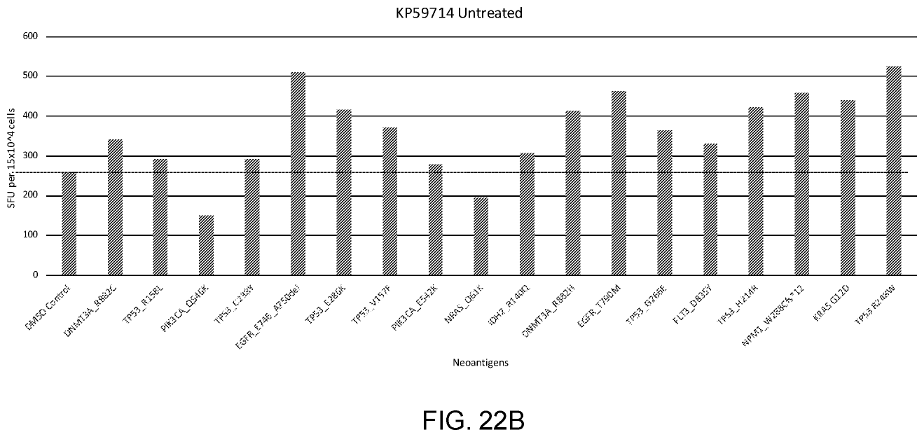

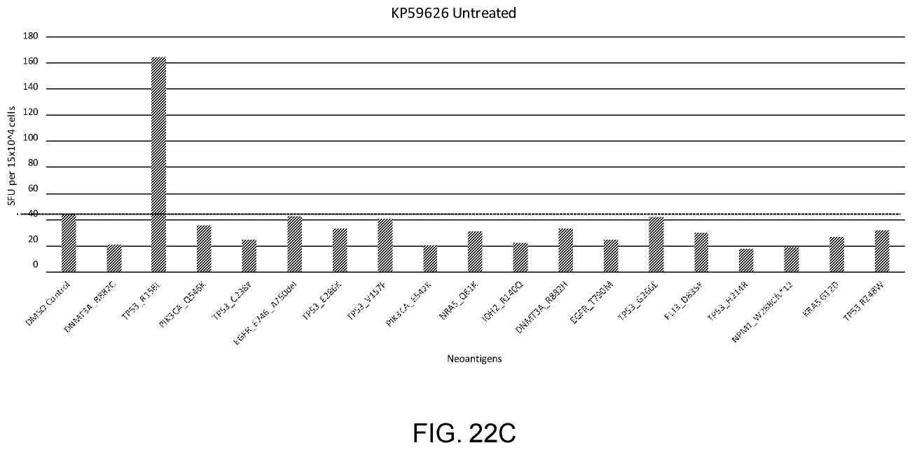

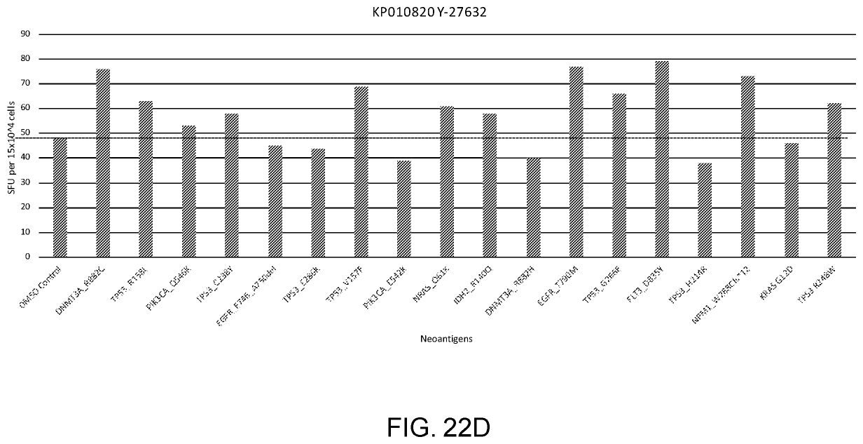

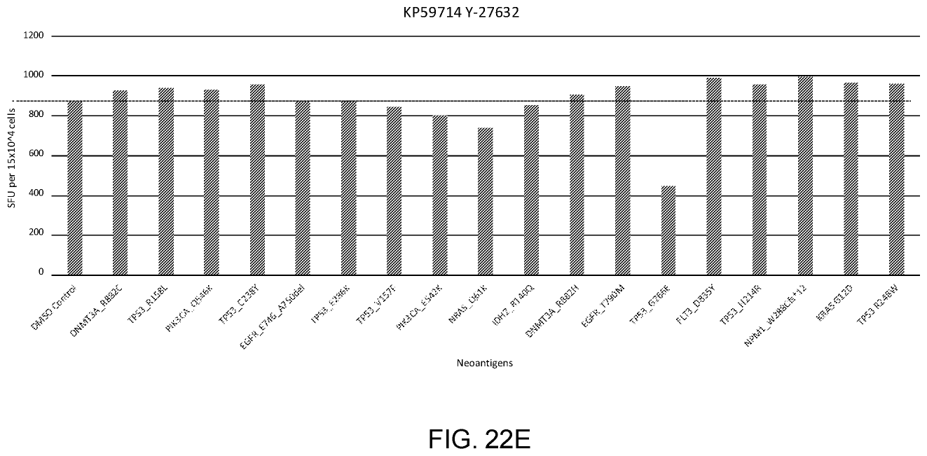

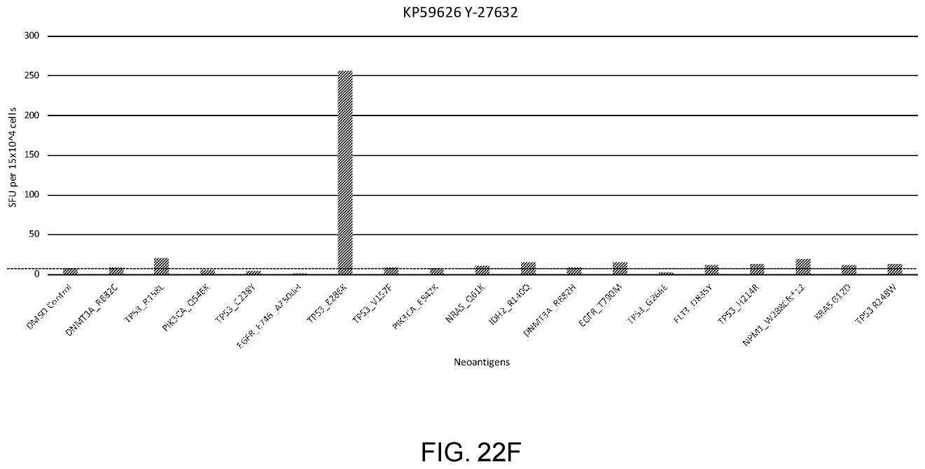

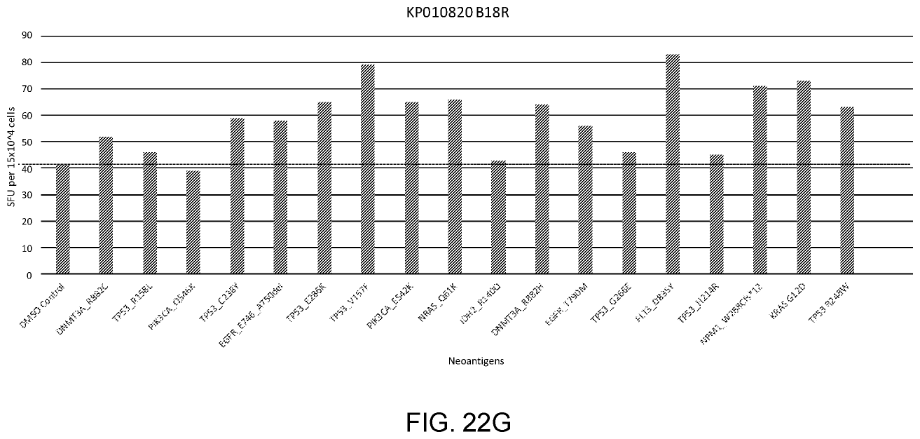

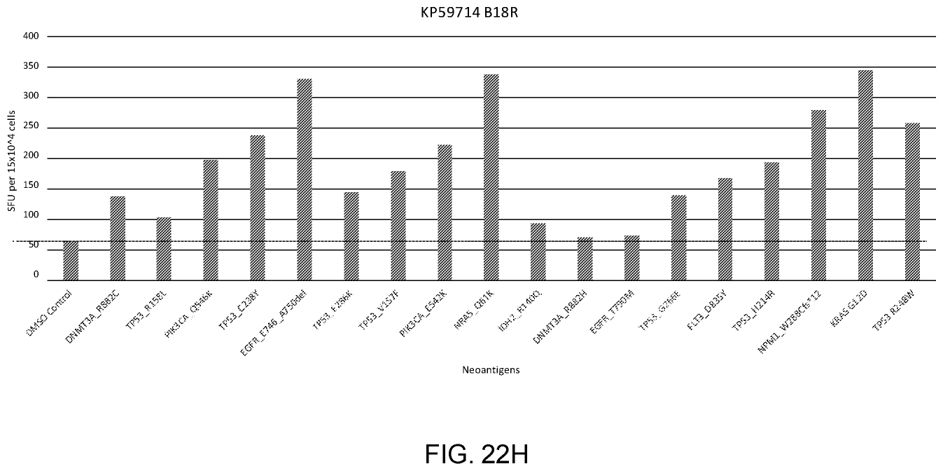

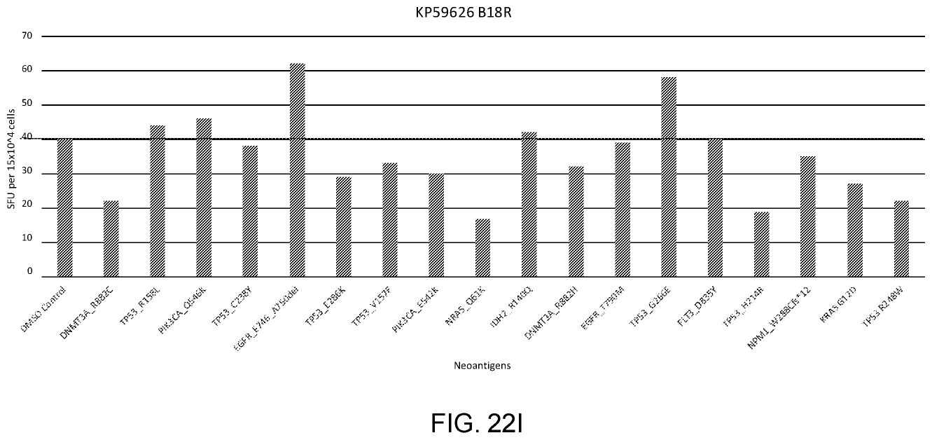

The effectiveness of the ROCK inhibitors promoted an investigation into the underlying mechanism that provided the improved response. An experiment was conducted using three donors with three conditions: control process, ROCK inhibitor added in with DCs and nonadherent cells at day 1 at 10 μM, and B18R added in with DCs and nonadherent cells at day 1 at 500 ng/mL (Millipore Sigma Aldrich). An ELISpot was conducted as before resulting in an average of five responses in control, 10 responses for Y-27632, and 11 for B18R. Individual counts are provided in FIGS. 22A-22I and Table 15. The results indicate that the two treatments have similar impacts but very different forms and targets which suggests an inhibition of apoptosis mechanism. Prolonging the life and number of the transfected DCs has a direct impact on antigen presentation to T cells with more contacts leading to more priming. Importantly, the results do not rule out other mechanisms and these may be contributing to the increased response rate as well. If the transfection did not have any negative impact on DCs, either of these two treatments may have a measurable positive effect on priming.

TABLE 15
List of Number of Positive and Average
Responses to the Model 21 Neoantigens

| | | C | D |
|---|---|---|---|
| A | B | # Positive | Average |
| KP010820 | Untreated | 5 | 6 |
| KP59714 | Untreated | 12 | — |
| KP59626 | Untreated | 1 | — |
| KP010820 | Y-27632 | 12 | 10 |
| KP59714 | Y-27632 | 10 | — |
| KP59626 | Y-27632 | 9 | — |
| KP010820 | B18R | 13 | 11 |
| KP59714 | B18R | 16 | — |
| KP59626 | B18R | 3 | — |

### T Cell Response to Neoantigen Polylinker

Intercellular cytokine staining (ICS) was performed, and the results were negative for intracellular cytokines for all six samples tested with the neoantigen polylinker. To analyze T cell responses to antigen by FACS, cells are collected from the peptide PBMC no DC no mRNA process or mRNA T-cell process on days 14 and 28 as indicated in the text. Cells are washed twice with 37° C. RPMI 1640 in an equal volume to collected culture media. 1×10⁶ cells per well are plated in V-bottom 96-well plates (Corning) in T cell media without cytokines in a 100 μl volume (DC media, 10% HS, 2 mM L-Glutamine). Either peptide antigens resuspended in DMSO or DMSO only, depending on conditions, were made up such that the final concentration when 100 μl is added to plated cells is 1 μg/mL in DC media, 10% HS, 2 mM L-Glutamine and protein transport inhibitor (containing monensin) at the final concentration of 4 μl per 6 mL (Becton, Dickinson and Company, MA). A fluorescently labeled antibody against CD107a (R&D systems) is also included 50 ul per 1 mL final concentration. The plate is then incubated overnight at 37° C., 5% CO₂ humidified chamber. Cells are pelleted at 330 g for 5 min and washed twice with 200 μl of −/− Dulbecco's Phosphate Buffered Saline (ThermoFisher). A live cell stain Zombie Aqua from BioLegend was added and washed according to manufacturer's instructions. Cell pellets are resuspended with the pooled indicated amounts of fluorescently labeled antibodies to cell surface targets. After a 15-minute incubation at room temperature, cells are washed twice with 200 μl of PBS with 0.1% sera. The cells are fixed and permeabilized using Cyto-Fast Fix-Perm Buffer Set (BioLegend, CA) according to manufacturer's instructions. Fluorescent antibodies (Table 12) against intracellular targets are added as indicated to 50 μl of perm buffer, added to cells and incubated at room temperature for 20 minutes. Cells are washed twice as before and resuspended in 200 μl flow buffer PBS 2% FBS and then run on the NovoCyte 3000. Cell debris was eliminated from the analysis by gating on forward scatter and side scatter. Single cells were selected by comparing forward scatter height and forward scatter area.

TABLE 16
Antibodies Surface and Intracellular Targets

| Antibody | Target | Company | Catalogue # | Volume μl |
|---|---|---|---|---|
| PE-Cy5-CD107a | Surface | BD | 555802 | 50 μl/mL |
| | | Pharmigen | | |
| BV570 CD4 | Surface | Biolegend | 300534 | 2 |
| PE-Cy7 CD8 | Surface | Biolegend | 300914 | 2 |
| APC-Cy7 CD3 | Surface | Biolegend | 300426 | 2 |
| BV421 TNFα | Intracellular | Biolegend | 502932 | 5 |
| BV785 IFNγ | Intracellular | Biolegend | 502541 | 5 |
| PE-IL2 | Intracellular | Biolegend | 500307 | 5 |

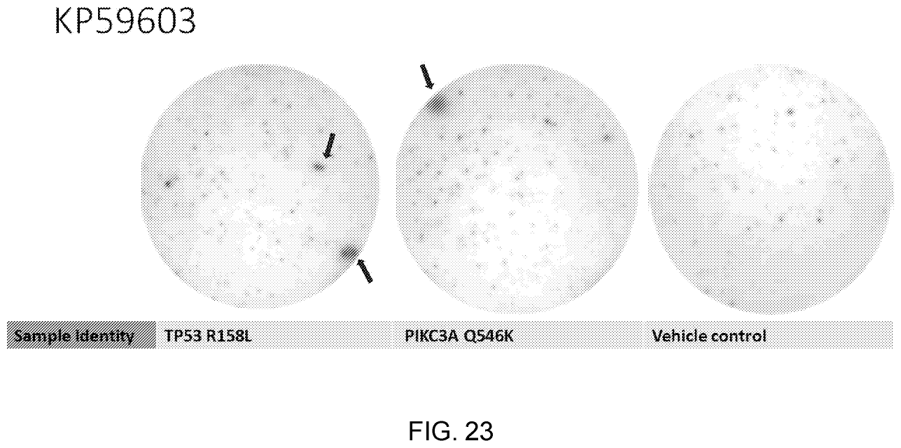

The results were positive for an LMP2A polylinker. Specific epitopes in LMP2A were then selected and placed into the polylinker outlined in FIG. 15A as a control for the polylinker construct function. An ICCS assay has a limit of detection above 2% of cells being reactive; below this limit the assay does not reliably differentiate the vehicle control from peptide added samples. However, the ELISpot can be used down to 0.1% of cells being reactive. The ELISpot is also able to identify individual IFNγ cell clusters as indicated by the arrows in FIG. 23.

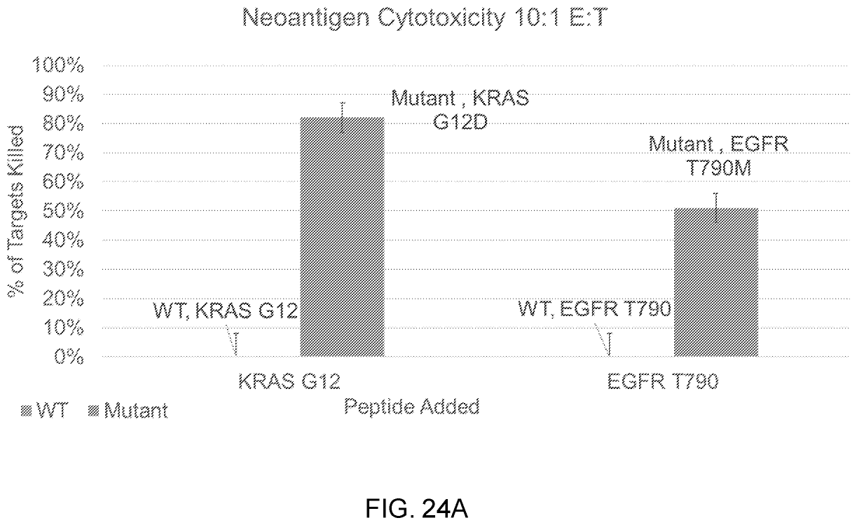

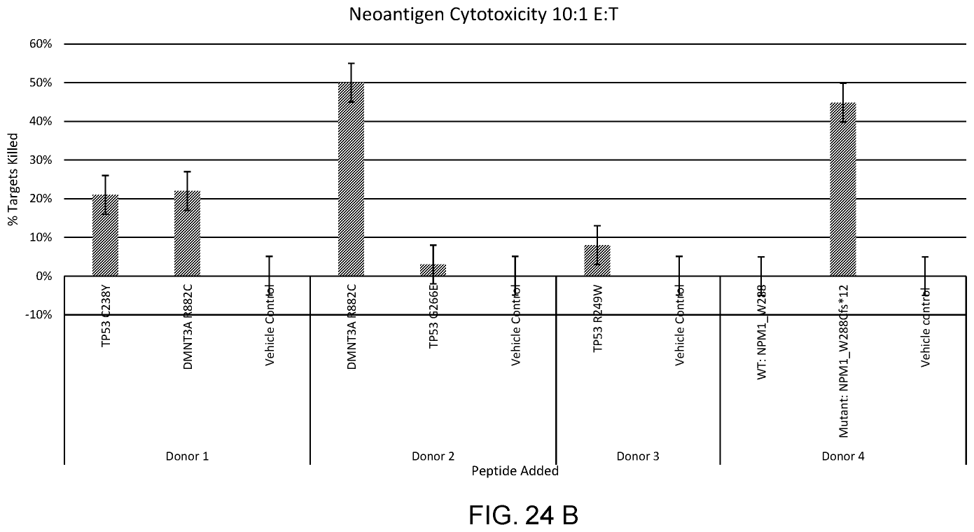

Lastly, a cytotoxic potential was determined using the multi-neoantigen construct as shown in FIGS. 24A-24B. Activated T cells from the patient such as PHA blasts are transfected with mRNA for each of the neoantigens. PHA blasts are used because they proliferate rapidly and have matching HLA to the T-cell product culture. Effector cells from the product culture are then added to antigen loaded targets and cell death in the target cells is measured by a viability stain gated on CSFE labeled targets. The germline (wild type) amino acid sequence is also tested. If a reactivity is found against these self-sequences, then the degree of response is taken into consideration. Marginal reactivity as compared to neoantigen sequence can still be used. For example, a culture that is positive for several germline sequences on IFNγ but has no cytolytic activity against those sequences but does so against the neoantigen will be used.

Having established a model of a multi-targeting T-cell product, the product is in pre-clinical testing using patient samples. A colorectal cancer patient with progressive disease provided a blood sample part was sent to Guardant Health and the rest cryopreserved. Results are provided in Table 11. Examination of these results indicates that there are eight mutations that resulted in changes to the amino acid sequence. To provide readily interpretable results these mutations were selected but the process is not limited to these types of mutations.

As outlined previously and as in FIG. 15 a neoantigen polylinker sequence was generated using 21 amino acids (but not limited to) with the amino acid change in the center flanked by 10 amino acids corresponding to the person's germline sequence upstream and downstream of the mutation. The sequence was ordered synthesized from ThermoFisher on a Monday, received Thursday, and cloned into a plasmid and sequenced on Friday with a larger preparation of plasmid completed on that Saturday. The plasmid was linearized on the same day and in vitro transcribed to RNA for transfection. In parallel to receiving the sequence and sending it out for synthesis previously stored matching PBMCs underwent the DC differentiation process. Monocytes were isolated on the same day as receiving the sequence, differentiated for 5 days and then matured overnight on the Saturday of RNA production in time to be transfected and combined with previously frozen T-cells from the patient on the following Sunday. Three weeks of culture follow as previously described, at which time antigen reactivity assessment is performed. An IFNγ ELISpot was performed using peptide pepmixes as antigen, but this is not limited to peptides as the polylinker mRNA used at the start can be transfected into PBMCs matching the patient which serves as an alternate source of antigen.

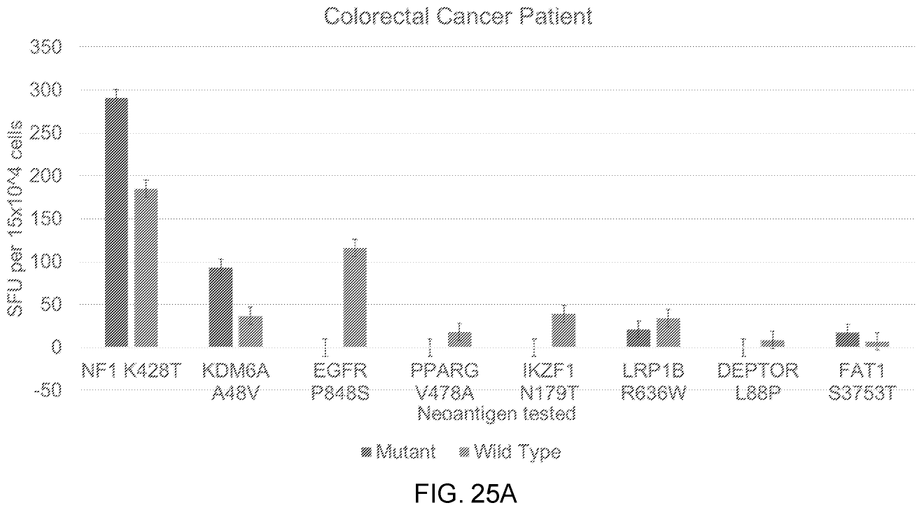

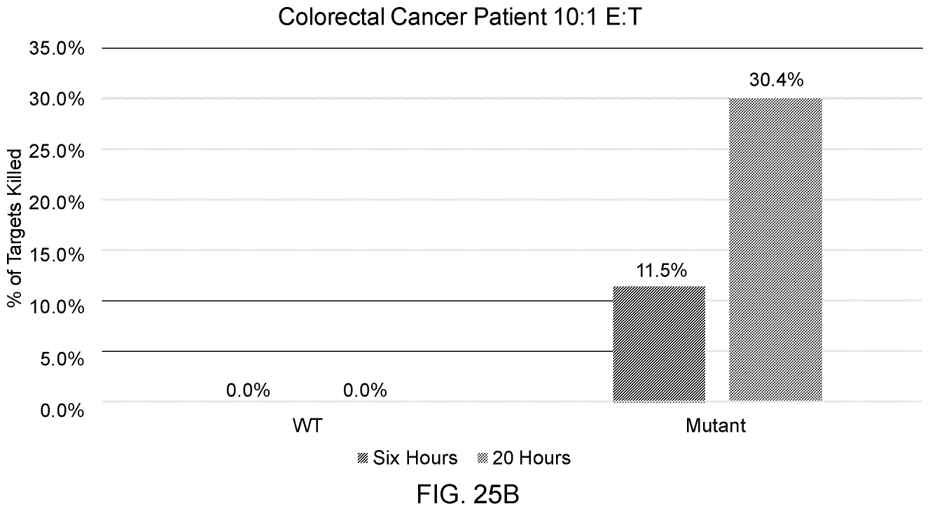

FIG. 25A indicates a product with multiple specificities to targeted neoantigens. The most response was to NF1 K428T. Mutations in this gene are closely associated with lung cancer and it is interesting to note that this patient indicated they had 30-35 cigarette pack years. The mutation KDM6A A48V, LRP1 B R636W and FAT1 S3753T had a measurable response as well. Wild-type pepmixes produced measurable responses in these mutation sites however they are reduced in comparison to that of the mutant in NF1 K428T, KDM6A A48V, FAT1 S3753T. The functional impact of these results is assessed by cytotoxicity assay, the results of which are set forth in FIG. 25B. The CSFE PHA blast cytotoxicity assay was performed as described. Experimental effector cells from the run were combined with fluorescently labeled target donor matched PHA blasts that had been loaded with either vehicle control, a mixture of pepmixes encoding each of the eight mutations or a mixture of pepmixes encoding each of the eight germline sequences (wildtype) corresponding to the sties of the eight somatic mutations. A 10:1 ratio of effector cells to targets cells was used and incubated for 20 hours under cell culture conditions (37° C., 5% CO2). The fraction of dead target cells at the end of 5 hours and at the end of 20 hours is provided. Background from vehicle is subtracted from both mutant and wild-type sequences. Wild-type indicates germline sequences. Mutant indicates somatic mutations.

A mixture of all germline sequence pepmixes or all somatic mutations pepmixes was loaded into matching PHA blasts. Assaying combined pepmixes reflects the combined presentation of antigen from DCs at the start of the process. It also maximizes sensitivity by an aggregate signal. The effector and targets mixture was assayed at six hours and again at 20 hours. No cytotoxicity was measured for the germline sequences. Significant cytotoxicity was detected against the mutant starting at 11.5% of killed targets at six hours and an almost tripling of targets killed to 30.4% at 20 hours. Cytotoxicity supports that there is no functional impact of the detected IFNγ release upon exposure to germline sequences. The approach of multiple parameter release testing will lead to safer more effective adoptive cell therapies.

### Real Time Cytotoxicity Assay

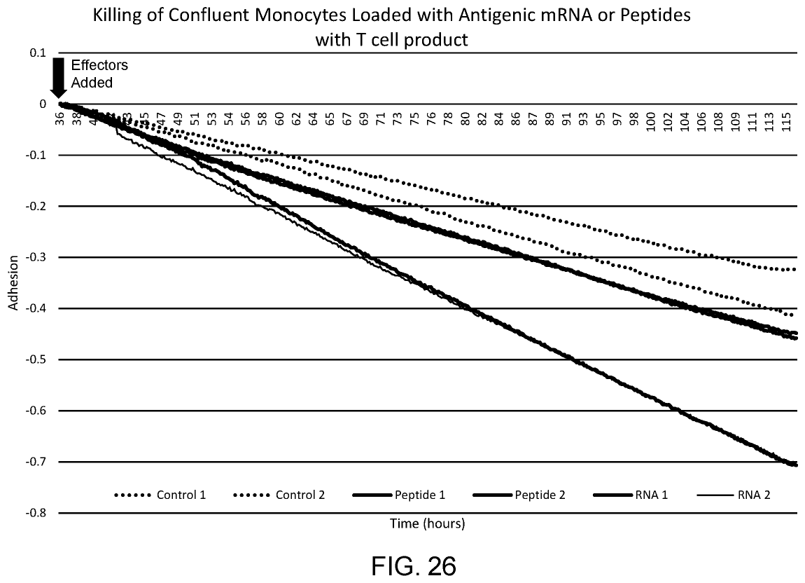

The killing potential of the manufactured T cell product can further be analyzed utilizing the Agilent xCelligence™ real time cell adhesion instrument “RTCA”. This instrument uses plates with electrodes inserted into the growth surface of a tissue culture compatible plate. It measures the adhesion of cells to the bottom of the well by monitoring impedance, which is an indicator of size, shape, polarity, and number of cells. A baseline reading is taken without cells, cells are then added allowed to adhere and then the impedance is read again. This can be used to measure the ability of T cells to kill targets. When T cells specific for an antigen are added to these adherent cells and that same specific antigen is presented through HLA class I by adherent targets the CD8+ T cells will kill these cells. The impedance falls from the maximum of the adherent cells as they perish. An example of this experiment is provided in FIG. 26. A T cell product targeting the model 21 neoantigen construct was produced. Matching donor PBMCs of the aforementioned T cell product had monocytes separated out by negative selection (no antibody targeting to the monocytes) (Stemcell). The monocytes were then nucleofected using the techniques described herein with the model 21 neoantigen mRNA and subsequently plated into the xCelligence™ E-plate in RPMI1640 media 5% human sera and placed into cell culture conditions overnight (37° C., 5% CO2). Negatively selected monocytes from this same donor were also plated in a separate well but were nucleofected with a sham mRNA (one not targeted by product). They had pepmixes added to them corresponding to the 21 neoantigens at 1 ug/mL each and plated in RPMI1640 5% human sera media and placed into cell culture conditions overnight (37° C., 5% CO2). Two sample wells were tested for each condition. The T cell product was added the next day to each of the groups. The cell index of combined monocytes and T cells was set to 0 and the loss of adhesion was tracked in real time. The nucleofected cells were strongly killed while the peptide group was not as strongly killed. This is an important experiment as it demonstrates that cells expressing neoantigen in an endogenously produced manner are killed more effectively than that of the exogenously chemically produced pepmixes. This more closely mimics what would happen within a patient who has cancer.

### T Cell Phenotypes

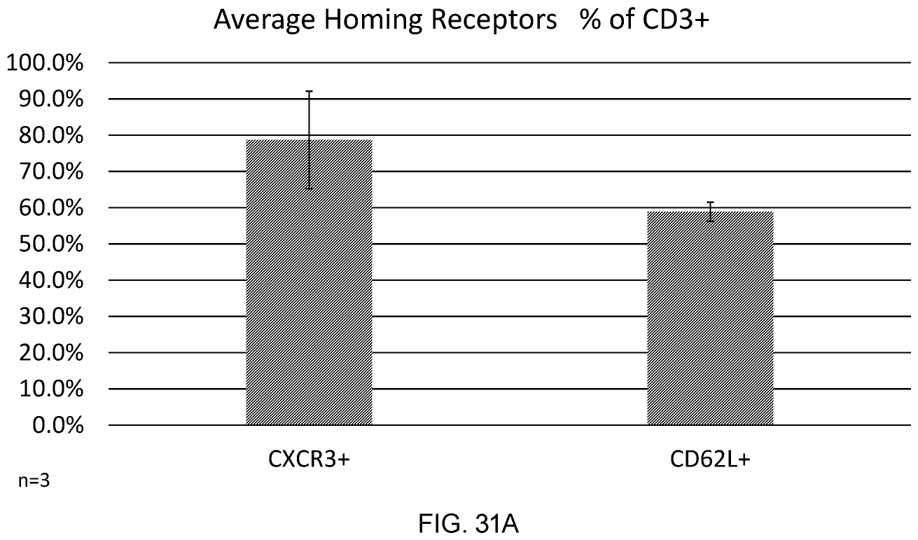

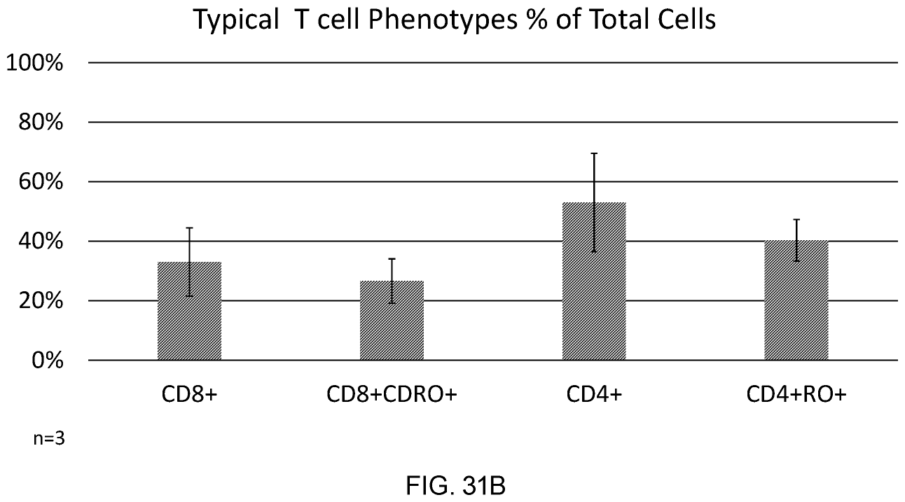

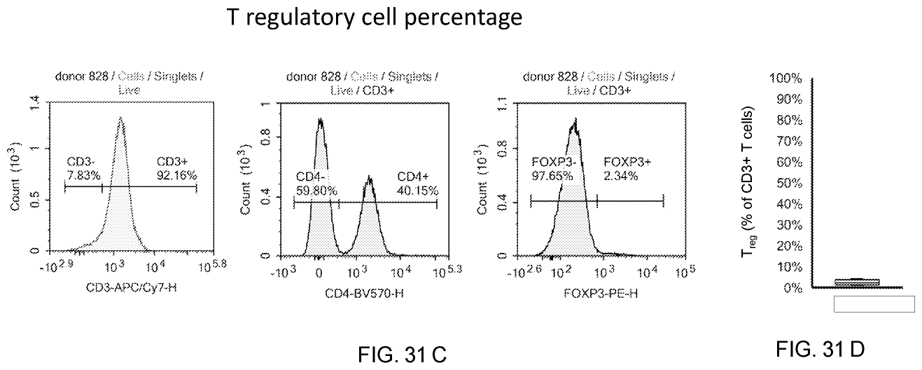

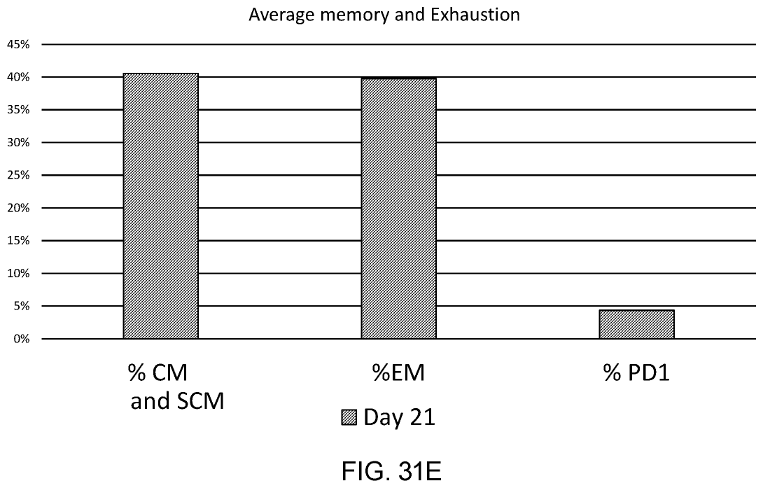

A detailed analysis of T cell phenotypes including memory and exhaustion markers has been conducted with a memory marker FACS panel including: live/dead stain, CD3, CD4, CD8, CD45RO, CD45RA, CD197, CD28, CD122, CD127, CD183, CD95, and CD62L. There is a significant population of memory cells present as indicated by the markers CD197, CD45RO, CD62L, and CD95. The peptide PBMC no DC no mRNA T cell Process typically results in 35-40% of memory cells present in the culture at day 21. However, 25-35% of the T cells are effector memory T cells with the phenotype CD197−, CD45RO⁺, CD62L⁻, and CD95⁺. The DC process, as a result of effective priming, lowers the fraction of effector memory T cells and increases the number of central memory T cells with the phenotype CD197⁺, CD45RO⁺, CD62L⁺, and CD95⁺ (Hikono 2007). The significance of a higher fraction of central memory cells can improve the longevity of the treatment and its efficacy. Central memory T cells are longer lasting than effector memory cells and are known to maintain long term immunity (Huster 2006; Olson 2013; Seder 2013). It is also theorized that priming with DCs can convert exhausted cells into active cells. The typical fraction of CD3+ cells that express CD183 is 80%. Having a majority cells with this marker predicts that T cells will effectively traffic into tumor sites (FIG. 31A).

Importantly in our detailed analysis of the T cell phenotypes that are present in the mRNA or peptide T cell production processes indicate that the fraction of T regulatory cells is on average at or below 2.5% of CD3⁺ cells FIGS. 31C-31D. This is a very small percentage compared to other putative cell therapies including derivation from tumor infiltrating lymphocytes or products ex vivo expanded by IL-2. Tregs have suppressive capacity. High Treg percentage is expected to correlate with poor efficacy of the product.

For this experiment, starting cancer patient's T cells were PD-1^{hi}, and after the process, most T cells were PD-1^{lo}, which is indicative of a conversion from a more effective treatment (Jiang 2018). On average, our production process results in significant enrichment in memory T cells as compared to starting with cells from normal donors. Exhaustion of T cells comes from over activation, T_{reg} activity, and immunosuppressive cytokines such as IL-11. In the mRNA or peptide T-cell production process, cells are activated and undergo many rounds of replication, but do not result in PD-1^{hi}, indicating that the T cells are not overactivated or exhausted (FIGS. 31B, 31E and Table 17). FIGS. 31B and 31E shows the major memory T cell subsets, as determined by flow cytometry, are central memory (CM), effector memory (EM), and exhaustion by PD-1. For this experiment, CM cells are defined as CD3⁺/CD45RO⁺/CD62L⁺ and EM cells are defined as CD3⁺/CD45RO⁺/CD62L⁻. At day 21, on average, 41% percent of the cells are CM, 40% of the cells are EM, and there were less than 5% PD-1 cells. The average for each was calculated from six processes using four different healthy donors.

TABLE 17
The Average Percentage of CM, EM, and PD1 Cells at Day 21

| | % of CD3⁺ with CD62L | | |
|---|---|---|---|
| | % CM | % EM | % PD1 |
| Day 0 | 14% | 23% | 0% |
| Day 21 | 41% | 40% | 4% |

### T Cell Exhaustion Markers

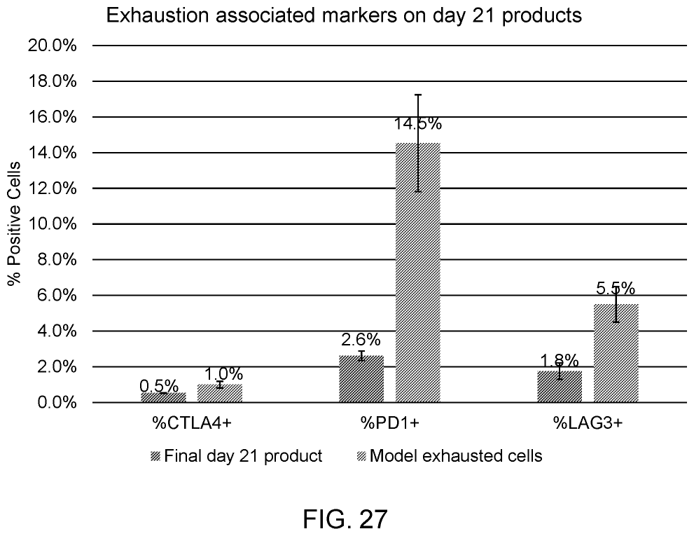

In this example the percent of cells that are positive for one or more exhaustion markers was determined in the final T cell product for three separate donors. T cell exhaustion is a functional definition in which T cells with an antigen specific TCR fail to activate when challenged with that specific antigen. Research regarding T cell exhaustion have identified proteins whose expression is corelated with the exhausted phenotype and include PD-1, CTLA4 and LAG3. The T cell product at the end of manufacture using the mRNA T-cell process was assayed using FACS. The immune fluorescent antibodies for PD-1, CTLA4 and LAG3 from Becton-Dickinson cat. No. 561272, 555853, 565716 were tested separately using 1 million cells each and 5 ul of antibody used for each. Cells were stained for 30 min on ice, washed twice with PBS and resuspended in PBS for running on the Novocyte™ flow cytometer. Results indicate on average 2.6% PD-1+, 0.5% CTLA4+, 1.8% LAG3+ FIG. 27. A positive control for exhaustion was generated by treating three different donor PBMC with superantigen PMA lonomycin at 1× concentration (Thermofisher) on day 1, day 7, day 14 of culture in RPMI1640 with 5% human sera. Repeated stimulations and over stimulation are the main drivers of T cell exhaustion and as in FIG. 27 these cells were on average 14.5% PD-1+, 1% CTLA4+, 5.5% LAG3+. Comparing the product and the positive controls shows 5.5×PD-1+, 2×CTLA4, 3×LAG3. Final T cell product does not have an exhausted phenotype.

Priming PBMCs Alone with Cationic Lipids and mRNA

In this example the cationic lipid Lipofectamine was tested as the method of antigen transfer. Here mRNA encoding antigen is mixed with lipofectamine and added directly to PBMCs in culture. This is alternative to the use of nucleofection of mRNA encoding antigen into DCs. It is assumed that APCs in the PBMCs will take up the mRNA and present antigen to the rest of the T cells. This method has some efficiency generating T-cell compositions specific to EBV antigens but has little efficacy generating T cells against neoantigens.

For the comparison of the peptide-based PBMC Process and the modified mRNA-based process, an experiment was conducted at a small scale using PBMCs from two healthy donors stimulated with Epstein-Barr Virus (EBV) latent antigens LMP1, LMP2 and EBNA1.

On day zero of the process, PBMCs are thawed and 1×10⁶ of them are placed in CellGenix® GMP DC medium, 10% human AB sera, 2 mM L-Glutamine (complete DC medium) with human IL-7 (3753 U/mL) and IL-15(525 U/mL) in a G-Rex 24® well plate. Next, for the peptide-based process, pepmixes for the antigens are added to the culture at 0.1 μg/μL/peptide concentration. For the modified mRNA-based process, the lipid nanoparticle Lipofectamine® MessengerMAX™ is combined with Opti-MEM™ reduced-serum medium, followed by a 10-minute incubation at room temperature. The mRNA for the three antigens at a total amount of 2 μg is combined with the Opti-MEM™ reduced-serum medium as well and added to the Lipofectamine® MessengerMAX™ for an additional 5-minute incubation at room temperature. Following the second incubation, the mixture containing mRNA-Lipofectamine® complexes is added to the PBMCs in the G-Rex 24® well plate for transfection. The G-Rex plate is stored in a 5% CO₂ 37° C. humidified incubator between each feeding/stimulation day. The culture is fed with fresh complete DC medium containing human IL-7 (3753 U/mL) and IL-15 (525 U/mL) on day 3. On day 7, the day 0 process is repeated and 1×10⁶ fresh PBMCs are combined with either the antigen pepmixes or the mRNA-Lipofectamine® complexes and added to the same well in the G-Rex 24® well plate for a second stimulation. The culture is fed with fresh complete DC medium containing human IL-7 (3753 U/mL) and IL-15 (525 U/mL) on day 9. A polyclonal stimulation of the cell culture is performed on day 11 with the help of ImmunoCult Human CD3/CD28/CD2 T cell activator at a 7.5 μL/0.5×10⁶ cells/mL concentration. On day 14, the culture is transferred to a G-Rex 6® well plate and complete DC medium containing human IL-7 (3753 U/mL) and IL-15 (525 U/mL) is added to the cell culture for an 8× dilution. Additional medium is added for a 1.5× dilution on days 16 and 18. Finally, the cells are harvested on day 21.

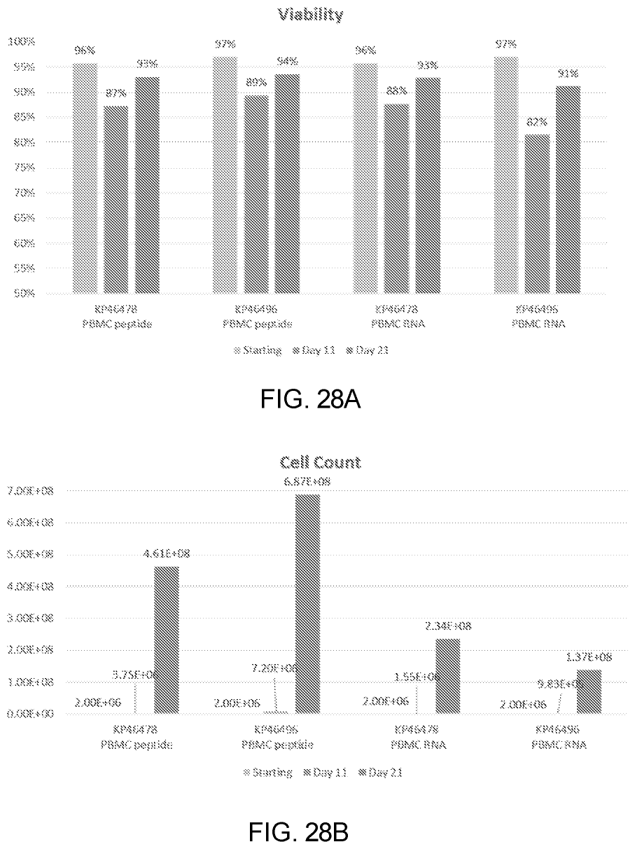

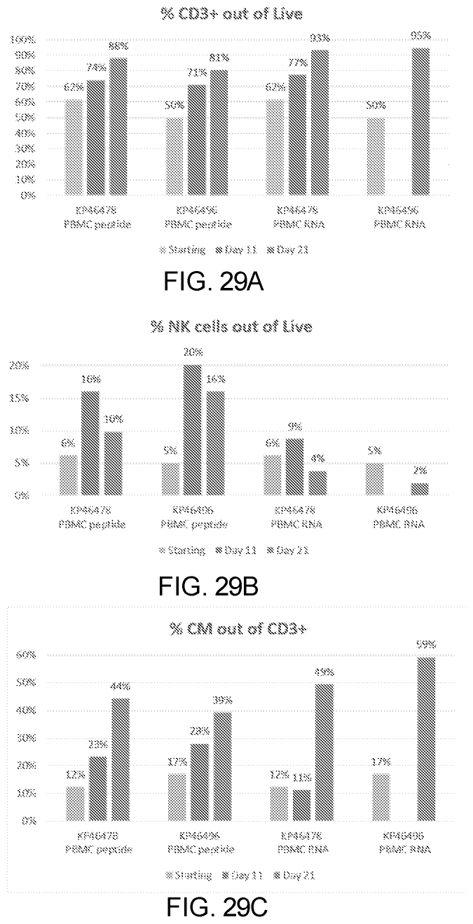

A set of assays was performed on these samples, including cell count and viability assessment, a flow cytometry phenotype assessment, and a killing assay. The cell count and viability were checked on days 0, 11 and 21, and the flow cytometry phenotype assessment and killing assay were performed on day 21 at the end of the process. The viability for the samples with the modified RNA-based process was comparable to the peptide-based PBMC process, and all the samples had above 91% viability on day 21 (FIG. 28A). While the overall cell yield was higher in the peptide-based process samples (FIG. 28B), the percentage of CD3⁺ cells was higher in the mRNA T-cell production process samples (FIG. 29A), which would ensure a more target-specific response. Notably, the modified process had significantly less CD3⁻CD56⁺ NK cells, which are not desirable in our final product. Specifically, the modified RNA-based product had only 2-4% NK cells compared to 10-16% NK cells in the peptide-based product (FIG. 29B). Additionally, the modified RNA-based process produced a significantly higher percentage of central memory T cells, a higher percentage of which would ensure a longer-lasting tumor response. The T cells within the modified RNA-based product were 49-59% CD45RO⁺CD62L⁺ central memory, while the ones within the peptide-based product were 39-44% CD45RO⁺CD62L⁺ central memory (FIG. 29C).

When it comes to the cytotoxic function of the final product of the two processes, the mRNA T-cell production process exhibited a better cytotoxicity profile (FIGS. 30A-30B). The cytotoxicity was measured by conducting a 22-hour killing assay where lymphoblastoid cell lines (LCLs) were used as target cells, and the product of the peptide- or RNA-based process was used as effector cells in a 10:1 effector to target ratio. The target cells were labeled with carboxyfluorescein succinimidyl ester (CFSE) and incubated with LMP1, LMP2 and EBNA1 antigens at 1 μg/μL/peptide concentration for positive control. For optimal killing, the positive control groups would not differ from the test groups, as the effector cells should respond to the endogenously processed antigens on the surface of LCLs regardless of the presence of additional antigens. For negative control, LCLs with CFSE only group was added to ensure that the CFSE was not causing the target cells' death. The death of the target cells was measured with two markers—7-Aminoactinomycin D (7-AAD) and Annexin V. 7-AAD detects dead cells by binding to the DNA within them, while Annexin V detects dead, as well as apoptotic cells by binding to phosphatidylserine on the cell membrane. The modified RNA-based process produced a higher percentage of dead target cells, as well as a more optimal cytotoxicity profile, as there was no significant difference between the test conditions and the positive controls measured by 7-AAD (FIG. 30A). Additionally, there was a significantly higher percentage of apoptotic target cells and a more optimal cytotoxicity profile in the RNA-based process samples as measured by Annexin V (FIG. 30B).

Overall, the mRNA T-cell production process yields a more favorable phenotype of cells, as well as significantly improves the cells' cytotoxicity profile. Importantly, however, this modified process does not produce as many cells as the peptide-based process. It is likely that the modified priming with lipid nanoparticles and mRNA allows for the growth and expansion of only highly specific T cells within the starting PBMC population. The success of this modified process is especially striking considering that transfection of nonadherent cells using lipid nanoparticles has been proven to be challenging and unsuccessful in most cases. This further demonstrates the robustness of the mRNA T-cell production process, as it generates improved priming in combination with a challenging transfection method.

### Example 7: Closed System T Cell Process

The following example provides details regarding a closed system process for producing T cells according to the present disclosure. Each of the methods described in Examples 1-6 can be conducted within the closed system. A closed system process refers to a system in which whole blood, collected in a bag, enters into the system, and the output are purified T cells, also contained within a bag. Each part of the process is performed by a machine comprised of connected sterile tubing. In contrast, an open system is typically performed under a sterile laminar flow hood, but flasks, tubes, and reagents are exposed to the environment. The advantages of using a closed system versus an open system is the minimization of risk of contamination and better-quality control. Reagents are provided in bags from the manufacturers and blood and cells are provided in IV bags. Importantly, the closed-system process does not alter the phenotype and/or activity of T cells as compared to open-system process.

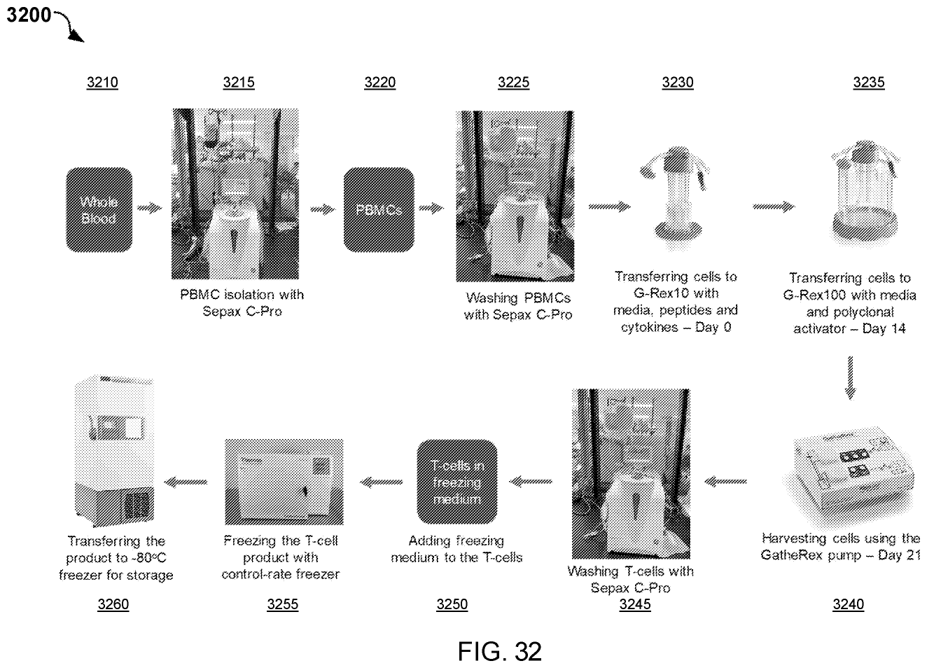

FIG. 32 depicts a process 2600 of producing purified T cells using a closed system (shown in FIG. 33A) from a whole blood sample. The process 2600 can begin in step 2610 comprising collecting a whole blood sample in a transfer bag and attaching the bag to a Sepax™ kit through sterile welding or spike ports. The process continues to step 2615 where PBMCs are isolated from whole blood using a Sepax™ C-Pro unit (GE Healthcare Lifesciences, now Cytiva) with reagents prepared under open-system sterile conditions in a class II grade A biosafety cabinet. In all subsequent steps, the reagents, intermediates, and final products are kept in bags that are attached to the Sepax™ C-Pro unit through sterile tube welding. The PBMCs are isolated by running the NeatCell protocol on Sepax, which uses a density gradient medium, specifically Ficoll-Paque® (GE/Cytiva), to isolate mononuclear cells from blood diluted in a 1:1 with saline in a transfer bag. This protocol can isolate PBMCs from up to 120 mL blood. However, when the whole blood volume is more than 120 mL, the PBMC isolation process is started by first running the SmartRedux protocol on the Sepax. This protocol helps reduce the starting volume of blood by removing most of the red blood cells. SmartRedux can reduce the volume to less than 120 mL, which can then be used in the PBMC isolation process with the NeatCell protocol. The PBMCs are then resuspended in a cryopreservation medium and frozen using a control-rate liquid nitrogen freezer. The PBMCs are then transferred to a −80° C. freezer for storage.

The process continues in steps 2620-2625 where the frozen PBMCs are thawed with DNase and serum-containing media using the CultureWash protocol on the Sepax C-Pro unit. The process then continues in step 2630 where the cells are plated in a G-Rex®10M-CS cell culture device in CellGenix DC medium containing cytokines and peptides. The process then continues in step 2635 where the cells are fed fresh DC medium containing cytokines on day 3, followed by the addition of another set of thawed and washed PBMCs in DC medium with peptides and cytokines on day 7. The cells are then fed additional DC medium on days 9 and 11 before the addition of a polyclonal activator. On day 14, the cells are added to a G-Rex®100M-CS, to ensure the rapid expansion of antigen-primed T cells.

The process then continues to step 2640 where the T cells are harvested. The cells were harvested from the G-Rex®100M-CS unit using the GatheRex™ pump. The process then continues to step 2645 where the T cells are washed with using CultureWash protocol on the Sepax C-Pro unit. The process then continues to step 2650 where a freezing medium is added to the cells. The process then continues to step 2655 where the T cells are cryopreserved in a freezing medium using a control-rate liquid nitrogen freezer. Lastly, the process continues in step 2660 where the T cells are transferred to a −80° C. freezer for storage for future use.

The process can further include loading each dendritic cell (DC) with antigens (peptides, mRNA, or cell lysates) in separate chambers on the closed system DC, ensuring parity of representation and a broader antigen response profile (FIG. 33B).

PBMC Based Process No DC or mRNA

A PBMC process without using DCs was developed using the closed system. With the closed system, it is difficult to add reagents; however, there is a need to minimize perturbations to the system to reduce potential sources of contamination. The volume of the flasks used also had a maximum fixed holding of 100 mL. A number of starting cells were required to proliferate in logarithmic phase for 21 days but not too many as to require a large amount of media that needed to be replenished. Consistent log phase is also important to the health of the culture as T cells that are too dense have Fas-FasL mediated fratricide and are an indication that there was less competition for nutrients amongst the cells. Lastly, peptides are expensive so minimizing the amount required for the experiments was an important consideration. Table 18 provided below provides the experimental details.

TABLE 18
Experimental Details for the Closed System Process

| | | Media | | | | Antigen per | | Density |
|---|---|---|---|---|---|---|---|---|
| Exp. | Cells/well/stim | volume, ml | | Antigen | | million cells | | Cells/ml |
| SD1 | 4 × 10⁶ | 2.5 | ml | 0.1 | μg/peptide/ml | 0.0625 | μg/peptide | 1.6 x 10⁶ |
| SD2 | 2 × 10⁶ | 2.5 | ml | 0.1 | μg/peptide/ml | 0.125 | μg/peptide | 0.8 x 10⁶ |
| SD3 | 1 × 10⁶ | 2.5 | ml | 0.1 | μg/peptide/ml | 0.25 | μg/peptide | 0.4 x 10⁶ |
| SD4 | 0.5 × 10⁶ | 2.5 | ml | 0.1 | μg/peptide/ml | 0.5 | μg/peptide | 0.2 × 10⁶ |
| D1A1 | 4 × 10⁶ | 2.5 | ml | 0.1 | μg/peptide/ml | 0.0625 | μg/peptide | 1.6 x 10⁶ |
| D2A2 | 2 × 10⁶ | 2.5 | ml | 0.05 | μg/peptide/ml | 0.0625 | μg/peptide | 0.8 x 10⁶ |
| D3A3 | 1 × 10⁶ | 2.5 | ml | 0.025 | μg/peptide/ml | 0.0625 | μg/peptide | 0.4 x 10⁶ |
| D1V1 | 4 × 10⁶ | 5.0 | ml | 0.05 | μg/peptide/ml | 0.0625 | μg/peptide | 0.8 x 10⁶ |
| D2V2 | 2 × 10⁶ | 2.5 | ml | 0.05 | μg/peptide/ml | 0.0625 | μg/peptide | 0.8 x 10⁶ |
| D3V3 | 1 × 10⁶ | 1.25 | ml | 0.05 | μg/peptide/ml | 0.0625 | μg/peptide | 0.8 x 10⁶ |
| D4V4 | 0.5 × 10⁶ | 0.625 | ml | 0.05 | μg/peptide/ml | 0.0625 | μg/peptide | 0.8 x 10⁶ |

### Seeding Experiments

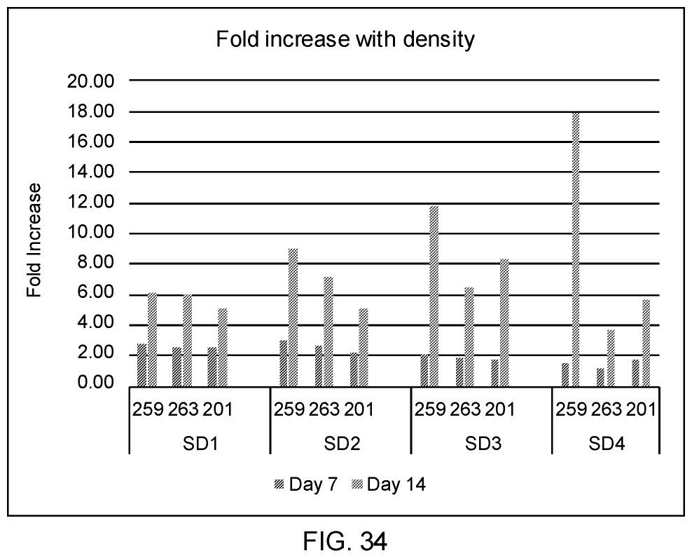

In this experiment, the goal was to optimize to a cell density of 4×10⁶ cells/mL (SD1). The seeding density of 0.4×10⁶ cells/mL (SD3) was found on average to have the highest fold increase on day 14 indicating the maximum growth rate (FIG. 34 and Table 19).

TABLE 19
The fold increase over starting cell number from
day 0 to day 14 as varied by seeding density for three donors

| | | Day 7 | Day 14 |
|---|---|---|---|
| SD1 | 259 | 2.84 | 6.17 |
| | 263 | 2.52 | 5.96 |
| | 201 | 2.53 | 5.08 |
| SD2 | 259 | 3.05 | 9 |
| | 263 | 2.69 | 7.15 |
| | 201 | 2.26 | 5.07 |
| SD3 | 259 | 2.07 | 11.81 |
| | 263 | 1.84 | 6.46 |
| | 201 | 1.76 | 8.3 |
| SD4 | 259 | 1.47 | 17.84 |
| | 263 | 1.14 | 3.7 |
| | 201 | 1.72 | 5.68 |

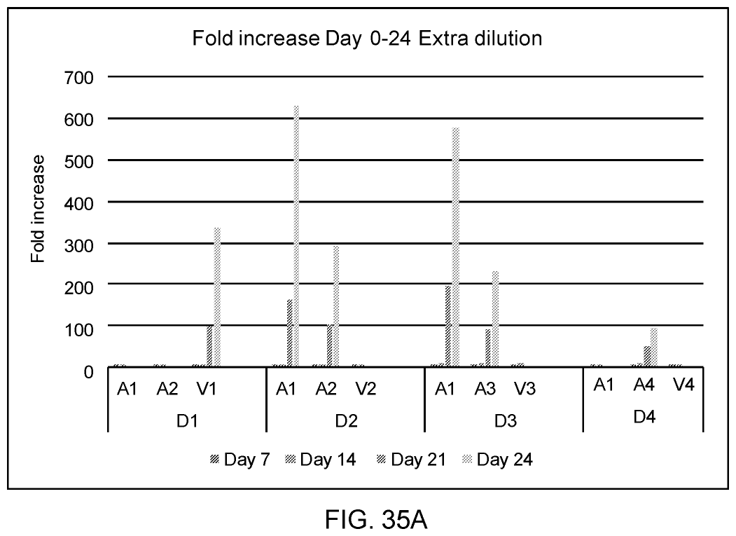

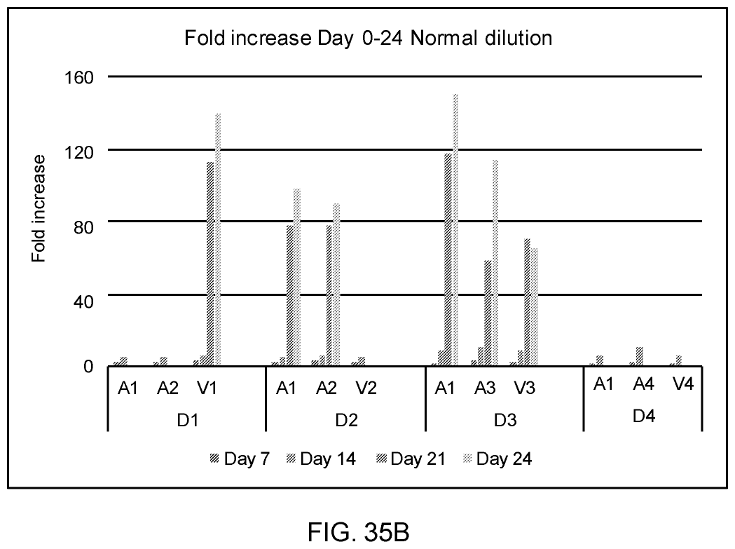

Day 14 is the point at which the culture is moved from the GREX10 to the GREX100 and provides a good point to analyze cells. The polyclonal activator CD2/CD28/CD3 is added, causing rapid proliferation as all cells are activated (not just those cells that are specific for antigen). This led to a comparison of a 1:1 volume dilution of the culture to 1:8 dilution at day 14. The extra media led to a fivefold increase in the fold change of cell numbers in the D3A1 group which includes seeding density SD3 (FIGS. 35A-351B and Tables 20A-20B). The additional nutrients improved the growth rate.

TABLE 20A
The fold increase of cell number at
different time points for Extra Diluted Samples

| Donor KP58201: Extra Dilution | | | | | |
|---|---|---|---|---|---|
| | | Day 7 | Day 14 | Day 21 | Day 24 |
| D1 | A1 | 2.53 | 5.08 | — | — |
| | A2 | 2.53 | 5.08 | — | — |
| | V1 | 2.98 | 6.12 | 97.30 | 338.26 |
| D2 | A1 | 2.26 | 5.07 | 162.51 | 630.23 |
| | A2 | 2.99 | 5.92 | 102.59 | 291.58 |
| | V2 | 2.26 | 5.07 | — | — |
| D3 | A1 | 1.76 | 8.30 | 194.50 | 579.19 |
| | A3 | 3.17 | 10.15 | 91.86 | 232.14 |
| | V3 | 1.93 | 8.80 | — | — |
| D4 | A1 | 1.72 | 5.68 | — | — |
| | A4 | 2.21 | 10.53 | 51.61 | 94.40 |
| | V4 | 0.76 | 6.35 | — | — |

TABLE 20B
The fold increase of cell number at
different time points for Normal Diluted Samples

| Donor KP58201: Normal Dilution | | | | | |
|---|---|---|---|---|---|
| | | Day 7 | Day 14 | Day 21 | Day 24 |
| D1 | A1 | 2.53 | 5.08 | — | — |
| | A2 | 2.53 | 5.08 | — | — |
| | V1 | 2.98 | 6.12 | 112.61 | 139.24 |
| D2 | A1 | 2.26 | 5.07 | 77.89 | 98.07 |
| | A2 | 2.99 | 5.92 | 77.71 | 89.87 |
| | V2 | 2.26 | 5.07 | — | — |
| D3 | A1 | 1.76 | 8.30 | 117.81 | 151.08 |
| | A3 | 3.17 | 10.15 | 58.28 | 113.73 |
| | V3 | 1.93 | 8.80 | 70.70 | 65.14 |
| D4 | A1 | 1.72 | 5.68 | — | — |
| | A4 | 2.21 | 10.53 | — | — |
| | V4 | 0.76 | 6.35 | — | — |

### Cell Phenotype

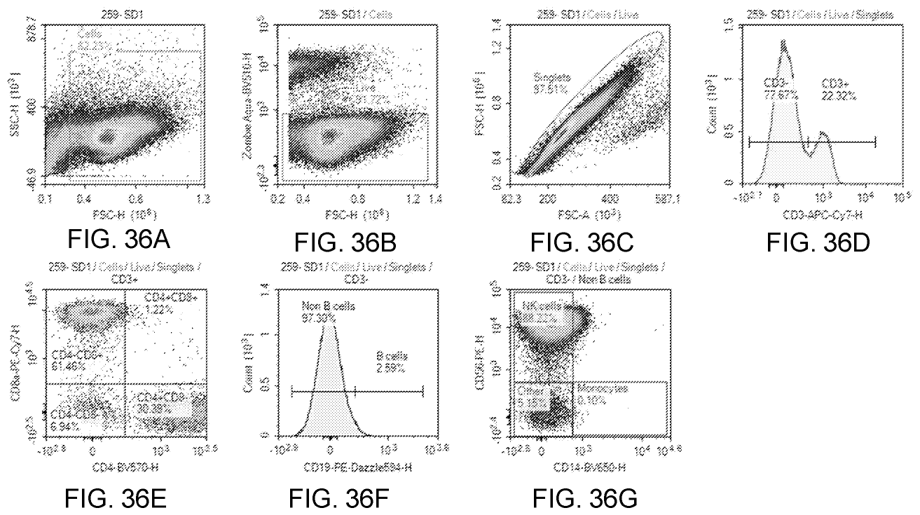

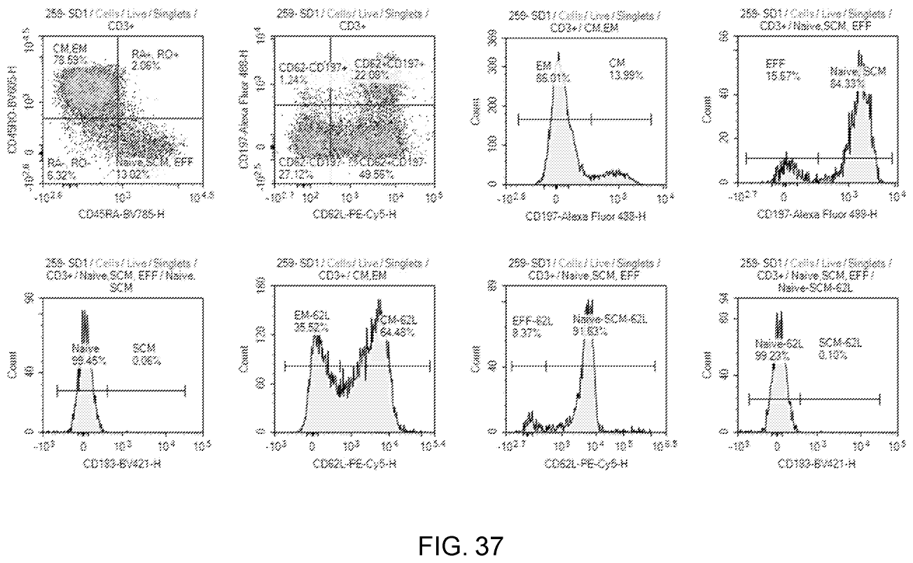

FIG. 36 shows plots depicting flow cytometry surface stain gating strategy using Donor 259 at day 14 as an example. FIG. 37 shows plots depicting flow cytometry gating strategy for memory T cell phenotypes using Donor 259 at day 14 as an example.

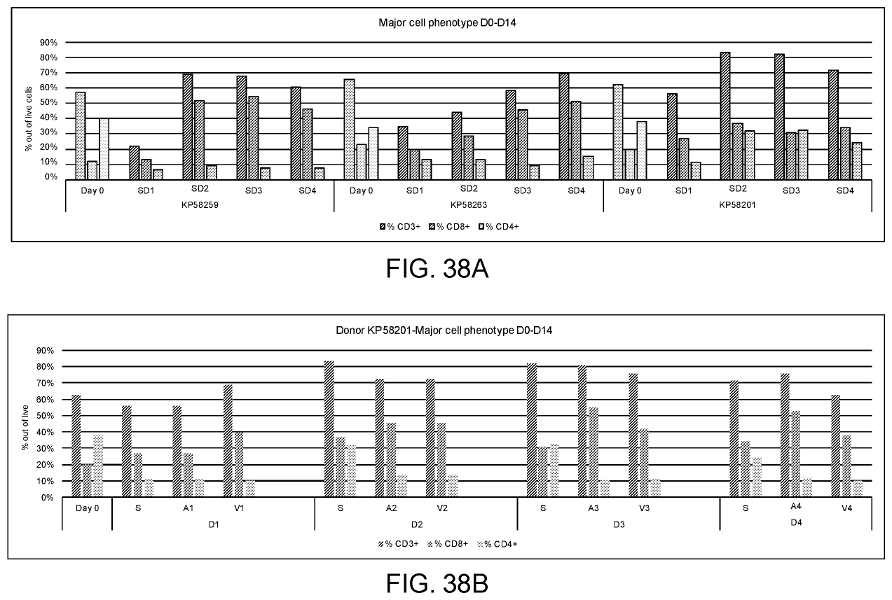

The phenotype of the resulting cells is important and ideally there should 100% CD3⁺ T cells and a balanced mix of CD4⁺ and CD8⁺ cells. FIGS. 38A-38B detail the influence of seeding density on T cell phenotypes. Reductions in cells per well while keeping the antigen and media volume the same resulted in increased CD3⁺, CD8⁺ and CD4⁺ cells. There is a corresponding decrease in non-T cell populations (FIG. 38A). The effect of reduction in concentration of antigen or volume of media according to number of cells used per well resulted in increased CD8⁺ cells and decreased CD4⁺ cells. A balanced CD8/CD4 ratio was found using 0.1 μg/mL antigens with 2.5 mL media volume (FIG. 38B). This antigen concentration did result in higher fractions of non-T cells as shown in FIG. 38B however SD3 has a low fraction of non-T cells.

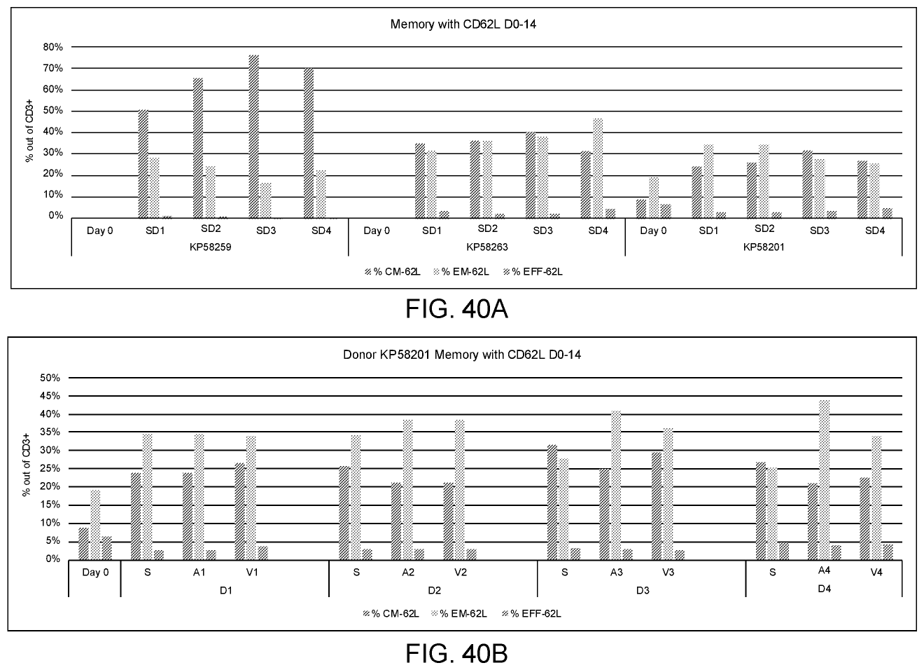

Further experiments at this seeding density denoted by SD3 with varying antigen conditions were then performed. The memory phenotype of the cells resulting from changes in seeding density are given in FIG. 39A. The memory component of the product is critical as it allows for sustained responses and lasting remission in treated patients. Central memory lasts the longest and was the highest fraction in SD3. The effect on memory phenotypes of reduction in concentration of antigen or volume of media according to number of cells used per well is given in FIG. 39B. For a given density, central memory goes down with reduction in antigen but is comparable with adjusting media volume. Effector memory which is short term goes up with both adjusting antigen as well as media volume. FIGS. 40A-40B show exemplary graphs of memory T cell phenotypes at different seeding densities for three donors for FIGS. 38A-38B.

### Cytokine Production

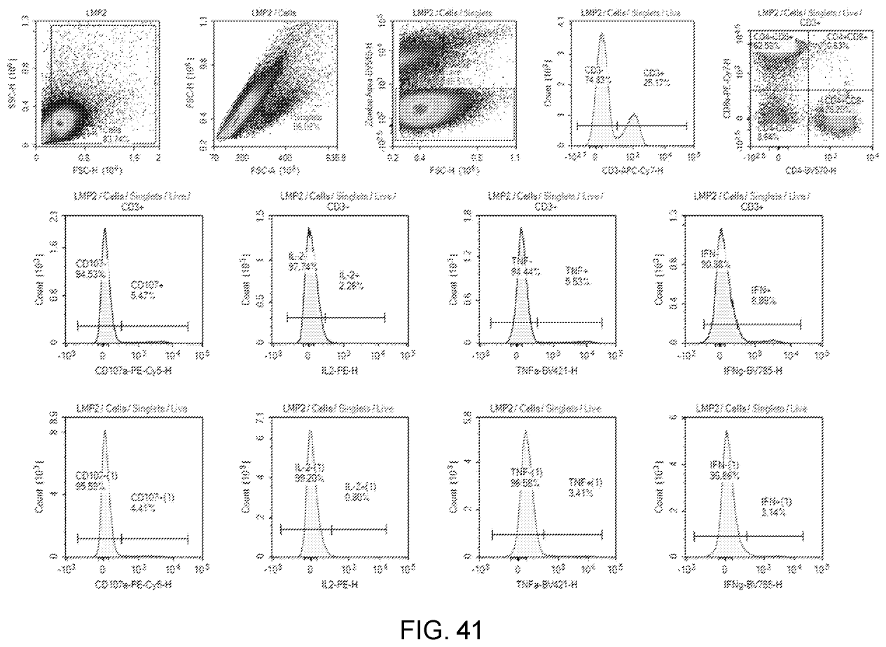

FIG. 41 shows plots depicting flow cytometry gating strategy for identifying cytokine producing T cells with an illustrative example of T cells reactive to a viral antigen LMP2A.

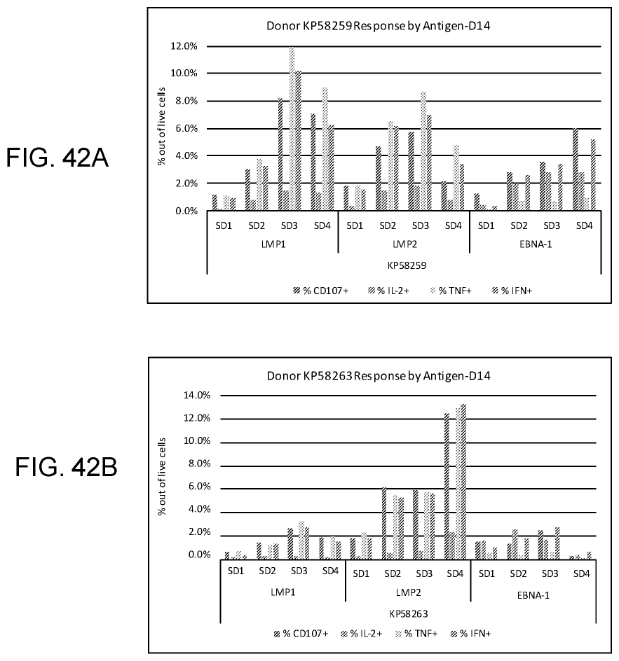

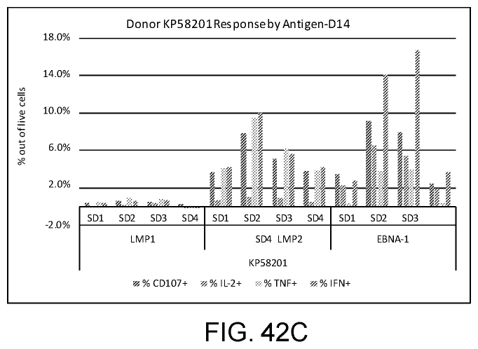

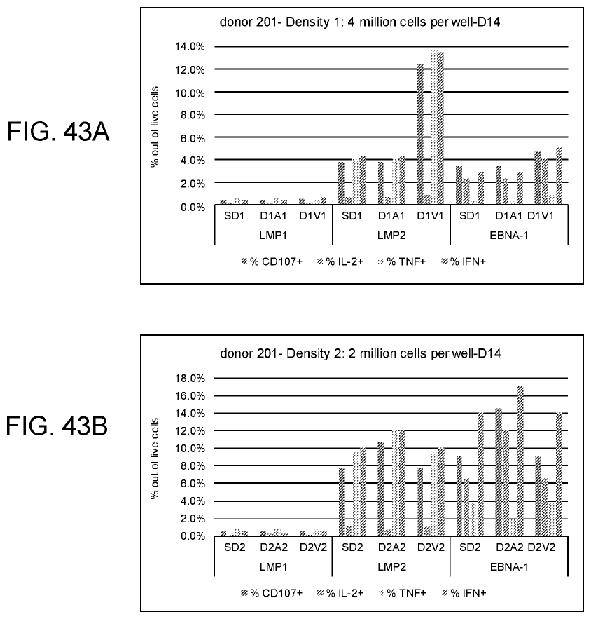

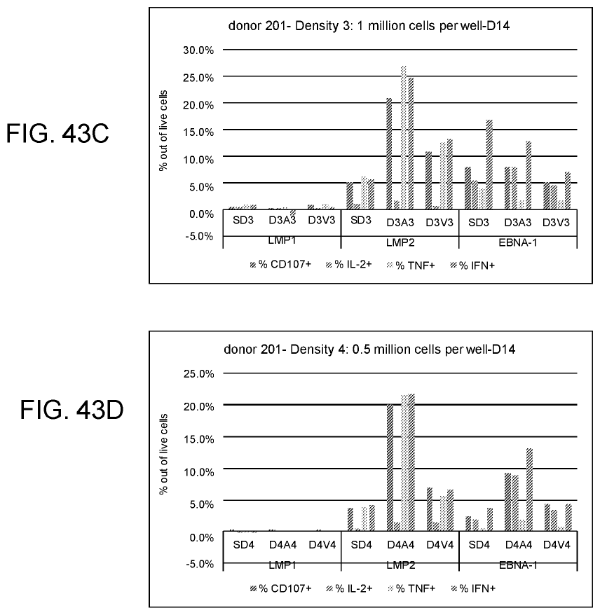

Antigen reactivity of the cells resulting from changes in seeding density are given in FIGS. 42A-42C. IFNγ production is an indicator of the strength of the response in a given population of T cells and is extremely important to the effectiveness of the T cell treatment. This also applies to the cytokines TNFα, IL-2 and the cytolytic capacity indicator CD107a. Response to LMP1 is best with density SD3 for all three donors (1×10⁶ cells/well with 0.1 μg/peptide/ml in 2.5 ml media). Response to LMP2 is more donor dependent and is best with either SD2, SD3 or SD4 (2×10⁶, 1×10⁶, or 0.5×10⁶ cells/well with 0.1 μg/peptide/ml in 2.5 ml media). Response to EBNA1 is best with either SD3 or SD4 depending on the donor (1×10⁶ or 0.5×10⁶ cells/well with 0.1 μg/peptide/ml in 2.5 ml media). The effect on cytokine production of reduction in concentration of antigen or volume of media according to number of cells used per well is given in FIGS. 43A-43D. Reducing antigen concentration affects LMP1 and EBNA1 responses negatively but LMP2 response increased with reducing antigen. Overall, the data indicated that the best condition is SD3A1 which is 1×10⁶ cells with 0.1 μg/peptide/ml antigen and 2.5 ml media per well with extra dilution protocol after day 14 polyclonal stim.

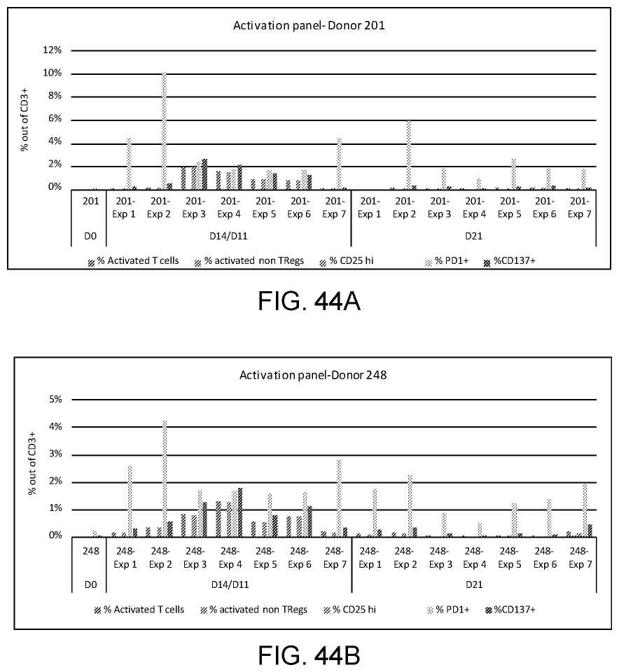

Observations on day 21 showed comparable results. These optimizations have decreased the PBMC no DC process timeframe from 28 days of culture to 21 days of culture as there are enough cells and with high enough antigen reactivity to be used for the treatment. Importantly, there are minimal amounts of T regulatory cells present in the culture (FIGS. 44A-44B). These cells act to suppress immune responses and would be detrimental to the efficacy of the treatment.

### Modifications in Antigen Stimulation

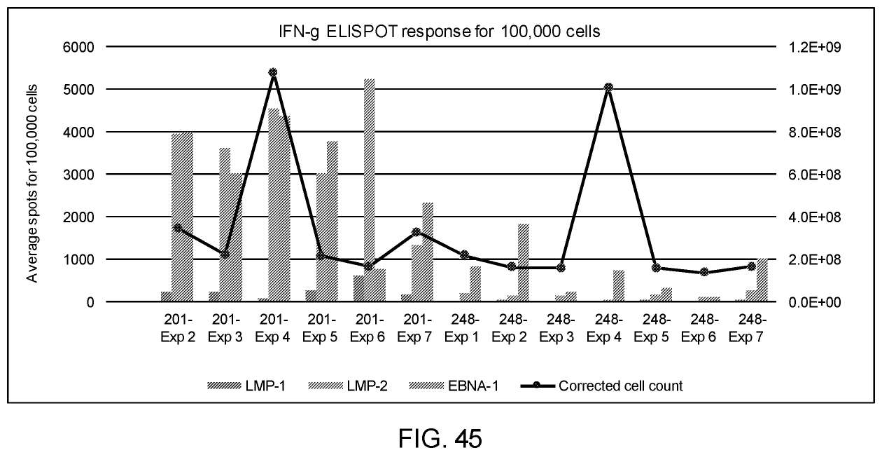

Experiments where the schedule of antigen addition such as the use of two versus three stimulations are detailed in Tables 18-21. These experiments also were assessed for fold change, memory phenotypes as before; however, the most significant results were seen in the IFNγ release in response to antigen tested at day 21 (FIG. 45, Table 22). FIG. 45 shows the IFNγ release in response to antigen as measured by ELISpot at day 21. Responses to the peptide PBMC no DC process three EBV pepmixes are provided for two donors per 100,000 cells. The final number of cells on day 21 is provided in the line. Tables 22-23 show the IFNγ release in response to antigen as measured by ELISpot at day 21.

TABLE 21
Experimental Parameters Showing the Modifications in Antigen
Stimulation and Other Parameters

| | Cells/ | | | | Poly- | | |
|---|---|---|---|---|---|---|---|
| | well/ | Antigen | | Antigen | clonal | Feed- | Cell |
| Exp. | stim | stim | Antigen | amount | stim | ing* | harvest |
| 1 | 4.5 × 10⁶ | Day 0, 7 | All | 0.1 μg/ | Day 14 | R1 | Day 21 |
| | | | three | peptide/ml | | | |
| 2 | 4.5 × 10⁶ | Day 1, 8 | All | 0.1 μg/ | Day 14 | R1 | Day 21 |
| | | | three | peptide/ml | | | |
| 3 | 4.5 × 10⁶ | Day 0, 7 | All | 0.1 μg/ | Day 11 | R2 | Day 21 |
| | | | three | peptide/ml | | | |
| 4 | 4.5 × 10⁶ | Day 0, 7 | All | 0.1 μg/ | Day 11 | 1:8, | Day 21 |
| | | | three | peptide/ml | | 1:2, | |
| | | | | | | 1:2 | |
| 5 | 4.5 × 10⁶ | Day 0, | All | 0.1 μg/ | Day 11 | R2 | Day 21 |
| | | 3, 7 | three | peptide/ml | | | |
| 6 | 4.5 × 10⁶ | Day 0, | One per | 0.3 μg/ | Day 11 | R2 | Day 21 |
| | | 3, 7 | stim | peptide/ml | | | |
| 7 | 4.5 × 10⁶ | Day 0, 7 | All | 0.1 μg/ | Day 14 | R1 | Day 21 |
| | | | three- | peptide/ml | | | |
| | | | MM | | | | |

TABLE 22
Experimental Details for INF-g
Release in Response to Antigen

| | | LMP-1 | LMP-2 | EBNA-1 |
|---|---|---|---|---|
| 201-Exp 2 | Resting | 240 | 3956 | 3995 |
| 201-Exp 3 | Day 11 poly stim | 254 | 3633 | 3019 |
| 201-Exp 4 | Extra dilution | 94 | 4560 | 4381 |
| 201-Exp 5 | Three stims | 279 | 3019 | 3770 |
| 201-Exp 6 | Three individual stims | 609 | 5259 | 774 |
| 201-Exp 7 | Master mix | 180 | 1346 | 2333 |
| 248-Exp 1 | Control | 24 | 198 | 849 |
| 248-Exp 2 | Resting | 49 | 150 | 1845 |
| 248-Exp 3 | Day 11 poly stim | 36 | 140 | 251 |
| 248-Exp 4 | Extra dilution | 25 | 44 | 758 |
| 248-Exp 5 | Three stims | 45 | 178 | 334 |
| 248-Exp 6 | Three individual stims | 38 | 106 | 103 |
| 248-Exp 7 | Master mix | 63 | 266 | 1011 |

TABLE 23
Experimental Details for INF-g Release in Response to Antigen

| | | Corrected | | | | | % spot |
|---|---|---|---|---|---|---|---|
| | | cell count | LMP-1 | LMP-2 | EBNA-1 | Total | forming |
| | | Day 21 | Count corrected for cells on Day 21 | | | spots | cells |
| 201-Exp 2 | Resting | 3.48E+08 | 8.34E+05 | 1.38E+07 | 1.39E+07 | 2.85E+07 | 8% |
| 201-Exp 3 | Day 11 | 2.21E+08 | 5.60E+05 | 8.02E+06 | 6.66E+06 | 1.52E+07 | 7% |
| | poly stim | | | | | | |
| 201-Exp 4 | Extra | 1.08E+09 | 1.01E+06 | 4.91 E+07 | 4.72E+07 | 9.74E+07 | 9% |
| | dilution | | | | | | |
| 201-Exp 5 | Three | 2.15E+08 | 6.00E+05 | 6.49E+06 | 8.11E+06 | 1.52E+07 | 7% |
| | stims | | | | | | |
| 201-Exp 6 | Three | 1.65E+08 | 1.01E+06 | 8.69E+06 | 1.28E+06 | 1.10E+07 | 7% |
| | individual | | | | | | |
| | stims | | | | | | |
| 201-Exp 7 | Master mix | 3.27E+08 | 5.89E+05 | 4.41E+06 | 7.64E+06 | 1.26E+07 | 4% |
| 248-Exp 1 | Control | 2.21E+08 | 5.24E+04 | 4.36E+05 | 1.87E+06 | 2.36E+06 | 1% |
| 248-Exp 2 | Resting | 1.61E+08 | 7.87E+04 | 2.42E+05 | 2.98E+06 | 3.30E+06 | 2% |
| 248-Exp 3 | Day 11 | 1.59E+08 | 5.76E+04 | 2.23E+05 | 3.99E+05 | 6.80E+05 | 0% |
| | poly stim | | | | | | |
| 248-Exp 4 | Extra | 1.01E+09 | 2.53E+05 | 4.43E+05 | 7.67E+06 | 8.36E+06 | 1% |
| | dilution | | | | | | |
| 248-Exp 5 | Three | 1.61E+08 | 7.23E+04 | 2.85E+05 | 5.36E+05 | 8.94E+05 | 1% |
| | stims | | | | | | |
| 248-Exp 6 | Three | 1.37E+08 | 5.14E+04 | 1.46E+05 | 1.40E+05 | 3.38E+05 | 0% |
| | individual | | | | | | |
| | stims | | | | | | |
| 248-Exp 7 | Master mix | 1.67E+08 | 1.04E+05 | 4.44E+05 | 1.69E+06 | 2.24E+06 | 1% |

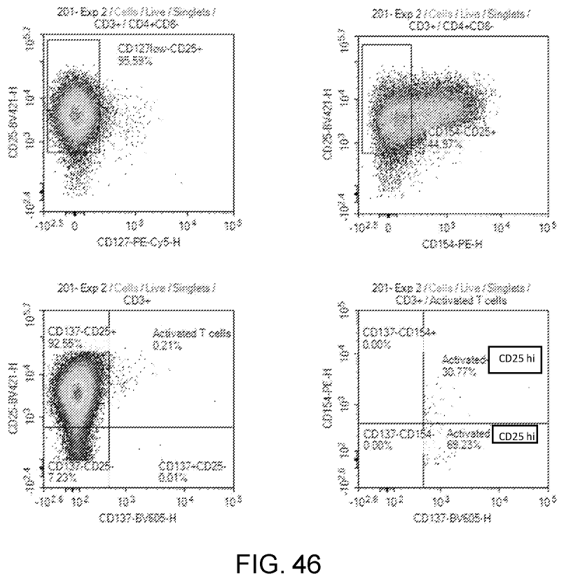

Flow cytometry surface stain gating strategy using Donor 201 at day 21 is shown in FIG. 46. T-regs by markers of T cell activation CD25(I121R), CD137(4-1-BB) and CD154 (CD40L). Activated T cells are measured by CD25 and then divided into T-regs and non-I-regs CD3⁺ I cells by CD154⁻CD137⁺. Additional Treg phenotype markers can be applied here as well, i.e., FOXP3, CD25+hi CD127−. Percentages are of the fraction of the parent population indicated and not total percent of cells.

Experiment 1 is the standard for comparison to the peptide PBMC no DC Process except for the antigen concentration changed from 3 pig/peptide/mL to 0.1 pig/peptide/mL. Prior experiments had indicated antigen reduction was beneficial to the health of the culture and can be provided if necessary. The results in FIG. 46 demonstrate that the optimum condition in terms of highest production of cells and highest fraction of antigen reactive cells to be experiment 4.

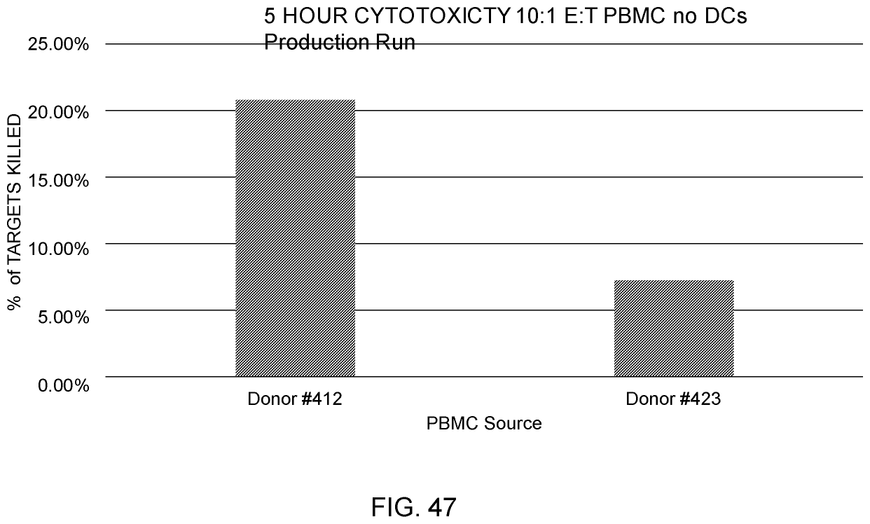

The results in FIG. 47 demonstrate that the PBMC no DCs or mRNA used closed system process produces I-cells with significant cytotoxic potential. PBMCs from two donors were put through a full manufacturing closed system process and at full scale targeting the three EBV antigens. The CSFE cytotoxicity test was performed using PHA blasts and peptides from the LMP2a antigen with Donor 412 killing 20.8% of targets and Donor 423 killing 7.28% of targets. In Donor 412 the fraction of cells producing IFNγ in response to antigen was 3.5% for LMP2a, 1% for LMP1, and <0.5% for EBNA1. In Donor 423 the fraction of cells producing IFNγ in response to antigen was 2% for LMP2a, 0.5% for LMP1, and <0.5% for EBNA1.

### Alternative Closed System Method—Welding PBMCs to Cassettes

In a modification of Process 2600 the closed system dendritic cell (“DC”) culture shown in FIG. 32 after step 2615 the bag of PBMCs is welded to the cassette and pumped in using the peristatic pump. The cassette is mounted at a final volume of 13-15 mL of 37° C. RPMI. After an hour of incubation, lateral flow is applied to transfer the non-adhered PBMC into an appropriate container where it will be washed (step 2645) and frozen as (steps 2650 to 2660). Adherent cells bound to the cassette are then cultured using lateral flow in DC differentiation media as according to the DC culture procedure. On day 5, maturation media is pumped into the cassette replacing the DC differentiation media. On day 6 matured DCs are harvested by pumping cold PBS over the cells and incubating the cassette on ice for 30 minutes. Closed system loading of the monocytes from PBMC's onto the polystyrene cartridge and the release of DC's post maturation from the polystyrene cartridge is achieved tubing and peristaltic pumps (i.e.—completely closed system) in contrast to manual loading and removal. In one embodiment a sterile air bubble was added to assist in the removal of cells off the surface. By causing space that allows for differential shear forces to lift the cells from the polystyrenes, this air bubble assists greatly in the removal of cells from the surface. In an alternative embodiment, the cartridge is rocked with the air bubble to release cells. In an alternative embodiment, the peristaltic pump is reversed in short cycles to move the air bubble back and forth over the surface to release cells. In preferred embodiments, the seeding of the monocytes onto the polystyrene is performed with the peristaltic pump at a low flow rate 5 to 9 mL/minute (7 mL per minute) and harvest is achieved at a higher peristaltic pump flow rate of 11-18 mL/per minute (14.6 mL per minute). This was then followed by transfer to a bag or into a 4D Nucleofector for RNA T cell production process or a G-Rex10 in the case of peptide T cell production process. The matching cells previously frozen are thawed, both bags' contents combined into the washing protocol of the Sepax C-Pro. Cells are transferred to the G-Rex10 (step 2630).

A further modification is after the DCs are matured but before harvesting, cationic lipids or other lipid-based technologies containing mRNA for transfection can be pumped into the cassette. After maximum expression is reached the cells are harvested. The matured untransfected cells post-harvest can be transferred to nucleofector 4D system that is compatible with sterile welding and bags (e.g., Lonza) with cationic lipids containing mRNA. After the DCs are combined with the previously frozen cells and washed on the Sepax C-Pro before transfer to the G-REX 10 for culture.

In a modification of Process 2600 the closed system DC culture shown in FIG. 32 after step 2615 the bag of PBMCs is welded to the cassette and pumped in using the peristatic pump. The cassette is mounted at a final volume of 50 mL of 37 C RPMI. After an hour of incubation lateral flow is applied to transfer the PBMC into an appropriate container where it will be washed (step 2645) and frozen as (steps 2650 to 2660). Adherent cells to the cassette are then cultured using lateral flow in DC differentiation media as according to the DC culture procedure. On day 5 maturation media is pumped into the cassette replacing the DC differentiation media. On day 6 matured DCs are harvested by pumping cold PBS over the cells and incubating the cassette on ice for 30 minutes followed by transfer to a bag. The matching cells previously frozen are thawed, both bags' contents combined into the washing protocol of the Sepax C-Pro. Cells are transferred to the G-Rex10 (step 2630).

A further modification is after the DCs are matured but before harvesting, cationic lipids containing mRNA for transfection are pumped into the cassette. After maximum expression is reached the cells can be harvested. The matured untransfected cells post-harvest can be transferred to nucleofector 4D system that is compatible with sterile welding and bags (e.g., Lonza) with cationic lipids containing mRNA. After the DCs are combined with the previously frozen cells and washed on the Sepax C-Pro before transfer to the G-REX 10 for culture.

The lipid composition of lipid-based nanoparticles used for the mRNA delivery may contain single and/or multiple lipid groups within the formulation. The lipid groups include: Cationic lipids: DOSPA 2,3-dioleyloxy-N-[2-(sperminecarboxamido)ethyl]-N,N-dimethyl-1-propanaminium trifluoroacetate, DOTMA 1,2-di-O-octadecenyl-3-trimethyl ammonium propane, DOTAP 1,2-Dioleoyl-3-trimethyalammoniumpropane, DC-Cholesterol 3β-[N—(N′,N′-dimethylaminoethane)-carbamoyl] cholesterol, Ionizablelipids:SM-1029-Heptadecanyl8-((2-hydroxyethyl)(6-oxo-6-(undecyloxy)hexyl)amino)octanoate, ALC-0315 4-hydroxybutyl)azanediyl)bis(hexane-6,1-diyl)bis(2-hexyldecanoate) DLin-MC3-DMA, (6Z,9Z,28Z,31Z)-heptatriaconta-6,9,28,31-tetraen-19-yl4-(dimethylamino) butanoate, DODMA 1,2-Dioleyloxy-3-dimethylamino propane. Helper lipids: Cholesterol (1R,3aS,3bS,7S,9aR,9bS,11 aR)-9a,11a-Dimethyl-1-[(2R)-6-methylheptan-2-yl]-2,3,3a,3b,4,6,7,8,9,9a,9b,10,11,11a-tetradecahydro-1H-cyclopenta[a]phenanthren-7-ol DSPC 1,2-distearoyl-sn-glycero-3-phosphocholine, DOPE 1,2-Dimyristoyl-sn-glycerophosphoethanolamine. Stealth lipids: PEG-DMG (R)-2,3-bis(myristoyloxy)propyl-1-(methoxy poly (ethylene glycol) 2000) carbamate and ALC-0159 2-[(polyethylene glycol)-2000]-N,N-ditetradecylacetamide. In some embodiments, mannose carbohydrates will be included to the formulation. The addition of mannose carbohydrates assists in the binding to the dendritic cells (“DCs”) by using the mannose receptor on the DCs.

Another example of a “cassette” based closed system is detailed in FIG. 33D. The cassette from FIG. 33C is modified such that the top portion of the cassette is made of a silicone membrane instead of rigid plastic. This modification moves the oxygen and CO₂ exchange from the silicone tubing to the cassette itself. This allow for two changes: constant lateral flow is no longer required, and the cassette can be used for combined DC and non-adherent cells culture. The lateral flow was needed to move gases through the tubing and has the disadvantage of potentially picking up cells floating in the cassette and carries them through to the waste. The G-REX® growth chamber is used for growing T cells, and it uses a silicone cup to put the cells into for gas exchange leading to better T cell growth. The membrane allows them to be proximal to a gas exchange surface by collecting onto the bottom of the silicone cup. In FIG. 33D the plastic on the bottom of the cassette is used for the monocytes to adhere to which is required for their isolation. Later after they have differentiated, they no longer require a surface to adhere to, T cells can be added to the culture and the entire cassette flipped over 180° so that the cells in culture can rest on the silicone membrane.

Taken together, these results demonstrate that the mRNA or peptide T cell production process can be performed using the closed system.

Upon completion of the process the cells are washed thoroughly and undergo release testing. The purified cells should be free of DCs, have no exogenous cytokines remaining or human sera. Purified cells with transfusion appropriate freezing media are packed into IV bags and frozen using a control rate freezer. Cells are sent on dry ice to an outpatient clinic where they will be diluted with physiological saline to lower the percent DMSO of the infusion. The patient goes to an outpatient clinic and is infused with the cells over the course of hours. Patients are monitored that day. No further treatments should be required.

### Example 8: Knockout of B2M Gene in Cells Produced from the mRNA T-Cell Production

The following example describes incorporating gene editing techniques into the mRNA T-cell production process to facilitate knockout of the β2-microglobulin (B2M) gene. B2M protein forms a heterodimer with HLA class I proteins and is required for HLA class I presentation on the cell surface. Suppression of the B2M gene prevents an immune response from cytotoxic T cells by depleting all HLA class I molecules. The absence of missing MHC I molecules can also serve to slow or prevent the clearance of MHC mismatched engrafted cells, essentially host versus graft.

After the production of allogenic T cells described in Examples 1-7 above, the present example describes how to knockout the B2M gene in the T cells. The source of the allogeneic cells can be from another donor (i.e., not from the patient) who has partially matched MHC and whose cells efficiently produce cytokines and killed target cells expressing the target neoantigen. Alternatively, B2M can be knocked out before the T cell expansion with polyclonal CD3/CD28/CD2 T-cell activator (StemCell technologies).

A protein RNA complex consisting of recombinant Cas9 protein with a guide RNA against B2M is transferred to the allogenic cells at scale either by cationic lipids, electroporation, or calcium phosphate within a large bioreactor. After washing the transfected allogenic cells, the cells can then be returned to culture conditions for 24 hours after which they will be washed and placed into suitable freezing bags with Cryostor® freezing media (StemCell Technologies) and released for treatment. The use of transient Cas9 prevents persistent Cas9 activation which can kill cells, produce an inflammatory reaction in the recipient and potentially have oncogenic effects by increasing DNA damage within cells.

Knocking out β2-microglobulin by CRISPR/Cas9 could also be applied to cause a defect in MHC class I. Within an allogenic environment, disruption or removal of MHC Class I could result in an increase in cellular half-life within the patient, giving the patient an opportunity to mount their own immune response. Previously selected COVID-19 T-cells are used for knocking out the β2-microglobulin by CRISPR/Cas9. A commercial kit is used to knockout β2-microglobulin (OriGene, P/N=KN207587RB). Following the manufacture's knock-out protocol, the knocked-out T-cells were screened by comparing the half-life in a digital killing or MLR assay. FIG. 49A The cells were plated into xCelligence Real-Time Cell Analysis (RTCA) cartridge and then exposed to matched, partial matched, and fully mismatched allogenic PBMCs, and it was determined whether there was a difference in half-life between the β2-microglobulin knock-out vs the pre-knock-out T-cells. The added allogenic cells could either kill the T-cells or cause MLR proliferation. The RTCA readout is the impedance vs time.

This process was performed in a mouse model where T-cell line targeting LMP2a derived from the mRNA T cell production process was used as the parent cell line “G-LMP2.” One million cells are injected on day 1 into BALB/c mice and bled every five days. FIG. 49B shows an exemplary graph of fraction of transplanted cells indicating rate of clearance of human T-cell lines in BALB/c mice. FIG. 49B shows a CRISPR knockout (KO) of B2M resulting in loss of MHC I expression on the G-LMP2 background “G-B2M KO,” a transient transfection of PD-L1 mRNA in G-LMP2 “G-PDL1,” a combined knockout and PD-L1 transiently expressing cells “G-B2M-PDL1”. Each cell type is labelled fluorescently, and blood is analyzed by flow cytometry.

Direct comparison of allogeneic cells with and without MHC I post transplantation is expected to result in longer half lives in the blood in MHC I knockouts. This would result in longer term protection. Alternatively, they can be applied as a bridge for the time after the blood draw of a patient who will have an autologous product made and infusion of that autologous product.

### Example 9: Expression of Molecules for the Improvement of Immune Cell Homing and Reversal of Tumor Microenvironment

The following example describes incorporating into the mRNA T cell process transient expression of molecules designed for improved sustainability of activity. Tumors evade the endogenous immune system by creating an immunosuppressive microenvironment. Mechanisms include expression of immune modulating surface receptors or secreted proteins by tumor cells and recruitment of immunosuppressive tumor infiltrating lymphocytes including (CD4+ Foxp3+) regulatory T cells and regulatory NK cells. To counteract the immunosuppressive microenvironment, tumor specific T cells can be modified to express pro-inflammatory signals. These include but are not limited to secreted cytokines (IL-2, IL-7, IL-12, IL-15, IL-18, IL-21, IFNα, IFNβ, IFNγ, TNFα, and others), secreted chemokines (CCL2, CCL5, CCL9, CCL10, CCL11, CCL12, CCL13, CCL19 CCL21, and others), cytokine receptors for all of the above cytokines, chemokine receptors for all of the above chemokines, and costimulatory molecules (CD28, CD40L, 4-1 BB, OX40, CD46, CD27, ICOS, HVEM, LIGHT, DR3, GITR, CD30, TIM1, SLAM, CD2, CD226, and others). Negative immune checkpoint regulators (PD-1, PD-L1, CTLA-4, Fas, FasL, LAG3, B7-1, B7-H1, CD160, BTLA, LAIR1, TIM3, 2B4, TIGIT, TGFβ, TGFβ receptor, IL-4 receptor, IL-10 receptor, VEGF receptor, and others) can be converted to proinflammatory molecules through fusion of their extracellular domain with the intracellular signaling domain of a costimulatory protein (for example Fas fused with CD28, CD40L, 4-1 BB, OX40, ICOS, or others).

Alternatively, T cells can be made to secrete antibodies, single chain antibodies (scFv's), Fab fragments, or bispecific T cell engagers to block targets including αvβ8 integrin, PD-1, PD-L1, CTLA-4, Fas, FasL, LAG3, B7-1, B7-H1, CD160, BTLA, LAIR1, TIM3, 2B4, TIGIT, TGFβ, TGFβ receptor, IL-4 receptor, IL-10 receptor, VEGF receptor, and others. T cells can also be made to directly modify the tumor microenvironment by expressing enzymes that alter the extracellular matrix (heparinase, catalase, matrix metalloproteinases, hyaluronidase, RHEB, and others).

T cells with modified genomic DNA (including CAR-T cells) have been shown to be effective against some forms of cancer. However, permanent modification of the genome comes with substantial risks. Cells programmed to be hyperinflammatory by overexpression of costimulatory molecules or reduced expression coinhibitory receptors can lead to a hyperimmune response such as cytokine release syndrome or graft-versus-host disease. Furthermore, using a lentivirus, retrovirus, CRISPR/Cas9 or other means of integrating genes into the genome or deleting genes can result in off-target effects which can lead to misregulation of endogenous genes with unintended consequences including possible oncogenic transformation of the modified cells. Another drawback of genome modified T cells is that these cells are clonal and thus can only respond to one or a very small number of tumor antigens. Additionally, the cell engineering timetable is on the order of months to years, too long for many cancer patients. Therefore, this is not a practical strategy for generating personalized, patient-specific T cells to multiple neoantigens presented in the context of patient-specific HLA proteins. An alternative approach to DNA modification is to transfect tumor-specific T cells with mRNA resulting in transient expression of the desired gene or genes, as RNA is rapidly degraded and there is no permanent modification of the genome. This limit intended effects to the therapeutic time window.

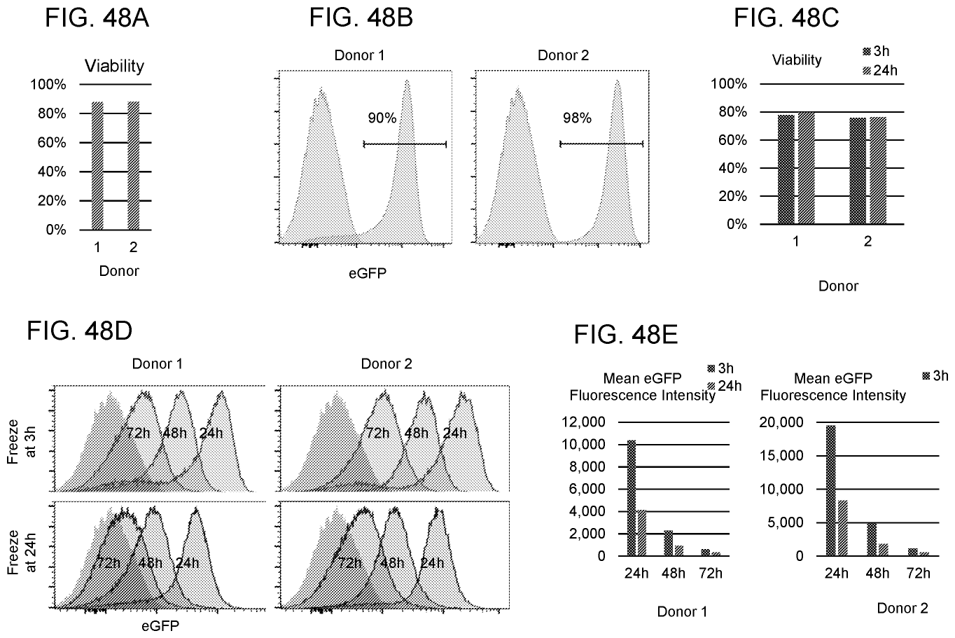

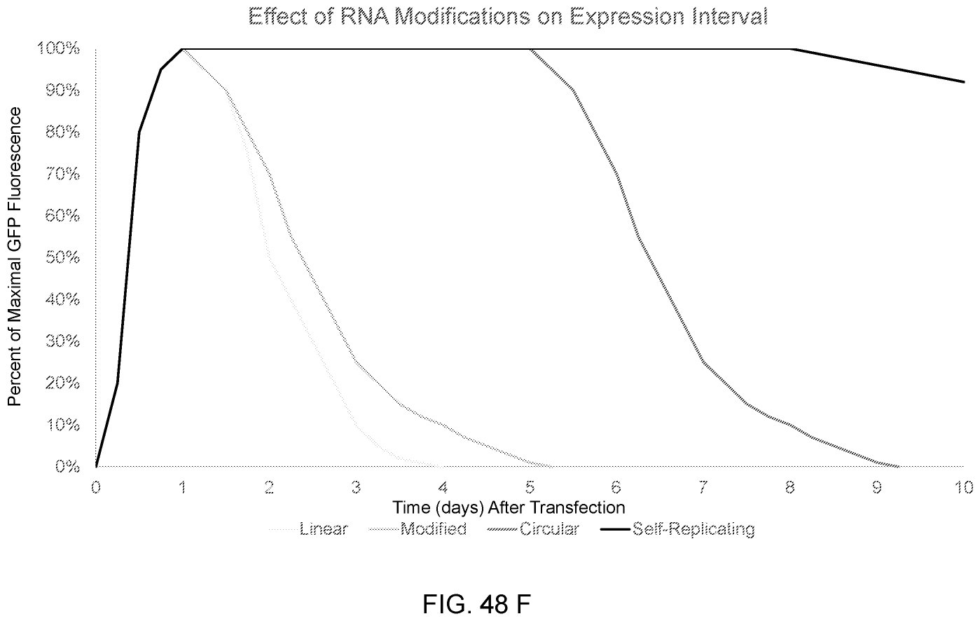

T cells are difficult to transfect. However, using Lonza 4D-Nucleofection T cells can be transfected with mRNA at very high efficiency and viability FIGS. 48A-48B. As RNA expression is transient, the T cell product will be transfected with RNA at the final step of production following stimulation and priming. Messenger RNA transfection leads to a peak in protein product expression at ˜24 h post-transfection. Protein expression rapidly declines over time but is still present at >72 hours post-transfection FIGS. 48D-48E.

Electroporation is toxic to cells. To maximize yield and viability cells must be allowed to recover in cell culture. Addition of small molecule inhibitors (including but not limited to Rho kinase and ROCK inhibitors) to the cell culture media during this recovery period can increase the viability of electroporated cells. However, keeping these cells in cell culture for an extended period will reduce the amount of time that these cells express the desired mRNA in vivo following injection into patients. Therefore, it is optimal to freeze cells as soon as they recover from electroporation. Indeed, T cells frozen at 3 hours post-nucleofection expressed higher levels of mRNA product for an extended period as compared to the same cells frozen at 24 hours post-nucleofection and had similar viability FIGS. 48C-48E.

To increase the half-life of transfected mRNA, the RNA can be modified to increase its stability. These modifications include but are not limited to modifying the 5′UTR, modifying the 3′UTR, using alternative nucleotides (such as 5-methoxy-UTP), modifying the RNA cap, using circular RNA, or using self-replication RNA (FIG. 48F).

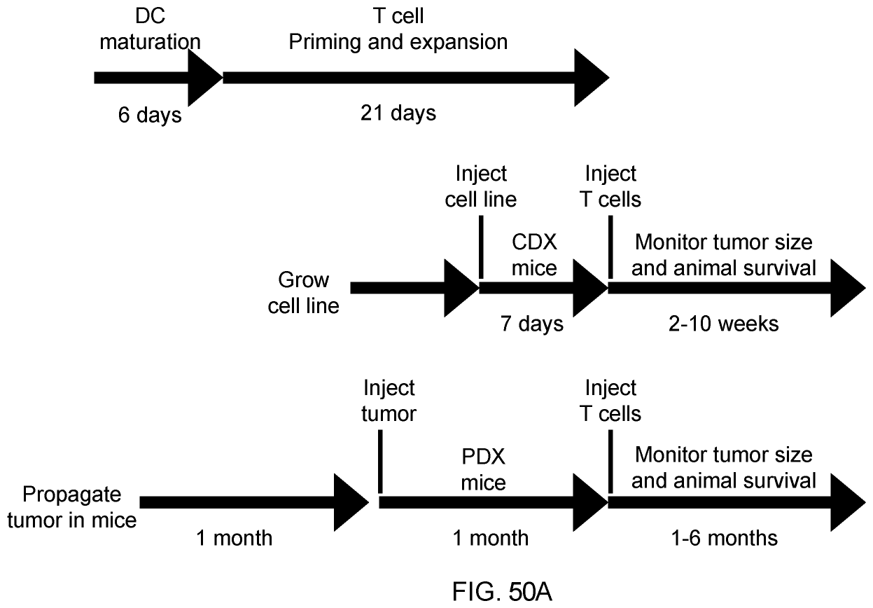

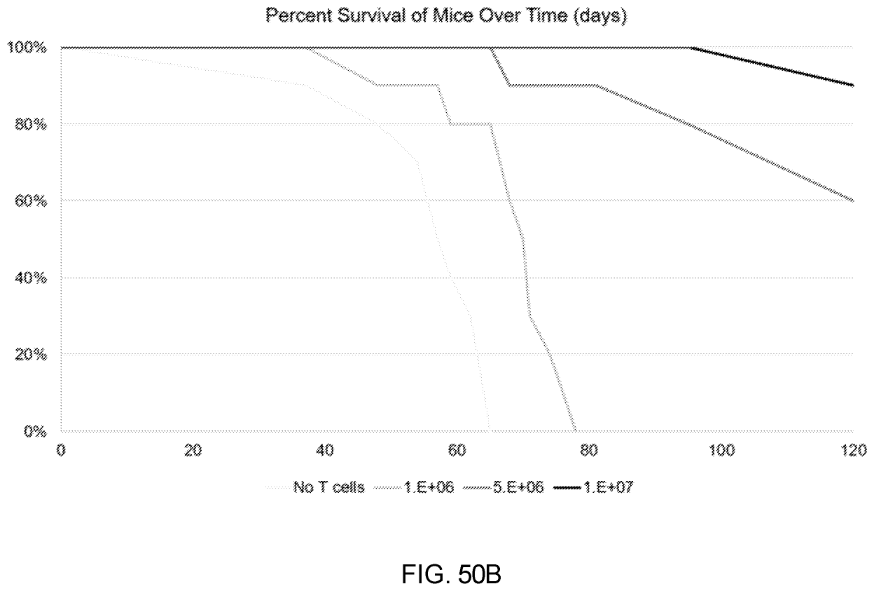

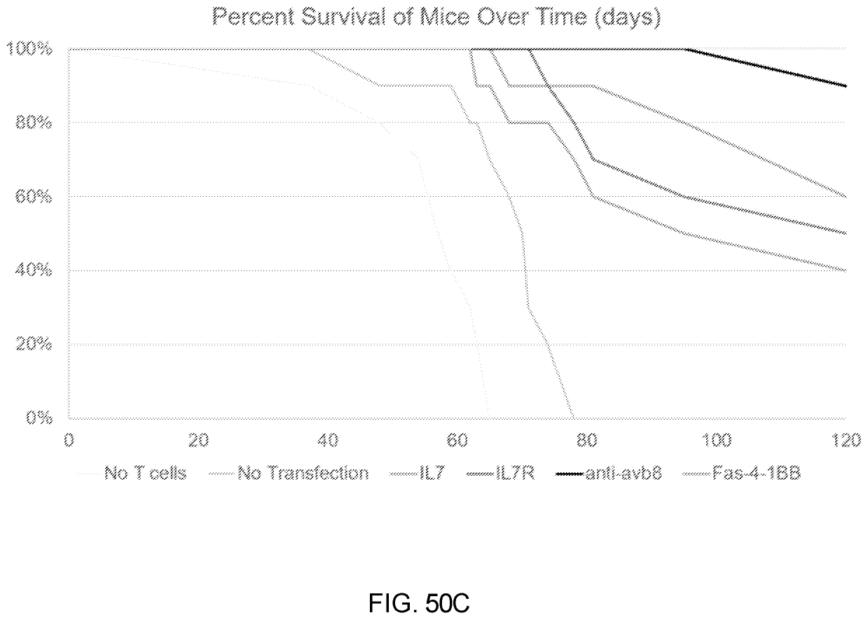

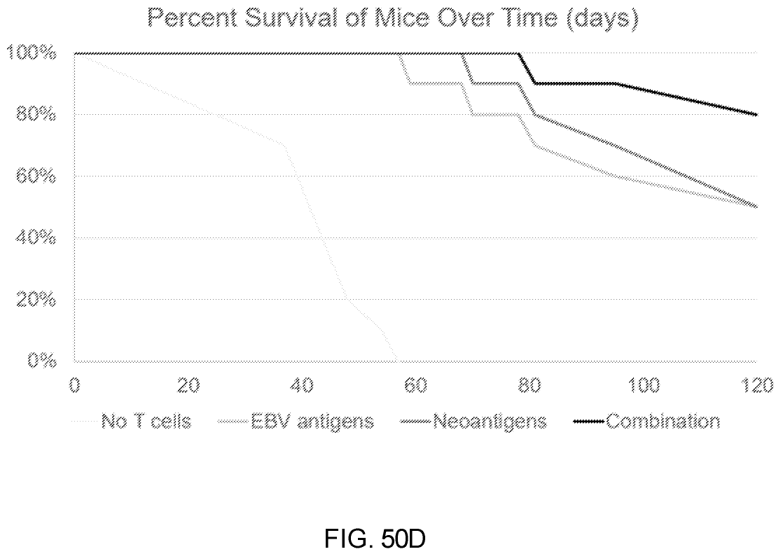

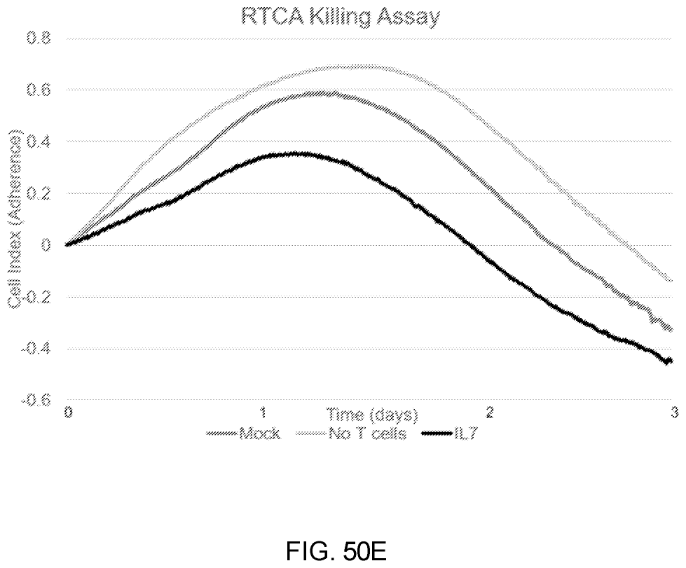

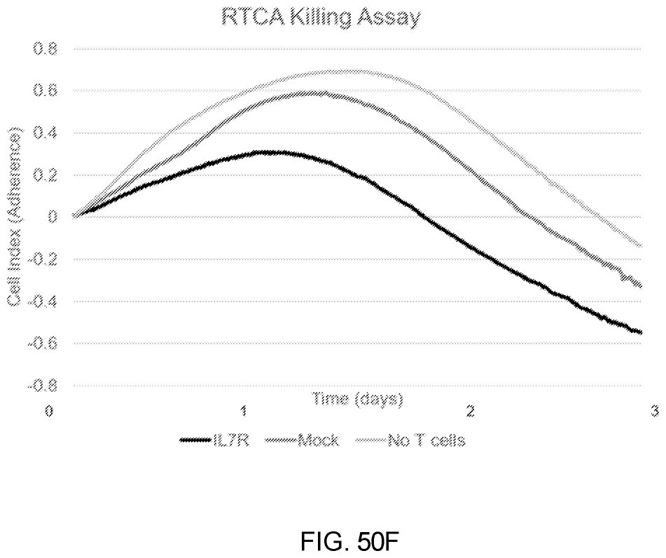

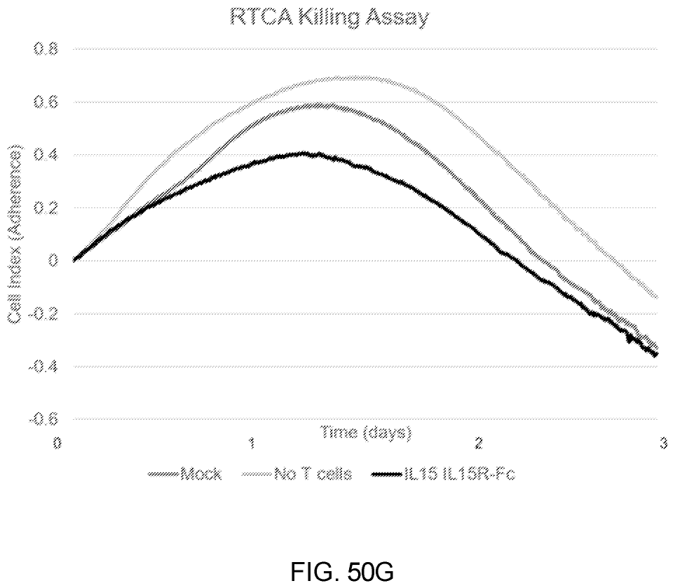

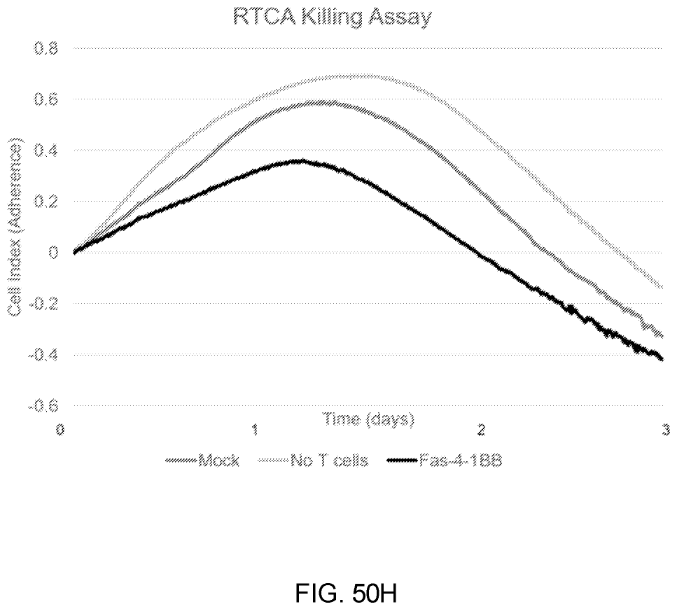

This following example demonstrates improved anti-tumor activity of T cells modified transiently with mRNA compared to unmodified T cells generated with the mRNA T cell process (FIGS. 50A-50C). To test the efficacy of T cells, Cell line Derived Xenograft (CDX) or Patient Derived Xenograft (PDX) mice are generated using tumor cells from patient Z (FIG. 50A). FIG. 50B shows the dose response in percent survival of mice treated with different numbers of T cells derived from patient Z using the mRNA T cell process. T cells are modified using mRNA encoding for human IL7, IL7R, a secreted single chain antibody (scFvs) against αvβ8 integrin, IL15 in combination with a fusion protein for IL15R and the Fc region of IgG to allow for efficient secretion and stability of IL15, and a fusion protein containing the extracellular domain of Fas and the intracellular domain of 4-1 BB. The mRNA is produced as before with the necessary non-coding components to facilitate translation and purification. After the production of an autologous T cell product with blood from donor Z (according to the mRNA T cell production process examples 1-7) the purified T cell product is pumped into a closed system Lonza nucleofector and transfected with 2 μg of mRNA per 1 million cells. The T cell product is then washed and placed into suitable freezing bags with Cryostor freezing media. A portion of the tumor resected from donor Z is engrafted onto an immunodeficient mouse such there is continuous blood supply from the mouse to the tumor such that the tumor proliferates. The cells from donor Z that had previously been produced to target the mutations present in tumor Z are then injected into the mouse bearing tumor Z at 1 million cells per mouse. This will consist of six groups: untreated mice, mice treated with unmodified T cell, mice treated with T cells modified with human IL7 mRNA, mice treated with T cells modified with human IL7R mRNA, mice treated with T cells modified with a secreted single chain antibody (scFvs) against αvβ8 integrin, and mice treated with T cells modified with the Fas-4-1 BB fusion protein.

Expressing human IL-7, IL-7R, a secreted single chain antibody (scFvs) to αvβ8 integrin, human IL15 in combination with the IL15R-Fc fusion protein, and Fas-4-1 BB fusion protein improve the function of the T cells as measured by increased rate of tumor shrinkage, higher number of infiltrating human T cells within the tumor, and increased survival time for transfected cells versus untransfected cells and negative control (FIG. 50C).

EBV+ lymphoma can be treated in two ways using the mRNA T cell process: (1) using mRNA for EBV genes including the combination of LMP1, LMP2, and EBNA1 and/or (2) using neoantigens to mutated endogenous genes specific to each patient's lymphoma. A combination therapy may be advantageous as it allows for further diversity of the lymphoma-specific T cell repertoire and makes it makes it more difficult for the lymphoma cells to acquire resistance be silencing any individual genes. T cells can be primed separately using dendritic cells (DCs) transfected with mRNA to EBV antigens and DCs transfected with neoantigens. These T cells would then be combined and administered together. A second method is to transfect DCs with mRNA to both EBV antigens and neoantigens together using separate mRNAs or one mRNA containing both sets of antigens resulting in a single T cell product with specificity to both EBV antigens and neoantigens.

In this example CDX mice (FIG. 50A) are generated using the Raji cell line. Raji were derived from an EBV+ Burkitt Lymphoma and have been sequenced to identify numerous neoantigens. T cells from HLA-matched donors are generated using the mRNA T cell process using mRNA for EBV antigens (LMP1, LMP2, and EBNA1), neoantigens, or a combination of both. Shown is the survival of Raji CDX mice treated with these T cells (FIG. 50D).

In this example, T cell product from donors HLA-matched to Raji lymphoma cells were generated using the mRNA T cell process targeting EBV antigens (LMP1, LMP2, and EBNA1). Cells were then modified with transient mRNA transfection with either human IL7 (FIG. 50E), IL7R (FIG. 50F), IL15 in combination with a fusion protein for IL15R and the Fc region of IgG to allow for efficient secretion and stability of IL15 (FIG. 50G), or Fas-4-1 BB fusion protein (FIG. 50H). Modified T cells were more efficient at killing Raji lymphoma compared to no T cells and mock transfected I cells as measured using the Real Time Cell Analyzer (RICA). Sequences of some of the mRNAs referred to herein are detailed in Table 24.

TABLE 24
Sequences of mRNA constructs

| Gene | Sequence of RNA Translated Region |
|---|---|
| IL7 | AUGUUCCAUGUUUCUUUUAGAUAUAUCUUUGGAAUUCCUCCACUGAUCCUUGUUCUGCUGCCUGUCACAUCA |
| | UCUGAGUGCCACAUUAAAGACAAAGAAGGUAAAGCAUAUGAGAGUGUACUGAUGAUCAGCAUCGAUGAAUUG |
| | GACAAAAUGACAGGAACUGAUAGUAAUUGCCCGAAUAAUGAACCAAACUUUUUUAGAAAACAUGUAUGUGAU |
| | GAUACAAAGGAAGCUGCUUUUCUAAAUCGUGCUGCUCGCAAGUUGAAGCAAUUUCUUAAAAUGAAUAUCAGU |
| | GAAGAAUUCAAUGUCCACUUACUAACAGUAUCACAAGGCACACAAACACUGGUGAACUGCACAAGUAAGGAA |
| | GAAAAAAACGUAAAGGAACAGAAAAAGAAUGAUGCAUGUUUCCUAAAGAGACUACUGAGAGAAAUAAAAACU |
| | UGUUGGAAUAAAAUUUUGAAGGGCAGUAUAUGA |
| IL7R | AUGACAAUUCUAGGUACAACUUUUGGCAUGGUUUUUUCUUUACUUCAAGUCGUUUCUGGAGAAAGUGGCUAU |
| | GCUCAAAAUGGAGACUUGGAAGAUGCAGAACUGGAUGACUACUCAUUCUCAUGCUAUAGCCAGUUGGAAGUG |
| | AAUGGAUCGCAGCACUCACUGACCUGUGCUUUUGAGGACCCAGAUGUCAACAUCACCAAUCUGGAAUUUGAA |
| | AUAUGUGGGGCCCUCGUGGAGGUAAAGUGCCUGAAUUUCAGGAAACUACAAGAGAUAUAUUUCAUCGAGACA |
| | AAGAAAUUCUUACUGAUUGGAAAGAGCAAUAUAUGUGUGAAGGUUGGAGAAAAGAGUCUAACCUGCAAAAAA |
| | AUAGACCUAACCACUAUAGUUAAACCUGAGGCUCCUUUUGACCUGAGUGUCGUCUAUCGGGAAGGAGCCAAU |
| | GACUUUGUGGUGACAUUUAAUACAUCACACUUGCAAAAGAAGUAUGUAAAAGUUUUAAUGCACGAUGUAGCU |
| | UACCGCCAGGAAAAGGAUGAAAACAAAUGGACGCAUGUGAAUUUAUCCAGCACAAAGCUGACACUCCUGCAG |
| | AGAAAGCUCCAACCGGCAGCAAUGUAUGAGAUUAAAGUUCGAUCCAUCCCUGAUCACUAUUUUAAAGGCUUC |
| | UGGAGUGAAUGGAGUCCAAGUUAUUACUUCAGAACUCCAGAGAUCAAUAAUAGCUCAGGGGAGAUGGAUCCU |
| | AUCUUACUAACCAUCAGCAUUUUGAGUUUUUUCUCUGUCGCUCUGUUGGUCAUCUUGGCCUGUGUGUUAUGG |
| | AAAAAAAGGAUUAAGCCUAUCGUAUGGCCCAGUCUCCCCGAUCAUAAGAAGACUCUGGAACAUCUUUGUAAG |
| | AAACCAAGAAAAAAUUUAAAUGUGAGUUUCAAUCCUGAAAGUUUCCUGGACUGCCAGAUUCAUAGGGUGGAU |
| | GACAUUCAAGCUAGAGAUGAAGUGGAAGGUUUUCUGCAAGAUACGUUUCCUCAGCAACUAGAAGAAUCUGAG |
| | AAGCAGAGGCUUGGAGGGGAUGUGCAGAGCCCCAACUGCCCAUCUGAGGAUGUAGUCAUCACUCCAGAAAGC |
| | UUUGGAAGAGAUUCAUCCCUCACAUGCCUGGCUGGGAAUGUCAGUGCAUGUGACGCCCCUAUUCUCUCCUCU |
| | UCCAGGUCCCUAGACUGCAGGGAGAGUGGCAAGAAUGGGCCUCAUGUGUACCAGGACCUCCUGCUUAGCCUU |
| | GGGACUACAAACAGCACGCUGCCCCCUCCAUUUUCUCUCCAAUCUGGAAUCCUGACAUUGAACCCAGUUGCU |
| | CAGGGUCAGCCCAUUCUUACUUCCCUGGGAUCAAAUCAAGAAGAAGCAUAUGUCACCAUGUCCAGCUUCUAC |
| | CAAAACCAGUGA |
| IL15 | AUGGAAACCGACACACUGCUGCUGUGGGUGCUGCUUCUUUGGGUGCCCGGCUCUACAGGCAACUGGGUCAAC |
| | GUGAUCAGCGACCUGAAGAAGAUCGAGGACCUGAUCCAGAGCAUGCACAUCGACGCCACACUGUACACCGAG |
| | AGCGACGUGCACCCUAGCUGUAAAGUGACCGCCAUGAAGUGCUUUCUGCUGGAACUGCAAGUGAUCAGCCUG |
| | GAAAGCGGCGACGCCAGCAUCCACGACACCGUGGAAAACCUGAUCAUCCUGGCCAACGACAGCCUGAGCAGC |
| | AACGGCAAUGUGACCGAGUCCGGCUGCAAAGAGUGCGAGGAACUGGAAGAGAAGAAUAUCAAAGAGUUCCUG |
| | CAGAGCUUCGUGCACAUCGUGCAGAUGUUCAUCAACACCAGCUGAUGAUGA |
| IL15R-Fc | AUGGAUAGACUGACCAGCAGCUUCCUGCUGCUGAUCGUGCCUGCCUACGUGCUGAGCAUCACCUGUCCUCCA |
| | CCUAUGAGCGUGGAACACGCCGACAUCUGGGUCAAGAGCUACAGCCUGUACAGCAGAGAGCGGUACAUCUGC |
| | AACAGCGGCUUCAAGAGAAAGGCCGGCACCAGCAGCCUGACCGAGUGUGUGCUGAACAAGGCCACCAAUGUG |
| | GCCCACUGGACCACACCUAGCCUGAAGUGCAUCAGAGAGCCCAAGAGCUGCGACAAGACCCACACCUGUCCA |
| | CCUUGUCCUGCUCCAGAACUGCUCGGCGGACCUUCCGUGUUCCUGUUUCCUCCAAAGCCUAAGGACACCCUG |
| | AUGAUCAGCAGAACCCCUGAAGUGACCUGCGUGGUGGUGGAUGUGUCUCACGAGGACCCCGAAGUGAAGUUC |
| | AAUUGGUACGUGGACGGCGUGGAAGUGCACAACGCCAAGACCAAGCCUAGAGAGGAACAGUACAACAGCACC |
| | UACAGAGUGGUGUCCGUGCUGACCGUGCUGCACCAGGAUUGGCUGAACGGCAAAGAGUACAAGUGCAAGGUG |
| | UCCAACAAGGCCCUGCCUGCUCCUAUCGAGAAAACCAUCAGCAAGGCCAAGGGCCAGCCUAGGGAACCCCAG |
| | GUUUACACACUGCCUCCAAGCAGGGACGAGCUGACCAAGAAUCAGGUGUCCCUGACCUGCCUGGUCAAGGGC |
| | UUCUACCCUUCCGAUAUCGCCGUGGAAUGGGAGAGCAAUGGCCAGCCUGAGAACAACUACAAGACAACCCCU |
| | CCUGUGCUGGACAGCGACGGCUCAUUCUUCCUGUACUCCAAGCUGACAGUGGACAAGAGCAGAUGGCAGCAG |
| | GGCAACGUGUUCAGCUGCAGCGUGAUGCACGAGGCCCUGCACAACCACUACACCCAGAAGUCCCUGAGCCUG |
| | UCUCCUGGCAAGUGAUGAUGA |
| FAS-4-1BB | AUGCUCGGAAUCUGGACACUGCUGCCUCUGGUGCUGACAAGCGUGGCCAGACUGAGCAGCAAGAGCGUGAAC |
| | GCCCAAGUGACCGACAUCAACAGCAAAGGCCUGGAACUGAGAAAGACCGUGACCACCGUGGAAACCCAGAAC |
| | CUGGAAGGCCUGCACCACGACGGCCAGUUCUGUCACAAACCUUGUCCACCUGGCGAGCGGAAGGCCAGAGAU |
| | UGCACAGUGAAUGGCGACGAGCCUGACUGCGUGCCCUGUCAAGAGGGCAAAGAGUACACCGACAAGGCCCAC |
| | UUCAGCAGCAAGUGCAGACGGUGCAGACUGUGCGACGAAGGCCACGGACUGGAAGUGGAAAUCAACUGCACC |
| | CGGACACAGAACACCAAGUGCCGGUGCAAGCCCAACUUCUUCUGCAACAGCACCGUGUGCGAGCACUGCGAC |
| | CCUUGUACCAAGUGCGAACACGGCAUCAUCAAAGAGUGCACCCUGACCUCCAACACGAAGUGCAAAGAGGAA |
| | GGCAGCAGAAGCAACCUCGGCUGGCUGUGUCUGCUGCUGCUCCCCAUUCCUCUGAUCGUGUGGGUCAAGCGG |
| | GGCAGAAAGAAGCUGCUGUACAUCUUCAAGCAGCCCUUCAUGCGGCCCGUGCAGACCACACAAGAGGAAGAU |
| | GGCUGCUCCUGCAGAUUCCCCGAGGAAGAAGAAGGCGGCUGCGAGCUGUAAUGAUGA |

### Example 10: Treating or Preventing with T Cells Encoding and/or Expressing a TCR that Binds to a Neoantigen Associated with a Patient's Cancer

The following examples provides a description regarding how the mRNA or peptide I-cell production process can be implemented for treating or preventing cancer in a subject in need thereof.

Prior to beginning chemotherapy or tumor excision, a patient diagnosed with cancer will have blood drawn. A portion of the blood sample will be sequenced to determine the neoantigens associated with the patient's cancer, and another portion will be used to produce T cells having neoantigens associated with the patient's cancer using the methods disclosed herein, for example as described in Examples 1-7.

Purified T cells will be combined with a transfusion freezing media, packed into an IV bag, and frozen using a control rate freezer. When ready for use, the cells will be diluted with physiological saline to lower the percent DMSO present in the T cells. The patient will be infused with the T cells over the course of several hours, during which the patient will be continuously monitored. It is contemplated that in some circumstances, the patient will require no further treatment after administration of the T cells.

### Example 11: Enrichment and Amplification of Several TCRs Specific for a Patient Antigen for Subsequent Transfection and Infusion

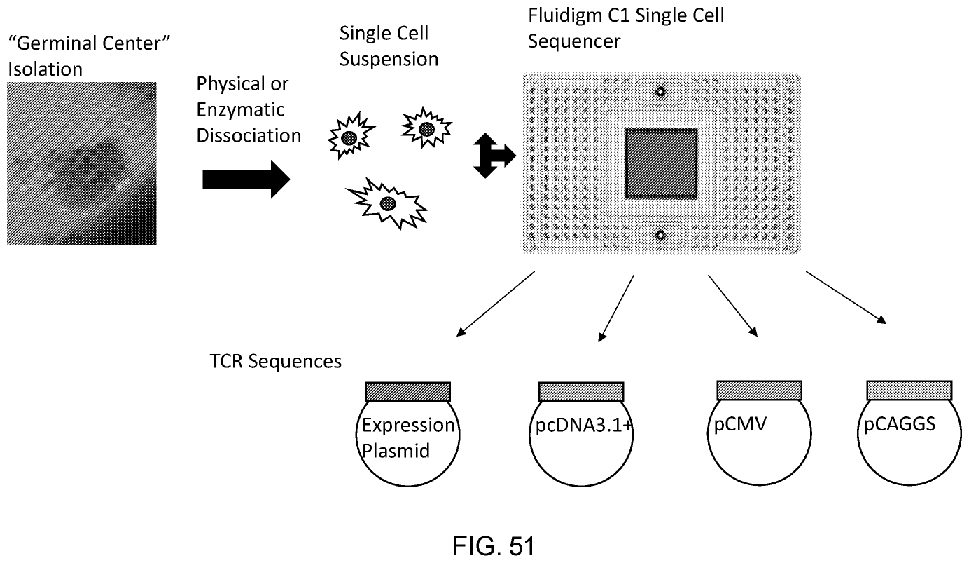

The following example describes how the mRNA T-cell production process can be utilized to isolate multiple TCR sequences specific to a neoantigen. The combined TCRs can be applied to autologous or allogenic cells derived from the mRNA T-cell production process. The procedure begins by using the mRNA T-cell production process for production of an autologous cell product as previously described. Between days 7 to 10 or earlier in the procedure, the DCs and T-cells having been synapsing, they begin to form large clusters of rapidly proliferating T-cells (FIG. 51). The clusters are akin to the germinal centers found naturally in the body. At the center of each germinal center is a DC presenting antigen and, in this case, antigens comprising a patient's specific neoantigens.

All T cells synapsing with the DC are specific for that mixture of patient neoantigens. A germinal center is then removed from culture and dissociated to a single cell suspension by a combination of physical disruption by pipette and use of chelating molecules to remove salts necessary for cell-cell adhesion thereby dissociating the cells. After the cells undergo single cell DNA sequencing, in this case by the Fluidigm C1 system as depicted in FIG. 51. A microfluid chip is used to isolate and perform the sequencing. The sequencing results will provide a number of genomes equal to the number of cells inserted, which in this case is 96. These 96 genomes will contain genetic evidence of TCR rearrangement to identify T-cells and the sequence of a given TCR. The TCR sequences are synthesized and placed into an expression plasmid. The expression plasmid will be compatible with restriction enzyme based cloning or homologous recombination-based cloning such as pcDNA 3.1⁺ or an inducible plasmid such as pTREx-DEST30 or pTREx-DEST30 31. The plasmids can be used directly by bulk transfection into a T cell line derived from the original patient that has had its endogenous TCR removed so as not to interfere with the introduced TCR. Each T cell has an equal chance of taking up one of the plasmids and therefore, it is likely every TCR plasmid will be expressed at some level. The resulting T-cells target a patient's neoantigens at the repertoire level. This is unique from the methods previously described as it guarantees a broad response instead of relying on clonal expansion which may narrow the number of TCRs available in the product simply due to differing growth rates. After, the T cells can be infused into the patient as previously described.

### Example 12: Production of a T Cell Product with a Response Profile Associated with Successful Viral Clearance

The following example provides details regarding the identification of a variety of COVID-19 specific viral antigens and generation of therapeutic T cells that recognize these antigens using the DC process according to embodiments disclosed therein.

The 2019 novel coronavirus SARS-CoV-2, which can cause acute respiratory disease, first emerged in December 2019 and swept the globe, resulting in over 723,205 deaths as of Oct. 19, 2021. The severity of the associated Coronavirus Disease 2019 (COVID-19) appears to follow a course of illness comprised of two distinct stages. First, the incubation and non-severe stage, where a specific adaptive immune response is required to eliminate the virus and to halt disease progression. The second stage, or severe stage, occurs when the virus propagates causing the damaged cells to induce innate inflammation in the lungs, largely mediated by pro-inflammatory macrophages and granulocytes. This leads to cytokine/chemokine releases, known as a cytokine storm, causing an acute respiratory distress syndrome (ARDS) and multiple organ failure, ultimately leading to death in severe cases.

To date, vaccines, antibodies and immune therapies for COVID-19 have focused primarily on the Spike Protein (S) and a relatively narrow response. Naturally arising and circulating variants of SARS-CoV-2 S protein have altered antigenicity. These mutations occur due to adaption in immune experienced populations especially during prolonged infection. Due to these mutations, supplemented antibody mediated immunity, such as convalescent sera or therapeutic monoclonal antibodies, might have reduced efficacy. Vaccine solely directed at the S protein thus appear to be less effective.

In contrast, human T cell responses to SARS-CoV-2, such as generation of virus-specific CD4⁺ and CD8⁺ T cells, may play an important role in vaccine design and evaluation. T cells recognize viral antigens as peptides through their antigen receptor, TCR, bound to MHC. Once a viral antigen is recognized, CD4⁺ T-cells are activated, differentiating into helper T-cell subsets, whereas CD8⁺ T-cells differentiate into cytotoxic T-cells with the help of CD4⁺ T-cells. Previous studies have characterized the CD4⁺ and CD8⁺ T cells responses towards the 25 proteins encoded in the SARS-CoV-2 genome between COVID-19 naïve donors and patients who cleared mild COVID-19, suggesting many potential CD4+ T cell targets in SARS-CoV-2 where the pattern of immunodominance for M, spike, and N proteins were clearly co-dominant. However, in CD8⁺ cells, the spike protein was also a target but was not dominant and other proteins like M, nsp6, ORF3a, and N was just as strongly recognized. This suggests the use of antigens such as M and N in addition to the spike, which vaccines use solely, would better mimic the natural SARS-CoV-2-specific T cell response observed in mild to moderate COVID-19 disease. Even with numerous vaccine options, it is unknown whether vaccination will successfully and durably create immunity let alone mimic the natural immune response of T cells with just the spike protein as a target. Furthermore, there have been zero studies or trials demonstrating whether the vaccines will prevent the spread of SARS-CoV-2 from vaccinated to non-vaccinated people. Moreover, there are certain populations such as the elderly, chronically ill, cancer patients, and immunocompromised for whom vaccination may prove inadequate. Additionally, healthcare workers in all channels have significant risks of transmission that a guaranteed immunity could prevent.

In this example, a therapeutic approach was developed to address the limitations of current approaches by using the presently disclosed technology to create a series of allogeneic T cell lines representing the most common MHC in the U.S. population (including African Americans, Hispanics, Caucasians, and Asians) selected to respond to a number of SARS-CoV-2 proteins. Each T cell line targets one of the COVID-19 epitopes associated with successful clearance of the virus. For each patient, a cocktail of T-cell lines with matching or partial matching MHC is selected such that it covers multiple viral proteins. The partial matching MHC must be specific for an epitope in COVID-19 that binds the MHC of the patient. Both CD4⁺ and CD8⁺ lines can be generated and used in this cocktail. Compared with vaccines and other therapies that focus on the S protein, the use of T cells for the viral clearance of SARS-CoV-2 that were selected to recognize more antigens than just the S protein affords a broader and more diverse T cell response and therefore better efficacy. Also, this guarantees both CD4⁺ and CD8⁺ responses which is known to be associated with an efficient immune response.

This therapy can be applied to any patient as long as there is at least one T-cell line containing an MHC binding epitope matched to the patient. Upon examining the frequency of HLA alleles present in the human population, it is possible to cover roughly 90% of the population with matches to at least two alleles at separate loci, and 20% of the population with HLA matches to all six alleles with a limited set of T-cell lines, approximately 10-16. This example is not intended to set a minimum or maximum number of lines. These allogeneic T cells will have been created in vitro and demonstrated to kill cells presenting COVID-19 antigens as well as to contain cells of the memory phenotype. By HLA typing the patient upon receipt of a positive NAAT test for SARS-CoV-2, one can then select one of these T cell lines off the shelf and infuse into the patient at a time when the viral load is low and has infected only a relatively small number of cells. Such an approach has been successful in management of post-transplant viral complications due to EBV and CMV activation. Because the patient will not have to be immunosuppressed to undergo the therapy, people with underlying conditions such as cancer patients or otherwise immunocompromised patients, can safely participate in this line of treatment.

By administering the therapeutic T cells upon diagnosis, it may be possible to eliminate the virus before the patient has developed a significant viral load and prevent the progression of the disease to the final stages of acute respiratory distress syndrome (ARDS) and thereby lower the mortality rate. As the innate immune response drives ARDS and most who develop ARDS do so within a week of primary insult, it may be possible to circumvent natural immunity by introducing the adaptive immune response through T cells that have a specific controlled response and will be more capable of stopping the spread of the virus through the alveolar epithelium. In turn, this will limit the release of cytokines by the innate immune system preventing ARDS. Previous studies have shown the circulating time of partial match T cells is at least seven to 14 days using allogeneic T cells, whereas a full match will last much longer. Therefore, this partial match provides coverage of the virus while the patient develops their own productive adaptive and a memory immune response, either with their own cells or by partial chimera with the allogeneic T cell lines. Thus, some level of immune memory to the SARS-CoV-2 virus is likely to be achieved.

### Identification of Viral Antigens

In order to identify which viral antigens were most likely to produce a response for pilot experiments, the COVID-19 antigen response pattern of T-cells of patients who cleared COVID-19 with minimal side effects was compared to people naïve for COVID-19 infection. In particular, these antigens included Cov-2 S, M, N, 3a, 7a, 8. Further experiments on patient's T-cells who died from COVID-19 or had a severe adverse reaction will be used to eliminate epitopes associated with adverse events through comparison with those with minimal adverse events. We were able to use the DC processes described herein to engineer a diverse T cell response from SARS-CoV-2 naïve healthy donor's PBMCs to these antigens with a comparable response pattern to that of the T Cells from cleared COVID-19 patients FIG. 52. The resulting T cells reactive with these antigens were both CD4⁺ and CD8⁺ T cells and included a high percentage of central memory T cells. This indicates the ability to create a protective immune response to a virus without the patient going through the underlying disease. Furthermore, this approach will provide a durable protection due to the robust central memory cells preventing future reinfection.

### Generation of DCs

Whole blood samples from healthy adult donors were obtained by blood draw or apheresis, and PBMCs were isolated from the blood by Ficoll separation. To make DCs, PBMCs were plated onto tissue culture grade plastic 6 well plates in RPMI 1640 media at a density of 700,000 cells/cm² and moved into a 5% CO₂ 37° C. humidified incubator for an hour. Cells were then washed with PBS twice at 2 mL per 10 cm². Post washing DC differentiation media consisting of DC media as the base, 10% human sera, 2 mM Glutamax, human IL-4, human GM-CSF at 800 U/mL and 500 U/mL respectively were added to the wells containing the adherent cells at 2 mL per well of a 6-well plate. Cells were moved into 5% CO₂ 37° C. humidified incubator. Starting the next day and then every other day after that half of the media was removed, centrifuged at 330×g, and resuspended in fresh media of equal volume and added to the culture. On day 5, all the media was removed, centrifuged at 330×g and resuspended with maturation media and added to the culture. Maturation media was Cellgenix GMP DC media with 10% human AB sera with glutamine and a maturation cocktail of PGE₂ 1 μg/mL, human IL-6, IL-1β, TNFα at 1000 U/mL. Cells were incubated overnight in a 5% CO₂ 37° C. humidified incubator. The next day media was removed, centrifuged at 330×g and still adherent cells having ice cold PBS 2 mL per well in a 6-well added, incubated on ice for 30 minutes, vigorously washed using the PBS present in the well and combined with the fraction removed from the well initially. The cells were then counted using the Nexcelom automated counting chamber using AOPI following the instructions for the AOPI cell number and viability stain given by the manufacturer.

On day 6 of the adherent cells (typically monocytes) to DC differentiation and maturation, the harvested dendritic cells (“DCs”) were combined with COVID-19 viral peptides, S, M, N, 3a, 7a, 8, and S+ (all peptides combined). The non-adherent cell fraction was thawed using anti-aggregate from Immunospot and combined with DCs in the ratio of 2:1 nonadherent cells (T-cells) to DCs. The total volume was 1 mL at a cell density of 3×10⁶ cells/mL using Cellgenix GMP DC Medium, 10% human AB sera, 2 mM L-Glutamine with human IL-7 and IL-15 at 3753 U/mL and 525 U/mL respectively. Peptides resuspended in DMSO were added so each peptide was at the final concentration of 0.1 μg/mL. The plate was the brand G-Rex from Wilson Wolf such as the G-24. The culture was moved to a 5% CO2 37° C. humidified incubator. Every two days half of the media was exchanged for fresh media without disturbing the cells. On day 7, the process was repeated by thawing PBMCs and combining with new DCs and peptides, adding to current culture. On day 14, polyclonal CD3/CD28/CD2 T Cell Activator was added to culture 15 ul/ml with fresh media containing cytokines and placed back into a 5% CO₂ 37° C. humidified incubator.

AIM and phenotyping Flow Cytometry assays were conducted on Days 14, 21, and 28 as well as cell count and viability. 1×10⁶ cells per well were plated in 2 separate 96-wells U bottom plates for AIM and phenotype assay. A stimulation with an equimolar amount of DMSO was performed as negative control for both assays and cells were stained with antibody cocktails for 15 min at room temperature in the dark. After the final wash, cells were resuspended in 200 μl FACS buffer and samples were analyzed using FlowJo software.

### T Cell Epitopes

In order to accomplish the allogenic therapy, we identified which T cell epitopes would most likely to be reactive with approximately 50% accuracy by using an MHC class I binding predictor MHCnetpan on the full SARS-CoV-2 amino acid sequence for the top 50% most common MHC alleles in the population. As such, the frequency of people expressing at least two of the MHC alleles covers most of the population. By looking at all the proteins as opposed to just the classic surface proteins to which antibody vaccines are generated, we have identified T cell epitopes that are present in sites critical to the viral replication and viability of virus so that the virus cannot easily mutate to escape.

### Peptides

Peptides were chosen based upon the most common peptide response for CD4⁺ and CD8⁺ T cells in cleared COVID-19 patients. By developing 15-mer overlapping peptides across the protein domains of interest and testing by ELISpot on a PBMC panel representing the top 50% most common MHC alleles, the MHC Class I (CD8⁺ T cell) peptides to target were confirmed, and the MHC Class II (CD4⁺ T cell) peptides were identified across the SARS-CoV-2 virus peptidome. Furthermore, it will be determined which HLAs can be covered with a given SARS-CoV-2 protein, thus determining how many different HLAs will be required to protect the U.S. population.

### Patients and T Cell Response

To model a productive immune response against SARS-CoV-2 and identify further T cell epitopes that will be productive, physical measurements of T cell responses from lymphocytes isolated from the blood of the patients previously infected with COVID-19 will be performed. The presence of patient populations who survived and are no longer infected with COVID-19 provides insights into the most likely promising targets. Targets to avoid will be determined from patients who died from COVID-19 or its complications. To ensure the validity of the results, nucleic acid amplification test (NAAT) confirmed patients from whom PBMCs have been collected after viral clearance in the Yale Biorepository and other community-based sample sources will be used. This investigation of a T cell response would reveal phenotypic information regarding different classes of T cell memory, regulatory cells, effector cells, and the distribution of CD4⁺ and CD8⁺ populations against the virus at the antigen and peptide level. Because PBMCs have been collected from the patient at diagnosis and every three days thereafter until viral clearance, such information will help reveal the temporal development of the immune response in select patients retrospectively as the response developed, which will in turn guide and refine the T cell therapy and vaccine. From these combined experiments, the exact proteins and their epitopes involved in clearing COVID-19 can be sequentially narrowed down. Therefore, the final product will be an off-the-shelf therapy of pre-produced lines of T cells specific to categories of people according to their MHC tissue typing, offering at least one week of adoptive viral protection, in view of previous studies showing protection lasting up to 2 weeks using allogeneic T cells and allographs.

### Cell Differentiation

In order to develop the pre-produced allogeneic lines of T cells, apheresis on patients or normal donors will be performed and drawn into a tube connected to the disclosed closed manufacturing production system (FIG. 32). In such closed system the product will never be in contact with the air from initial venipuncture to obtain patients' blood, through separation, stimulation and growth, to collection, and administration to patients. This closed system has been utilized previously in EBV patients with 90% of the runs meeting release specifications (Table 25).

TABLE 25
Exemplary Release Specifications for Closed System

| | | 2 | | | | |
|---|---|---|---|---|---|---|
| | | NHL-26 | | 4 | | |
| | | BioOptions | | NHL-25 | 5 | |
| | | 180-292 | | BioOptions | NHL-13 | |
| | | Follicular B | 3 | 160-720 | BioOptions | |
| | 1 | cell | NHL-14 | Follicular | 180-290 | 6 |
| | NHL-28 | lymph- | BioOptions | T cell | Follicular | NHL-24 |
| | BioOptions | oma, | 180-295 | lymphoma, | lymphoma, | BioOptions |
| | 160-737 | no | Follicular | in | partial | 160-739 |
| | Recurrent | treatment | lymphoma, | remission | remission | DLBCL, |
| | DLBCL, on | (EDTA | status | (EDTA | (EDTA | partial |
| | treatment | draw) | unknown | draw) | draw) | remission |
| % CD3+ | 83.5 | 92.2 | 96.5 | 93.4 | 94.1 | 73.6 |
| at day 28 | | | | | | |
| ELISpot | +3 (LMP1) | +3 (LMP1) | +3 (LMP1) | +3 (LMP1) | +3 (LMP1) | +0 (LMP1) |
| Score | +3 (LMP2) | +2/3 (LMP2) | +4 (LMP2) | +4 (LMP2) | +3 (LMP2) | +3 (LMP2) |
| at day 28 | +4 | +4 | +3 | +1 | +4 | +4 |
| | (EBNA1) | (EBNA1) | (EBNA1) | (EBNA1) | (EBNA1) | (EBNA1) |
| % viability | 72.7 | 82.7 | 89.6 | 88.5 | 87.4 | 83.6 |
| at day 28 | | | | | | |
| Fold | 29.7 | 39.7 | 38.2 | 73.4 | 87.7 | 38.9 |
| Expansion | | | | | | |
| Starting | 2.97E+09 | 3.97E+09 | 3.82E+09 | 7.34E+09 | 8.77E+09 | 3.89E+09 |
| with | | | | | | |
| 1.00E+08 | | | | | | |

| | | | | | | 12 |
|---|---|---|---|---|---|---|
| | | | | | | NHL-11 |
| | | | | | | BioOptions |
| | | | | | | 160-742 |
| | | 8 | | | | Well |
| | 7 | NHL-19 | | 10 | 11 | differentiated |
| | NHL-9 | BioOptions | 9 | NHL-12 | NHL-20 | lymphocytic |
| | BioOptions | 180-296 | NHL-17 | BioOptions | MSKCC | lymphoma or |
| | 160-736 | B cell | BioOptions | 160-718 | EBV005 | marginal |
| | Tonsillar | lymphoma | 160-728 | Follicular | EBER + | zone |
| | DLBCL, on | (SLL), in | DLBCL, in | lymphoma, | DLBCL | lymphoma, |
| | treatment | remission | remission | on treatment | (presumed) | on treatment |
| % CD3+ | 97.2 | 94.5 | 88.6 | 88.2 | 93.9 | 91.9 |
| at day 28 | | | | | | |
| ELISpot | +2/3 (LMP1) | +2/3 (LMP1) | +3 (LMP1) | +2/3 (LMP1) | +1 (LMP1) | 0 (LMP1) |
| Score | +4 (LMP2) | +4 (LMP2) | +2/3 (LMP2) | +3 (LMP2) | 0/+1 (LMP2) | +3 (LMP2) |
| at day 28 | +2/3 | +3 | +2/3 | +3 | 0 | 0 |
| | (EBNA1) | (EBNA1) | (EBNA1) | (EBNA1) | (EBNA1) | (EBNA1) |
| % viability | 85.7 | 79.7 | 77.3 | 79.7 | 83.7 | 75.4 |
| at day 28 | | | | | | |
| Fold | 17.8 | 15.1 | 35.2 | 19.1 | 106.4 | 13.7 |
| Expansion | | | | | | |
| Starting | 1.78E+09 | 1.51E+09 | 3.52E+09 | 1.91E+09 | 1.06E+10 | 1.37E+09 |
| with | | | | | | |
| 1.00E+08 | | | | | | |

| | | 14 | | | |
|---|---|---|---|---|---|
| | | NHL-23 | | | |
| | | BioOptions | | | |
| | | 180-294 | | | |
| | 13 | Follicular | 15 | 16 | 17 |
| | NHL-27 | lymphoma | NHL-10 | NHL-22 | NHL-21 |
| | BioOptions | with | BioOptions | BioOptions | BioOptions |
| | 160-730 | transfor- | 180-293 | 851-1-179 | 160-729 |
| | B cell | mation | Follicular | DLBCL, on | DLBCL with |
| | lymphoma | to DLBCL, | lymphoma, | treatment | nasopharyngeal |
| | (SLL), on | on | on | (EDTA | involvement, |
| | treatment | treatment | treatment | draw) | in remission |
| % CD3+ | 38.9* | 91.4 | 95.5 | 95.3 | 78.0 |
| at day 28 | | | | | |
| ELISpot | +2/3 (LMP1) | 0 (LMP1) | 0 (LMP1) | +1/2 (LMP1) | 0 (LMP1)* |
| Score | +0 (LMP2) | +1 (LMP2) | +1 (LMP2) | +2 (LMP2) | 0 (LMP2)* |
| at day 28 | +3 | 0 | 0 | +0 | 0 |
| | (EBNA1) | (EBNA1) | (EBNA1) | (EBNA1) | (EBNA1)* |
| % viability | 87.1 | 85.1 | 83.5 | 89.1 | 86.7 |
| at day 28 | | | | | |
| Fold | 77.1 | 48.3 | 186.6 | 75.0 | 75.5 |
| Expansion | | | | | |
| Starting | 7.71E+09 | 4.83E+09 | 1.87E+10 | 7.50E+09 | 7.55E+09 |
| with | | | | | |
| 1.00E+08 | | | | | |

Meets release specification CD3 > 70%
Meets release specification ELISpot score of at least + 1 to at least one antigen
Meets release specification Viability >70%
* Too low to pass release specification

| ELISpot score | # of spots/10⁵ cells |
|---|---|
| 0 | 0-24 |
| +1 | 25-49 |
| +2 | 50-99 |
| +3 | 100-499 |
| +4 | >500 |

Note:
Scores based on Boland et al 2013

For production of allogeneic cell lines, the aforementioned DC process is repeated for each of the selected epitopes and for each MHC associated with that epitope. On day 21 of the process the T-cells that contain a TCR reactive to our selected epitope and MHC are identified by single cell IFNγ ELISpot or single cell sorting of IFNγ releasing cells. These are assays in which day 21 cells are incubated with the selected antigen and activation is measured by IFNγ release. Following identification cells are undergo a process of clonal expansion. Each T cell with its unique TCR grows into a large population of identical T-cells numbering potentially in the trillions. This is accomplished by growing the T-cells in flasks using Cellgenix GMP DC Medium, 10% human AB sera, 2 mM L-Glutamine with human IL-7 and IL-15 at 3753 U/mL and 525 U/mL respectively. These cells are then frozen down and banked for later use using STEMCELL Cryostor 10.

### Assays

The assays performed for release are to test the percentage of CD3⁺ T cells, cell viability, memory and phenotype by FACS, T cells activation via T cell receptor (TCR) dependent activation induced marker (AIM) assay, killing, and an antigen specific IFNγ response by ELISpot. If the T cells pass this rigorous testing, the cells are infused into the patient, offering coverage of the virus while the patient develops their own productive adaptive and a memory immune response, either with their own cells or by partial chimera with the allogeneic T cell lines.

### Results

The T cell products generated from the DC-based manufacturing process have a recognition pattern of a patient who has successfully cleared SARS-CoV-2 virus. Peptides were chosen based upon their AIM response for COVID-19 positive patients between CD4⁺(FIG. 52A) and CD8⁺(FIG. 52B) cells.

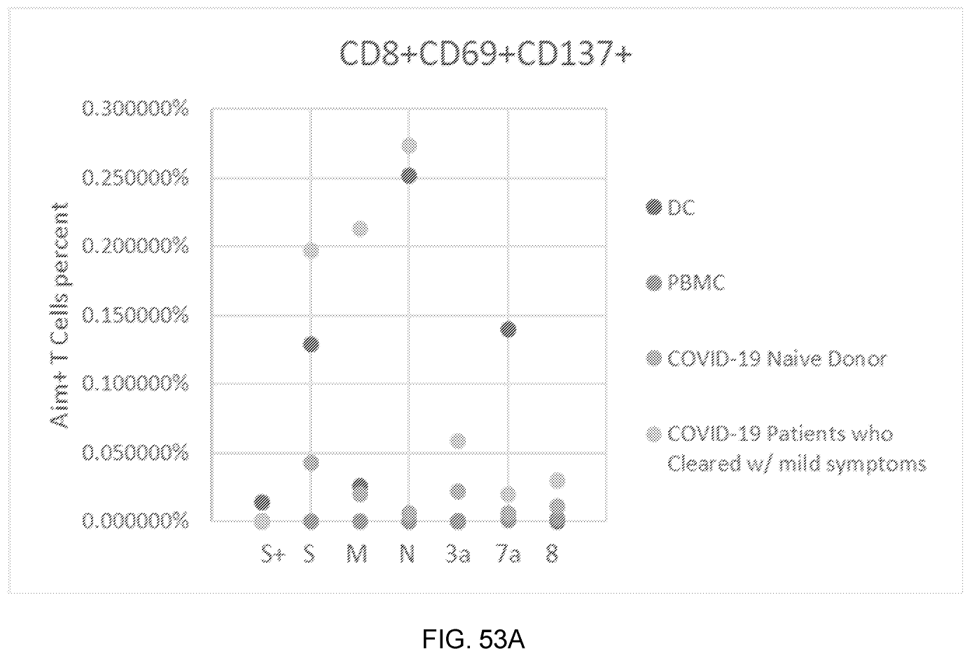

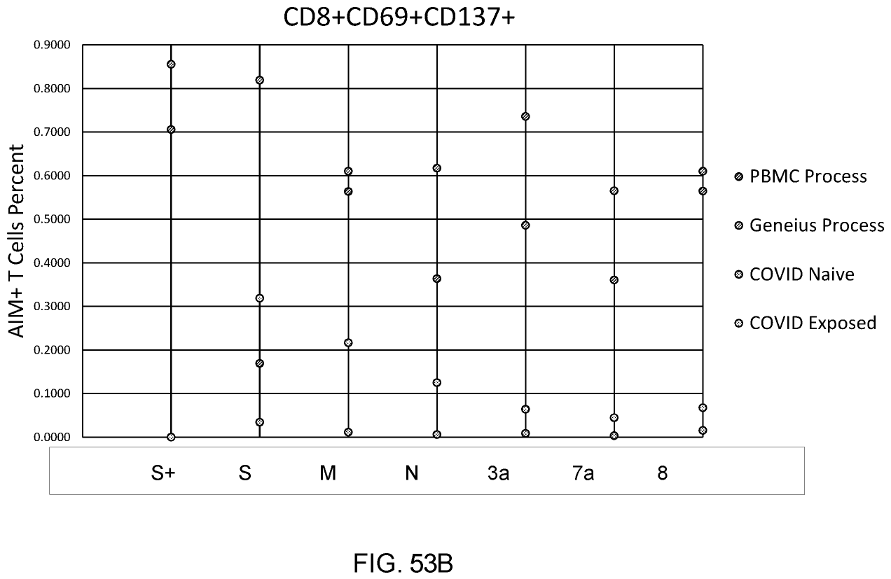

In order to probe the reactivity of various peptides (S, M, N, 3a, 7a, 8, and S+: all antigens together), we utilized TCR dependent AIM assays to identify and quantify SARS-CoV-2-specific CD4⁺ and CD8⁺ T cells in unexposed donors comparing DC and PBMC no DC derived T Cells. SARS-CoV-2-specific CD4⁺ T cells were measured as percentage of AIM⁺ (OX40⁺CD137⁺) CD4⁺ T cells (FIG. 53A) and SARS-CoV-2-specific CD8+ T cells were measured as percentage of AIM⁺(CD69⁺CD137⁺) CD8⁺ T cells (FIG. 53B), after background subtraction. Both panels showed higher response for DCs and cleared patients' samples towards antigens S, M, and N whereas for PBMCs and Naïve donors, there was no response for all antigens. The longevity of SARS-CoV-2 immunological memory was measured as a percentage of CD3⁺CD62L⁺CD197⁺ T Cell populations (FIG. 52C). DC memory is more than 3 times higher than in the PBMC group T cells. The results from this study demonstrate that stimulating T cells with COVID-19 specific viral antigens using DC process, can provide a robust T cell population.

In the case of CD4⁺ and CD8⁺ T cell responses, patterns of antigen specificity were observed between DC and PBMC no DC derived T cells, comparing both to COVID-19 naïve donors and to patients who has successfully cleared SARS-CoV-2 virus. Similar patterns of antigen specificity were found between the DCs and successfully cleared SARS-CoV-2 patients, and PBMCs and COVID-19 naïve donors. This indicates the manufacturing process disclosed herein is able to engineer a diverse T cell response from SARS-CoV-2 naïve healthy donor PBMCs-derived DCs to these antigens with a comparable response pattern to that of the T cells from cleared COVID-19 patients.

Furthermore, the resulting T cells reactive with these antigens are both CD4⁺ and CD8⁺ T cells and include a high percentage of central memory T cells. DC derived T cells have 3 times as much memory as PBMC derived T cells, further indicating that stimulating T cells with COVID-19 specific viral antigens in the DC process can create a robust T cell population with durable memory. The T cell product can be injected into a patient, providing durable T cell activity without prior exposure to COVID-19 antigens.

### Example 13: Combined Use of Autoloqous Adoptive T-Cell Therapy and RNA Vaccine

The following example demonstrates how an RNA vaccine can be combined with the autologous adoptive T cell therapy generated from the DC process for increased efficacy of the therapy. The principal behind their combined use is the ability of an RNA vaccine to induce in vivo T-cell responses that act either to prime the collected PBMCs against the antigens encoded by the RNA vaccine and/or to boost the responses of adopted T-cells in vivo. The boost can occur by two mechanisms, either by re-stimulation of adopted T-cells that are known to have a previous response to encoded antigens or by generation of endogenous immune responses that not previously been known to be responsive in the adopted T-cells. In this example, the adopted T cells are still considered the mechanism of action of the therapy and the RNA vaccine acts in support of this mechanism of action.

When the RNA vaccine is used before collection of PBMCs from a patient, it serves to increase the number of starting T-cells specific for an antigen upon collection. This logarithmically increases the final fraction of T-cells specific to said antigen at the end of the DC process. This improves the efficacy of the therapy assuming more T-cells against targeted disease associated antigens leads to increased efficacy. This is the “priming” strategy. When the RNA vaccine is used after the infusion of end-product T-cells from the DC process it acts to re-stimulate adopted T-cells specific for the antigens it encodes to increase and prolong the immune response against selected antigens. The neoantigen re-challenge will also stimulate the development of memory T-cells for a long-lasting response. This is the “boost” strategy.

This example is not meant to limit the vaccine sequence. In the event of a high tumor mutational burden there could be thousands of mutations. An entire viral genome could be covered by just a few antigens. To simplify or improve production of the cellular therapy in this case, RNA encompassing all the mutations or virus can be produced and used as an RNA vaccine initially. Following vaccination assays would be performed which indicate which antigens in the vaccine had provoked a response. Another RNA construct containing just the reactive antigens would be manufactured for use in the subsequent DC process. Using a round of positive selection as described is beneficial for several reasons. For 30 antigen targets it requires approximately 3 kb of mRNA. Synthesis and cloning of nucleotide sequences is efficient below 5 kb. To cover all sequences in terms of high mutational load could require 30+ separate mRNAs, a significant manufacturing challenge with current technology. Also, as the mass used either for RNA vaccine or transfer to DCs is constant, this lowers the effective concentration of each antigen. This is sufficient for memory T cell response but not for the generation of new responses. The first requires only one activation while the second requires multiple rounds of repriming. Having only reactive antigens in the DC process significantly raises the chances for a product with T cells reactive against multiple antigens and in significant number. The inflection points of therapy efficacy, number of antigens, numbers of reactive antigens and number of RNAs will need to be empirically determined. In another event after the infusion of the adopted T-cells a different RNA from the one used in the DC process can be applied as an RNA vaccine. This RNA vaccine may only encode sequences that have or have not demonstrated reactivity in the adoptive T-cell product depending on if a boost or expansion of number of targets is required.

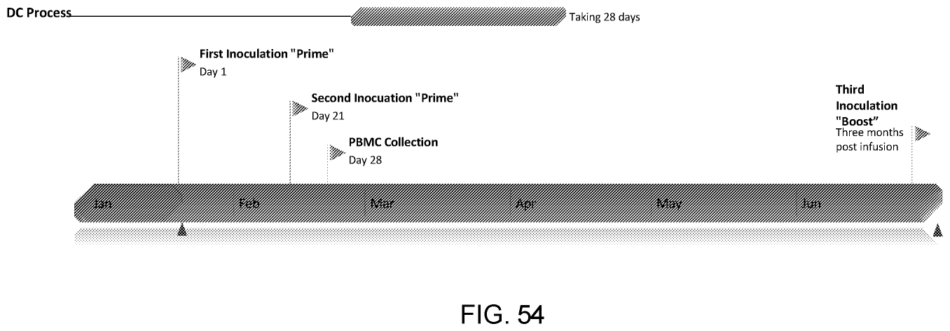

The timing of inoculation depends on the date of PBMC collection and the date of infusion of the T-cell product (see FIG. 54). For an RNA vaccine priming strategy inoculation would need to be at least two weeks before PBMC collection for the DC process. Several inoculations before PBMC collection can spaced out over the course of months depending on responses. For an RNA vaccine boosting strategy initial inoculation could begin two weeks to a month after infusion of adopted T-cells. Further inoculations would be spaced out over the course of months or years as necessary to maintain immunity.

The RNA vaccine in this example is the same sequence as that is transferred into DCs. It is GMP, optimized for mammalian expression and simplifies the production of the therapy by having one RNA for all parts. For use as a vaccine, it can be injected intravenously, intradermal, intranodal, sprayed intranasally, within the tumor either as a naked RNA or encapsulated in lipid nanoparticles, cationic lipids, protamine or proteins and have either a net negative or positive charge. The example here is a colorectal cancer patient who has twenty mutations resulting in changes in amino acid sequence. Sequencing occurred at the time of diagnosis. The primary tumor was excised and treated with local chemotherapy but no other treatments have been applied before or during the disclosed therapy.

### Manufacturing of GMP RNA

Production of an RNA vaccine begins with sequencing of the DNA or RNA of the colorectal cancer patient including liquid biopsy, tumor sequencing, RNA-seq or another sequencing technology. Once the twenty neoantigens present in the patient's cancer have been determined, an mRNA construct is designed according to the sequence specifications previously mentioned. It is produced with the molecules outlined previously including modified nucleotides, 5′ cap etc. The production of the mRNA follows a series of steps to ensure that the product meets GMP specifications. All reagents used are derived from sources that do not contain any contaminants and are produced with defined media and not natural sources. These best practices are outlined in guidance ICH Q7. The mRNA will also undergo the purification process as previously mentioned. Following purification, the mRNA is encapsulated with lipid nanoparticles, or cationic proteolipids such as protamine, with/or carrier proteins and small molecules. This step is necessary to ensure efficient expression of the mRNA in the body and to target the right subset of cells, in this case being dendritic cells (“DCs”). Naked mRNA could also be directly used.

### Inoculation

In this example, both the “priming” strategy and “boost” strategy are used for the timetable of RNA inoculation for the colorectal cancer patient. Upon completion of the production of the RNA vaccine, the PBMC collection date is set in such a way that inoculation of 30 ug of mRNA is injected on day 1 followed by another injection on day 21 and PBMCs to be used for the DC process are collected on day 28. The DC production process as previously outlined is followed with the exception that gene transfer to DCs is accomplished in vitro by direct introduction of the RNA vaccine. The T-cell product is infused into the patient on day 56 post initial inoculation. The adopted T-cells remain stimulated from the DC process for at least 30 days. If the RNA vaccine is applied too soon after T-cell infusion it could lead to over activation of the T-cells resulting in their death and regulatory suppression by T-regulatory cells. The RNA vaccine “boost” inoculation occurs three months after infusion of the T-cell product.

### Assay Results

In this example, the impact of the use of the RNA vaccine strategy can be monitored by IFNγ ELISpot for each of the neoantigens at the major steps in the timetable. Before inoculation the patient may have measurable IFNγ releasing cells for some of the neoantigens, however, because of T-cell exhaustion of a cancer patient it will be low and for most neoantigens it will be entirely negative. On day 28 post “prime” inoculation some of the neoantigens negative at day 1 will become positive. After undergoing the DC process for all twenty neoantigens there will be a substantial increase in frequency of IFNγ producing cells and number of neoantigens positive for IFNγ production. Without the priming step these two parameters will be reduced as the starting material has already begun the process of T-cell activation. The impact of the “boost” inoculation following T-cell infusion will be seen at six months to a year post T-cell infusion. Compared to control, the “boost” will have increased frequency of IFNγ producing cells and a higher proportion of the T-cells specific to a patient's neoantigens being of the memory phenotype.

### Example 14: A Multi Antigen Vaccine Against Viruses Including SARS COV2

In this example an mRNA vaccine simultaneously targeting Cov-2 Spike (S), VME1 (M), NCAP (N), 3a, 7a, 8 is produced. A disadvantage of current vaccines is that they target only one viral protein by producing the full recombinant protein within DCs transfected with mRNA vaccine. Granted they do have multiple epitopes for a given antigen however using the technology disclosed here a vaccine can be produced that target multiple viral proteins all within the same mRNA construct. This is important as there is a limited amount of mRNA that reaches endogenous dendritic cells and if several separate mRNAs encoding antigen were simply combined there would low efficiency of T-cell or B-cell priming for any of the given antigen. This would result in very significant variability in the responses of each person. They may target epitopes that are not critical for the virus life cycle and can therefore be mutated leading to loss of efficacy of the vaccination. Dose response curves measuring antibody titer to Cov-2 indicate a narrow therapeutic window for the vaccine using a single Cov-2 protein, the spike. It would be extremely difficult to determine a therapeutic window for multiple proteins.

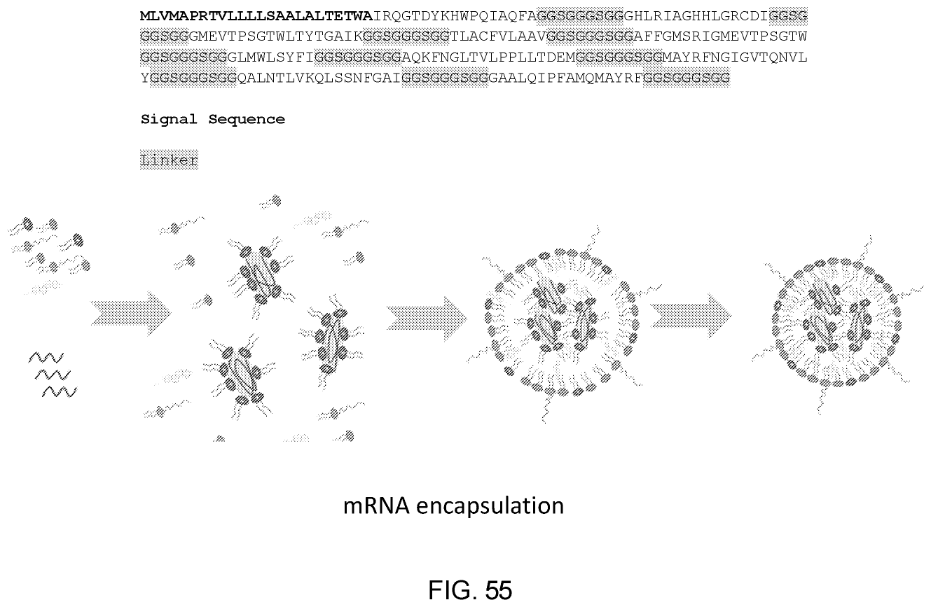

The technology disclosed herein selects specific epitopes varying from the minimal essential amino acids for a given epitope or can include 11, 12, 13, 14, 15 flanking amino acids around that epitope. In this example epitopes corresponding to the reportedly most immunogenic epitopes across S, M, N. These have the strongest antibody titer responses and bind to a multiplicity of HLA alleles. It would also be possible for any given person to produce a fully “personalized” Cov-2 vaccine. This would be accomplished by HLA allele typing a person and selecting epitopes across the Cov-2 genome that would most likely generate a T-cell or B-cell response and placing them in the same manner as in this example. The immunogenic epitopes are listed in Table 26. They are combined into a single mRNA vaccine sequence in FIG. 55.

TABLE 26
Immunogenic epitopes of
S, M, N SARS-Cov-2 proteins

| Protein | Immunogenic Sequence |
|---|---|
| SARS-CoV-2 | IRQGTDYKHWPQIAQFA |
| Antigen Peptide | AFFGMSRIGMEVTPSGTW |
| NCAP | GMEVTPSGTWLTYTGAIK |
| SARS-CoV-2 | GHLRIAGHHLGRCDI |
| Antigen Peptide | TLACFVLAAV |
| VME1 | GLMWLSYFI |
| SARS-CoV-2 | AQKFNGLTVLPPLLTDEM |
| Antigen Peptide | MAYRFNGIGVTQNVLY |
| SPIKE | QALNTLVKQLSSNFGAI |
| | GAALQIPFAMQMAYRF |

Various embodiments of the present technology are set forth herein below.
Para. A. A method of generating a population of T cells expressing one or more T cell receptors (TCRs) that specifically bind one or more antigens, comprising: (i). obtaining a blood sample from a subject with cancer or a viral infection; (ii). identifying one or more antigens associated with the cancer or the viral infection; (iii). preparing one or more mRNA molecules encoding the one or more antigens associated with the cancer or the viral infection; (iv). isolating monocytes from peripheral blood mononuclear cells (PBMCs) of the blood sample and preserving a remainder of cells from the sample, the remainder of cells comprising T cells; (v). differentiating the isolated monocytes into dendritic cells; (vi). transfecting the dendritic cells with the one or more mRNA molecules; and (vii). stimulating the T cells from the remainder of cells by contacting them with the transfected dendritic cells, thereby generating a population of T cells that express one or more TCRs that specifically bind the one or more antigens associated with the cancer or the viral infection.
Para. B. The method of Para. A, wherein the one or more antigens are cancer neoantigens.
Para. C. The method of Para. B., wherein the cancer neoantigens are selected from the neoantigens set forth in Tables 1-9 and 11.
Para. D. The method of Para. A, wherein the one or more antigens are viral antigens.
Para. E. The method of any one of Paras. A-D, wherein the one or more antigens are identified by sequencing cell free deoxyribonucleic acid (cfDNA) associated with the cancer or the viral infection.
Para. F. The method of Para. E, wherein the sequencing comprises next generation sequencing.
Para. G. The method of any one of Paras. A-F, wherein the one or more antigens are about 15 to about 50 amino acids in length.
Para. H. The method of any one of Paras. A-G, wherein the mRNA is at least about 80% pure.
Para. I. The method of any one of Paras. A-H, wherein the one or more mRNA molecules comprise coding sequences for a plurality of the antigens each separated by a polylinker.
Para. J. The method of Para. I, wherein the polylinker comprises an amino acid sequence of GGSGGGSS.
Para. K. The method of any one of Paras. A-J, wherein the one or more mRNA molecules each comprise a signal peptide, a 5′ untranslated region (UTR), a 3′ untranslated region (UTR), and/or a polyadenine (poly (A)) tail.
Para. L. The method of any one of Paras. A-K, wherein the differentiating the isolated monocytes into dendritic cells of step (v) occurs in media containing one or more cytokines.
Para. M. The method of Para. L, wherein the one or more cytokines comprise GM-CSF and IL-4.
Para. N. The method of Para. M, wherein the one or more cytokines further comprise IL-1β, IL-6, TNF-α, and/or PGE₂.
Para. O. The method of any one of Paras. A-N, wherein all or substantially all of the monocytes are differentiated into dendritic cells in step (v).
Para. P. The method of any one of Paras. A-O, wherein the method further comprises incubating the dendritic cells of step (v) with one or more antigen peptides associated with the cancer or the viral infection prior to step (vii).
Para. Q. The method of any one of Paras. A-P, wherein the transfecting the dendritic cells with the one or more mRNA molecules of step (vi) is by cation lipid transfection, lipofection, or nucleofection.
Para. R. The method of any one of Paras. A-Q, wherein the ratio of the dendritic cells to the T cells in step (vii) is about 1:2 to about 1:4.
Para. S. The method of any one of Paras. A-R, wherein the stimulating the T cells of step (vii) occurs in media containing cytokines.
Para. T. The method of Para. S, wherein the cytokines comprise IL-7 and IL-15.
Para. U. The method of any one of Paras. A-T, wherein the stimulating the T cells of step (vii) is repeated for 2, 3, 4, or more times.
Para. V. The method of any one of Paras. A-U, wherein the method further comprises stimulating the T cells of step (vii) with tetrameric antibodies that bind CD3, CD28, and CD2.
Para. W. The method of any one of Paras. A-V, wherein the T cells have a deletion or disruption in an endogenous β2-microglobulin (B2M) gene.
Para. X. The method of any one of Paras. A-W, wherein the T cells are further exposed to one or more apoptosis inhibitors during step (vii).
Para. Y. The method of Para. X, wherein the one or more apoptosis inhibitors are selected from the group consisting of 10058-F4, 4′-methoxyflavone, AZD5438, BAG1 (72-end) protein, BAX Inhibiting peptide, BEPP monohydroxychloride, BI-6C9, BTZO, Bongkrekic acid, CTP inhibitor, CTX1, Calpeptin, Clofarabine, Clusterin nuclear form protein, Combretastatin A4, Cyclic Pifithrin-a hydroxybromide, EM20-25, Fasentin, Ferrostatin-1, GNF-2, IM-54, Ischemin-CalbiochemA cell permeable azobenezene, Liproxstatin-1, MDL28170, Mdivi-1, Mitochondrial Fusion Promoter, N-Ethylmaleimide, N-Ethylmaleimide, NS3694, NSCI, Necrostatin-1, Oridonin, PD151746, PDI inhibitor 16F16, Pentostatin, Pifithrin-a, Pifithrin-a p-Nitro Cyclic, Pifithrin-u, S-15176 difumarate, UCF-101, p53-Snail binding inhibitor GH25, TW-37, and Z-VAD-FMK
Para. Z. The method of any one of Paras. A-Y, wherein the T cells are further exposed to one or more Rho-associated protein kinase (ROCK) inhibitors at the initiation of step (vii).
Para. AA. The method of Para. Z, wherein the one or more ROCK inhibitors are selected from the group consisting of Y-27632 2HCI, Thiazovivin, Fasudil (HA-1077) HCI, GSK429286A, RKI-1447, Azaindole 1 (TC-S 7001), GSK269962A HCI, Netarsudil (AR-13324), Y-39983 HCI, ZINC00881524, KD025 (SLx-2119), Ripasudil (K-115), Hydroxyfasudil (HA-1100) AT13148, AMA-0076, AR-1286, ATS907, DE-104, INS-115644, INS-117548, PG324, Y-39983; RKI-983, SNJ-1656, Wf-563, Azabenzimidazole-aminofurazans, H-1152P, XD-4000, HMN-1152, Rhostatin, 4-(1-aminoakyl)-N-(4-pyridl)cyclohexane-carboamides, BA-207, BA-215, BA-285, BA-1037, Ki-23095, VAS-012, quinazoline, Netarsudil, and ITRI-E-212
Para. AB. The method of any one of Paras. A-AA, wherein the dendritic cells and the T cells are cultured in a single closed system bioreactor.
Para. AC. A population of T cells derived from the method of any one of Paras. A-AB.
Para. AD. The population of T cells of Para. AC, wherein the T cells comprise naïve T cells, CD4⁺ T cells, CD8⁺ T cells, central memory T cells, stem cell memory T cells, effector memory T cells, or any combination thereof.
Para. AE. The population of T cells of Para. AC or AD, wherein at least about 70% of the T cells are CD3⁺.
Para. AF. The population of T cells of Para. AC or AD, wherein at least about 70% of the T cells are central memory T cells.
Para. AG. The population of T cells of Para. AC or AD, wherein at least about 70% of the T cells are effector memory T cells.
Para. AH. The population of T cells of Para. AC or AD, wherein at least about 70% of the T cells are CD4⁺ T cells.
Para. AI. The population of T cells of Para. AC or AD, wherein at least about 70% of the T cells are CD8⁺ T cells.
Para. AJ. The population of T cells of Para. AC or AD, wherein the population comprises no or substantially no markers of exhaustion including but not limited to cells positive for at least one of PD-1, LAG3, TIM-3, CTLA4, BTLA, TIGIT.
Para. AK. A method of generating a population of T cells expressing one or more T cell receptors (TCRs) that specifically bind an antigen, comprising: (i). transfecting a population of dendritic cells with one or more mRNA molecules encoding one or more antigens; and (ii). stimulating a population of naïve T cells by contacting them with the transfected dendritic cells of step (i), thereby generating a population of T cells that express one or more T cells receptors that specifically bind the one or more antigens encoded by the one or more mRNA molecules.
Para. AL. The method of Para. AK, wherein the antigen is a cancer neoantigen.
Para. AM. The method of Para. AK, wherein the antigen is a viral antigen.
Para. AN. The method of any one of Paras. AK-AM, wherein the ratio of the dendritic cells to the T cells in step (ii) is about 1:2 to about 1:4.
Para. AO. An isolated engineered T cell comprising T cell receptors (TCRs) targeting a plurality of cancer neoantigens selected from the neoantigens set forth in Tables 1-9 and 11.
Para. AP. The T cell of Para. AO, wherein the T cell secretes tumor necrosis factor alpha (TNFα) and/or interferon gamma (IFNγ) when exposed to any of the plurality of neoantigens.
Para. AQ. The T cell of Para. AO or AP, wherein the T cell comprises a disruption or deletion in an endogenous β2-microglobulin (B2M) gene.
Para. AR. The T cell of any one of Paras. AO-AQ, wherein the T cell is further engineered to transiently express one or more proteins that modify a tumor microenvironment.
Para. AS. The T cell of Para. AR, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1 BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.
Para. AT. The T cell of any one of Para. AR, wherein the one or more proteins comprise one or more exogenous enzymes that alter an extracellular matrix.
Para. AU. The T cell of any one of Paras. AR-AT, wherein the transient expression is by transfecting the T cell with one or more mRNA molecules encoding the one or more proteins that modify a tumor microenvironment.
Para. AV. The T cell of Para. AU, wherein the one or more mRNA molecules are linear RNA, circularized RNA, or self-replicating RNA.
Para. AW. A population of engineered T cells comprising T cell receptors (TCRs) targeting one or more antigens, the population comprising less than 5% regulatory T cells, less than 5% exhausted T cells, and more memory T cells than effector T cells.
Para. AX. The population of T cells of Para. AW, wherein the population of T cells comprises more than 50% memory T cells.
Para. AY. The population of T cells of Para. AW or AX, wherein the population of T cells comprises at least half a billion T cells.
Para. AZ. The population of T cells of any one of Paras. AW-AY, wherein the population of T cells comprises a plurality of T cells transiently expressing one or more proteins that modify a tumor microenvironment.
Para. BA. The population of T cell of Para. AZ, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.
Para. BB. The population of T cells of any one of Para. AZ, wherein the one or more proteins comprise one or more exogenous enzymes that alter an extracellular matrix.
Para. BC. The population of T cells of any one of Paras. AZ-BB, wherein the transient expression is by transfecting the T cells with one or more mRNA molecules encoding the one or more proteins that modify a tumor microenvironment.
Para. BD. The population of T cells of Para. BC, wherein the one or more mRNA molecules are linear RNA, circularized RNA, or self-replicating RNA.
Para. BE. The population of T cells of any one of Paras. AW-BD, wherein each T cell in the population of T cells comprises a disruption or deletion in an endogenous β2-microglobulin (B2M) gene.
Para. BF. A method of treating cancer in a subject in need thereof, comprising: (i). obtaining a blood sample from the subject; (ii). identifying one or more neoantigens associated with the subject's cancer; (iii). preparing one or more mRNA molecules encoding the one or more neoantigens; (iv). isolating monocytes from peripheral blood mononuclear cells (PBMCs) of the blood sample and preserving a remainder of cells from the sample, the remainder of cells comprising T cells; (v). differentiating the isolated monocytes into dendritic cells; (vi). transfecting the dendritic cells with the one or more mRNA molecules; (vii). stimulating the T cells from the remainder of cells by contacting them with the transfected dendritic cells, thereby generating a population of T cells that express one or more T cells receptors (TCRs) that specifically bind the one or more neoantigens associated with the cancer; and (viii). administering all or a portion of the resultant population of T cells to the subject.
Para. BG. The method of Para. BF, wherein the cancer is selected from the group consisting of colon cancer, lung cancer, pancreatic cancer, acute myeloid leukemia (AML), melanoma, bladder cancer, hematologic cancer, and glioblastoma
Para. BH. A method of treating cancer in a subject in need thereof, comprising: (i). identifying two or more neoantigens associated with the subject's cancer; and (ii). administering to the subject a population of T cells, the population of T cells comprising a plurality of T cells that each express two or more T cell receptors (TCRs) that specifically bind at least two of the two or more neoantigens and further comprise a deletion or disruption in an endogenous β2-microglobulin (B2M) gene.
Para. BI. The method of Para. BH, wherein the cancer is selected from the group consisting of colon cancer, lung cancer, pancreatic cancer, acute myeloid leukemia (AML), melanoma, bladder cancer, hematologic cancer, and glioblastoma.
Para. BJ. A method of treating a viral infection in a subject in need thereof, comprising: (i). identifying two or more viral antigens associated with the subject's viral infection; and (ii). administering to the subject a plurality of T cells expressing two or more T cell receptors (TCRs) that specifically bind the two or more viral antigens.
Para. BK. The method of Para. BJ, wherein the viral infection is caused by a virus selected from the group consisting of cytomegalovirus, Epstein-Barr virus, hepatitis B virus, human papillomavirus, adenovirus, herpes virus, human immunodeficiency virus, influenza virus, human respiratory syncytial virus, vaccinia virus, varicella-zoster virus, yellow fever virus, Ebola virus, SARS-CoV, MERS-CoV, SARS-CoV-2, Eastern equine encephalitis virus, and Zika virus.
Para. BL. A method of transiently expressing one or more proteins that modify a tumor microenvironment in a T cell, comprising transfecting the T cell with one or more mRNA molecules encoding the one or more proteins that modify a tumor microenvironment.
Para. BM. The method of Para. BL, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1 BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.
Para. BN. The method of Para. BL or BM, wherein the one or more mRNA molecules are linear RNA, circularized RNA, or self-replicating RNA.
Para. BO. A method of altering a tumor microenvironment in a subject, comprising administering to the subject a population of T cells transiently expressing one or more proteins that modify the tumor microenvironment.
Para. BP. The method of Para. BO, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1 BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.
Para. BQ. The method of Para. BO or BP, wherein the transient expression is by transfecting the T cells with one or more mRNA molecules encoding the one or more proteins that modify a tumor microenvironment.
Para. BR. The method of Para. BQ, wherein the one or more mRNA molecules are linear RNA, circularized RNA, or self-replicating RNA.
Para. BS. A method of preparing a composition comprising dendritic cells encoding and/or expressing one or more neoantigens associated with a subject's cancer, comprising: (i). obtaining a blood sample from the subject; (ii). sequencing cell free deoxyribonucleic acid (cfDNA) derived from the blood sample to identify one or more neoantigens associated with the subject's cancer; (iii). preparing an mRNA encoding the one or more neoantigens associated with the subject's cancer or a peptide corresponding to the one or more neoantigens associated with the subject's cancer; (iv). isolating monocytes from peripheral blood mononuclear cells (PBMCs) of the blood sample; (v). differentiating the isolated monocytes into dendritic cells; and (vi). combining the dendritic cells with the mRNA or peptide from step (iii) to obtain dendritic cells encoding and/or expressing the one or more neoantigens associated with the subject's cancer.
Para. BT. A composition comprising one or more T cells encoding and/or expressing a T cell receptor (TCR) that binds to a neoantigen associated with a subject's cancer, wherein the one or more T cells comprise one or more CD4⁺ T cell, one or more CD8⁺ T cell, one or more CD3⁺ T cell, and wherein the CD4⁺ T cells and CD8⁺ T cells are present in the composition in a ratio of about 1:1, about 1:2, or about 1:4.
Para. BU. The composition of Para. BT, wherein the composition comprises about 80%, by weight, of a total weight of the composition, the one or more T cells encoding and/or expressing the TCR.
Para. BV. The composition of claim Para. BT or BU, wherein the composition comprises less than about 20%, by weight, of any cell other than the one or more T cells encoding and/or expressing the TCR.
Para. BW. The composition of any one of Paras. BT-BV, wherein the one or more T cells comprise a naïve T cell, a central memory T cell, a stem cell memory T cell, an effector memory T cell, an NK cell, or any combination thereof.
Para. BX. The composition of any one of Paras. BT-BV, wherein the composition comprises greater than about 70%, by weight, of a total weight of the composition, CD3⁺ and CD8⁺ T cells or CD3⁺ and CD4⁺ T cells.
Para. BY. The composition of any one of Paras. BT-BV, wherein the composition comprises greater than about 70%, by weight, of the total weight of the composition, central memory T cells.
Para. BZ. The composition of any one of Paras. BT-BV, wherein the composition comprises greater than about 70%, by weight, of the total weight of the composition, effector memory T cells.
Para. CA. The composition of any one of Paras. BT-BV, wherein the composition comprises greater than about 70%, by weight, of a total weight of the composition, CD4⁺ T cells.
Para. CB. The composition of any one of Paras. BT-BV, wherein the composition comprises greater than about 70%, by weight, of a total weight of the composition, CD8⁺ T cells.
Para. CC. The composition of any one of Paras. BT-BV, wherein the composition comprises greater than about 70%, by weight, of a total weight of the composition, CD3⁺ T cells.
Para. CD. The composition of any one of Paras. BT-CC, wherein the composition comprises no or substantially no markers of exhaustion including but not limited to cells positive for at least one of PD-1, LAG3, TIM-3, CTLA4, BTLA, TIGIT.
Para. CE. The composition of any one of Paras. BT-CD, further comprising a pharmaceutically acceptable carrier, pharmaceutically acceptable excipient, and/or pharmaceutically acceptable diluent.
Para. CF. The composition of any one of Paras. BT-CE, wherein the neoantigen is selected from the group consisting of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53 R273C, TP53 273H, TP53 R282W, and TP53 V157F.
Para. CG. A method of treating cancer in a subject in need thereof, comprising administering to the subject the composition of any one of Paras. BT-CF.

### REFERENCES

1. Cross et al. The evolutionary landscape of colorectal tumorigenesis. Nat Ecol and Evol 2(10):1661-1672 (2018) (doi:10.1038/s41559-018-0642-z)
2. Oldfield et al. Molecular events in the natural history of pancreatic cancer. Trends Cancer 3(5):336-346 (2017) (doi:10.1016/j.trecan.2017.04.005)
3. Spranger et al. Melanoma-intrinsic β-catenin signaling prevents anti-tumour immunity. Nature. 523(7559):231-235 (2015) (doi:10.1038/nature14404)
4. Blank et al. Cancer Immunology. The “cancer immunogram.” Science 352(6286):658-660 (2016) (doi:10.1126/science.aaf2834)
5. Poch et al. Systemic immune dysfunction in pancreatic cancer patients. Langenbecks Arch Surg 392(3):353-358 (2007) (doi:10.1007/s00423-006-0140-7)
6. Ohm & Carbone. Immune dysfunction in cancer patients. Oncology 16(Suppl 1):11-18 (2002)
7. Robert et al. Nivolumab in previously untreated melanoma without BRAF mutation. N Engl J Med 372(4):320-330 (2015) (doi:10.1056/NEJMoa1412082)
8. Ferris et. al. Nivolumab for recurrent squamous-cell carcinoma of the head and neck. N Engl J Med 375(19):1856-1867(2016) (doi:10.1056/NEJMoa1602252)
9. Hodi et al. Improved survival with ipilimumab in patients with metastatic melanoma. N Engl J Med 363(8):711-723 (2010) (doi:10.1056/NEJMoa1003466)
10. Lee et al. T cells expressing CD19 chimeric antigen receptors for acute lymphoblastic leukaemia in children and young adults: a phase 1 dose-escalation trial. Lancet 385(9967):517-528 (2020) (doi:10.1016/S0140-6736(14)61403-3)
11. Lo et al. Immunologic recognition of a shared p53 mutated neoantigen in a patient with metastatic colorectal cancer. Cancer Immunol Res 7(4):534-543 (2019) (doi:10.1158/2326-6066.CIR-18-0686)
12. Restifo et al. Adoptive immunotherapy for cancer: harnessing the T cell response. Nat Rev Immunol 12(4):269-281 (2012) (doi:10.1038/nri3191)
13. Cafri et al. Memory T cells targeting oncogenic mutations detected in peripheral blood of epithelial cancer patients. Nat Commun 10(1):449 (2019) (doi:10.1038/s41467-019-08304-z)
14. Gottschalk et al. Generating CTLS against the subdominant Epstein-Barr virus LMP1 antigen for the adoptive immunotherapy of EBV-associated malignancies. Blood 101(5):1905-1912 (2003) (doi:10.1182/blood-2002-05-1514)
15. Bollard et al. Sustained complete responses in patients with lymphoma receiving autologous cytotoxic T lymphocytes targeting Epstein-Barr virus latent membrane proteins. J Clin Oncol 32(8):798-808 (2014) (doi:10.1200/JCO.2013.51.5304)
16. Riddell et al. Restoration of viral immunity in immunodeficient humans by the adoptive transfer of T cell clones. Science 257(5067):238-241 (1992) (doi: 10.1126/science.1352912)
17. Zill et al. The landscape of actionable genomic alterations in cell-free circulating tumor DNA from 21,807 advanced cancer patients. Clin Cancer Res 24(15):3528-3538 (2018) (doi:10.1158/1078-0432.CCR-17-3837)
18. Alter et al. CD107a as a functional marker for the identification of natural killer cell activity. J Immunol Methods 294(1-2):15-22 (2004) (doi:10.1016/j.jim.2004.08.008)
19. Betts & Koup. Detection of T-cell degranulation: CD107a and b. Methods Cell Biol 75:497-512 (2004) (doi:10.1016/s0091-679×(04)75020-7)
20. Tau et al. Regulation of IFN-γ signaling Is essential for the cytotoxic activity of CD8(+) T cells. J Immunol 167(10):5574-5582 (2001) (doi:10.4049/jimmunol.167.10.5574)
21. Moshirfar et al. Use of Rho kinase Inhibitors in ophthalmology: a review of the literature. Med Hypothesis Discov Innov Ophthalmol 7(3):101-111 (2018)
22. Rao & Epstein. Rho GTPase/Rho kinase inhibition as a novel target for the treatment of glaucoma. BioDrugs 21(3):167-177 (2007) (doi:10.2165/00063030-200721030-00004)
23. Warren et al. Highly efficient reprogramming to pluripotency and directed differentiation of human cells using synthetic modified mRNA. Cell Stem Cell 7(5):618-630 (2010) (doi:10.1016/j.stem.2010.08.012)
24. Kreiter et al. Increased antigen presentation efficiency by coupling antigens to MHC class I trafficking signals. J Immunol 180(1):309-318 (2008) (doi:10.4049/jimmunol.180.1.309)
25. Lemberg et al. Intramembrane proteolysis of signal peptides: an essential step in the generation of HLA-E epitopes. J Immunol 167(11):6441-6446 (2001) (doi:10.4049/jimmunol.167.11.6441)
26. Reddy Chichili et al. Linkers in the structural biology of protein-protein interactions. Protein Sci 22(2):153-167 (2013) (doi:10.1002/pro.2206)
27. Hikono et al. Activation phenotype, rather than central-or effector-memory phenotype, predicts the recall efficacy of memory CD8⁺ T cells. J Exp Med 204(7):1625-1636 (2007) (doi:10.1084/jem.20070322)
28. Huster et al. Unidirectional development of CD8⁺ central memory T cells into protective Listeria-specific effector memory T cells. Eur J Immunol36(6):1453-1464 (2006) (doi:10.1002/eji.200635874)
29. Olson et al. Effector-like CD8⁺ T cells in the memory population mediate potent protective immunity. Immunity 38(6):1250-1260 (2013) (doi:10.1016/j.immuni.2013.05.009)
30. Seder et al. Protection against malaria by intravenous immunization with a nonreplicating sporozoite vaccine. Science 341(6152):1359-1365 (2013) (doi:10.1126/science.1241800)
31. Jiang et al. Signatures of T cell dysfunction and exclusion predict cancer immunotherapy response. Nat Med 24(10):1550-1558 (2018) (doi:10.1038/s41591-018-0136-1)
32. Albershardt et al. Therapeutic efficacy of PD1/PDL1 blockade in B16 melanoma is greatly enhanced by immunization with dendritic cell-targeting lentiviral vector and protein vaccine. Vaccine 38(17):3369-3377 (2020) (doi:10.1016/j.vaccine.2020.02.034)
33. Bonsack et al. Performance evaluation of MHC class-I binding prediction tools based on an experimentally validated MHC-peptide binding data set. Cancer Immunol Res 7(5):719-736 (2019) (doi:10.1158/2326-6066.CIR-18-0584)
34. Freeman et al. Regulation of innate CD8⁺ T cell activation mediated by cytokines. Proc Natl Acad Sci USA 109(25):9971-9976 (2012) (doi:10.1073/pnas.1203543109)
35. Green et al. IFN-γ from CD4 T cells is essential for host survival and enhances CD8 T cell function during Mycobacterium tuberculosis infection. J Immunol 190(1):270-277 (2013) (doi:10.4049/jimmunol.1200061)
36. Borst et al. CD4⁺ T cell help in cancer immunology and immunotherapy. Nat Rev Immunol 18(10):635-647 (2018) (doi:10.1038/s41577-018-0044-0)
37. Crowther et al. Genome-wide CRISPR-Cas9 screening reveals ubiquitous T cell cancer targeting via the monomorphic MHC class I-related protein MR1. Nat Immunol 21 (2):178-185 (2020) (doi:10.1038/s41590-019-0578-8)
38. Wienert et al. In vitro-transcribed guide RNAs trigger an innate immune response via the RIG-1 pathway. PLoS Biol 16(7):e2005840 (2018) (doi:10.1371/journal.pbio.2005840)
39. Vaidyanathan et al. Uridine depletion and chemical modification increase Cas9 mRNA activity and reduce immunogenicity without HPLC purification. Mol Ther Nucleic Acids 12:530-542 (2018) (doi:10.1016/j.omtn.2018.06.010)
40. Kim et al. CRISPR RNAs trigger innate immune responses in human cells. Genome Res 28(3):367-373 (2018) (doi:10.1101/gr.231936.117)
41. Mccarthy et al. Natural deletions in the SARS-CoV-2 spike glycoprotein drive antibody escape. BioRxiv, 19 Dec. 2020 (doi:10.1101/2020.11.19.389916)
42. Shi et al. Neutralizing antibodies targeting SARS-CoV-2 spike protein. Stem Cell Res 50:102125 (2020) (doi:10.1016/j.scr.2020.102125)
43. Derwall et al. The acute respiratory distress syndrome: pathophysiology, current clinical practice, and emerging therapies. Expert Rev of Respir Med 12(12):1021-1029 (2018) (doi:10.1080/17476348.2018.1548280)
44. O'Reilly et al. Therapeutic advantages provided by banked virus-specific T-cells of defined HLA-restriction. Bone Marrow Transplant 54(Suppl 2):759-764 (2019) (doi:10.1038/s41409-019-0614-1)
45. Brudno et al. Allogeneic T cells that express an anti-CD19 chimeric antigen receptor induce remissions of B-cell malignancies that progress after allogeneic hematopoietic stem-cell transplantation without causing graft-versus-host disease. J Clin Oncol34(10):1112-1121 (2016) (doi:10.1200/JCO.2015.64.5929)
46. Lin et al. Long-term acceptance of major histocompatibility complex mismatched cardiac allografts induced by CTLA41g plus donor-specific transfusion. J Exp Med 178(5):1801-1806 (1993) (doi:10.1084/jem.178.5.1801)
47. Dong et al. An interactive web-based dashboard to track COVID-19 in real time. Lancet Infect. Dis. 20:533-534 (2020) (doi:10.1016/S1473-3099(20)30120-1)
48. Grifoni et al. Targets of T cell responses to SARS-CoV-2 coronavirus in humans with COVID-19 disease and unexposed individuals. Cell 181(7):1489-1501 (2020) (doi:10.1016/j.cell.2020.05.015)
49. Pritchard et al. Exploration of peptides bound to MHC class I molecules in melanoma. Pigment Cell and Melanoma Res 28(3):281-294 (2015) (doi:10.1111/pcmr.12357)
50. Jarmalavicius et al. High immunogenicity of the human leukocyte antigen peptidomes of melanoma tumor cells. J Biol Chem 287(40):33401-33411 (2012) (doi:10.1074/jbc.M112.358903)

I/We claim:

1. A method of generating a population of T cells expressing one or more T cell receptors (TCRs) that specifically bind one or more antigens, comprising:
(i). obtaining a blood sample from a subject with cancer or a viral infection;
(ii). identifying one or more antigens associated with the cancer or the viral infection;
(iii). preparing one or more mRNA molecules encoding the one or more antigens associated with the cancer or the viral infection;
(iv). isolating monocytes from peripheral blood mononuclear cells (PBMCs) of the blood sample and preserving a remainder of cells from the sample, the remainder of cells comprising T cells;
(v). differentiating the isolated monocytes into dendritic cells;
(vi). transfecting the dendritic cells with the one or more mRNA molecules; and
(vii). stimulating the T cells from the remainder of cells by contacting them with the transfected dendritic cells,
thereby generating a population of T cells that express one or more TCRs that specifically bind the one or more antigens associated with the cancer or the viral infection.

2. The method of claim 1, wherein the one or more antigens are cancer neoantigens.

3. The method of claim 2, wherein the cancer neoantigens are selected from the neoantigens set forth in Tables 1-9 and 11.

4. The method of claim 1, wherein the one or more antigens are viral antigens.

5. The method of any one of claims 1-4, wherein the one or more antigens are identified by sequencing cell free deoxyribonucleic acid (cfDNA) associated with the cancer or the viral infection.

6. The method of claim 5, wherein the sequencing comprises next generation sequencing.

7. The method of any one of claims 1-6, wherein the one or more antigens are about 15 to about 50 amino acids in length.

8. The method of any one of claims 1-7, wherein the mRNA is at least about 80% pure.

9. The method of any one of claims 1-8, wherein the one or more mRNA molecules comprise coding sequences for a plurality of the antigens each separated by a polylinker.

10. The method of claim 9, wherein the polylinker comprises an amino acid sequence of GGSGGGSS.

11. The method of any one of claims 1-10, wherein the one or more mRNA molecules each comprise a signal peptide, a 5′ untranslated region (UTR), a 3′ untranslated region (UTR), and/or a polyadenine (poly (A)) tail.

12. The method of any one of claims 1-11, wherein the differentiating the isolated monocytes into dendritic cells of step (v) occurs in media containing one or more cytokines.

13. The method of claim 12, wherein the one or more cytokines comprise GM-CSF and IL-4.

14. The method of claim 13, wherein the one or more cytokines further comprise IL-1β, IL-6, TNF-α, and/or PGE₂.

15. The method of any one of claims 1-14, wherein all or substantially all of the monocytes are differentiated into dendritic cells in step (v).

16. The method of any one of claims 1-15, wherein the method further comprises incubating the dendritic cells of step (v) with one or more antigen peptides associated with the cancer or the viral infection prior to step (vii).

17. The method of any one of claims 1-16 wherein the transfecting the dendritic cells with the one or more mRNA molecules of step (vi) is by cation lipid transfection, lipofection, or nucleofection.

18. The method of any one of claims 1-17, wherein the ratio of the dendritic cells to the T cells in step (vii) is about 1:2 to about 1:4.

19. The method of any one of claims 1-18, wherein the stimulating the T cells of step (vii) occurs in media containing cytokines.

20. The method of claim 19, wherein the cytokines comprise IL-7 and IL-15.

21. The method of any one of claims 1-20, wherein the stimulating the T cells of step (vii) is repeated for 2, 3, 4, or more times.

22. The method of any one of claims 1-21, wherein the method further comprises stimulating the T cells of step (vii) with tetrameric antibodies that bind CD3, CD28, and CD2.

23. The method of any one of claims 1-22, wherein the T cells have a deletion or disruption in an endogenous β2-microglobulin (B2M) gene.

24. The method of any one of claims 1-23, wherein the T cells are further exposed to one or more apoptosis inhibitors during step (vii).

25. The method of claim 24, wherein the one or more apoptosis inhibitors are selected from the group consisting of 10058-F4, 4′-methoxyflavone, AZD5438, BAG1 (72-end) protein, BAX Inhibiting peptide, BEPP monohydroxychloride, BI-6C9, BTZO, Bongkrekic acid, CTP inhibitor, CTX1, Calpeptin, Clofarabine, Clusterin nuclear form protein, Combretastatin A4, Cyclic Pifithrin-a hydroxybromide, EM20-25, Fasentin, Ferrostatin-1, GNF-2, IM-54, Ischemin-CalbiochemA cell permeable azobenezene, Liproxstatin-1, MDL28170, Mdivi-1, Mitochondrial Fusion Promoter, N-Ethylmaleimide, N-Ethylmaleimide, NS3694, NSCI, Necrostatin-1, Oridonin, PD151746, PDI inhibitor 16F16, Pentostatin, Pifithrin-a, Pifithrin-a p-Nitro Cyclic, Pifithrin-u, S-15176 difumarate, UCF-101, p53-Snail binding inhibitor GH25, TW-37, and Z-VAD-FMK

26. The method of any one of claims 1-25, wherein the T cells are further exposed to one or more Rho-associated protein kinase (ROCK) inhibitors at the initiation of step (vii).

27. The method of claim 26, wherein the one or more ROCK inhibitors are selected from the group consisting of Y-27632 2HCI, Thiazovivin, Fasudil (HA-1077) HCI, GSK429286A, RKI-1447, Azaindole 1 (TC-S 7001), GSK269962A HCI, Netarsudil (AR-13324), Y-39983 HCI, ZINC00881524, KD025 (SLx-2119), Ripasudil (K-115), Hydroxyfasudil (HA-1100) AT13148, AMA-0076, AR-1286, ATS907, DE-104, INS-115644, INS-117548, PG324, Y-39983; RKI-983, SNJ-1656, Wf-563, Azabenzimidazole-aminofurazans, H-1152P, XD-4000, HMN-1152, Rhostatin, 4-(1-aminoakyl)-N-(4-pyridl)cyclohexane-carboamides, BA-207, BA-215, BA-285, BA-1037, Ki-23095, VAS-012, quinazoline, Netarsudil, and ITRI-E-212

28. The method of any one of claims 1-27, wherein the dendritic cells and the T cells are cultured in a single closed system bioreactor.

29. A population of T cells derived from the method of any one of claims 1-28.

30. The population of T cells of claim 29, wherein the T cells comprise naïve T cells, CD4⁺ T cells, CD8⁺ T cells, central memory T cells, stem cell memory T cells, effector memory T cells, or any combination thereof.

31. The population of T cells of claim 29 or 30, wherein at least about 70% of the T cells are CD3⁺.

32. The population of T cells of claim 29 or 30, wherein at least about 70% of the T cells are central memory T cells.

33. The population of T cells of claim 29 or 30, wherein at least about 70% of the T cells are effector memory T cells.

34. The population of T cells of claim 29 or 30, wherein at least about 70% of the T cells are CD4⁺ T cells.

35. The population of T cells of claim 29 or 30, wherein at least about 70% of the T cells are CD8⁺ T cells.

36. The population of T cells of claim 29 or 30, wherein the population comprises no or substantially no markers of exhaustion including but not limited to cells positive for at least one of PD-1, LAG3, TIM-3, CTLA4, BTLA, TIGIT.

37. A method of generating a population of T cells expressing one or more T cell receptors (TCRs) that specifically bind an antigen, comprising:
(i). transfecting a population of dendritic cells with one or more mRNA molecules encoding one or more antigens; and
(ii). stimulating a population of naïve T cells by contacting them with the transfected dendritic cells of step (i),
thereby generating a population of T cells that express one or more T cells receptors that specifically bind the one or more antigens encoded by the one or more mRNA molecules.

38. The method of claim 37, wherein the antigen is a cancer neoantigen.

39. The method of claim 37, wherein the antigen is a viral antigen.

40. The method of any one of claims 37-39, wherein the ratio of the dendritic cells to the T cells in step (ii) is about 1:2 to about 1:4.

41. An isolated engineered T cell comprising T cell receptors (TCRs) targeting a plurality of cancer neoantigens selected from the neoantigens set forth in Tables 1-9 and 11.

42. The T cell of claim 41, wherein the T cell secretes tumor necrosis factor alpha (TNFα) and/or interferon gamma (IFNγ) when exposed to any of the plurality of neoantigens.

43. The T cell of claim 41 or 42, wherein the T cell comprises a disruption or deletion in an endogenous β2-microglobulin (B2M) gene.

44. The T cell of any one of claims 41-43, wherein the T cell is further engineered to transiently express one or more proteins that modify a tumor microenvironment.

45. The T cell of claim 44, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1 BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.

46. The T cell of claim 44, wherein the one or more proteins comprise one or more exogenous enzymes that alter an extracellular matrix.

47. The T cell of any one of claims 44-46, wherein the transient expression is achieved by transfecting the T cell with one or more mRNA molecules encoding the one or more proteins that modify a tumor microenvironment.

48. The T cell of claim 47, wherein the one or more mRNA molecules are linear RNA, circularized RNA, or self-replicating RNA.

49. A population of engineered T cells comprising T cell receptors (TCRs) targeting one or more antigens, the population comprising less than 5% regulatory T cells, less than 5% exhausted T cells, and more memory T cells than effector T cells.

50. The population of T cells of claim 49, wherein the population of T cells comprises more than 50% memory T cells.

51. The population of T cells of claim 49 or 50, wherein the population of T cells comprises at least half a billion T cells.

52. The population of T cells of any one of claims 49-51, wherein the population of T cells comprises a plurality of T cells transiently expressing one or more proteins that modify a tumor microenvironment.

53. The population of T cell of claim 52, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.

54. The population of T cells of claim 52, wherein the one or more proteins comprise one or more exogenous enzymes that alter an extracellular matrix.

55. The population of T cells of any one of claims 52-54, wherein the transient expression is by transfecting the T cells with one or more mRNA molecules encoding the one or more proteins that modify a tumor microenvironment.

56. The population of T cells of claim 55, wherein the one or more mRNA molecules are linear RNA, circularized RNA, or self-replicating RNA.

57. The population of T cells of any one of claims 49-56, wherein each T cell in the population of T cells comprises a disruption or deletion in an endogenous β2-microglobulin (B2M) gene.

58. A method of treating cancer in a subject in need thereof, comprising:
(i). obtaining a blood sample from the subject;
(ii). identifying one or more neoantigens associated with the subject's cancer;
(iii). preparing one or more mRNA molecules encoding the one or more neoantigens;
(iv). isolating monocytes from peripheral blood mononuclear cells (PBMCs) of the blood sample and preserving a remainder of cells from the sample, the remainder of cells comprising T cells;
(v). differentiating the isolated monocytes into dendritic cells;
(vi). transfecting the dendritic cells with the one or more mRNA molecules;
(vii). stimulating the T cells from the remainder of cells by contacting them with the transfected dendritic cells, thereby generating a population of T cells that express one or more T cells receptors (TCRs) that specifically bind the one or more neoantigens associated with the cancer; and
(viii). administering all or a portion of the resultant population of T cells to the subject.

59. The method of claim 58, wherein the cancer is selected from the group consisting of colon cancer, lung cancer, pancreatic cancer, acute myeloid leukemia (AML), melanoma, bladder cancer, hematologic cancer, and glioblastoma.

60. A method of treating cancer in a subject in need thereof, comprising:
(i). identifying two or more neoantigens associated with the subject's cancer; and
(ii). administering to the subject a population of T cells, the population of T cells comprising a plurality of T cells that each express two or more T cell receptors (TCRs) that specifically bind at least two of the two or more neoantigens and further comprise a deletion or disruption in an endogenous β2-microglobulin (B2M) gene.

61. The method of claim 60, wherein the cancer is selected from the group consisting of colon cancer, lung cancer, pancreatic cancer, acute myeloid leukemia (AML), melanoma, bladder cancer, hematologic cancer, and glioblastoma.

62. A method of treating a viral infection in a subject in need thereof, comprising:
(i). identifying two or more viral antigens associated with the subject's viral infection; and
(ii). administering to the subject a plurality of T cells expressing two or more T cell receptors (TCRs) that specifically bind the two or more viral antigens.

63. The method of claim 62, wherein the viral infection is caused by a virus selected from the group consisting of cytomegalovirus, Epstein-Barr virus, hepatitis B virus, human papillomavirus, adenovirus, herpes virus, human immunodeficiency virus, influenza virus, human respiratory syncytial virus, vaccinia virus, varicella-zoster virus, yellow fever virus, Ebola virus, SARS-CoV, MERS-CoV, SARS-CoV-2, Eastern equine encephalitis virus, and Zika virus.

64. A method of transiently expressing one or more proteins that modify a tumor microenvironment in a T cell, comprising transfecting the T cell with one or more mRNA molecules encoding the one or more proteins that modify a tumor microenvironment.

65. The method of claim 64, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1 BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.

66. The method of claim 64 or 65, wherein the one or more mRNA molecules are linear RNA, circularized RNA, or self-replicating RNA.

67. A method of altering a tumor microenvironment in a subject, comprising administering to the subject a population of T cells transiently expressing one or more proteins that modify the tumor microenvironment.

68. The method of claim 67, wherein the one or more proteins are selected from the group consisting of IL-2, IL-7, IL-12, IL-15, IL-18, IL-21, IFNα, IFNβ, IFNγ, TNFα, IL-2R, IL-7R, IL-12R, IL-15R, IL-18R, IL-21R, IFNα receptor, IFNβ receptor, IFNγ receptor, TNFα receptor, CCL2, CCL5, CCL9, CCL10, CCL11, CCL12, CCL13, CCL19, CCL21, CCR2b, CCR2, CCR7, CXCR3, CXCR4, CD28, CD40L, 4-1 BB, OX40, CD46, CD27, ICOS, HVEM, LIGHT, DR3, GITR, CD30, TIM1, SLAM, CD2, CD226, anti-PD-1, anti-PD-L1, anti-CTLA-4, anti-Fas, anti-FasL, anti-LAG3, anti-B7-1, anti-B7-H1, anti-CD160, anti-BTLA, anti-LAIR1, anti-TIM3, anti-2B4, anti-TIGIT, anti-TGFβ receptor, anti-IL-4 receptor, anti-IL-10 receptor, anti-VEGF receptor, anti-αvβ8, and a fusion protein thereof.

69. The method of claim 67 or 68, wherein the transient expression is by transfecting the T cells with one or more mRNA molecules encoding the one or more proteins that modify a tumor microenvironment.

70. The method of claim 69, wherein the one or more mRNA molecules are linear RNA, circularized RNA, or self-replicating RNA.

71. A method of preparing a composition comprising dendritic cells encoding and/or expressing one or more neoantigens associated with a subject's cancer, comprising:
(i). obtaining a blood sample from the subject;
(ii). sequencing cell free deoxyribonucleic acid (cfDNA) derived from the blood sample to identify one or more neoantigens associated with the subject's cancer;
(iii). preparing an mRNA encoding the one or more neoantigens associated with the subject's cancer or a peptide corresponding to the one or more neoantigens associated with the subject's cancer;
(iv). isolating monocytes from peripheral blood mononuclear cells (PBMCs) of the blood sample;
(v). differentiating the isolated monocytes into dendritic cells; and
(vi). combining the dendritic cells with the mRNA or peptide from step (iii) to obtain dendritic cells encoding and/or expressing the one or more neoantigens associated with the subject's cancer.

72. A composition comprising one or more T cells encoding and/or expressing a T cell receptor (TCR) that binds to a neoantigen associated with a subject's cancer, wherein the one or more T cells comprise one or more CD4⁺ T cell, one or more CD8⁺ T cell, one or more CD3⁺ T cell, and wherein the CD4⁺ T cells and CD8⁺ T cells are present in the composition in a ratio of about 1:1, about 1:2, or about 1:4.

73. The composition of claim 72, wherein the composition comprises about 80%, by weight, of a total weight of the composition, the one or more T cells encoding and/or expressing the TCR.

74. The composition of claim 72 or 73, wherein the composition comprises less than about 20%, by weight, of any cell other than the one or more T cells encoding and/or expressing the TCR.

75. The composition of any one of claims 72-74, wherein the one or more T cells comprise a naïve T cell, a central memory T cell, a stem cell memory T cell, an effector memory T cell, an NK cell, or any combination thereof.

76. The composition of any one of claims 72-74, wherein the composition comprises greater than about 70%, by weight, of a total weight of the composition, CD3⁺ and CD8⁺ T cells or CD3⁺ and CD4⁺ T cells.

77. The composition of any one of claims 72-74, wherein the composition comprises greater than about 70%, by weight, of the total weight of the composition, central memory T cells.

78. The composition of any one of claims 72-74, wherein the composition comprises greater than about 70%, by weight, of the total weight of the composition, effector memory T cells.

79. The composition of any one of claims 72-74, wherein the composition comprises greater than about 70%, by weight, of a total weight of the composition, CD4⁺ T cells.

80. The composition of any one of claims 72-74, wherein the composition comprises greater than about 70%, by weight, of a total weight of the composition, CD8⁺ T cells.

81. The composition of any one of claims 72-74, wherein the composition comprises greater than about 70%, by weight, of a total weight of the composition, CD3⁺ T cells.

82. The composition of any one of claims 72-81, wherein the composition comprises no or substantially no markers of exhaustion including but not limited to cells positive for at least one of PD-1, LAG3, TIM-3, CTLA4, BTLA, TIGIT.

83. The composition of any one of claims 72-82, further comprising a pharmaceutically acceptable carrier, pharmaceutically acceptable excipient, and/or pharmaceutically acceptable diluent.

84. The composition of any one of claims 72-83, wherein the neoantigen is selected from the group consisting of KRAS G12A, KRAS G12C, KRAS G12D, KRAS G12R, KRAS G12S, KRAS G12V, KRAS G13D, KRAS G13C, KRAS Q61K, TP53E285K, TP53 G245S, TP53 R158L, TP53 R175H, TP53 R248Q, TP53 R248W, TP53R273C, TP53 273H, TP53 R282W, and TP53 V157F.

85. A method of treating cancer in a subject in need thereof, comprising administering to the subject the composition of any one of claims 72-84.


SEQUENCE LISTING (139 sequences)

SEQ ID NO: 1 - DNA, 8 bp, Artificial Sequence
  polylinker
ggsgggss

SEQ ID NO: 2 - protein, 27 aa, Artificial Sequence
  KRAS 27mer WT
MTEYKLVVVGAGGVGKSALTIQLIQNH

SEQ ID NO: 3 - protein, 27 aa, Artificial Sequence
  KRAS 27mer G12D
MTEYKLVVVGADGVGKSALTIQLIQNH

SEQ ID NO: 4 - protein, 20 aa, Artificial Sequence
  DMNT3A R882C germline sequence
VHYTDVSNMSRLARQRLLGR

SEQ ID NO: 5 - protein, 21 aa, Artificial Sequence
  DMNT3A R882C mutated sequence
VHYTDVSNMSCLARQRLLGRS

SEQ ID NO: 6 - protein, 20 aa, Artificial Sequence
  TP53 R158L germline sequence
DSTPPPGTRVRAMAIYKQSQ

SEQ ID NO: 7 - protein, 21 aa, Artificial Sequence
  TP53 R158L mutated sequence
DSTPPPGTRVLAMAIYKQSQH

SEQ ID NO: 8 - protein, 20 aa, Artificial Sequence
  PIK3CA Q546K germline sequence
KAISTRDPLSQITEQEKDFL

SEQ ID NO: 9 - protein, 21 aa, Artificial Sequence
  PIK3CA Q546K mutated sequence
KAISTRDPLSKITEQEKDFLW

SEQ ID NO: 10 - protein, 20 aa, Artificial Sequence
  TP53 C238Y germline sequence
DCTTIHYNYMCNSSCMGGMN

SEQ ID NO: 11 - protein, 21 aa, Artificial Sequence
  TP53 C238Y mutated sequence
DCTTIHYNYMYNSSCMGGMNR

SEQ ID NO: 12 - protein, 20 aa, Artificial Sequence
  EGFR E76_A750del germline sequence
VKIPVAIKELREATSPKANK

SEQ ID NO: 13 - protein, 20 aa, Artificial Sequence
  EGFR E76_A750del mutated sequence
VKIPVAIKEATSPKANKEIL

SEQ ID NO: 14 - protein, 20 aa, Artificial Sequence
  TP53 E286K germline sequence
ACPGRDRRTEEENLRKKGEP

SEQ ID NO: 15 - protein, 21 aa, Artificial Sequence
  TP53 E286K mutated sequence
ACPGRDRRTEKENLRKKGEPH

SEQ ID NO: 16 - protein, 20 aa, Artificial Sequence
  TP53 V157F germline sequence
VDSTPPPGTRVRAMAIYKQS

SEQ ID NO: 17 - protein, 21 aa, Artificial Sequence
  TP53 V157F mutated sequence
VDSTPPPGTRFRAMAIYKQSQ

SEQ ID NO: 18 - protein, 20 aa, Artificial Sequence
  PIK3CA E542K germline sequence
KAISTRDPLSEITEQEKDFL

SEQ ID NO: 19 - protein, 21 aa, Artificial Sequence
  PIK3CA E542K mutated sequence
KAISTRDPLSKITEQEKDFLW

SEQ ID NO: 20 - protein, 20 aa, Artificial Sequence
  NRAS Q61K germline sequence
CLLDILDTAGQEEYSAMRDQ

SEQ ID NO: 21 - protein, 21 aa, Artificial Sequence
  NRAS Q61K mutated sequence
CLLDILDTAGKEEYSAMRDQY

SEQ ID NO: 22 - protein, 20 aa, Artificial Sequence
  IDH2 R140Q germline sequence
KMWKSPNGTIRNILGGTVFR

SEQ ID NO: 23 - protein, 21 aa, Artificial Sequence
  IDH2 R140Q mutated sequence
KMWKSPNGTIQNILGGTVFRE

SEQ ID NO: 24 - protein, 20 aa, Artificial Sequence
  DNMT3A R882H germline sequence
VHYTDVSNMSRLARQRLLGR

SEQ ID NO: 25 - protein, 21 aa, Artificial Sequence
  DNMT3A R882H mutated sequence
VHYTDVSNMSHLARQRLLGRS

SEQ ID NO: 26 - protein, 20 aa, Artificial Sequence
  EGFR T790M germline sequence
ICLTSTVQLITQLMPFGCLL

SEQ ID NO: 27 - protein, 21 aa, Artificial Sequence
  EGFR T790M mutated sequence
ICLTSTVQLIMQLMPFGCLLD

SEQ ID NO: 28 - protein, 20 aa, Artificial Sequence
  TP53 G266E germline sequence
TLEDSSGNLLGRNSFEVRVC

SEQ ID NO: 29 - protein, 21 aa, Artificial Sequence
  TP53 G266E mutated sequence
TLEDSSGNLLERNSFEVRVCA

SEQ ID NO: 30 - protein, 20 aa, Artificial Sequence
  FLT3 D835Y germline sequence
VKICDFGLARDIMSDSNYVV

SEQ ID NO: 31 - protein, 21 aa, Artificial Sequence
  FLT3 D835Y mutated sequence
VKICDFGLARYIMSDSNYVVR

SEQ ID NO: 32 - protein, 20 aa, Artificial Sequence
  TP53 H241R germline sequence
EYLDDRNTFRHSVVVPYEPP

SEQ ID NO: 33 - protein, 21 aa, Artificial Sequence
  TP53 H241R mutated sequence
EYLDDRNTFRRSVVVPYEPPE

SEQ ID NO: 34 - protein, 20 aa, Artificial Sequence
  NPM1 W288Cfs*30 germline sequence
NCFRMTDQEAIQDLWQWRKS

SEQ ID NO: 35 - protein, 21 aa, Artificial Sequence
  NPM1 W288Cfs*30 mutated sequence
MTDQEAIQDLCLAVEEVSLRK

SEQ ID NO: 36 - protein, 20 aa, Artificial Sequence
  KRAS G12D germline sequence
YKLVVVGAGGVGKSALTIQL

SEQ ID NO: 37 - protein, 21 aa, Artificial Sequence
  KRAS G12D mutated sequence
YKLVVVGADGVGKSALTIQLI

SEQ ID NO: 38 - protein, 20 aa, Artificial Sequence
  KRAS G12V germline sequence
MTEYKLVVVGAGGVGKSALT

SEQ ID NO: 39 - protein, 21 aa, Artificial Sequence
  KRAS G12V mutated sequence
YKLVVVGAVGVGKSALTIQLI

SEQ ID NO: 40 - protein, 20 aa, Artificial Sequence
  TP53 R175H germline sequence
QSQHMTEVVRRCPHHERCSD

SEQ ID NO: 41 - protein, 21 aa, Artificial Sequence
  TP53 R175H mutated sequence
QSQHMTEVVRHCPHHERCSDS

SEQ ID NO: 42 - protein, 20 aa, Artificial Sequence
  TP53 R248Q germline sequence
CNSSCMGGMNRRPILTIITL

SEQ ID NO: 43 - protein, 21 aa, Artificial Sequence
  TP53 R248Q mutated sequence
CNSSCMGGMNQRPILTIITLE

SEQ ID NO: 44 - protein, 20 aa, Artificial Sequence
  TP53 R248W germline sequence
CNSSCMGGMNRRPILTIITL

SEQ ID NO: 45 - protein, 21 aa, Artificial Sequence
  TP53 R248W mutated sequence
CNSSCMGGMNWRPILTIITLE

SEQ ID NO: 46 - DNA, 2145 bp, Artificial Sequence
  Multi-neoantigen construct
  misc_feature (1)..(66): 5′UTR
  misc_feature (67)..(138): signal peptide
  misc_feature (202)..(228): linker
  misc_feature (292)..(318): linker
  misc_feature (382)..(408): linker
  misc_feature (472)..(498): linker
  misc_feature (562)..(588): linker
  misc_feature (652)..(678): linker
  misc_feature (742)..(768): linker
  misc_feature (832)..(858): linker
  misc_feature (922)..(948): linker
  misc_feature (1012)..(1038): linker
  misc_feature (1102)..(1128): linker
  misc_feature (1192)..(1218): linker
  misc_feature (1282)..(1308): linker
  misc_feature (1372)..(1398): linker
  misc_feature (1462)..(1488): linker
  misc_feature (1552)..(1578): linker
  misc_feature (1642)..(1668): linker
  misc_feature (1732)..(1758): linker
  misc_feature (1822)..(1848): linker
  misc_feature (1912)..(1938): linker
  misc_feature (2002)..(2010): stop codons
  misc_feature (2011)..(2145): Hu Beta globin 3?UTR
taatacgactcactatagggagacccaagctggctagcgtttaaacttaagcttggtaccgccaccatgctggttatggcccctagaacagtcctgctgctgctgtctgcagccctggctctgacagaaacatgggccgtgcactacaccgacgtgagcaacatgagctgcctggccaggcagaggctgctgggcaggagcggcggcagcggcggcggcagcggcggcgacagcacccccccccccggcaccagggtgctggccatggccatctacaagcagagccagcacggcggcagcggcggcggcagcggcggcaaggccatcagcaccagggaccccctgagcaagatcaccgagcaggagaaggacttcctgtggggcggcagcggcggcggcagcggcggcgactgcaccaccatccactacaactacatgtacaacagcagctgcatgggcggcatgaacaggggcggcagcggcggcggcagcggcggcaaggtgaagatccccgtggccatcaaggaggccaccagccccaaggccaacaaggagatcctgggcggcagcggcggcggcagcggcggcgcctgccccggcagggacaggaggaccgagaaggagaacctgaggaagaagggcgagccccacggcggcagcggcggcggcagcggcggcgtggacagcacccccccccccggcaccaggttcagggccatggccatctacaagcagagccagggcggcagcggcggcggcagcggcggcaaggccatcagcaccagggaccccctgagcaagatcaccgagcaggagaaggacttcctgtggggcggcagcggcggcggcagcggcggctgcctgctggacatcctggacaccgccggcaaggaggagtacagcgccatgagggaccagtacggcggcagcggcggcggcagcggcggcaagatgtggaagagccccaacggcaccatccagaacatcctgggcggcaccgtgttcagggagggcggcagcggcggcggcagcggcggcgtgcactacaccgacgtgagcaacatgagccacctggccaggcagaggctgctgggcaggagcggcggcagcggcggcggcagcggcggcatctgcctgaccagcaccgtgcagctgatcatgcagctgatgcccttcggctgcctgctggacggcggcagcggcggcggcagcggcggcaccctggaggacagcagcggcaacctgctggagaggaacagcttcgaggtgagggtgtgcgccggcggcagcggcggcggcagcggcggcgtgaagatctgcgacttcggcctggccaggtacatcatgagcgacagcaactacgtggtgaggggcggcagcggcggcggcagcggcggcgagtacctggacgacaggaacaccttcaggaggagcgtggtggtgccctacgagccccccgagggcggcagcggcggcggcagcggcggcatgaccgaccaggaggccatccaggacctgtgcctggccgtggaggaggtgagcctgaggaagggcggcagcggcggcggcagcggcggctacaagctggtggtggtgggcgccgacggcgtgggcaagagcgccctgaccatccagctgatcggcggcagcggcggcggcagcggcggctacaagctggtggtggtgggcgccgtgggcgtgggcaagagcgccctgaccatccagctgatcggcggcagcggcggcggcagcggcggccagagccagcacatgaccgaggtggtgaggcactgcccccaccacgagaggtgcagcgacagcggcggcagcggcggcggcagcggcggctgcaacagcagctgcatgggcggcatgaaccagaggcccatcctgaccatcatcaccctggagggcggcagcggcggcggcagcggcggctgcaacagcagctgcatgggcggcatgaactggaggcccatcctgaccatcatcaccctggagtgatgatgaagctcgctttcttgctgtccaatttctattaaaggttcctttgttccctaagtccaactactaaactgggggatattatgaagggccttgagcatctggattctgcctaataaaaaacatttattttcattgcaa

SEQ ID NO: 47 - protein, 645 aa, Artificial Sequence
  Multi-neoantigen construct
  MISC_FEATURE (1)..(24): signal sequence
MLVMAPRTVLLLLSAALALTETWAVHYTDVSNMSCLARQRLLGRSGGSGGGSGGDSTPPPGTRVLAMAIYKQSQHGGSGGGSGGKAISTRDPLSKITEQEKDFLWGGSGGGSGGDCTTIHYNYMYNSSCMGGMNRGGSGGGSGGKVKIPVAIKEATSPKANKEILGGSGGGSGGACPGRDRRTEKENLRKKGEPHGGSGGGSGGVDSTPPPGTRFRAMAIYKQSQGGSGGGSGGKAISTRDPLSKITEQEKDFLWGGSGGGSGGCLLDILDTAGKEEYSAMRDQYGGSGGGSGGKMWKSPNGTIQNILGGTVFREGGSGGGSGGVHYTDVSNMSHLARQRLLGRSGGSGGGSGGICLTSTVQLIMQLMPFGCLLDGGSGGGSGGTLEDSSGNLLERNSFEVRVCAGGSGGGSGGVKICDFGLARYIMSDSNYVVRGGSGGGSGGEYLDDRNTFRRSVVVPYEPPEGGSGGGSGGMTDQEAIQDLCLAVEEVSLRKGGSGGGSGGYKLVVVGADGVGKSALTIQLIGGSGGGSGGYKLVVVGAVGVGKSALTIQLIGGSGGGSGGQSQHMTEVVRHCPHHERCSDSGGSGGGSGGCNSSCMGGMNQRPILTIITLEGGSGGGSGGCNSSCMGGMNWRPILTIITLE

SEQ ID NO: 48 - protein, 268 aa, Artificial Sequence
  SARS CoV-2 mRNA vaccine
  MISC_FEATURE (1)..(24): signal sequence
  MISC_FEATURE (42)..(50): linker
  MISC_FEATURE (66)..(74): linker
  MISC_FEATURE (93)..(101): linker
  MISC_FEATURE (112)..(120): linker
  MISC_FEATURE (139)..(147): linker
  MISC_FEATURE (157)..(165): linker
  MISC_FEATURE (184)..(192): linker
  MISC_FEATURE (209)..(217): linker
  MISC_FEATURE (235)..(243): linker
  MISC_FEATURE (260)..(268): linker
MLVMAPRTVLLLLSAALALTETWAIRQGTDYKHWPQIAQFAGGSGGGSGGGHLRIAGHHLGRCDIGGSGGGSGGGMEVTPSGTWLTYTGAIKGGSGGGSGGTLACFVLAAVGGSGGGSGGAFFGMSRIGMEVTPSGTWGGSGGGSGGGLMWLSYFIGGSGGGSGGAQKFNGLTVLPPLLTDEMGGSGGGSGGMAYRFNGIGVTQNVLYGGSGGGSGGQALNTLVKQLSSNFGAIGGSGGGSGGGAALQIPFAMQMAYRFGGSGGGSGG

SEQ ID NO: 49 - protein, 27 aa, Artificial Sequence
  BRAF 27mer WT
DLTVKIGDFGLATVKSRWSGSHQFEQL

SEQ ID NO: 50 - protein, 27 aa, Artificial Sequence
  BRAF 27mer V600E
DLTVKIGDFGLATEKSRWSGSHQFEQL

SEQ ID NO: 51 - protein, 27 aa, Artificial Sequence
  KRAS 27mer G12V
MTEYKLVVVGAVGVGKSALTIQLIQNH

SEQ ID NO: 52 - protein, 27 aa, Artificial Sequence
  KRAS 27mer G13D
MTEYKLVVVGAGDVGKSALTIQLIQNH

SEQ ID NO: 53 - protein, 27 aa, Artificial Sequence
  TP53 27mer WT
IYKQSQHMTEVVRRCPHHERCSDSDGL

SEQ ID NO: 54 - protein, 27 aa, Artificial Sequence
  TP53 27mer R175H
IYKQSQHMTEVVRHCPHHERCSDSDGL

SEQ ID NO: 55 - protein, 27 aa, Artificial Sequence
  PIK3CA 27mer WT
KAISTRDPLSEITEQEKDFLWSHRHYC

SEQ ID NO: 56 - protein, 27 aa, Artificial Sequence
  PIK3CA 27mer E545K
KAISTRDPLSEITKQEKDFLWSHRHYC

SEQ ID NO: 57 - protein, 27 aa, Artificial Sequence
  TP53 27mer WT
SFEVRVCACPGRDRRTEEENLRKKGEP

SEQ ID NO: 58 - protein, 27 aa, Artificial Sequence
  TP53 27mer R282W
SFEVRVCACPGRDWRTEEENLRKKGEP

SEQ ID NO: 59 - protein, 27 aa, Artificial Sequence
  TP53 27mer WT
NYMCNSSCMGGMNRRPILTIITLEDSS

SEQ ID NO: 60 - protein, 27 aa, Artificial Sequence
  TP53 27mer R248Q
NYMCNSSCMGGMNQRPILTIITLEDSS

SEQ ID NO: 61 - protein, 27 aa, Artificial Sequence
  PIK3CA 27mer WT
EALEYFMKQMNDAHHGGWTTKMDWIFH

SEQ ID NO: 62 - protein, 27 aa, Artificial Sequence
  PIK3CA 27mer H1047R
EALEYFMKQMNDARHGGWTTKMDWIFH

SEQ ID NO: 63 - protein, 27 aa, Artificial Sequence
  TP53 27mer WT
SSGNLLGRNSFEVRVCACPGRDRRTEE

SEQ ID NO: 64 - protein, 27 aa, Artificial Sequence
  TP53 27mer R273H
SSGNLLGRNSFEVHVCACPGRDRRTEE

SEQ ID NO: 65 - protein, 27 aa, Artificial Sequence
  KRAS 27mer G12C
MTEYKLVVVGACGVGKSALTIQLIQNH

SEQ ID NO: 66 - protein, 27 aa, Artificial Sequence
  TP53 27mer R273C
SSGNLLGRNSFEVCVCACPGRDRRTEE

SEQ ID NO: 67 - protein, 27 aa, Artificial Sequence
  PIK3CA 27mer WT
EQLKAISTRDPLSEITEQEKDFLWSHR

SEQ ID NO: 68 - protein, 27 aa, Artificial Sequence
  PIK3CA 27mer E542K
EQLKAISTRDPLSKITEQEKDFLWSHR

SEQ ID NO: 69 - protein, 27 aa, Artificial Sequence
  TP53 27mer R248W
NYMCNSSCMGGMNWRPILTIITLEDSS

SEQ ID NO: 70 - protein, 27 aa, Artificial Sequence
  KRAS 27mer G12A
MTEYKLVVVGAAGVGKSALTIQLIQNH

SEQ ID NO: 71 - protein, 27 aa, Artificial Sequence
  FBXW7 27mer WT
ECIHTLYGHTSTVRCMHLHEKRVVSGS

SEQ ID NO: 72 - protein, 27 aa, Artificial Sequence
  FBXW7 27mer R465C
ECIHTLYGHTSTVCCMHLHEKRVVSGS

SEQ ID NO: 73 - protein, 27 aa, Artificial Sequence
  TP53 27mer WT
IHYNYMCNSSCMGGMNRRPILTIITLE

SEQ ID NO: 74 - protein, 27 aa, Artificial Sequence
  TP53 27mer G245S
IHYNYMCNSSCMGSMNRRPILTIITLE

SEQ ID NO: 75 - protein, 27 aa, Artificial Sequence
  SMAD4 27mer WT
VTVDGYVDPSGGDRFCLGQLSNVHRTE

SEQ ID NO: 76 - protein, 27 aa, Artificial Sequence
  SMAD4 27mer R361H
VTVDGYVDPSGGDHFCLGQLSNVHRTE

SEQ ID NO: 77 - protein, 27 aa, Artificial Sequence
  FBXW7 27mer R465H
ECIHTLYGHTSTVHCMHLHEKRVVSGS

SEQ ID NO: 78 - protein, 27 aa, Artificial Sequence
  KRAS 27mer G12S
MTEYKLVVVGASGVGKSALTIQLIQNH

SEQ ID NO: 79 - protein, 27 aa, Artificial Sequence
  PIK3CA 27mer WT
QEAEREEFFDETRRLCDLRLFQPFLKV

SEQ ID NO: 80 - protein, 27 aa, Artificial Sequence
  PIK3CA 27mer R88Q
QEAEREEFFDETRQLCDLRLFQPFLKV

SEQ ID NO: 81 - protein, 27 aa, Artificial Sequence
  FBXW7 27mer WT
QCLHVLMGHVAAVRCVQYDGRRVVSGA

SEQ ID NO: 82 - protein, 27 aa, Artificial Sequence
  FBXW7 27mer R505C
QCLHVLMGHVAAVCCVQYDGRRVVSGA

SEQ ID NO: 83 - protein, 27 aa, Artificial Sequence
  SMAD 27mer R361C
VTVDGYVDPSGGDCFCLGQLSNVHRTE

SEQ ID NO: 84 - protein, 29 aa, Artificial Sequence
  EGFR 27mer WT
EGEKVKIPVAIKELREATSPKANKEILDE

SEQ ID NO: 85 - protein, 26 aa, Artificial Sequence
  EGFR 27mer E746_A750del
EGEKVKIPVAIKEATSPKANKEILDE

SEQ ID NO: 86 - protein, 27 aa, Artificial Sequence
  EGFR 27mer WT
VKTPQHVKITDFGLAKLLGAEEKEYHA

SEQ ID NO: 87 - protein, 27 aa, Artificial Sequence
  EGFR 27mer L858Q
VKTPQHVKITDFGQAKLLGAEEKEYHA

SEQ ID NO: 88 - protein, 27 aa, Artificial Sequence
  TP53 27mer WT
LWVDSTPPPGTRVRAMAIYKQSQHMTE

SEQ ID NO: 89 - protein, 27 aa, Artificial Sequence
  TP53 27mer R158L
LWVDSTPPPGTRVLAMAIYKQSQHMTE

SEQ ID NO: 90 - protein, 27 aa, Artificial Sequence
  TP53 27mer WT
QLWVDSTPPPGTRVRAMAIYKQSQHMT

SEQ ID NO: 91 - protein, 27 aa, Artificial Sequence
  TP53 27mer V157F
QLWVDSTPPPGTRFRAMAIYKQSQHMT

SEQ ID NO: 92 - protein, 27 aa, Artificial Sequence
  TP53 27mer R273L
SSGNLLGRNSFEVLVCACPGRDRRTEE

SEQ ID NO: 93 - protein, 27 aa, Artificial Sequence
  EGFR 27mer WT
LLGICLTSTVQLITQLMPFGCLLDYVR

SEQ ID NO: 94 - protein, 27 aa, Artificial Sequence
  EGFR 27mer T790M
LLGICLTSTVQLIMQLMPFGCLLDYVR

SEQ ID NO: 95 - protein, 27 aa, Artificial Sequence
  KRAS 27mer G13C
MTEYKLVVVGAGCVGKSALTIQLIQNH

SEQ ID NO: 96 - protein, 27 aa, Artificial Sequence
  KRAS 27mer G12R
MTEYKLVVVGARGVGKSALTIQLIQNH

SEQ ID NO: 97 - protein, 27 aa, Artificial Sequence
  GNAS 27mer WT
ADYVPSDQDLLRCRVLTSGIFETKFQV

SEQ ID NO: 98 - protein, 27 aa, Artificial Sequence
  GNAS 27mer R201C
ADYVPSDQDLLRCCVLTSGIFETKFQV

SEQ ID NO: 99 - protein, 27 aa, Artificial Sequence
  TP53 27mer WT
YECGECGKSFSKYASFSNHQRVHTEKK

SEQ ID NO: 100 - protein, 27 aa, Artificial Sequence
  TP53 27mer R282W
YECGECGKSFSKYVSFSNHQRVHTEKK

SEQ ID NO: 101 - protein, 27 aa, Artificial Sequence
  MYD88 27mer WT
FALSLSPGAHQKRLIPIKYKAMKKEFP

SEQ ID NO: 102 - protein, 27 aa, Artificial Sequence
  MYD88 27mer L265P
FALSLSPGAHQKRPIPIKYKAMKKEFP

SEQ ID NO: 103 - protein, 27 aa, Artificial Sequence
  EZH2 27mer WT
IKDPVQKNEFISEYCGEIISQDEADRR

SEQ ID NO: 104 - protein, 27 aa, Artificial Sequence
  EZH2 27mer Y641N
IKDPVQKNEFISENCGEIISQDEADRR

SEQ ID NO: 105 - protein, 27 aa, Artificial Sequence
  EZH2 27mer Y641F
IKDPVQKNEFISEFCGEIISQDEADRR

SEQ ID NO: 106 - protein, 27 aa, Artificial Sequence
  MYD88 27mer WT
RRMVVVVSDDYLQSKECDFQTKFALSL

SEQ ID NO: 107 - protein, 27 aa, Artificial Sequence
  MYD88 27mer S243N
RRMVVVVSDDYLQNKECDFQTKFALSL

SEQ ID NO: 108 - protein, 27 aa, Artificial Sequence
  FLT3 27mer WT
GKVVKICDFGLARDIMSDSNYVVRGNA

SEQ ID NO: 109 - protein, 27 aa, Artificial Sequence
  FLT3 27mer D835Y
GKVVKICDFGLARYIMSDSNYVVRGNA

SEQ ID NO: 110 - protein, 27 aa, Artificial Sequence
  FLT3 27mer D835E
GKVVKICDFGLAREIMSDSNYVVRGNA

SEQ ID NO: 111 - protein, 27 aa, Artificial Sequence
  FLT3 27mer D835H
GKVVKICDFGLARHIMSDSNYVVRGNA

SEQ ID NO: 112 - protein, 27 aa, Artificial Sequence
  NPM1 27mer WT
FINYVKNCFRMTDQEAIQDLWQWRKSL

SEQ ID NO: 113 - protein, 31 aa, Artificial Sequence
  NPM1 27mer W288Cfs*12
FINYVKNCFRMTDQEAIQDLCLAVEEVSLRK

SEQ ID NO: 114 - protein, 27 aa, Artificial Sequence
  DNMT3A 27mer WT
GFPVHYTDVSNMSRLARQRLLGRSWSV

SEQ ID NO: 115 - protein, 27 aa, Artificial Sequence
  DNMT3A 27mer R882H
GFPVHYTDVSNMSHLARQRLLGRSWSV

SEQ ID NO: 116 - protein, 27 aa, Artificial Sequence
  DNMT3A 27mer R882C
GFPVHYTDVSNMSCLARQRLLGRSWSV

SEQ ID NO: 117 - protein, 27 aa, Artificial Sequence
  IDH2 27mer WT
KLKKMWKSPNGTIRNILGGTVFREPII

SEQ ID NO: 118 - protein, 27 aa, Artificial Sequence
  IDH2 27mer R140Q
KLKKMWKSPNGTIQNILGGTVFREPII

SEQ ID NO: 119 - protein, 27 aa, Artificial Sequence
  IDH1 27mer WT
RLVSGWVKPIIIGRHAYGDQYRATDFV

SEQ ID NO: 120 - protein, 27 aa, Artificial Sequence
  IDH1 27mer R132C
RLVSGWVKPIIIGCHAYGDQYRATDFV

SEQ ID NO: 121 - protein, 27 aa, Artificial Sequence
  NRAS 27mer WT
GETCLLDILDTAGQEEYSAMRDQYMRT

SEQ ID NO: 122 - protein, 27 aa, Artificial Sequence
  NRAS 27mer Q61K
GETCLLDILDTAGKEEYSAMRDQYMRT

SEQ ID NO: 123 - protein, 27 aa, Artificial Sequence
  KIT 27mer WT
GRITKICDFGLARDIKNDSNYVVKGNA

SEQ ID NO: 124 - protein, 27 aa, Artificial Sequence
  KIT 27mer D816Y
GRITKICDFGLARYIKNDSNYVVKGNA

SEQ ID NO: 125 - RNA, 465 nt, Artificial Sequence
  IL7 RNA translated region
auguuccauguuucuuuuagauauaucuuuggaauuccuccacugauccuuguucugcugccugucacaucaucugagugccacauuaaagacaaagaagguaaagcauaugagaguguacugaugaucagcaucgaugaauuggacaaaaugacaggaacugauaguaauugcccgaauaaugaaccaaacuuuuuuagaaaacauguaugugaugauacaaaggaagcugcuuuucuaaaucgugcugcucgcaaguugaagcaauuucuuaaaaugaauaucagugaagaauucaauguccacuuacuaacaguaucacaaggcacacaaacacuggugaacugcacaaguaaggaagaaaaaaacguaaaggaacagaaaaagaaugaugcauguuuccuaaagagacuacugagagaaauaaaaacuuguuggaauaaaauuuugaagggcaguauauga

SEQ ID NO: 126 - RNA, 1380 nt, Artificial Sequence
  IL7R RNA translated region
augacaauucuagguacaacuuuuggcaugguuuuuucuuuacuucaagucguuucuggagaaaguggcuaugcucaaaauggagacuuggaagaugcagaacuggaugacuacucauucucaugcuauagccaguuggaagugaauggaucgcagcacucacugaccugugcuuuugaggacccagaugucaacaucaccaaucuggaauuugaaauauguggggcccucguggagguaaagugccugaauuucaggaaacuacaagagauauauuucaucgagacaaagaaauucuuacugauuggaaagagcaauauaugugugaagguuggagaaaagagucuaaccugcaaaaaaauagaccuaaccacuauaguuaaaccugaggcuccuuuugaccugagugucgucuaucgggaaggagccaaugacuuuguggugacauuuaauacaucacacuugcaaaagaaguauguaaaaguuuuaaugcacgauguagcuuaccgccaggaaaaggaugaaaacaaauggacgcaugugaauuuauccagcacaaagcugacacuccugcagagaaagcuccaaccggcagcaauguaugagauuaaaguucgauccaucccugaucacuauuuuaaaggcuucuggagugaauggaguccaaguuauuacuucagaacuccagagaucaauaauagcucaggggagauggauccuaucuuacuaaccaucagcauuuugaguuuuuucucugucgcucuguuggucaucuuggccuguguguuauggaaaaaaaggauuaagccuaucguauggcccagucuccccgaucauaagaagacucuggaacaucuuuguaagaaaccaagaaaaaauuuaaaugugaguuucaauccugaaaguuuccuggacugccagauucauaggguggaugacauucaagcuagagaugaaguggaagguuuucugcaagauacguuuccucagcaacuagaagaaucugagaagcagaggcuuggaggggaugugcagagccccaacugcccaucugaggauguagucaucacuccagaaagcuuuggaagagauucaucccucacaugccuggcugggaaugucagugcaugugacgccccuauucucuccucuuccaggucccuagacugcagggagaguggcaagaaugggccucauguguaccaggaccuccugcuuagccuugggacuacaaacagcacgcugcccccuccauuuucucuccaaucuggaauccugacauugaacccaguugcucagggucagcccauucuuacuucccugggaucaaaucaagaagaagcauaugucaccauguccagcuucuaccaaaaccaguga

SEQ ID NO: 127 - RNA, 411 nt, Artificial Sequence
  IL15 RNA translated region
auggaaaccgacacacugcugcugugggugcugcuucuuugggugcccggcucuacaggcaacugggucaacgugaucagcgaccugaagaagaucgaggaccugauccagagcaugcacaucgacgccacacuguacaccgagagcgacgugcacccuagcuguaaagugaccgccaugaagugcuuucugcuggaacugcaagugaucagccuggaaagcggcgacgccagcauccacgacaccguggaaaaccugaucauccuggccaacgacagccugagcagcaacggcaaugugaccgaguccggcugcaaagagugcgaggaacuggaagagaagaauaucaaagaguuccugcagagcuucgugcacaucgugcagauguucaucaacaccagcugaugauga

SEQ ID NO: 128 - RNA, 957 nt, Artificial Sequence
  IL15R-Fc RNA translated region
auggauagacugaccagcagcuuccugcugcugaucgugccugccuacgugcugagcaucaccuguccuccaccuaugagcguggaacacgccgacaucugggucaagagcuacagccuguacagcagagagcgguacaucugcaacagcggcuucaagagaaaggccggcaccagcagccugaccgagugugugcugaacaaggccaccaauguggcccacuggaccacaccuagccugaagugcaucagagagcccaagagcugcgacaagacccacaccuguccaccuuguccugcuccagaacugcucggcggaccuuccguguuccuguuuccuccaaagccuaaggacacccugaugaucagcagaaccccugaagugaccugcguggugguggaugugucucacgaggaccccgaagugaaguucaauugguacguggacggcguggaagugcacaacgccaagaccaagccuagagaggaacaguacaacagcaccuacagagugguguccgugcugaccgugcugcaccaggauuggcugaacggcaaagaguacaagugcaagguguccaacaaggcccugccugcuccuaucgagaaaaccaucagcaaggccaagggccagccuagggaaccccagguuuacacacugccuccaagcagggacgagcugaccaagaaucaggugucccugaccugccuggucaagggcuucuacccuuccgauaucgccguggaaugggagagcaauggccagccugagaacaacuacaagacaaccccuccugugcuggacagcgacggcucauucuuccuguacuccaagcugacaguggacaagagcagauggcagcagggcaacguguucagcugcagcgugaugcacgaggcccugcacaaccacuacacccagaagucccugagccugucuccuggcaagugaugauga

SEQ ID NO: 129 - RNA, 705 nt, Artificial Sequence
  FAS-4-IBB RNA translated region
augcucggaaucuggacacugcugccucuggugcugacaagcguggccagacugagcagcaagagcgugaacgcccaagugaccgacaucaacagcaaaggccuggaacugagaaagaccgugaccaccguggaaacccagaaccuggaaggccugcaccacgacggccaguucugucacaaaccuuguccaccuggcgagcggaaggccagagauugcacagugaauggcgacgagccugacugcgugcccugucaagagggcaaagaguacaccgacaaggcccacuucagcagcaagugcagacggugcagacugugcgacgaaggccacggacuggaaguggaaaucaacugcacccggacacagaacaccaagugccggugcaagcccaacuucuucugcaacagcaccgugugcgagcacugcgacccuuguaccaagugcgaacacggcaucaucaaagagugcacccugaccuccaacacgaagugcaaagaggaaggcagcagaagcaaccucggcuggcugugucugcugcugcuccccauuccucugaucgugugggucaagcggggcagaaagaagcugcuguacaucuucaagcagcccuucaugcggcccgugcagaccacacaagaggaagauggcugcuccugcagauuccccgaggaagaagaaggcggcugcgagcuguaaugauga

SEQ ID NO: 130 - protein, 17 aa, Artificial Sequence
  SARS-CoV-2 antigen peptide NCAP
IRQGTDYKHWPQIAQFA

SEQ ID NO: 131 - protein, 18 aa, Artificial Sequence
  SARS-CoV-2 antigen peptide NCAP
AFFGMSRIGMEVTPSGTW

SEQ ID NO: 132 - protein, 18 aa, Artificial Sequence
  SARS-CoV-2 antigen peptide NCAP
GMEVTPSGTWLTYTGAIK

SEQ ID NO: 133 - protein, 15 aa, Artificial Sequence
  SARS-CoV-2 Antigen Peptide VME1
GHLRIAGHHLGRCDI

SEQ ID NO: 134 - protein, 10 aa, Artificial Sequence
  SARS-CoV-2 Antigen Peptide VME1
TLACFVLAAV

SEQ ID NO: 135 - protein, 9 aa, Artificial Sequence
  SARS-CoV-2 Antigen Peptide VME1
GLMWLSYFI

SEQ ID NO: 136 - protein, 18 aa, Artificial Sequence
  SARS-CoV-2 Antigen Peptide SPIKE
AQKFNGLTVLPPLLTDEM

SEQ ID NO: 137 - protein, 16 aa, Artificial Sequence
  SARS-CoV-2 Antigen Peptide SPIKE
MAYRFNGIGVTQNVLY

SEQ ID NO: 138 - protein, 17 aa, Artificial Sequence
  SARS-CoV-2 Antigen Peptide SPIKE
QALNTLVKQLSSNFGAI

SEQ ID NO: 139 - protein, 16 aa, Artificial Sequence
  SARS-CoV-2 Antigen Peptide SPIKE
GAALQIPFAMQMAYRF